(19) United States
(12) Patent Application Publication
(10) Pub. No.: US 2025/0223584 A2
(43) Pub. Date: Jul. 10, 2025

(54) COMPOSITIONS AND METHODS FOR EFFICIENT IN VIVO DELIVERY

(71) Applicants: David R. Liu, Cambridge, MA (US); Thomas J. Cahill, III, Cambridge, MA (US); Philip DeSouza, Cambridge, MA (US); Aditya Raguram, Cambridge, MA (US); Samagya Banskota, Cambridge, MA (US); Meirui An, Cambridge, MA (US)

(72) Inventors: David R. Liu, Cambridge, MA (US); Thomas J. Cahill, III, Cambridge, MA (US); Philip DeSouza, Cambridge, MA (US); Aditya Raguram, Cambridge, MA (US); Samagya Banskota, Cambridge, MA (US); Meirui An, Cambridge, MA (US)

(73) Assignees: The Broad Institute, Inc., Cambridge, MA (US); President and Fellows of Harvard College, Cambridge, MA (US)

(21) Appl. No.: 18/715,578

(22) PCT Filed: Dec. 2, 2022
(86) PCT No.: PCT/US2022/080856

§ 371 Date: May 31, 2024

Related U.S. Application Data
(60) Provisional application No. 63/423,372, filed on Nov. 7, 2022.
(60) Provisional application No. 63/298,626, filed on Jan. 11, 2022.
(60) Provisional application No. 63/298,621, filed on Jan. 11, 2022.
(60) Provisional application No. 63/298,611, filed on Jan. 11, 2022.
(60) Provisional application No. 63/285,995, filed on Dec. 3, 2021.

(51) Int. Cl. C12N 15/10; C07K 14/005; C12N 9/22; C12N 15/11; C12N 15/86

(52) U.S. Cl. CPC C12N 15/102; C07K 14/005; C12N 9/22; C12N 15/111; C12N 15/86; C07K 2319/033; C07K 2319/09; C07K 2319/095; C07K 2319/50; C12N 2310/20; C12N 2740/13022; C12N 2740/13023; C12N 2740/13045

(57) ABSTRACT

Disclosed herein are compositions, methods, kits, and systems relating to efficient delivery of cargos (e.g., therapeutic cargos) into cells, for instance, for in vivo delivery. The present disclosure provides lipid-containing particles (e.g., virus-like particles) for delivering therapeutic cargos. The present disclosure also provides polynucleotides encoding the lipid-containing particles provided herein, which may be useful for producing said lipid-containing particles. Also provided are methods for editing nucleic acid molecules in cells using the lipid-containing particles provided herein, as well as cells and kits comprising the lipid-containing particles.

### RELATED APPLICATIONS

This application is a national stage filing under 35 U.S.C. § 371 of International PCT Application PCT/US2022/080856, filed Dec. 2, 2022, which claims priority under 35 U.S.C. § 119(e) to U.S. Provisional Application, U.S. Ser. No. 63/285,995, filed Dec. 3, 2021, U.S. Provisional Application, U.S. Ser. No. 63/298,621, filed Jan. 11, 2022, U.S. Provisional Application, U.S. Ser. No. 63/298,611, filed Jan. 11, 2022, U.S. Provisional Application, U.S. Ser. No. 63/298,626, filed Jan. 11, 2022, and U.S. Provisional Application, U.S. Ser. No. 63/423,372, filed Nov. 7, 2022, each of which is incorporated herein by reference.

### FEDERALLY SPONSORED RESEARCH

This invention was made with government support under Grant Nos. UG3AI150551, U01AI142756, R35GM118062, RM1HG009490, R01EY009339, and T32GM095450 awarded by the National Institutes of Health. The government has certain rights in the invention.

### REFERENCE TO AN ELECTRONIC SEQUENCE LISTING

The contents of the electronic sequence listing (B119570157US01-SEQ-TNG.xml; Size: 248,262 bytes; and Date of Creation: May 29, 2024) is herein incorporated by reference in its entirety.

### BACKGROUND

Retroviruses can be an attractive scaffold for viral-like particles (VLPs). Retroviral capsids generally lack the rigid symmetry requirements of many non-enveloped icosahedral viruses (Zhang et al., 2015), suggesting increased structural flexibility to incorporate non-native protein cargos. Additionally, retrovirus tropisms can be modulated by pseudotyping virions with different envelope glycoproteins, which could enable targeting of VLPs to specific cell types (Cronin et al., 2005). Previous work has demonstrated that fusing a desired protein cargo to the C-terminus of retroviral gag polyproteins is sufficient to direct packaging of that cargo protein within retroviral particles (Kaczmarczyk et al., 2011; Voelkel et al., 2010). More recently, similar strategies have been applied to package Cas9 RNPs within retroviral particles (Mangeot et al., 2019). However, VLPs that have therapeutic levels of in vivo delivery efficiency remain needed.

### SUMMARY

In one aspect, the disclosure provides a lipid-containing particle that comprises (a) a human endogenous retroviral (HERV) envelope protein, a humanized viral envelope protein, or a non-immunogenic cell fusion molecule; (b) a fusion protein comprising a plasma membrane localization protein coupled to a nuclear export sequence (NES); and (c) a therapeutic cargo.

In another aspect, the disclosure provides a composition comprising (a) a first nucleic acid molecule encoding a human endogenous retroviral (HERV) envelope protein, a humanized viral envelope protein, or a non-immunogenic cell fusion molecule; and (b) a second nucleic acid molecule encoding a fusion protein comprising a plasma membrane localization protein coupled to a nuclear export sequence (NES) and cargo, wherein the cargo comprises a therapeutic cargo or a binding partner for a therapeutic cargo. In another aspect, the present disclosure provides a nucleic acid molecule encoding a fusion protein comprising a plasma membrane localization protein coupled to a nuclear export sequence (NES) and cargo, wherein the cargo comprises a therapeutic cargo or a binding partner for a therapeutic cargo

In yet another aspect, the specification describes a lipid-containing particle comprising (a) a human endogenous retroviral (HERV) envelope protein, (b) a humanized viral envelope protein or a non-immunogenic cell fusion molecule; (c) a fusion protein comprising a plasma membrane localization protein coupled to a cleavable linker; and (d) a therapeutic cargo. In some embodiments, the fusion protein comprises an NES.

In another aspect, the disclosure provides a method of producing a lipid-containing particle described herein comprising the steps of (a) providing a system expressing (i) the human endogenous retroviral (HERV) envelope protein, a humanized viral envelope protein, or a non-immunogenic cell fusion molecule; (ii) the fusion protein comprising a plasma membrane localization protein coupled to a nuclear export sequence (NES); and (iii) the cargo, wherein the system generates the lipid-containing particle; and optionally (b) harvesting and purifying the lipid-containing particle.

In a further aspect, the disclosure describes a method of producing a lipid-containing particle described herein comprising (a) providing a system expressing (i) the human endogenous retroviral (HERV) envelope protein, a humanized viral envelope protein, or a non-immunogenic cell fusion molecule; (ii) the fusion protein comprising a plasma membrane localization protein coupled to a cleavable linker; and (iii) the cargo, wherein the system generates the lipid-containing particle; and optionally (b) harvesting and purifying the lipid-containing particle. In some embodiments, the fusion protein comprises an NES.

In another aspect, the disclosure provides a method of producing a lipid-containing particle described herein comprising (a) providing a system expressing the fusion protein comprising (i) the humanized retroviral structural protein; a human endogenous retroviral (HERV) structural protein, optionally HERV gag; the pleckstrin homology (PH) domain; or the non-immunogenic plasma membrane recruitment protein; and (ii) the nuclear export sequence (NES), wherein the system generates the lipid-containing particle; and optionally (b) harvesting and purifying the lipid-containing particle.

In still another aspect, the disclosure provides a method of producing a lipid-containing particle described herein, comprising (a) providing a system expressing the fusion protein comprising (i) the humanized retroviral structural protein; the human endogenous retroviral (HERV) structural protein, optionally HERV gag; a pleckstrin homology (PH) domain; or a non-immunogenic plasma membrane recruitment protein, (ii) the cleavable linker, and (iii) the cargo; and wherein the system generates the lipid-containing particle; and optionally (b) harvesting and purifying the lipid-containing particle. In some embodiments, the fusion protein further comprises an NES.

In various embodiments, the plasma membrane localization protein comprises a human endogenous retroviral (HERV) structural protein, optionally HERV gag; a humanized viral structural protein; a pleckstrin homology (PH) domain; or a non-immunogenic plasma membrane recruitment protein.

The therapeutic cargo, in various embodiments, can comprise a nuclease, a base editor, a prime editor, an epigenetic editor, a restriction endonuclease (optionally a Type IIS restriction enzyme), a recombinase, a transcription factor, an antibody, a chimeric antigen receptor, a T cell receptor, an organelle, a nucleic acid molecule, a DNA, a RNA, a retrotransposon, a reverse transcriptase, an oligonucleotide, an aptazyme, an aptamer, a ribozyme, a small molecule compound, or any combination thereof. In some embodiments, the therapeutic cargo does not comprise a nuclease, a reverse transcriptase, a base editor, or a prime editor. In some embodiments, the therapeutic cargo is a protein (e.g., a nuclease, a base editor, a prime editor, an epigenetic editor, a restriction endonuclease (optionally a Type IIS restriction enzyme), a recombinase, a transcription factor, an antibody, a chimeric antigen receptor, a T cell receptor, or a reverse transcriptase). In some embodiments, the therapeutic cargo is a nucleic acid molecule (e.g., DNA, RNA, a retrotransposon, an aptazyme, an aptamer, or a ribozyme). In some embodiments, the therapeutic cargo is a an epigenetic editor, a restriction endonuclease (optionally a Type IIS restriction enzyme), a recombinase, a transcription factor, an antibody, a chimeric antigen receptor, a T cell receptor, an organelle, a nucleic acid molecule, a DNA, a RNA, a retrotransposon, an oligonucleotide, an aptazyme, an aptamer, a ribozyme, a small molecule compound, or any combination thereof.

In various embodiments, the fusion protein can comprise the plasma membrane localization protein, the NES, and the therapeutic cargo arranged in order from N-terminus to C-terminus. The fusion protein can further comprise a cleavable linker, optionally wherein the cleavable linker is positioned between the plasma membrane localization protein and the therapeutic cargo, optionally, wherein the cleavable linker is positioned between the NES and the therapeutic cargo, optionally wherein the fusion protein further comprises a nuclear localization sequence (NLS) C-terminal of the cleavable linker. In some embodiments, the NLS is positioned within the fusion protein such that it is still attached to the therapeutic cargo after cleavage of the cleavable linker, allowing delivery of the therapeutic cargo to the nucleus of a cell.

In various embodiments, the plasma membrane localization protein can be a human endogenous retroviral (HERV) structural protein, optionally HERV gag; a humanized viral structural protein; a pleckstrin homology (PH) domain; or a non-immunogenic plasma membrane recruitment protein.

In various embodiments, the therapeutic cargo is a nuclease, a base editor, a prime editor, an epigenetic editor, a restriction endonuclease (optionally a Type IIS restriction enzyme), a recombinase, a transcription factor, an antibody, a chimeric antigen receptor, a T cell receptor, an organelle, a nucleic acid molecule, a DNA, a RNA, a retrotransposon, a reverse transcriptase, an oligonucleotide, an aptazyme, an aptamer, a ribozyme, or a small molecule compound, or any combination thereof.

In another aspect, the present disclosure provides lipid-containing particles comprising a lipid membrane encapsulating a protein core, wherein the protein core comprises a group-specific antigen (gag) protease (pro) polyprotein and a cleavage product, wherein the lipid-containing particle further comprises a therapeutic cargo, wherein the therapeutic cargo is present inside of the protein core, and wherein the cleavage product comprises (i) a sequence of a gag nucleocapsid protein and (ii) a nuclear export sequence (NES), and lacks the therapeutic cargo. In some embodiments, the therapeutic cargo is fused to a nuclear localization sequence (NLS).

In another aspect, the present disclosure provides lipid-containing particles comprising a lipid membrane encapsulating a protein core, wherein the protein core comprises a group-specific antigen (gag) protease (pro) polyprotein and a fusion protein, wherein the fusion protein comprises a sequence of a gag nucleocapsid protein, a therapeutic cargo, a cleavable linker, and a nuclear export sequence (NES), and wherein the cleavable linker is located between the therapeutic cargo and the NES.

In another aspect, the present disclosure provides a population of lipid-containing particles, wherein the population comprises lipid-containing particles comprising a lipid membrane encapsulating a protein core, wherein the protein core comprises a group-specific antigen (gag) protease (pro) polyprotein and a fusion protein, wherein the population comprises lipid-containing particles comprising a therapeutic cargo, wherein the therapeutic cargo is present inside the protein core, wherein a ratio of an amount of the therapeutic cargo present within the inside of the protein core versus an amount of the fusion protein is at least 1.5 amongst the population of lipid-containing particles, wherein the fusion protein comprises a first polypeptide and a second polypeptide, and wherein the first polypeptide comprises a sequence of a gag nucleocapsid protein, and the second polypeptide comprises a sequence of the therapeutic cargo.

In another aspect, the present disclosure provides compositions comprising (i) a first polynucleotide comprising a nucleic acid sequence encoding a group-specific antigen (gag) protease (pro) polyprotein; (ii) a second polynucleotide comprising a nucleic acid sequence encoding a fusion protein, wherein the fusion protein comprises: a sequence of a gag nucleocapsid protein, a therapeutic cargo, a cleavable linker, and a nuclear export sequence (NES), and wherein the cleavable linker is located between the therapeutic cargo and the NES. In some embodiments, a composition is a pharmaceutical composition.

In another aspect, the present disclosure provides fusion proteins comprising a sequence of a gag nucleocapsid protein, a therapeutic cargo, a cleavable linker, and a nuclear export sequence (NES); wherein the cleavable linker is located between the therapeutic cargo and the NES.

In another aspect, the present disclosure provides methods of using the lipid-containing particles provided herein, for example, in a method of nucleic acid editing.

In another aspect, the present disclosure provides cells for producing the lipid-containing particles provided herein. In some embodiments, the cells comprise any of the nucleic acids encoding components of the lipid-containing particles provided herein.

In another aspect, the present disclosure provides kits comprising any of the lipid-containing nanoparticles, nucleic acid sequences, fusion proteins, and compositions provided herein.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a schematic base editing viral-like particles (BE-VLP). As shown in the figure, base editor protein is fused to the C-terminus of murine leukemia virus (MLV) gag polyprotein via a linker that is cleaved by the MLV protease upon particle maturation.

FIG. 1B shows two graphs summarizing the base editing efficiencies of version 1 (v1) BE-VLPs. Adenine base editing efficiencies of v1 BE-VLPs at two genomic loci (termed as “HEK2” and “HEK3,” respectively) in HEK293T cells. The protospacer positions of the target adenines are denoted by subscripts (i.e., A5=adenine at position 5), where the PAM is positions 21-23. Data are shown as individual data points and mean±s.e.m for n=3 independent biological replicates.

FIG. 2A shows schematics of v1 engineered VLPs (eVLPs) and v2 eVLPs. More efficient linker cleavage in v2 BE-VLPs can lead to improved cargo release after VLP maturation.

FIG. 2B shows a graph summarizing the adenine base editing efficiencies of v1 and v2 BE-eVLPs at position A7 of the BCL11A enhancer site in HEK293T cells.

FIG. 2C shows a schematic demonstrating improved localization of cargo, as enabled in v3 eVLPs in producer cells leads to more efficient incorporation into eVLPs.

FIG. 2D shows a schematic demonstrating that installing a 3×NES motif upstream of the cleavable linker can encourage cytoplasmic localization of gag-3×NES-cargo in producer cells but nuclear localization of free adenine base editor (ABE) cargo in transduced cells.

FIG. 2E shows a graph summarizing adenine base editing efficiencies of v2.4 and v3 BE-eVLPs at position A7 of the BCL11A enhancer site in HEK293T cells.

FIG. 2F shows a schematic demonstrating that the optimal gag-cargo:gag-pro-pol stoichiometry can balance the amount of cargo protein per particle with the amount of MMLV protease required for efficient particle maturation.

FIG. 2G shows a graph summarizing adenine base editing efficiencies of v3.4 eVLPs with different gag-ABE:gag-pro-pol stoichiometries at position A7 of the BCL11A enhancer site in HEK293T cells. Legend denotes % gag-ABE plasmid of the total amount of gag-ABE and gag-pro-pol plasmids. As shown in FIGS. 2B, 2E, and 2G, values and error bars reflect mean±s.e.m. of n=3 independent biological replicates. Data were fit to four-parameter logistic curves using nonlinear regression.

FIG. 3A is a graph quantifying amount of BE molecules per eVLP by anti-Cas9 and anti-MLV (p30) ELISA. Values and error bars reflect mean±s.e.m. of n=3 independent replicates.

FIG. 3B is a graph quantifying relative sgRNA abundance by RT-qPCR using sgRNA-specific primers, normalized relative to v1 sgRNA abundance. Values and error bars reflect mean±s.e.m. of n=3 technical replicates.

FIGS. 3C-3D shows graphs comparing editing efficiencies with v1, v2.4, v3.4, and v4 BE-eVLPs at the BCL11A enhancer site in HEK293T cells (FIG. 3C) and at the Dnmt1 site in NIH 3T3 cells (FIG. 3D). Values and error bars reflect mean±s.e.m. of n=3 independent biological replicates. Data were fit to four-parameter logistic curves using nonlinear regression.

FIG. 3E is a graph summarizing adenine base editing efficiencies in HEK293T cells of either single v4 BE-eVLPs targeting the HEK2 or BCL11A enhancer loci separately, or multiplex v4 BE-eVLPs targeting both loci simultaneously.

FIG. 3F is a graph summarizing adenine base editing efficiencies of FuG-B2-pseudotyped v4 BE-eVLPs in Neuro-2a cells or 3T3 fibroblasts.

FIG. 3G shows graphs summarizing adenine base editing efficiencies at three on-target genomic loci and their corresponding Cas-dependent off-target sites in HEK293T cells treated with v4 BE-eVLPs or ABE8e plasmid. OT1=off-target site 1, OT2=off-target site 2, OT3=off-target site 3.

FIG. 3H is a graph summarizing Cas-independent off-target editing frequencies at six off-target R-loops in HEK293T cells treated with v4 BE-eVLPs or ABE8e plasmid. OTRL=off-target R-loop.

FIG. 3I is a graph quantifying the amount of molecules of BE-encoding DNA per v4 BE-eVLP detected by qPCR of lysed eVLPs or lysis buffer only.

FIG. 3J is a graph quantifying the amount of BE-encoding DNA detected by qPCR of lysate from HEK293T cells that were either treated with v4 BE-eVLPs or transfected with BE-encoding plasmids. As shown in FIGS. 3E-3J, data are shown as individual data points and mean±s.e.m for n=3 independent biological replicates.

FIG. 4A is a graph summarizing the correction efficiencies of the COL7A1 (R185X) mutation in patient-derived primary human fibroblasts.

FIG. 4B is a graph summarizing the correction efficiencies of the Idua (W392X) mutation in primary mouse fibroblasts. As shown in FIGS. 4A-4B, values and error bars reflect mean±s.e.m. of n=3 independent biological replicates. Data were fit to four-parameter logistic curves using nonlinear regression.

FIG. 4C is a graph summarizing adenine base editing efficiencies at the B2M and CIITA loci in primary human T cells. Data are shown as individual data points and mean±s.e.m for n=3 independent biological replicates.

FIG. 5A is a schematic of P0 ICV injections of v4 BE-eVLPs. Dnmt1-targeting v4 BE-eVLPs were co-injected with a lentivirus encoding EGFP-KASH. Tissue was harvested 3 weeks post-injection, and cortex and mid-brain were separated. Nuclei were dissociated for each tissue and analyzed by high-throughput sequencing as bulk unsorted (all nuclei) or GFP+ nuclei.

FIG. 5B is a graph summarizing adenine base editing efficiencies at the Dnmt1 locus in bulk unsorted (all nuclei) and GFP+ populations. Data are shown as individual data points and mean±s.e.m for n=4 mice.

FIG. 6A is a schematic of systemic injections of BE-eVLPs. Pcsk9-targeting BE-eVLPs were injected retro-orbitally into 6- to 7-week-old C57BL/6J mice. Organs were harvested one week after injection, and the genomic DNA of unsorted cells was sequenced.

FIG. 6B is a graph summarizing adenine base editing efficiencies at the Pcsk9 exon 1 splice donor in the mouse liver after systemic injection of v1 BE-VLPs or v4 BE-eVLPs. Data are shown as individual data points and mean±s.e.m for n=3 mice (v1 BE-VLP and v4 BE-eVLP at 4×10¹¹ VLPs) or n=4 mice (v4 BE-eVLP at 7×10¹¹ eVLPs).

FIG. 6C is a graph summarizing adenine base editing efficiencies at the Pcsk9 exon 1 splice donor in the mouse heart, kidney, liver, lungs, muscle, and spleen after systemic injection of 7×10¹¹ v4 BE-eVLPs. Data are shown as individual data points and mean±s.e.m for n=4 mice (treated) or n=3 mice (untreated).

FIG. 6D is a graph summarizing quantification of DNA sequencing reads containing A·T-to-G·C mutations within protospacer positions 4-10 for the fourteen CIRCLE-seq-nominated off-target loci from the livers of v4 BE-eVLP-treated, AAV-treated, and untreated mice. Data are shown as individual data points and mean±s.e.m for n=4 mice (BE-eVLP), n=5 mice (AAV), or n=3 mice (untreated). vg=viral genomes.

FIG. 6E is a graph summarizing quantification of serum Pcsk9 levels as measured by ELISA. Data are shown as individual data points and mean±s.e.m for n=4 mice (treated) or n=3 mice (untreated).

FIG. 7A is a schematic of Rpe65 exon 3 surrounding the R44X mutation (labeled in the schematic and shown in gray), which can be corrected by an A·T-to-G·C conversion at position A6 in the protospacer (labeled in the schematic and underlined; PAM sequence (AGT) in italics). Sequences shown (top-bottom) correspond to SEQ ID NOs: 5-6.

FIG. 7B is a schematic of subretinal injections. Five weeks post-injection, phenotypic rescue was assessed via ERG, and tissues were subsequently harvested for sequencing.

FIG. 7C is a graph summarizing adenine base editing efficiencies at positions A3, A6, and A8 of the protospacer in genomic DNA harvested from rd12 mice. Data are shown as individual data points and mean±s.e.m for n=6 mice (both treated groups) or n=4 mice (untreated).

FIG. 7D is a graph summarizing allele frequency distributions of genomic DNA harvested from treated rd12 mice. Data are shown as mean±s.e.m for n=6 mice. 8e-LV=ABE8e-NG-LV, 8e-eVLP=v4 ABE8e-NG-eVLP.

FIG. 7E is a graph summarizing scotopic a-wave and b-wave amplitudes measured by ERG following overnight dark adaptation. Data are shown as individual data points and mean±s.e.m for n=8 mice (wild-type), n=6 mice (ABE8e-NG-LV and v4 ABE8e-NG-eVLP), or n=4 mice (untreated).

FIG. 7F is a graph summarizing adenine base editing efficiencies at positions A3, A6, and A8 of the protospacer in genomic DNA harvested from rd12 mice. Data are shown as individual data points and mean±s.e.m for n=6 mice (v4 ABE7.10-NG-eVLP) or n=4 mice (ABE7.10-NG-LV and untreated). P values were calculated using a two-sided t-test.

FIG. 7G is a graph summarizing allele frequency distributions of genomic DNA harvested from treated rd12 mice. Data are shown as mean±s.e.m for n=6 mice (v4 ABE7.10-NG-eVLP) or n=4 mice (ABE7.10-NG-LV and untreated). 7.10-LV=ABE7.10-NG-LV, 7.10-eVLP=v4 ABE7.10-NG-eVLP.

FIG. 7H is a graph summarizing scotopic a-wave and b-wave amplitudes measured by ERG following overnight dark adaptation. Data are shown as individual data points and mean±s.e.m for n=8 mice (wild-type), n=7 mice (v4 ABE7.10-NG-eVLP), n=5 mice (ABE7.10-NG-LV), or n=4 mice (untreated). P values were calculated using a two-sided t-test.

FIG. 7I shows images of Western blot of protein extracts from RPE tissues of wild-type, untreated, v4 ABE7.10-NG-eVLP-treated, and ABE7.10-NG-LV-treated mice.

FIG. 7J shows representative ERG waveforms from wild-type, untreated, ABE7.10-NG-LV-treated, and v4 ABE7.10-NG-eVLP-treated mice.

FIG. 8A shows images of immunoblot analysis of proteins from purified BE-VLPs using anti-Cas9, anti-p30, and anti-VSV-G antibodies, for validation of VLP production.

FIG. 8B is a graph summarizing adenine base editing efficiencies of v1 BE-VLPs at position A7 of the BCL11A enhancer site in HEK293T cells. Values and error bars reflect mean±s.e.m. of n=3 independent biological replicates. Data were fit to four-parameter logistic curves using nonlinear regression.

FIG. 8C is a schematic of an immature BE-VLP with ABE8e fused to the gag structural protein. Various MMLV protease cleavage sites were inserted between gag and ABE8e to determine the optimal cleavable sequence that promotes liberation of ABE8e from gag during proteolytic virion maturation. Arrows indicate the cleavage site. Sequences shown are PRSSLY (SEQ ID NO: 7), PALTP (SEQ ID NO: 8), VQAL (SEQ ID NO: 9), VLTQ (SEQ ID NO: 10), PLQVL (SEQ ID NO: 11), TLNIERR (SEQ ID NO: 12), TSTLL (SEQ ID NO: 13), and MENSS (SEQ ID NO: 14)

FIG. 8D shows representative images of Western blot evaluating cleaved ABE8e versus full-length gag-ABE8e in purified v2 BE-eVLP variants.

FIG. 8E is a graph summarizing densitometry-based quantification of the cleaved ABE8e fraction from western blots. Data are shown as individual data points and mean values±s.e.m. for n=3 technical replicates.

FIG. 9A shows schematics of v2.4 and v3 BE-eVLP constructs. Three HIV NESs were fused to either the C-terminus or N-terminus of the gag-ABE fusion. A protease cleavable linker was incorporated between ABE and the NES sequences such that the final BE cargo would be devoid of NESs following proteolytic virion maturation. Sequences shown are TSTLL (SEQ ID NO: 13), MENSS (SEQ ID NO: 14), MSKLL (SEQ ID NO: 15), ATVVS (SEQ ID NO: 16), PLQVL (SEQ ID NO: 11), TLNIERR (SEQ ID NO: 12), IRKIL (SEQ ID NO: 17), and FLDG (SEQ ID NO: 18).

FIG. 9B shows a representative immunofluorescence image of producer cells transfected with the v2.4 gag-ABE construct or the v3.4 gag-3×NES-ABE construct. After 48 h post-transfection, cells were fixed in paraformaldehyde and stained with anti-tubulin antibody to stain the cytoskeleton, DAPI for nuclei staining, and anti-Cas9 antibody to visualize gag-ABE fusion, as shown in the legend provided. Scale bars denote 50 μm.

FIG. 9C shows a graph summarizing automated image analysis-based quantification of cytoplasmic localization of the v2.4 gag-ABE construct or the v3.4 gag-3×NES-ABE construct. Data are shown as individual data points and mean values±s.e.m. for n=3 technical replicates. P values were calculated using a two-sided t-test.

FIG. 10A shows a representative negative-stain transmission electron micrograph (TEM) of v4 BE-eVLPs. Scale bar denotes 200 nm.

FIGS. 10B-10C show quantification of protein content for v1, v2.4, v3.4, and v4 BE-eVLPs that was measured by anti-Cas9 or anti-MLV (p30) ELISA. Data are shown as individual data points and mean values±s.e.m. for n=3 technical replicates.

FIG. 10D shows a graph comparing editing efficiencies with particle number-normalized v1, v2.4, v3.4, and v4 BE-VLPs at the BCL11A enhancer site in HEK293T cells. Data are shown as mean values±s.e.m. for n=3 biological replicates.

FIG. 10E shows a graph summarizing cell viability after v4 BE-eVLP treatment of HEK293T cells and NIH 3T3 fibroblasts. Data are shown as mean values±s.e.m. for n=3 biological replicates.

FIG. 10F is a graph summarizing indels frequencies generated by v1 Cas9-VLP and v4 Cas9-eVLPs at the EMX1 locus in HEK293T cells. Data are shown as mean values±s.e.m. for n=3 biological replicates. Data were fit to four-parameter logistic curves using nonlinear regression.

FIG. 10G shows a graph summarizing adenine base editing efficiencies of VSV-G-pseudotyped v4 BE-eVLPs in Neuro-2a cells or 3T3 fibroblasts. Data are shown as individual data points and mean values±s.e.m. for n=3 biological replicates.

FIG. 11A shows an experimental timeline for the orthogonal R-loop assay.

FIG. 11B is a graph summarizing on-target editing controls for the orthogonal R-loop experiment. Data are shown as individual data points and mean values±s.e.m. for n=3 biological replicates.

FIG. 11C is a graph summarizing cell viability following v4 BE-eVLP treatment of RDEB fibroblasts. Data are shown as mean values±s.e.m. for n=3 biological replicates.

FIG. 11D is a graph quantifying DNA sequencing reads containing A·T-to-G·C mutations within protospacer positions 4-10 for ten previously identified off-target loci from the genomic DNA of v4-BE-eVLP-treated RDEB patient-derived fibroblasts. The dotted grey line represents the highest observed background mutation rate of 0.1%. Data are shown as individual data points and mean values±s.e.m. for n=3 biological replicates.

FIGS. 12A-12B show flow cytometry analysis for nuclei sorting from the mouse brain after P0 ICV injection, related to FIGS. 5A-5B. FIG. 12A shows representative flow cytometry graphs. Singlet nuclei were gated based on FSC/BSC ratio and DyeCycle Ruby signal. The first row demonstrates the gating strategy on a GFP-negative sample. Bulk nuclei correspond to events that passed gate D for singlet nuclei. FIG. 12B is a graph summarizing percentage of GFP-positive nuclei measured by flow cytometry following P0 ICV injection. Data are shown as mean values±s.e.m. for n=3 biological replicates.

FIG. 13A shows graphs summarizing plasma aspartate transaminase (AST) and alanine transaminase (ALT) levels one week after v4 BE-eVLP injection.

FIGS. 13B-13C show representative images of histopathological assessment by haematoxylin and eosin staining of livers at 1-week post-injection of (FIG. 13B) untreated mice and (FIG. 13C) v4 BE-eVLP treated mice. A representative example of each is shown. Scale bars denote 50 μm.

FIGS. 14A-14C show results of sequencing analysis of RPE cDNA after v4 BE-eVLP or lentivirus treatment. FIG. 14A shows that v4 BE-eVLP and lentivirus treatment led to 50-60% of A·T-to-G·C conversion at the target adenine (A6) of the Rpe65 transcript. Data are shown as individual data points and mean values±s.e.m. for n=6 (ABE8e-NG-LV, ABE8e-NG-eVLP, and ABE7.10-NG-eVLP), or n=4 (ABE7.10-NG-LV and untreated) replicates. FIGS. 14B-14C show off-target A-to-G RNA editing by v4 BE-eVLPs and lentiviruses as measured by high-throughput sequencing of the Mcm3ap (FIG. 14B) and Perp (FIG. 14C) transcripts. Data are shown as individual data points and mean values±s.e.m. for n=6 (ABE8e-NG-LV, ABE8e-NG-eVLP, and ABE7.10-NG-eVLP), or n=4 (ABE7.10-NG-LV and untreated) replicates.

### DETAILED DESCRIPTION

The practice of some methods disclosed herein employ, unless otherwise indicated, conventional techniques of immunology, biochemistry, chemistry, molecular biology, microbiology, cell biology, genomics, and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### Definitions

As used in the specification and claims, the singular forms “a,” “an,” and “the” include plural references unless the context clearly dictates otherwise. For example, the term “a chimeric transmembrane receptor polypeptide” includes a plurality of chimeric transmembrane receptor polypeptides.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “antigen,” as used herein, refers to a molecule or a fragment thereof capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen.

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e., IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)₂, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” “nucleic acid”, and “nucleic acid molecule” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g., altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), microRNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The terms “target polynucleotide” and “target nucleic acid,” as used herein, refer to a nucleic acid or polynucleotide which is targeted by a cargo of the present disclosure. A target polynucleotide can be DNA (e.g., endogenous or exogenous). DNA can refer to template to generate mRNA transcripts and/or the various regulatory regions which regulate transcription of mRNA from a DNA template. A target polynucleotide can be a portion of a larger polynucleotide, for example a chromosome or a region of a chromosome. A target polynucleotide can refer to an extrachromosomal sequence (e.g., an episomal sequence, a minicircle sequence, a mitochondrial sequence, a chloroplast sequence, etc.) or a region of an extrachromosomal sequence. A target polynucleotide can be RNA. RNA can be, for example, mRNA which can serve as template encoding for proteins. A target polynucleotide comprising RNA can include the various regulatory regions which regulate translation of protein from an mRNA template. A target polynucleotide can encode for a gene product (e.g., DNA encoding for an RNA transcript or RNA encoding for a protein product) or comprise a regulatory sequence which regulates expression of a gene product. In general, the term “target sequence” refers to a nucleic acid sequence on a single strand of a target nucleic acid. The target sequence can be a portion of a gene, a regulatory sequence, genomic DNA, cell free nucleic acid including cfDNA and/or cfRNA, cDNA, a fusion gene, and RNA including mRNA, miRNA, rRNA, and others. A target polynucleotide, when targeted by a cargo, can result in altered gene expression and/or activity. A target polynucleotide, when targeted by a cargo, can result in an edited nucleic acid sequence. A target nucleic acid can comprise a nucleic acid sequence that may not be related to any other sequence in a nucleic acid sample by a single nucleotide substitution. A target nucleic acid can comprise a nucleic acid sequence that may not be related to any other sequence in a nucleic acid sample by a 2, 3, 4, 5, 6, 7, 8, 9, or 10 nucleotide substitutions. In some embodiments, the substitution may not occur within 5, 10, 15, 20, 25, 30, or 35 nucleotides of the 5′ end of a target nucleic acid. In some embodiments, the substitution may not occur within 5, 10, 15, 20, 25, 30, 35 nucleotides of the 3′ end of a target nucleic acid.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The term “regulating” with reference to expression or activity, as used herein, refers to altering the level of expression or activity. Regulation can occur at the transcriptional level, post-transcriptional level, translational level, and/or post-translational level.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “variant,” when used herein with reference to a polypeptide, refers to a polypeptide related, but not identical, to a wild type polypeptide, for example either by amino acid sequence, structure (e.g., secondary and/or tertiary), activity (e.g., enzymatic activity) and/or function. Variants include polypeptides comprising one or more amino acid variations (e.g., mutations, insertions, and deletions), truncations, modifications, or combinations thereof compared to a wild type polypeptide. Variants also include derivatives of the wild type polypeptide and fragments of the wild type polypeptide.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

A Cas protein referred to herein can be a type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon-optimized homologue of a Cas protein. A Cas protein can be enzymatically inactive, partially active, constitutively active, fully active, inducible active and/or more active, (e.g., more than the wild type homologue of the protein or polypeptide.). A Cas protein can be a Type II Cas protein. A Cas protein can be Cas9. A Cas protein can be a Type V Cas protein. A Cas protein can be Cpf1 or Cas12a. A Cas protein can be C2c1. A Cas protein can be C2c3. A Cas protein can be a Type VI Cas protein. A Cas protein can be C2c2 or Cas13a. A Cas protein can be Cas13b. A Cas protein can be Cas13c. A Cas protein can be Cas13d. A Cas protein can be Cas14. A Cas protein (e.g., variant, mutated, enzymatically inactive and/or conditionally enzymatically inactive site-directed polypeptide) can bind to a target nucleic acid. A Cas protein (e.g., variant, mutated, enzymatically inactive and/or conditionally enzymatically inactive endoribonuclease) can bind to a target RNA or DNA.

The term “crRNA,” as used herein, can generally refer to a nucleic acid with at least about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes). crRNA can generally refer to a nucleic acid with at most about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes, S. aureus, etc.). crRNA can refer to a modified form of a crRNA that can comprise a nucleotide change such as a deletion, insertion, or substitution, variant, mutation, or chimera. A crRNA can be a nucleic acid having at least about 60% sequence identity to a wild type exemplary crRNA (e.g., a crRNA from S. pyogenes, S. aureus, etc.) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary crRNA sequence (e.g., a crRNA from S. pyogenes, S. aureus, etc.) over a stretch of at least 6 contiguous nucleotides.

The term “tracrRNA,” as used herein, can generally refer to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes, S. aureus, etc.). tracrRNA can refer to a modified form of a tracrRNA that can comprise a nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes, S. aureus, etc.) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes, S. aureus, etc.) sequence over a stretch of at least 6 contiguous nucleotides.

As used herein, a “guide nucleic acid” can refer to a nucleic acid that can hybridize to another nucleic acid. A guide nucleic acid can be RNA. A guide nucleic acid can be DNA. The guide nucleic acid can be programmed to bind to a sequence of nucleic acid site-specifically. The nucleic acid to be targeted, or the target nucleic acid, can comprise nucleotides. The guide nucleic acid can comprise nucleotides. A portion of the target nucleic acid can be complementary to a portion of the guide nucleic acid. The strand of a double-stranded target polynucleotide that is complementary to and hybridizes with the guide nucleic acid can be called the complementary strand. The strand of the double-stranded target polynucleotide that is complementary to the complementary strand, and therefore may not be complementary to the guide nucleic acid can be called noncomplementary strand. A guide nucleic acid can comprise a polynucleotide chain and can be called a “single guide nucleic acid.” A single guide nucleic acid can comprise a crRNA. A single guide nucleic acid can comprise a crRNA and a tracrRNA. A guide nucleic acid can comprise two polynucleotide chains and can be called a “double guide nucleic acid.” A double guide nucleic acid can comprise a crRNA and a tracrRNA. If not otherwise specified, the term “guide nucleic acid” can be inclusive, referring to both single guide nucleic acids and double guide nucleic acids.

A guide nucleic acid can comprise a segment that can be referred to as a “nucleic acid-targeting segment” or a “nucleic acid-targeting sequence.” A nucleic acid-targeting segment can comprise a sub-segment that can be referred to as a “protein binding segment” or “protein binding sequence” or “Cas protein binding segment”.

The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

As used herein, “nuclear localization domain” can refer to a nuclear localization signal or other sequence or domain capable of traversing a nuclear membrane, thereby entering the nucleus. A nuclear localization domain can be fused in-frame with a polypeptide, in which case the nuclear localization domain can be referred to as a “heterologous nuclear localization domain.”

As used herein, “nuclear export domain,” or “nuclear export sequence” (NES), or “nuclear export signal” (NES) can refer to a nuclear export signal or other sequence or domain that is present in a protein and capable of targeting the protein for export from the cell nucleus to the cytoplasm through the nuclear pore complex using nuclear transport. A nuclear export domain can be fused in-frame with a polypeptide, in which case the nuclear export domain can be referred to as a “heterologous nuclear export domain.”

In eukaryotic cells, transport of proteins between the nucleus and the cytoplasm can be mediated by transport factors in the karyopherin-β family, which are also known as importins and exportins. The direction of nuclear-cytoplasmic transport can be dictated by nuclear targeting signals within the cargo proteins. Nuclear localization sequences (NLSs) can direct proteins into the nucleus, whereas nuclear export sequences (NESs) can direct export of proteins from the nucleus to the cytoplasm. NESs can bind directly to the export karyopherin CRM1 (also known as exportin 1), which can escort cargo proteins through the nuclear pore complex.

In some embodiments, NESs that can be used in the subject compositions, methods, kits, or systems are peptides that are 8-15 residues long and conform loosely to the consensus of Φ1-X_{2,3}-Φ2-X_{2,3}-Φ3-X-Φ4, where Φn represents Leu, Val, Ile, Phe, or Met and X can be any amino acid. In some cases, an NES that can be used in the subject compositions, methods, kits, or systems of the present disclosure conforms to the expanded Kosugi consensus sequences (class 1a, Φ1-X₃-2-X₂-Φ3-X-Φ4; class 1b, Φ1-X₂-Φ2-X₂-Φ3-X-Φ4; class 1c, Φ1-X₃-Φ2-X₃-Φ3-X-Φ4; class 1d, Φ1-X₂-Φ2-X₃-Φ3-X-Φ4; class 2, Φ1-X-Φ2-X₂-Φ3-X-Φ4; class 3, Φ1-X₂-Φ2-X₃-Φ3-X₂-Φ4; where Φn=L, V, I, F, or M, and A, C, T, and W can be one of the Φn), such as those described in Xu D, et al. Mol Biol Cell. 2012 September; 23(18):3673-6. In some cases, an NES that can be used in the subject compositions, methods, kits, or systems of the present disclosure conforms to a NES consensus with three sequence patterns: Φ1-X_{1,2,3}-Φ2-[\hat{ }W]₂-Φ3-[\hat{ }W]-Φ4 (type 1), Φ1-X_{2,3}-Φ2-[\hat{ }W]₃-Φ3-[\hat{ }W]-Φ4 (type 2), and Φ1-X₂-Φ2-X [\hat{ }W]₂-Φ3-[\hat{ }W]₂-Φ4 (type 3), where [\hat{ }W] is any of the 20 amino acids except Trp, and Ala and Thr residues can be used only once at either position Φ1 or Φ2.

As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, or demyristoylation activity. An effector protein can modify a genomic locus. In some embodiments, a fusion protein does not comprise nuclease activity. In some embodiments, a fusion protein does not comprise deaminase activity. In some embodiments, a fusion protein does not comprise polymerase activity (e.g., reverse transcriptase activity).

As used herein, “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

### Delivery Vehicles

In some aspects, the present disclosure relates to delivery vehicles for delivery of therapeutic cargo and/or other molecules into a cell in vitro, ex vivo, or in vivo. In some cases, the delivery vehicles of the present disclosure have high efficiency for in vivo delivery of therapeutic cargo and/or other molecules into a cell of a subject. In some cases, the delivery vehicles of the present disclosure include lipid-containing particles, such as viral-like particles, exosomes, lipid nanoparticles, proteo-lipid vehicles, extracellular vesicle mimetics, and membrane vesicles. The delivery vehicles (e.g., lipid-containing particles) disclosed herein can be highly efficient for in vivo delivery of cargo upon administration into a subject, e.g., a high percentage of cargo loaded in the delivery vehicle is delivered to the cells of the subject, is delivered to the desired subcellular location (e.g., cell nucleus or cell cytoplasm) of the cells of the subject. In some cases, the delivery vehicles are used to deliver genome editing system into cells of a subject, and can have a high efficiency of in vivo gene editing carried out by the genome editing system. In some cases, the delivery vehicles are used to deliver an expression construct encoding a therapeutic protein (e.g., an antibody, a transcription factor, or a chimeric antigen receptor (CAR)) into cells of a subject, and can have a high efficiency of expression of the therapeutic protein in the subject.

In some cases, the lipid-containing particles provided herein comprise a lipid-based external layer enclosing a lumen. A cargo can be loaded in the lipid-containing particles inside the lumen. In some cases, a cargo is loaded in the lipid-containing particles by attaching to the external lipid-based layer. The external lipid-based layer can be a single lipid layer or lipid bilayer made of two layers of lipid molecules. In some cases, the lipid-containing particles have one or more fusion proteins inserted in or attached to the outside of the external lipid layer. The fusion protein can help fusion of the lipid-containing particles with membrane of a target cell, thus delivering the cargo loaded in the lipid-containing vesicles to the target cell.

Typical size of the lipid-containing particles is about 10 nm to about 1000 nm, such as about 10 nm to 50 nm, 10 nm to 100 nm, 10 nm to 200 nm, 10 nm to 300 nm, 10 nm to 400 nm, 10 nm to 500 nm, 10 nm to 600 nm, 10 nm to 800 nm, 20 nm to 50 nm, 20 nm to 100 nm, 20 nm to 200 nm, 20 nm to 300 nm, 20 nm to 400 nm, 20 nm to 500 nm, 20 nm to 600 nm, 20 nm to 800 nm, 50 nm to 100 nm, 50 nm to 200 nm, 50 nm to 300 nm, 50 nm to 400 nm, 50 nm to 500 nm, 50 nm to 600 nm, 50 nm to 800 nm, 100 nm to 200 nm, 100 nm to 300 nm, 100 nm to 400 nm, 100 nm to 500 nm, 100 nm to 600 nm, 100 nm to 800 nm, 200 nm to 300 nm, 200 nm to 400 nm, 200 nm to 500 nm, 200 nm to 600 nm, 200 nm to 800 nm, 400 nm to 600 nm, 400 nm to 800 nm, or 600 nm to 800 nm. In some cases, the lipid-containing particles comprise viral-like particles, lipid nanoparticles, or proteo-lipid vehicles, and have a size of about 10 nm to about 100 nm, such as about 10 nm to about 20 nm, about 10 nm to about 30 nm, about 10 nm to about 40 nm, about 10 nm to about 50 nm, about 10 nm to about 60 nm, about 10 nm to 80 nm, about 20 nm to about 30 nm, about 20 nm to about 40 nm, about 20 nm to about 50 nm, about 20 nm to about 60 nm, about 20 nm to 80 nm, about 40 nm to about 50 nm, about 40 nm to about 60 nm, or about 40 nm to 80 nm. In some cases, the lipid-containing particles comprise exosomes, and have a size of about 50 nm to 200 nm, such as about 50 nm to 80 nm, about 50 nm to 100 nm, about 50 nm to 120 nm, about 50 nm to 150 nm, about 50 nm to 160 nm, about 50 to 180 nm, about 60 nm to 80 nm, about 60 nm to 100 nm, about 60 nm to 120 nm, about 60 nm to 160 nm, about 60 nm to 160 nm, about 60 nm to 180 nm, about 80 nm to 100 nm, about 80 nm to 120 nm, about 80 nm to 160 nm, about 80 nm to 180 nm, about 80 nm to 180 nm, about 100 nm to 120 nm, about 100 nm to 150 nm, about 100 nm to 180 nm, about 120 nm to 150 nm, about 120 nm to 180 nm, about 150 nm to 180 nm, or about 150 nm to 200 nm.

In some aspects, provided herein is a delivery vehicle (e.g., a lipid-containing particle) that includes a cell fusion molecule (e.g., a human endogenous retroviral (HERV) envelope protein, a humanized viral envelope protein, or a non-immunogenic cell fusion molecule); a fusion protein comprising a plasma membrane localization protein (e.g., coupled to a nuclear export sequence (NES)); and a cargo (e.g., a therapeutic cargo or a binding partner for a therapeutic cargo).

In some aspects, provided herein is a delivery vehicle (e.g., a lipid-containing particle) that includes a cell fusion molecule (e.g., a human endogenous retroviral (HERV) envelope protein, a humanized viral envelope protein, or a non-immunogenic cell fusion molecule); and a fusion protein comprising a plasma membrane localization protein (e.g., coupled to a cleavable linker); and a cargo (e.g., a therapeutic cargo or a binding partner for a therapeutic cargo).

In some aspects, provided herein is a delivery vehicle (e.g., a lipid-containing particle) that includes a plasma membrane localization molecule (e.g., a humanized retroviral structural protein or a human endogenous retroviral (HERV) structural protein, e.g., HERV gag, a pleckstrin homology (PH) domain, or a non-immunogenic plasma mem-brane recruitment protein) and a nuclear export sequence (NES).

In some aspects, provided herein is a delivery vehicle (e.g., a lipid-containing particle) that includes a fusion protein comprising i) a plasma membrane localization molecule (e.g., a humanized retroviral structural protein; a human endogenous retroviral (HERV) structural protein, e.g., HERV gag; a pleckstrin homology (PH) domain, or a non-immunogenic plasma mem-brane recruitment protein), a cleavable linker, and a cargo (e.g., a therapeutic cargo or a binding partner for a therapeutic cargo).

### Fusion Protein

In aspects, disclosed herein are fusion proteins that are suitable for assembly of a cargo into a delivery vehicle, e.g., lipid-containing particle, and delivery of the cargo into a cell. The fusion protein can include a plasma membrane localization protein (e.g., retroviral gag protein, human endogenous retroviral gag protein, or a pleckstrin homology domain) fused to a cargo protein (e.g., a therapeutic cargo). In some cases, the fusion protein comprises a cargo that is a binding partner for a therapeutic cargo (e.g., the binding partner can directly bind the therapeutic cargo, or, e.g., the binding partner can bind to another molecule coupled to or interacting with the therapeutic cargo). For example, the fusion protein can comprise a plasma membrane localization protein (e.g., retroviral gag protein, human endogenous retroviral gag protein, or a pleckstrin homology domain) coupled to a nucleic acid binding protein that can bind, e.g., a nuclei acid molecule, e.g., RNA (e.g., mRNA) or DNA. In some cases, the fusion protein is suitable for delivery by a delivery vehicle disclosed herein.

A plasma membrane localization protein disclosed herein can be derived from a virus, human, or any other suitable source. In some cases, a plasma membrane localization protein is human endogenous protein.

In some cases, a fusion protein disclosed herein includes a nuclear localization sequence (NLS). In some cases, the NLS facilitates delivery of the fusion protein, or a cargo released from the fusion protein (for instance, released from the fusion protein following cleavage of a cleavable linker), into the nucleus of a target cell.

In some cases, a fusion polypeptide disclosed herein includes at least one NLS sequence, such as, 2 or more, 3 or more, 4 or more, or 5 or more NLS sequences. In some cases, one or more NLS sequences (2 or more, 3 or more, 4 or more, or 5 or more NLS sequences) are positioned at or near (e.g., within 50 amino acids of) the N-terminus and/or the C-terminus of the fusion protein. In some cases, one or more NLS sequences (2 or more, 3 or more, 4 or more, or 5 or more NLS sequences) are positioned at or near (e.g., within 50 amino acids of) the N-terminus of the fusion protein. In some cases, one or more NLS sequences (2 or more, 3 or more, 4 or more, or 5 or more NLS sequences) are positioned at or near (e.g., within 50 amino acids of) the C-terminus of the fusion protein. In some cases, one or more NLS sequences (3 or more, 4 or more, or 5 or more NLS sequences) are positioned at or near (e.g., within 50 amino acids of) both the N-terminus and the C-terminus of the fusion protein. In some cases, an NLS sequence is positioned at the N-terminus and an NLS sequence is positioned at the C-terminus of the fusion protein.

In some cases, a cargo protein is delivered as part of the fusion protein disclosed herein, e.g., operably linked to a structural protein (e.g., human endogenous retroviral (HERV) structural protein or a plasma membrane recruitment domain). In some embodiments, the one or more NLS sequences are positioned at or near the one or both ends of the cargo protein sequence of the fusion protein. For example, in some cases, one or more NLS sequences (2 or more, 3 or more, 4 or more, or 5 or more NLS sequences) are positioned at or near (e.g., within 50 amino acids of) the N-terminus and/or the C-terminus of the cargo protein sequence. In some cases, one or more NLS sequences (2 or more, 3 or more, 4 or more, or 5 or more NLS sequences) are positioned at or near (e.g., within 50 amino acids of) the N-terminus of the cargo protein sequence. In some cases, one or more NLS sequences (2 or more, 3 or more, 4 or more, or 5 or more NLS sequences) are positioned at or near (e.g., within 50 amino acids of) the C-terminus of the cargo protein sequence. In some cases, one or more NLS sequences (3 or more, 4 or more, or 5 or more NLS sequences) are positioned at or near (e.g., within 50 amino acids of) both the N-terminus and the C-terminus of the cargo protein sequence. In some cases, an NLS sequence is positioned at the N-terminus and an NLS sequence is positioned at the C-terminus of the cargo protein sequence.

In some cases, a fusion protein disclosed herein includes between 1 and 10 NLS sequences (e.g., 1-9, 1-8, 1-7, 1-6, 1-5, 2-10, 2-9, 2-8, 2-7, 2-6, or 2-5 NLS sequences). In some cases, a fusion protein includes (is fused to) between 2 and 5 NLS sequences (e.g., 2-4, or 2-3 NLSs). Non-limiting examples of NLS sequences include an NLS sequence derived from: the NLS of the SV40 virus large T-antigen, having the amino acid sequence PKKKRKV (SEQ ID NO: 19); the NLS from nucleoplasmin (e.g., the nucleoplasmin bipartite NLS with the sequence KRPAATKKAGQAKKKK (SEQ ID NO: 20); the c-myc NLS having the amino acid sequence PAAKRVKLD (SEQ ID NO: 21) or RQRRNELKRSP (SEQ ID NO: 22); the hRNPAI M9 NLS having the sequence NQSSNFGPMKGGNFGGRSSGPYGGGGQYFAKPRNQGGY (SEQ ID NO: 23); the sequence RMRIZFKNKGKDT AELRRRRVE V S VELRK AKKDEQILKRRN V (SEQ ID NO: 24) of the IBB domain from importin-alpha; the sequences VSRKRPRP (SEQ ID NO: 25) and PPKKARED (SEQ ID NO: 26) of the myoma T protein; the sequence PQPKKKPL (SEQ ID NO: 27) of human p53; the sequence SALIKKKKKMAP (SEQ ID NO: 28) of mouse c-abl IV; the sequences DRLRR (SEQ ID NO: 29) and PKQKKRK (SEQ ID NO: 30) of the influenza virus NS1; the sequence RKLKKKIKKL (SEQ ID NO: 31) of the Hepatitis virus delta antigen; the sequence REKKKFLKRR (SEQ ID NO: 32) of the mouse Mx1 protein; the sequence KRKGDE VDGVDEV AKKKS KK (SEQ ID NO: 33) of the human poly(ADP-ribose) polymerase; and the sequence RKCLQAGMNLEARKTKK (SEQ ID NO: 34) of the steroid hormone receptors (human) glucocorticoid, and sequences having at least 80% identity to the foregoing. In some cases, an NLS comprises the amino acid sequence MDSLLMNRRKFLY QFKNVRWAKGRRETYLC (SEQ ID NO: 35).

Other non-limiting examples of an NLS sequence include KRTADGSEFESPKKKRKV (SEQ ID NO: 36), KKTELQTTNAENKTKKL (SEQ ID NO: 37), KRGINDRNFWRGENGRKTR (SEQ ID NO: 38), RKSGKIAAIVVKRPRK (SEQ ID NO: 39), and MDSLLMNRRKFLYQFKNVRWAKGRRETYLC (SEQ ID NO: 35), SPKKKRKVEAS (SEQ ID NO: 40), AGCCCCAAGAAgAAGAGaAAGGTGGAGGCCAGC (SEQ ID NO: 41), and GPKKKRKVAAA (SEQ ID NO: 42), as well as any of those described in Cokol et al., EMBO Rep., 2000, 1(5): 411-415 and Freitas et al., Current Genomics, 2009, 10(8): 550-7; Lu, J., et la., Cell Commun Signal 19, 60 (2021); international publication no. WO/2001/038547, each of which is incorporated herein by reference in its entirety, and sequences having at least 80% identity to the foregoing.

In some cases, a fusion protein disclosed herein include a nuclear export sequence (NES). In some cases, the NES facilitates localization of the fusion protein in the cytosol of a target cell relative to the nucleus.

In some cases, a fusion polypeptide disclosed herein includes at least one NES sequences, such as, 2 or more, 3 or more, 4 or more, or 5 or more NES sequences. In some cases, one or more NES sequences (2 or more, 3 or more, 4 or more, or 5 or more NES sequences) are positioned at or near (e.g., within 50 amino acids of) the N-terminus and/or the C-terminus of the fusion protein. In some cases, the fusion protein disclosed herein comprises only one NES sequence. In some cases, the fusion protein disclosed herein comprises three NES sequences. In some cases, one or more NES sequences (2 or more, 3 or more, 4 or more, or 5 or more NES sequences) are positioned at or near (e.g., within 50 amino acids of) the N-terminus of the fusion protein. In some cases, one or more NES sequences (2 or more, 3 or more, 4 or more, or 5 or more NES sequences) are positioned at or near (e.g., within 50 amino acids of) the C-terminus of the fusion protein. In some cases, one or more NES sequences (3 or more, 4 or more, or 5 or more NES sequences) are positioned at or near (e.g., within 50 amino acids of) both the N-terminus and the C-terminus of the fusion protein. In some cases, an NES sequence is positioned at the N-terminus and an NES sequence is positioned at the C-terminus of the fusion protein.

In some cases, a cargo protein is delivered as part of the fusion protein disclosed herein, e.g., operably linked to a structural protein (e.g., human endogenous retroviral structural protein or a plasma membrane recruitment domain). In some embodiments, the one or more NES sequences are positioned at or near the one or both ends of the cargo protein sequence inside the fusion protein. For example, in some cases, one or more NES sequences (2 or more, 3 or more, 4 or more, or 5 or more NES sequences) are positioned at or near (e.g., within 50 amino acids of) the N-terminus and/or the C-terminus of the cargo protein sequence. In some cases, one or more NES sequences (2 or more, 3 or more, 4 or more, or 5 or more NES sequences) are positioned at or near (e.g., within 50 amino acids of) the N-terminus of the cargo protein sequence. In some cases, one or more NES sequences (2 or more, 3 or more, 4 or more, or 5 or more NES sequences) are positioned at or near (e.g., within 50 amino acids of) the C-terminus of the cargo protein sequence. In some cases, one or more NES sequences (3 or more, 4 or more, or 5 or more NES sequences) are positioned at or near (e.g., within 50 amino acids of) both the N-terminus and the C-terminus of the cargo protein sequence. In some cases, an NES sequence is positioned at the N-terminus and an NES sequence is positioned at the C-terminus of the cargo protein sequence. In some cases, the fusion protein disclosed herein comprises only one NES sequence. In some cases, the fusion protein comprises only one NES sequence, and the NES sequence is positioned at or near (e.g., within 50 amino acids of) the N-terminus of the cargo protein.

In some embodiments, the fusion protein comprises one NES sequence and two NLS sequences. In some cases of these embodiments, the NES sequence, NLS sequences, and the cargo protein sequence are positioned in an order from N-terminus to C-terminus as follows: NES-NLS-cargo protein-NLS. In some embodiments, the fusion protein comprises two or more NES sequences and two NLS sequences. In some cases of these embodiments, the NES sequences, NLS sequences, and the cargo protein sequence are positioned in an order from N-terminus to C-terminus as follows: n×NES (n>=2)-NLS-cargo protein-NLS.

In some cases, a fusion protein disclosed herein includes between 1 and 10 NES sequences (e.g., 1-9, 1-8, 1-7, 1-6, 1-5, 2-10, 2-9, 2-8, 2-7, 2-6, or 2-5 NES sequences). In some cases, a fusion protein includes (is fused to) between 2 and 5 NES sequences (e.g., 2-4, or 2-3 NESs).

In some cases, the NES sequence that can be used in the fusion protein comprise LQLPPLERLTL (SEQ ID NO: 43) derived from HIV-1 Rev protein, and sequences having at least 80% identity thereto. In some cases, the NES sequence comprises LALKLAGLDL (SEQ ID NO: 44) derived from PKIα, and sequences having at least 80% identity thereto. In some cases, the NES sequence disclosed herein comprises a sequence such as those described in la Cour T, et al., Nucleic Acids Res. 2003; 31(1):393-396; and Xu D, et al. Mol Biol Cell. 2012 September; 23(18):3673-6, each of which is incorporated herein by reference in its entirety. Any of the NES sequences described in the NES sequence database (NESdb©; prodata.swmed.edu/LRNes) can be used in a fusion protein disclosed herein, e.g., for the purpose of packaging a cargo protein into the lipid-containing particle, e.g., the viral-like particle.

In some cases, the fusion protein comprises a cleavable linker in between two or more components. For instance, the fusion protein can comprise a cleavable linker between a cargo protein sequence and a plasma membrane localization protein sequence (e.g., retroviral gag protein sequence). In some cases, the cleavable linker separates the plasma membrane localization protein sequence from a NLS sequence, and/or a NES sequence at its N-terminus or C-terminus. The cleavable linker can separate the cargo protein sequence from the plasma membrane localization protein sequence, NLS sequence, and/or NES sequence at its N-terminus or C-terminus. The cleavable linker sequence provided herein can be a cleavable sequence that is recognized and cleaved by a viral protease, a bacterial protease, or a eukaryotic protease (e.g., a protease derived from a plant, an animal, or a fungus). In some cases, the cleavable sequence is recognized by a retroviral protease (pro, e.g., pro derived from Moloney murine leukemia virus (MMLV) or Friend murine leukemia virus (FMLV)). Non-limiting examples of cleavable sequences that can be used in the fusion protein include TSTLLMENSS (SEQ ID NO: 1), PRSSLYPALTP (SEQ ID NO: 2), VQALVLTQ (SEQ ID NO: 3), and PLQVLTLNIERR (SEQ ID NO: 4), and sequences having at least 80% identity to the foregoing.

In some cases, the fusion protein disclosed herein also comprises one or more non-cleavable linkers that operably link components together. The non-cleavable linker can be any suitable linker sequence that is used for fusion protein construction, such as peptide linkers that consist of glycine (Gly) and serine (Ser) residues. In some embodiments, the non-cleavable linker comprises an amino acid sequence selected from the group consisting: (GS)x (SEQ ID NO: 45), (GGS)x (SEQ ID NO: 46), (GGGGS)x (SEQ ID NO: 47), (GGSG)x (SEQ ID NO: 48), and (SGGG)x (SEQ ID NO: 49), and wherein x is an integer from 1 to 50.

In some embodiments, the fusion protein (prior to cleavage) comprises the structure: [gag nucleocapsid polyprotein]-[3×NES]-[cleavable linker]-[NLS]-[therapeutic cargo]-[NLS].

In some cases, the fusion protein has one of the following configurations of components positioned in an order from N-terminus to C-terminus:
[plasma membrane localization protein]-[n*NES]-[cleavable linker]-[m₁*NLS]-[cargo protein]-[m₂*NLS];
[plasma membrane localization protein]-[cleavable linker]-[m₁*NLS]-[cargo protein]-[m₂*NLS]-[n*NES];
[plasma membrane localization protein]-[cleavable linker 1]-[m₁*NLS]-[cargo protein]-]-[m₂*NLS]-[cleavable linker 2]-[n*NES]; and
[plasma membrane localization protein]-[cleavable linker 1]-[m₁*NLS]-[cargo protein]-[m₂*NLS];
[m₁*NLS]-[cargo protein]-[m₂*NLS]-[cleavable linker]-[n*NES]-[plasma membrane localization protein];
[n*NES]-[m₁*NLS]-[cargo protein]-[m₂*NLS]-[cleavable linker]-[plasma membrane localization protein];
[n*NES]-[cleavable linker 1]-[m₁*NLS]-[cargo protein]-[m₂*NLS]-[cleavable linker 2]-[plasma membrane localization protein]; and
[m₁*NLS]-[cargo protein]-[m₂*NLS]-[cleavable linker]-[plasma membrane localization protein];
wherein n, m₁, and m₂ are integers in the range of from 0 to 10, respectively, and denote the number of repeats of the respective sequences they refer to. Non-cleavable linker sequence can be present or absent in any of the foregoing configurations between any two neighboring components.

### Cleavage of Fusion Protein (e.g., Gag-Therapeutic Cargo)

In some embodiments, the fusion protein (e.g., gag-therapeutic cargo) provided herein comprises (a) a sequence of a gag nucleocapsid protein, (b) a sequence of a therapeutic cargo, (c) a cleavable linker, and (d) a nuclear export sequence (NES). In some cases, the therapeutic cargo comprises a nuclease, a base editor, a prime editor, an epigenetic editor, a restriction endonuclease, a recombinase, a transcription factor, an antibody, a chimeric antigen receptor, a T cell receptor, an organelle, a nucleic acid molecule, a DNA, a RNA, a retrotransposon, a reverse transcriptase, an oligonucleotide, an aptazyme, an aptamer, a ribozyme, a small molecule compound, or any combination thereof. For instance, the therapeutic cargo can be a base editor comprising a nuclease domain. In certain embodiments, the therapeutic cargo does not comprise a nuclease, a polymerase (e.g., a reverse transcriptase), a base editor, or a prime editor. In some embodiments, the therapeutic cargo is a protein (e.g., a nuclease, a base editor, a prime editor, an epigenetic editor, a restriction endonuclease (optionally a Type IIS restriction enzyme), a recombinase, a transcription factor, an antibody, a chimeric antigen receptor, a T cell receptor, or a reverse transcriptase). In some embodiments, the therapeutic cargo is a nucleic acid molecule (e.g., DNA, RNA, a retrotransposon, an aptazyme, an aptamer, or a ribozyme). In some embodiments, the therapeutic cargo is a an epigenetic editor, a restriction endonuclease (optionally a Type IIS restriction enzyme), a recombinase, a transcription factor, an antibody, a chimeric antigen receptor, a T cell receptor, an organelle, a nucleic acid molecule, a DNA, a RNA, a retrotransposon, an oligonucleotide, an aptazyme, an aptamer, a ribozyme, a small molecule compound, or any combination thereof.

In some embodiments, the cleavable linker is located between the sequence of the therapeutic cargo and the NES. The fusion protein can be excised at the cleavable linker upon packaging into a lipid-containing particle provided herein (e.g., a viral-like particle), for instance, the cleavable linker can be recognized by the protease (pro) protein of the lipid-containing particle (e.g., viral-like particle). Upon cleavage of the fusion protein at the cleavable linker, the therapeutic cargo can be released as a separate protein into the inside of the protein core. As a result, in some cases, a lipid-containing particle (e.g., a viral-like particle) provided herein comprises a cleavage product comprising a gag nucleocapsid protein and NES, but lacking the therapeutic cargo. In some of these embodiments, as a result of the cleavage at the cleavable linker, there is a relatively small amount of the fusion protein in the lipid-containing particle that is not cleaved at the cleavable linker as compared to the amount of fusion protein that is originally packaged into the lipid-containing particle before the cleavage, for instance, at most about 50%, at most about 40%, at most about 35%, at most about 30%, at most about 25%, at most about 20%, at most about 18%, at most about 16%, at most about 14%, at most about 12%, at most about 10%, at most about 8%, at most about 6%, at most about 5%, at most about 4%, at most about 3%, at most about 2%, at most about 1%, at most about 0.8%, at most about 0.6%, at most about 0.5%, at most about 0.4%, at most about 0.3%, at most about 0.2%, at most about 0.1%, at most about 0.05%, or at most about 0.01% of the amount of the fusion protein before the cleavage in the lipid-containing particle. In some embodiments, there is no fusion protein left in the lipid-containing particle that is not cleaved at the cleavable linker.

In some embodiments, following cleavage, the fusion protein comprises the structure [gag nucleocapsid protein]-[3×NES]. In some embodiments, the cleavage product comprises the structure [NLS]-[therapeutic cargo]-[NLS].

In some embodiments, as a result of the cleavage of the fusion protein at the cleavable linker, there is a significant amount of therapeutic cargo present within the inside of the protein core of the lipid-containing particle, separate from the fusion protein or the cleavage product. In some embodiments, there is relatively more therapeutic cargo present within the inside of the protein core of the lipid-containing particle, as compared to the fusion protein in the lipid-containing particle. In some embodiments, a ratio of an amount of the therapeutic cargo present within the inside of the protein core versus an amount of the fusion protein is at least 1.5. In some cases, the ratio is at least 2, 3, 4, 5, 6, 7, 8, 9, 10, or 20. In some cases, the ratio is at least 100. In some cases, the ratio is at least 1000. In some cases, the ratio is at least 10,000. In some cases, the ratio is from about 1.5 to about 100, such as about 2 to about 100, about 5 to about 100, about 10 to about 100, about 2 to about 80, about 5 to about 80, about 10 to about 80, about 2 to about 60, about 5 to about 60, about 10 to about 60, about 2 to about 50, about 5 to about 50, about 10 to about 50, about 2 to about 40, about 5 to about 40, about 10 to about 40, about 2 to about 30, about 5 to about 30, about 10 to about 30, about 2 to about 20, about 5 to about 20, about 10 to about 20, about 20 to about 100, about 30 to about 100, about 40 to about 100, about 50 to about 100, or about 80 to about 100. In some cases, the ratio is from about 100 to about 1000, such as about 100 to about 800, about 100 to about 600, about 100 to about 500, about 100 to about 400, about 100 to about 300, about 100 to about 200, about 200 to about 1000, about 200 to about 800, about 200 to about 600, about 200 to about 500, about 200 to about 400, about 200 to about 300, about 400 to about 1000, about 400 to about 800, about 400 to about 600, or about 400 to about 500.

In some embodiments, there is at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.2%, at least about 99.5%, at least about 99.6%, at least about 99.7%, at least about 99.8%, at least about 99.9%, at least about 99.95%, or at least about 99.99% of the sequence of the therapeutic cargo in the lipid-containing particle is present in the form of the therapeutic cargo located within the inside of the protein core, which is separate from the fusion protein or the cleavage product. In some embodiments, there is about 60%, about 70%, about 80%, about 90%, about 91%, about 92%, about 93%, about 94%, about 95%, about 96%, about 97%, about 98%, about 99%, about 99.2%, about 99.5%, about 99.6%, about 99.7%, about 99.8%, about 99.9%, about 99.95%, about 99.99%, or about 100% of the sequence of the therapeutic cargo in the lipid-containing particle present in the form of the therapeutic cargo located within the inside of the protein core. The percentage of the sequence of the therapeutic cargo present in the form of separate therapeutic cargo located within the inside of the protein core can be calculated as the percentage of an amount of the therapeutic cargo located within the inside of the protein core versus the sum of the amount of the therapeutic cargo located within the inside of the protein core and an amount of the fusion protein in the lipid-containing particle that comprises the sequence of the therapeutic cargo.

An amount of the therapeutic cargo or the fusion protein within the lipid-containing particle can each be measured, for instance, by any techniques known to the skilled person in the art for quantitative measurement of proteins or protein sequences. For example, Western blotting or Jess blotting with an antibody against a sequence or an epitope of the therapeutic cargo can be conducted to distinguish and measure the amounts of the therapeutic cargo and the fusion protein.

In some cases, the ratio of an amount of the therapeutic cargo present within the inside of the protein core versus an amount of the fusion protein is measured at the level of a population of same or similar lipid-containing particles (e.g., viral-like particles), for instance, one or more preparations of such lipid-containing particles. In some aspects, provided herein is a population of lipid-containing particles (e.g., viral-like particles). In some embodiments of the population of lipid-containing particles, the lipid-containing particle comprises a lipid membrane encapsulating a protein core, and the protein core comprises a group-specific antigen (gag) protease (pro) polyprotein and a fusion protein that comprises a sequence of a therapeutic cargo and a sequence of a gag protein. In some of these embodiments, the lipid-containing particle further comprises a therapeutic cargo located within an inside of the protein core, separate from the fusion protein. In some embodiments, a ratio of an amount of the therapeutic cargo present within the inside of the protein core versus an amount of the fusion protein is at least 1.5 amongst the population of lipid-containing particles. In some embodiments, the ratio amongst the population of lipid-containing particles is at least 2, 3, 4, 5, 6, 7, 8, 9, 10, or 20. In some cases, the ratio amongst the population of lipid-containing particles is at least 100. In some cases, the ratio amongst the population of lipid-containing particles is at least 1000. In some cases, the ratio amongst the population of lipid-containing particles is at least 10,000. In some cases, the ratio amongst the population of lipid-containing particles is from 1.5 to 100, such as 2 to 100, 5 to 100, 10 to 100, 2 to 80, 5 to 80, 10 to 80, 2 to 60, 5 to 60, 10 to 60, 2 to 50, 5 to 50, 10 to 50, 2 to 40, 5 to 40, 10 to 40, 2 to 30, 5 to 30, 10 to 30, 2 to 20, 5 to 20, 10 to 20, 20 to 100, 30 to 100, 40 to 100, 50 to 100, or 80 to 100. In some cases, the ratio amongst the population of lipid-containing particles is from 100 to 1000, such as 100 to 800, 100 to 600, 100 to 500, 100 to 400, 100 to 300, 100 to 200, 200 to 1000, 200 to 800, 200 to 600, 200 to 500, 200 to 400, 200 to 300, 400 to 1000, 400 to 800, 400 to 600, or 400 to 500.

In some embodiments, amongst the population of lipid-containing particles, there is at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.2%, at least about 99.5%, at least about 99.6%, at least about 99.7%, at least about 99.8%, at least about 99.9%, at least about 99.95%, or at least about 99.99% of the sequence of the therapeutic cargo in the lipid-containing particle that is present in the form of the therapeutic cargo located within the inside of the protein core. In some embodiments, amongst the population of lipid-containing particles, there is about 60%, about 70%, about 80%, about 90%, about 91%, about 92%, about 93%, about 94%, about 95%, about 96%, about 97%, about 98%, about 99%, about 99.2%, about 99.5%, about 99.6%, about 99.7%, about 99.8%, about 99.9%, about 99.95%, about 99.99%, or about 100% of the sequence of the therapeutic cargo in the lipid-containing particle that is present in the form of the therapeutic cargo located within the inside of the protein core. The percentage of the sequence of the therapeutic cargo in the population of lipid-containing particles can be calculated as the percentage of an amount of the therapeutic cargo located within the inside of the protein core amongst the population of lipid-containing particles versus the sum of the amount of the therapeutic cargo located within the inside of the protein core and an amount of the fusion protein in the lipid-containing particle that comprises the sequence of the therapeutic cargo.

In some of these embodiments, at the population level, as a result of the cleavage at the cleavable linker, there is a relatively small amount of the fusion protein in the population of lipid-containing particles that is not cleaved at the cleavable linker as compared to the amount of fusion protein that is originally packaged into the population of lipid-containing particles before the cleavage, for instance, at most about 40%, at most about 35%, at most about 30%, at most about 25%, at most about 20%, at most about 18%, at most about 16%, at most about 14%, at most about 12%, at most about 10%, at most about 8%, at most about 6%, at most about 5%, at most about 4%, at most about 3%, at most about 2%, at most about 1%, at most about 0.8%, at most about 0.6%, at most about 0.5%, at most about 0.4%, at most about 0.3%, at most about 0.2%, at most about 0.1%, at most about 0.05%, or at most about 0.01% of the amount of the fusion protein that is originally packaged into the lipid-containing particle before the cleavage.

In other embodiments, at the population level, there is only trace amount of fusion protein left in the population of lipid-containing particles that is not cleaved at the cleavable linker, for instance, at most about 100, 80, 60, 50, 40, 30, 20, 10, 8, 6, 4, 2, or one such uncleaved fusion protein per each lipid-containing particle (e.g., viral-like particle), or at most about one such uncleaved fusion protein every 2, 5, 10, 20, 30, 40, 50, 60, 70, 80, 90, 100, 150, 200, 250, 300, 400, 500, 600, 700, 800, 900, 1000, 1200, 1500, 2000, 3000, 5000, 10\hat{ }4, 10\hat{ }5, or 10\hat{ }6 lipid-containing particles (e.g., viral-like particles) in a population.

In some embodiments, as a result of the cleavage of the fusion protein at the cleavable linker, there is a significant amount of therapeutic cargo present within the inside of the protein core of the lipid-containing particle, separate from the fusion protein or the cleavage product. In some embodiments, there is relatively more therapeutic cargo present within the inside of the protein core of the lipid-containing particle, as compared to the fusion protein in the lipid-containing particle. In some embodiments, a ratio of an amount of the therapeutic cargo present within the inside of the protein core versus an amount of the fusion protein is at least 1.5. In some cases, the ratio is at least 2, 3, 4, 5, 6, 7, 8, 9, 10, or 20. In some cases, the ratio is at least 100. In some cases, the ratio is at least 1000. In some cases, the ratio is at least 10,000. In some cases, the ratio is from 1.5 to 100, such as 2 to 100, 5 to 100, 10 to 100, 2 to 80, 5 to 80, 10 to 80, 2 to 60, 5 to 60, 10 to 60, 2 to 50, 5 to 50, 10 to 50, 2 to 40, 5 to 40, 10 to 40, 2 to 30, 5 to 30, 10 to 30, 2 to 20, 5 to 20, 10 to 20, 20 to 100, 30 to 100, 40 to 100, 50 to 100, or 80 to 100. In some cases, the ratio is from 100 to 1000, such as 100 to 800, 100 to 600, 100 to 500, 100 to 400, 100 to 300, 100 to 200, 200 to 1000, 200 to 800, 200 to 600, 200 to 500, 200 to 400, 200 to 300, 400 to 1000, 400 to 800, 400 to 600, or 400 to 500.

### Plasma Membrane Localization Protein

In some cases, the plasma membrane localization described herein forms basic structure of the delivery vehicles, e.g., at least part of the delivery vehicle. In some cases, the plasma membrane localization protein described herein also facilitates self-assembly of the delivery vehicles (e.g., VLPs). For instance, the plasma membrane localization protein can facilitate localization to the plasma membrane and packaging of the delivery vehicle (e.g., viral-like particle) by forming the membrane enclosure.

In some cases, the plasma membrane localization protein is a viral protein, e.g., derived from a virus. In some cases, the plasma membrane localization protein is a mammalian protein, e.g., derived from a mammal, e.g., human. In some cases, the plasma membrane localization protein is a human endogenous protein.

In some cases, the plasma membrane localization protein is a polyprotein derived from a virus, a homologue thereof, a fragment thereof, a variant thereof, or any combination thereof. For instance, the plasma membrane localization protein comprises a retroviral gag protein, e.g., a retroviral polyprotein that comprises one or more of a matrix (MA) polypeptide, an RNA-binding phosphoprotein polypeptide, a capsid (CA) polypeptide, or a nucleocapsid (NC) polypeptide. In some cases, the gag protein is derived from Friend murine leukemia virus (FMLV). In some cases, the retroviral gag polyprotein is a gag polyprotein of an alpha retrovirus, a beta retrovirus, a gamma retrovirus, a delta retrovirus, an epsilon retrovirus, or a spumavirus. In some cases, the retroviral gag polyprotein is a gag polyprotein of a human immunodeficiency virus.

Non-limiting examples of the plasma membrane localization protein comprises Human Papillomavirus (HPV) L1 protein, HPV L2 protein, Hepatitis B virus (HBV) core protein, Chikungunya virus (CHIKV) C-E3-E2-6k-E1, human immunodeficiency virus (HIV) gag-pol, HIV gag, Respiratory syncytial virus (RSV) M, RSV NP, Human metapneumovirus (HMPV) M, Influenza M1, Zika virus (ZIKV) C, ZIKV prM/M, Dengaue virus (DENV) C-prM, West Nile Virus (WNV) prME protein, WNV CprME protein, Filovirus VP40 or Z protein, Baculovirus P1 protein, Rotavirus VP7, Rotavirus VP2 protein, Rotavirus VP6 protein, SARS M protein, SARS E protein, SARS N protein, Porcine Circovirus Type 2 (PCV2) capsid, baculovirus VP2 protein, baculovirus VP5 protein, baculovirus VP3 protein, or baculovirus VP7 protein, Hepatitis C virus (HCV) core protein, Ebola nucleocapsid, Parovirus VP1 protein, Parovirus VP2 protein, Newcastle disease virus (NDV) M protein, hepatitis E virus (HeV) M protein, Nipah virus (NIV) M protein, Human polyomavirus 2 (JCPyV) VP1 protein, Human parainfluenza virus type 3 (HPIV3) M protein, HPIV3N protein, or Mumps virus (MuV) M proteins, a homologue thereof, a fragment thereof, a variant thereof, or any combination thereof.

In some cases, the plasma membrane localization protein sequence comprises a human endogenous retrovirus (HERV) gag protein. In some cases, the plasma membrane localization protein sequence comprises a pleckstrin homology (PH) domain. Non-limiting examples of the plasma membrane localization protein sequences can include those described in Table 2.

### Cell Fusion/Envelope Proteins

The cell fusion proteins disclosed herein can refer to proteins that are present on the external membrane of the delivery vehicle (e.g., is inserted in, attached to, or anchored in the lipid layer) and facilitate the fusion of the delivery vehicle with a membrane, e.g., a target cell membrane. In some cases, the cell fusion protein mediates tropism of the delivery vehicle, e.g., preferential fusion of the delivery vehicle into one or more certain types of cells. In some cases, the cell fusion protein results in mixing between lipids in the delivery vehicle and lipids in the target cell. In some cases, a lipid-containing particle includes a human endogenous retroviral (HERV) envelope protein, a humanized viral envelope protein, or a non-immunogenic cell fusion molecule. Non-limiting examples of HERV envelope proteins can include those described in Table 1.

In some cases, the cell fusion protein comprises a mammalian protein. In some cases, the cell fusion protein comprises a viral protein. In some embodiments, the cell fusion protein comprises a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the cell fusion proteins or fragments, and any combination thereof.

A non-immunogenic cell fusion protein provided herein can have reduced immunogenicity to a human subject as compared to a protein heterologous to the human subject. For instance, the non-immunogenic cell fusion protein can be humanized to reduced immunogenicity to a human subject. In some embodiments, the cell fusion proteins can be modified to reduce immunoreactivity. For instance, cell fusion proteins can be decorated with molecules that reduce immune interactions, such as PEG, such as described in Croyle M A, et al., J Virol. 2004 January; 78(2):912-21, which is incorporated herein by reference in its entirety. Thus, in some embodiments, the envelope protein comprises PEG, e.g., is a PEGylated polypeptide. Amino acid residues in the cell fusion proteins that are targeted by the immune system may be altered to be unrecognized by the immune system, such as described in Lech P J, et al., Virology. 2014 April; 454-455:237-46; and Kneissl S, et al., PLOS One. 2012; 7(10):e46667., each of which is incorporated herein by reference in its entirety. In some embodiments the protein sequence of the cell fusion protein is altered to resemble amino acid sequences found in humans (humanized). In some embodiments the protein sequence of the cell fusion protein is changed to a protein sequence that binds MHC complexes less strongly. In some embodiments, the cell fusion proteins are derived from viruses or organisms that do not infect humans (and which humans have not been vaccinated against), increasing the likelihood that a patient's immune system is naïve to the cell fusion proteins (e.g., there is a negligible humoral or cell-mediated adaptive immune response towards the cell fusion protein) (doi: 10.1006/mthe.2002.0550, doi: 10.1371/journal.ppat.1005641, doi: 10.1038/gt.2011.209, DOI 10.1182/blood-2014-02-558163). In some embodiments, glycosylation of the envelope protein is changed to alter immune interactions or reduce immunoreactivity.

In some cases, the cell fusion protein comprises a sequence chosen from a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein, or a derivative thereof.

In some cases, the cell fusion protein includes a mammalian protein. Examples of mammalian cell fusion protein may include, but are not limited to, a SNARE family protein such as vSNAREs and tSNAREs, a syncytin protein such as Syncytin-1, and Syncytin-2, myomaker, myomixer, myomerger, FGFRL1 (fibroblast growth factor receptor-like 1), Minion, an isoform of glyceraldehyde-3-phosphate dehydrogenase (GAPDH) (e.g., as disclosed in U.S. Pat. No. 6,099,857), a gap junction protein such as connexin 43, connexin 40, connexin 45, connexin 32 or connexin 37 (e.g., as disclosed in US 2007/0224176), Hap2, any protein capable of inducing syncytium formation between heterologous cells, a homologue thereof, a fragment thereof, a variant thereof, and a protein fusion comprising one or more proteins or fragments thereof. In some embodiments, the cell fusion protein comprises a curvature-generating protein, e.g., Epsin1, dynamin, or a protein comprising a BAR domain, such as those described in Kozlov et al., CurrOp StrucBio 2015 2015 August; 33:61-67; Zimmerberg et al., Nat Rev Mol Cell Biol. 2006 January; 7(1):9-19; Richard et al., Biochem J. 2011 Dec. 1; 440(Pt2): 185-193, each of which is incorporated herein by reference in its entirety.

In some cases, the cell fusion protein includes a non-mammalian protein, e.g., a viral cell fusion protein. In some embodiments, a viral cell fusion protein is a Class I viral membrane cell fusion protein, a Class II viral cell fusion protein, a Class III viral membrane cell fusion protein, a viral cell fusion protein, or other viral cell fusion protein, or a homologue thereof, a fragment thereof, a variant thereof, or a protein fusion comprising one or more proteins or fragments thereof. Examples of Class I viral cell fusion protein can be used in the VLPs disclosed herein include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and cell fusion proteins of filoviruses and coronaviruses. In embodiments, class II viral cell fusion proteins such as dengue E glycoprotein, have a structural signature of β-sheets forming an elongated ectodomain that refolds to result in a trimer of hairpins. In embodiments, the class II viral cell fusion proteins lacks the central coiled coil. Examples of Class II viral cell fusion protein can be used in the VLPs disclosed herein include, but are not limited to, tick bone encephalitis E (TBEV E), Semliki Forest Virus E1/E2, as well as cell fusion proteins derived from Sinbis, rubella virus, and dengue virus. In embodiments, class III viral cell fusion proteins such as the vesicular stomatitis virus G glycoprotein, combine structural signatures found in classes I and II. In embodiments, a class III viral cell fusion protein comprises a helices (e.g., forming a six-helix bundle to fold back the protein as with class I viral cell fusion proteins), and 3 sheets with an amphiphilic fusion peptide at its end, reminiscent of class II viral cell fusion proteins. Examples of Class III viral cell fusion protein can be used in the VLPs disclosed herein include, but are not limited to, rhabdovirus G (e.g., protein G of the Vesicular Stomatatis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G). In embodiments, class IV viral cell fusion proteins are fusion-associated small transmembrane (FAST) proteins (doi: 10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral cell fusion proteins are sufficiently small that they do not form hairpins (doi: 10.1146/annurev-cellbio-101512-122422, doi: 10.1016/j.devcel.2007.12.008).

Non-limiting examples of other viral cell fusion protein that can be used in the VLPs disclosed herein include, but are not limited to: viral syncytia proteins such as influenza hemagglutinin (HA) or mutants, or fusion proteins thereof; human immunodeficiency virus type 1 cell fusion protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, or any combination thereof. In some cases, the viral cell fusion protein comprises a Measles virus hemagglutinin (HA) protein and/or a measles virus fusion glycoprotein, Influenza virus neuraminidase (NA) protein, a Measles virus F protein, an Influenza virus HA protein, Moloney virus MLV-A protein, a Moloney virus MLV-E protein, a Baboon Endogenous retrovirus (BAEV) cell fusion protein, an Ebola virus glycoprotein, a foamy virus cell fusion protein, or a homologue thereof, a fragment thereof, a variant thereof, or any combination thereof.

Non-limiting examples of other viral cell fusion protein that can be used in the VLPs disclosed herein include, but are not limited to: hemagglutinin (HA) or neuroaminidase (NA) proteins derived from Orthomyxoviridae-Influenza A, E protein E1 and E2 subunits (included in a complex together and apart) of Togaviridae-CHIV; S,E, or MN protein from Cornaviridae-SARS and COVID19; F or G proteins from Paramyxoviridae-Nipah virus; GP protein from Filoviridae-Ebola; E protein from Flaviviridae-Dengue virus; Gn and Gc proteins (include in a complex together and apart) from Phenuviridae-Sandfly fever virus; GP protein from Arenavirida-Lassa virus; Gn and Gc proteins (included in a complex together and apart) from Hantaviridae-hantavirus; G protein from Bornaviridae-Borna disease virus; Gn and Gc proteins (included in a complex together and apart) from Bunyaviridae-Crimean-Congo hemorrhagic fever virus; S, M, or L proteins from Hepadnaviridae-Hepatitis B virus; cell fusion protein from Herpesviridae-Herpes Simplex Virus 1; EV protein from Poxviridae-Variola virus; S, L, or M proteins from Hepatitis D; or glycoprotein from Hepeviridae-Hepatitis E virus, or a homologue thereof, a fragment thereof, a variant thereof, and a protein fusion comprising one or more proteins or fragments thereof.

In some embodiments the cell fusion protein is derived from paramyxovirus. In some embodiments the cell fusion protein is a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein.

In some embodiments, the cell fusion protein is derived from poxviridae. Additional exemplary cell fusion proteins are disclosed in U.S. Pat. No. 9,695,446, US 2004/0028687, U.S. Pat. Nos. 6,416,997, 7,329,807, US 2017/0112773, US 2009/0202622, and US 2004/0009604, and International Patent Publication Nos. WO 2006/027202 and WO2020102709, each of which is incorporated herein by reference in its entirety.

In some embodiments, the cell fusion protein includes an EFF-1, AFF-1, gap junction protein, e.g., a connexin (such as Cn43, GAP43, CX43) (DOI: 10.1021/jacs.6b05191), other tumor connection proteins, a homologue thereof, a fragment thereof, a variant thereof, and a protein fusion comprising one or more proteins or fragments thereof.

Cell fusion proteins disclosed herein can be re-targeted by mutating amino acid residues in a fusion protein (e.g., the hemagglutinin protein). In some embodiments, the envelope protein is randomly mutated. In some embodiments, the envelope protein is rationally mutated. In some embodiments, the envelope protein is subjected to directed evolution.

Cell fusion proteins disclosed herein can be re-targeted by covalently conjugating a targeting-moiety. For instance, a cell fusion protein can be covalently conjugated to a targeting moiety by expression of a chimeric protein comprising the envelope protein linked to the targeting moiety. A target of the targeting moiety includes any peptide (e.g., a receptor) that is displayed on a target cell. In some examples the target is expressed at higher levels on a target cell than non-target cells.

Targeting moieties can be selected to target particular tissue types such as muscle, brain, liver, pancreas and lung for example, or to target a diseased tissue such as a tumour. In a particularly preferred embodiment of the present invention, the exosomes are targeted to brain tissue.

Specific non-limiting examples of targeting moieties include muscle specific peptide, discovered by phage display, to target skeletal muscle, a 29 amino acid fragment of Rabies virus glycoprotein that binds to the acetylcholine receptor or a fragment of neural growth factor that targets its receptor to target neurons and secretin peptide that binds to the secretin receptor can be used to target biliary and pancreatic epithelia. As an alternative, immunoglobulins and their derivatives, including scFv antibody fragments can also be expressed as a fusion protein to target specific antigens, such as VEGFR for cancer gene therapy. As an alternative, natural ligands for receptors can be expressed as fusion proteins to confer specificity, such as NGF which binds NGFR and confers neuron-specific targeting.

A targeting moiety can include, e.g., an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Cell fusion proteins may be re-targeted by non-covalently conjugating a targeting moiety to the fusion protein or targeting protein (e.g., the hemagglutinin protein). For example, the fusion protein can be engineered to bind the Fc region of an antibody that targets an antigen on a target cell, redirecting the fusion activity towards cells that display the antibody's target.

A targeting moiety can comprise, e.g., a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE® s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR® s.

In embodiments, the targeting moiety linked to the cell fusion protein binds a cell surface marker on the target cell, e.g., a protein, glycoprotein, receptor, cell surface ligand, agonist, lipid, sugar, class I transmembrane protein, class II transmembrane protein, or class III transmembrane protein.

In some cases, the delivery vehicles disclosed herein (e.g., VLPs, exosomes, or lipid nanoparticles) also display targeting moieties that are not conjugated to the cell fusion protein or other proteins in order to redirect the fusion activity of the delivery vehicle towards a cell that is bound by the targeting moiety, or to affect homing of the delivery vehicle toward the target cell.

### Viral-Like Particles (e.g., “Lipid-Containing Particles”)

In some aspects, disclosed herein are compositions, methods, and systems related to viral-like particles that can be utilized to deliver cargo into a cell.

A viral-like particle (VLP) disclosed herein can comprise one or more virus-derived proteins, such as a structural protein of VLPs and an envelope protein. In some cases, the virus-derived protein is present as part of a fusion protein that forms the VLP.

In some cases, the loading capacity of the VLPs disclosed herein has a loading capacity that is at least 2-fold, 3-fold, 4-fold, 5-fold, 6-fold, 7-fold, 8-fold, 9-fold, 10-fold, 12-fold, 14-fold, 16-fold, 18-fold, 20-fold, 30-fold, 40-fold, 50-fold, 60-fold, 80-fold, 100-fold higher than a conventional VLP.

### Structural Proteins of VLPs

In some cases, the structural protein described herein forms basic structure of the viral-like particle, e.g., at least part of the capsid that encapsulate the lumen of the VLP. Structural proteins of viral-like particle can include a plasma membrane localization protein. In some cases, the plasma membrane localization protein described herein also facilitates self-assembly of the VLP, e.g., facilitates localization of plasma membrane and packaging of the viral-like particle by forming the membrane enclosure. In some cases, the structural protein described herein facilitates releases of the VLP from the producer cell from which the VLP is produced.

In some cases, the structural protein of VLP (e.g., plasma membrane localization protein) is a viral protein, e.g., derived from a virus. In some cases, the structural protein of VLP is a mammalian protein, e.g., derived from a mammal, e.g., human. In some cases, the structural protein of VLP is a human endogenous protein.

In some cases, the structural protein of VLP (e.g., plasma membrane localization protein) is a polyprotein derived from a virus, a homologue thereof, a fragment thereof, a variant thereof, or any combination thereof. For instance, the structural protein of VLP (e.g., plasma membrane localization protein) comprises a retroviral gag protein, e.g., a retroviral polyprotein that comprises one or more of a matrix (MA) polypeptide, an RNA-binding phosphoprotein polypeptide, a capsid (CA) polypeptide, or a nucleocapsid (NC) polypeptide. In some cases, the gag protein is derived from Friend murine leukemia virus (FMLV). In some cases, the retroviral gag polyprotein is a gag polyprotein of an alpha retrovirus, a beta retrovirus, a gamma retrovirus, a delta retrovirus, an epsilon retrovirus, or a spumavirus. In some cases, the retroviral gag polyprotein is a gag polyprotein of a human immunodeficiency virus.

Non-limiting examples of the structural protein of VLP (e.g., plasma membrane localization protein) comprises Human Papillomavirus (HPV) L1 protein, HPV L2 protein, Hepatitis B virus (HBV) core protein, Chikungunya virus (CHIKV) C-E3-E2-6k-E1, human immunodeficiency virus (HIV) gag-pol, HIV gag, Respiratory syncytial virus (RSV) M, RSV NP, Human metapneumovirus (HMPV) M, Influenza M1, Zika virus (ZIKV) C, ZIKV prM/M, Dengaue virus (DENV) C-prM, West Nile Virus (WNV) prME protein, WNV CprME protein, Filovirus VP40 or Z protein, Baculovirus P1 protein, Rotavirus VP7, Rotavirus VP2 protein, Rotavirus VP6 protein, SARS M protein, SARS E protein, SARS N protein, Porcine Circovirus Type 2 (PCV2) capsid, baculovirus VP2 protein, baculovirus VP5 protein, baculovirus VP3 protein, or baculovirus VP7 protein, Hepatitis C virus (HCV) core protein, Ebola nucleocapsid, Parovirus VP1 protein, Parovirus VP2 protein, Newcastle disease virus (NDV) M protein, hepatitis E virus (HeV) M protein, Nipah virus (NIV) M protein, Human polyomavirus 2 (JCPyV) VP1 protein, Human parainfluenza virus type 3 (HPIV3) M protein, HPIV3N protein, or Mumps virus (MuV) M proteins, a homologue thereof, a fragment thereof, a variant thereof, or any combination thereof.

### Envelope Protein

In some cases, the VLPs disclosed herein comprise an external lipid-based membrane (“envelope”). In some cases, the envelope comprises a single layer of lipid. In some cases, the envelope comprises a lipid bilayer. In some cases, the envelope further comprises a cell-fusion protein (also termed as an “envelope protein” for a VLP) that is inserted in, attached to, or anchored in the lipid layer.

The envelope protein can facilitate the fusion of the VLP to a membrane, e.g., a cell membrane. In some cases, the envelope protein mediates tropism of the VLP, e.g., preferential fusion of the VLP into one or more certain types of cells. In some cases, the envelope protein results in mixing between lipids in the VLP and lipids in the target cell. The envelope protein can be any of the cell fusion proteins disclosed above. In some cases, the envelope protein can be a fusion protein comprising a targeting moiety disclosed above.

In some cases, the envelope protein of a VLP is engineered to pseudotype the VLP for certain properties, e.g., a specific tropism toward select group of cells. In some cases, the envelope protein of a VLP is a pseudotyping viral glycoprotein, such as, but not limited to, a Hepatitis B virus (HBV) glycoprotein, a Hepatitis C virus (HCV) glycoprotein, a Marburg virus glycoprotein, an Ebola virus glycoprotein, a VSV-G glycoprotein; and the target cell is a liver cell. In some cases, the envelope protein of a VLP is a pseudotyping viral glycoprotein, such as, but not limited to, a viral glycoprotein is selected from an influenza virus hemagglutinin, a SARS-COV glycoprotein, a respiratory syncytial virus glycoprotein, a human parainfluenza virus glycoprotein, and a VSV-G; and the target cell is a lung cell. In some cases, the envelope protein of a VLP is a pseudotyping viral glycoprotein, such as, but not limited to, a viral glycoprotein is a measles virus hemagglutinin and/or a measles virus fusion glycoprotein, and the target cell is a CD34⁺ cell. In some cases, the envelope protein of a VLP is a pseudotyping viral glycoprotein, such as, but not limited to, a viral glycoprotein is selected from a measles virus hemagglutinin and/or a measles virus fusion glycoprotein, an HTLV-1 glycoprotein, and a VSV-G glycoprotein; and the target cell is a CD8⁺ T cell. In some cases, the envelope protein of a VLP is a pseudotyping viral glycoprotein, such as, but not limited to, a viral glycoprotein is selected from a HIV-1 envelope, a HTLV-1 glycoprotein, a measles virus hemagglutinin, and a VSV-G glycoprotein; and the target cell is a CD4+ T cell. In some cases, the envelope protein of a VLP is a pseudotyping viral glycoprotein, such as, but not limited to, a Ross River virus glycoprotein or a VSV-G; and the target cell is a skeletal muscle cell. In some cases, the envelope protein of a VLP is a pseudotyping viral glycoprotein, such as, but not limited to, a viral glycoprotein is selected from an Ebola virus glycoprotein, a Marburg virus glycoprotein, and a VSV-G; and the target cell is an ocular cell (e.g., in a retinal cell, a photoreceptor cell, etc.). In some cases, the envelope protein of a VLP is a pseudotyping viral glycoprotein, such as, but not limited to, a viral glycoprotein is selected from an Ebola virus glycoprotein, a Marburg virus glycoprotein, and a VSV-G; and the target cell is an auditory cell (e.g., hair cells, cochlear cells, etc.). In some cases, the envelope protein of a VLP is a pseudotyping viral glycoprotein, such as, but not limited to, a viral glycoprotein is selected from aa rabies glycoprotein, a Mokola virus glycoprotein, a Semliki Forest virus glycoprotein, a Sindbis virus glycoprotein, a Venezuelan equine encephalitis virus glycoprotein, an influenza hemagglutinin glycoprotein, and a VSV-G; and wherein the target cell is a central nervous system cell (e.g., neurons (e.g., excitatory and inhibitory neurons); and glial cells (e.g., oligodendrocytes, astrocytes and microglia)).

### Nucleic Acid Editing Efficiency

In some embodiments, the lipid-containing particle (e.g., viral-like particle) provided herein has an improved nucleic acid editing efficiency when it is contacted with a cell to edit a nucleic acid molecule inside the cell.

For instance, in some cases, the lipid-containing particles provided herein that comprise a fusion protein that includes NES or a cleavage product that includes NES can have a higher nucleic acid editing efficiency, e.g., at least 10%, 20%, 30%, 40%, 50%, 60%, 70%, 80%, 90%, 100%, 120%, 150%, 180%, 200%, 220%, 250%, 280%, 300%, 400%, 500%, 600%, 800%, or 1000% higher, as compared to a corresponding lipid-containing particle that is otherwise the same but does not have an NES in the fusion protein or the cleavage product.

In some cases, the lipid-containing particles provided herein comprise a fusion protein that comprises a cleavable linker located between the sequence of the therapeutic cargo and the NES, and such lipid-containing particles have a higher nucleic acid editing efficiency, e.g., at least 10%, 20%, 30%, 40%, 50%, 60%, 70%, 80%, 90%, 100%, 120%, 150%, 180%, 200%, 220%, 250%, 280%, 300%, 400%, 500%, 600%, 800%, or 1000% higher, as compared to a corresponding lipid-containing particle that is otherwise the same but the sequence of the therapeutic cargo and the NES are located on the same side of the cleavable linker in the fusion protein.

In some cases, the lipid-containing particles provided herein comprise a cleavage product that comprises a sequence of a gag nucleocapsid protein and a nuclear export sequence (NES), and that lacks the therapeutic cargo, and such lipid-containing particles have a higher nucleic acid editing efficiency, e.g., at least 10%, 20%, 30%, 40%, 50%, 60%, 70%, 80%, 90%, 100%, 120%, 150%, 180%, 200%, 220%, 250%, 280%, 300%, 400%, 500%, 600%, 800%, or 1000% higher, as compared to a corresponding lipid-containing particle that is otherwise the same except that in the corresponding lipid-containing particle, the cleavage product comprises the gag nucleocapsid protein, but lacks both the NES and the therapeutic cargo.

In some cases, in the lipid-containing particles provided herein, a ratio of an amount of the therapeutic cargo present within the inside of the protein core versus an amount of the fusion protein is at least 1.5, and such lipid-containing particles have a higher nucleic acid editing efficiency, e.g., at least 10%, 20%, 30%, 40%, 50%, 60%, 70%, 80%, 90%, 100%, 120%, 150%, 180%, 200%, 220%, 250%, 280%, 300%, 400%, 500%, 600%, 800%, or 1000% higher, as compared to a corresponding lipid-containing particle that is otherwise the same except that in the corresponding lipid-containing particle, the ratio of the amount of the therapeutic cargo present within the inside of the protein core versus the amount of the fusion protein is lower than the ratio in the lipid-containing particles provided herein, e.g., lower than 1.5.

Nucleic acid editing efficiency of a lipid-containing particle provided herein can be measured using any suitable techniques known to a skilled artisan in the art. In some cases, it is measured by calculating the percentage of cells in which a target nucleic acid molecule is edited by a given population of lipid-containing particles that is contacted to a population of cells versus the total number of cells in the population. In some cases, the nucleic acid editing efficiency is normalized by an amount of the lipid-containing particles in the population, e.g., the number of the lipid-containing particles, the concentration of the lipid-containing particles, or the volume of a liquid composition comprising the lipid-containing particles that is contacted to the cells. In some cases, the nucleic acid editing efficiency of a lipid-containing particle provided herein is compared with a corresponding lipid-containing particle by an assay in which the nucleic acid editing performance of both lipid-containing particles are tested at a series of different amounts, e.g., numbers, concentrations, or volumes. As such, a dose-response curve can be plotted for the tested lipid-containing particles, and the relative nucleic acid editing efficiency can be derived from such dose-response curves. Exemplary methods of measuring nucleic acid editing efficiency include those described in the Examples of the present disclosure.

### Human Endogenous VLP and Humanized VLP

In some aspects, provided herein are viral-like particles that have reduced or no immunogenicity to human subjects, e.g., non-viral human endogenous viral-like particles (heVLPs), or humanized VLPs that comprise humanized viral components (e.g., humanized viral structural protein or humanized viral envelope protein).

Different from viral-like particles according to some embodiments of the present disclosure, heVLPs or humanized VLPs described herein can package protein cargo by integrating all production DNA into the genomic DNA of production cell lines. Once cell lines are created, protein delivery heVLPs can be produced in a constitutive or inducible fashion. Protein cargos are packaged into heVLP by fusing select human-endogenous GAG proteins or other plasma membrane localization proteins (also termed “plasma membrane recruitment domains” herein) to protein-based cargo.

The heVLP or humanized VLPs systems described herein have the potential to be simpler, more efficient and safer than conventional, artificially-derived lipid/gold nanoparticles and viral particle-based delivery systems because heVLPs or humanized VLPs are comprised of human-derived or humanized components. The cargo inside the particles may or may not be human derived, but the heVLP or humanized VLPs is derived from human or comprises human endogenous components or synthetic non-immunogenic components.

“Synthetic” components include surface scFv/nanobody/darpin peptides that have been demonstrated to not be immunostimulatory and can be used to enhance targeting and cellular uptake of heVLPs. This means that the exterior surface of the particle lacks components that can be significantly immunostimulatory, which can minimize immunogenicity and antibody neutralization of these particles.

In some cases, excluding cargo, the heVLPs provided herein do not contain exogenous viral components inherent to other VLPs and this represents a significant and novel advancement in technology. In addition, heVLPs can utilize (but do not require) chemical-based dimerizers, and heVLPs can have the ability to package and deliver cargo molecules including therapeutic or diagnostic agents, including biomolecules and chemicals, e.g., specialty single and/or double-stranded DNA molecules (e.g., plasmid, mini circle, closed-ended linear DNA, AAV DNA, episomes, bacteriophage DNA, homology directed repair templates, etc.), single and/or double-stranded RNA molecules (e.g., single guide RNA, prime editing guide RNA, messenger RNA, transfer RNA, long non-coding RNA, circular RNA, RNA replicon, circular or linear splicing RNA, micro RNA, small interfering RNA, short hairpin RNA, piwi-interacting RNA, toehold switch RNA, RNAs that can be bound by RNA binding proteins, bacteriophage RNA, internal ribosomal entry site containing RNA, etc.), proteins, chemical compounds and/or molecules (e.g., small molecules), and combinations of the above listed cargos (e.g. AAV particles).

The heVLPs described herein are different from conventional retroviral particles, virus-like particles (VLPs), exosomes and other previously described extracellular vesicles that can be loaded with cargo, at least because heVLPs can be produced by a strategic overexpression of human-derived components in human cells, heVLPs have a vast diversity of possible cargos and loading strategies, heVLPs lack a limiting DNA/RNA length constraint, heVLPs lack proteins derived from pol and exogenous gag, and heVLPs have unique mechanisms of cellular entry.

Described herein are compositions and methods for cargo delivery that can be used with a diverse array of protein and nucleic acid molecules, including genome editing, epigenome modulation, transcriptome editing and proteome modulation reagents, that are applicable to many disease therapies.

In some aspects, provided herein are engineered heVLPs, comprising a membrane comprising a phospholipid bilayer with one or more HERV-derived ENV/glycoprotein(s) (e.g., overexpressed from exogenous sources, such as plasmids or stably integrated transgenes, in heVLP production cells) (e.g., as shown in Table 1) or other human endogenous envelope protein on the external side; and a human endogenous GAG protein, other plasma membrane localization protein (e.g., as shown in Table 2), and/or biomolecule/chemical cargo disposed in the core of the heVLP on the inside of the membrane (e.g., in the lumen enclosed by the phospholipid bilayer).

In some aspects, provided herein are humanized VLPs, comprising a membrane comprising a phospholipid bilayer with one or more HERV-derived ENV/glycoprotein(s) (e.g., overexpressed from exogenous sources, such as plasmids or stably integrated transgenes, in heVLP production cells) (e.g., as shown in Table 1) or other human endogenous envelope protein on the external side; and a viral structural protein (e.g., a retroviral gag protein) on the inside of the membrane (e.g., in the lumen enclosed by the phospholipid bilayer).

In some aspects, provided herein are humanized VLPs, comprising a membrane comprising a phospholipid bilayer with one or more viral envelope proteins disclosed herein; and a human endogenous GAG protein, other plasma membrane localization protein, and/or biomolecule/chemical cargo disposed in the core of the heVLP on the inside of the membrane (e.g., in the lumen enclosed by the phospholipid bilayer).

The cargo may or may not be fused to a human-endogenous GAG or other plasma membrane localization protein. In some cases, the heVLP or humanized VLP does not comprise a non-human gag and/or pol protein. In some cases, the heVLP or humanized VLP does not express gag and/or pol proteins except for gag proteins that are encoded in the human genome or gag proteins that are encoded by a consensus sequence that is derived from gag proteins found in the human genome. Human-derived GAG or other plasma membrane localization proteins fused to cargo can be overexpressed from exogenous sources, such as plasmids or stably integrated transgenes, in heVLP production cells.

Human-endogenous GAG proteins and human pleckstrin homology (PH) domains can localize to biological membranes. PH domains can interact with phosphatidylinositol lipids and proteins within biological membranes, such as PIP2, PIP3, bg-subunits of GPCRs, and PKC. However, in addition to localizing to phospholipid bilayers, human-endogenous GAG proteins can also drive budding and particle formation. This dual functionality of human-endogenous GAG can enable packaging of cargo and budding/formation of particles. One such human-endogenous GAG protein used for this purpose is the human Arc protein that can be fused to protein-based cargo to recruit cargo to the cytosolic side of the phospholipid bilayer. These human-endogenous GAG phospholipid bilayer recruitment domains can be fused to the N-terminus or C-terminus of protein-based cargo via polypeptide linkers of variable length regardless of the location or locations of one or more nuclear localization sequence(s) (NLS) within the cargo. In some cases, the linker between protein-based cargo and the human-endogenous GAG phospholipid bilayer recruitment domain is a polypeptide linker 5-20, e.g., 8-12, e.g., 10, amino acids in length primarily composed of glycines and serines.

TABLE 1
Exemplary HERV envelope proteins

| | | | | Position in | |
|---|---|---|---|---|---|
| | | | | sequence entry | |
| # | HERV envelope | Gene name | Accession no. | (a) | |
| 1. | hENVH1 | envH/p62 | AJ289709.1 | 6313-8067 | (+) |
| 2. | hENVH2 | envH/p60 | AJ289710.2 | 5393-7084 | (+) |
| 3. | hENVH3 | envH/p59 | AJ289711.1 | 5204-6871 | (+) |
| 4. | hENVK1 | envK1 | AC074261.3 | 93508-95604 | (+) |
| 5. | hENVK2 | envK2/HML-2.HOM | AC072054.10 | 30365-32464 | (−) |
| 6. | hENVK3 | envK3/C19 | Y17833.1 | 5581-7680 | (+) |
| 7. | hENVK4 | envK4/K109 | AF164615.1 | 6412-8508 | (+) |
| 8. | hENVK5 | envK5/K113 | AY037928.1 | 6451-8550 | (+) |
| 9. | hENVK6 | envK6/K115 | AY037929.1 | 6442-8541 | (+) |
| 10. | hENVT | envT | AC078899.1 | 154738-156618 | (+) |
| 11. | hENVW | Syncytin-1 | AC000064.1 | 35879-37495 | (+) |
| 12. | hENVFRD | Syncytin-2 | AL136139.6 | 21355-22972 | (−) |
| 13. | hENVR | erv-3 | AC073210.8 | 54963-56978 | (−) |
| 14. | hENVR(b) | envRb | AC093488.1 | 78681-80225 | (+) |
| 15. | hENVF(c)2 | envFc2 | AC016222.4 | 85216-86963 | (+) |
| 16. | hENVF(c)1 | envFc1 | AL354685.2 | 46744-48717 | (−) |

| | | | | |
|---|---|---|---|---|
| *17. | hENVK_{con} | N/A | N/A | N/A |

(a) ‘+’ and ‘−‘ refer to the orientation within the sequence entry
*hENVK_{con} is a consensus sequence derived from ten proviral ENV sequences. The ENV sequences used to derive this consensus ENV sequence are from the following HERVs: HERV-K113, HERV-K101, HERV-K102, HERV-K104, HERV-K107, HERV-K108, HERV-K109, HERV-K115, HERV- K11p22, and HERV-K12q13.

TABLE 2
Exemplary plasma membrane recruitment domain

| # | Plasma Membrane Recruitment Domain | Substitution(s) |
|---|---|---|
| 1 | Pleckstrin homology domain of human | |
| | phospholipase Cδ1 (hPLCδ1) | |
| | Pleckstrin homology domain of Akt1 | E17K |
| | hArc | |
| | hGAGK_{con} | |
| | Pleckstrin homology domain of human 3- | |
| | phosphoinositide-dependent protein kinase 1 | |
| | (hPDPK1) | |
| | Human CD9 | |
| | Human CD47 | |
| | Human CD63 | |
| | Human CD81 | |

The human-endogenous GAG or other phospholipid bilayer recruitment domain can localize the cargo to the phospholipid bilayer and this protein cargo is packaged within heVLPs or humanized VLPs that bud off from the producer cell into extracellular space. In this application, the use of these human-endogenous GAG and other phospholipid bilayer recruitment domains is novel and unique in that these human-endogenous GAG and other proteins can facilitate for localization of cargo to the cytosolic face of the plasma membrane within the heVLP or humanized VLP production cells, and they also allow for cargo to localize to the nucleus of the transduced cells without the utilization of exogenous retroviral GAG or chemical and/or light-based dimerization systems. The heVLP delivery of Cas9, for example, is significantly more efficient with a fusion to a human-endogenous GAG protein compared to a fusion to a PH plasma membrane localization protein or no fusion at all.

heVLPs can also package and deliver a combination of DNA and RNA if heVLPs are produced via transient transfection of a production cell line. DNA that is transfected into cells will possess size-dependent mobility such that a fraction of the transfected DNA will remain in the cytosol while another fraction of the transfected DNA will localize to the nucleus.^{44 46} One fraction of the transfected DNA in the nucleus will expressed components needed to create heVLPs and the other fraction in the cytosol/near the plasma membrane will be encapsulated and delivered in heVLPs.

A combination of exogenous DNA, exogenous RNA, and protein (exogenous and/or endogenous protein) will be referred to as type 1 cargo (T1 heVLPs), exogenous RNA and protein (exogenous and/or endogenous protein) will be referred to as type 2 cargo (T2 heVLPs), a combination of exogenous DNA and proteins (exogenous and/or endogenous protein) will be referred to as type 3 cargo (T3 heVLPs), proteins (exogenous and/or endogenous protein) will be referred to as type 4 cargo (T4 heVLPs). Therefore, T1 contains DNA, RNA, +/−exogenous protein, T2 contains RNA+/−exogenous protein, T3 contains DNA+/−exogenous protein, and T4 is a particle with or without exogenous protein cargo. Hence, T4 without exogenous protein is considered an “empty particle” because there is no “exogenous cargo.” “Exogenous cargo” is cargo not endogenous to the producer cells that can be packaged and/or incorporated into heVLPs. In addition, T1-T4heVLPs can package exogenous chemical molecules in addition to the types of cargoes present in T1-T4heVLPs. RNA in this context, for example, could be single guide RNA (sgRNA), Clustered Regularly Interspaced Palindromic Repeat (CRISPR) RNA (crRNA), and/or mRNA coding for cargo. As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The cargo is limited by the diameter of the particles, e.g., which in some embodiments range from 150 nm to 500 nm.

Other non-limiting examples of heVLPs, human endogenous viral structural proteins, and plasma membrane localization proteins include those described in international publication no. WO 2020/252455, which is incorporated herein by reference in its entirety.

In some embodiments, in order for efficient recruitment of cargo into heVLPs or humanized VLPs, the cargo comprises a covalent or non-covalent connection to a human-endogenous GAG or other plasma membrane recruitment domain, preferably as shown in Table 2. Covalent connections, for example, can include direct protein-protein fusions generated from a single reading frame, inteins that can form peptide bonds, other proteins that can form covalent connections at R-groups and/or RNA splicing. Non-covalent connections, for example, can include DNA/DNA, DNA/RNA, and/or RNA RNA hybrids (nucleic acids base pairing to other nucleic acids via hydrogen bonding interactions), protein domains that dimerize or multimerize with or without the need for a chemical compound/molecule to induce the protein-protein binding (such as DmrA/DmrB/DmrC (Takara Bio), FKBP/FRB, dDZFs, and Leucine zippers), single chain variable fragments, nanobodies, affibodies, proteins that bind to DNA and/or RNA, proteins with quaternary structural interactions, optogenetic protein domains that can dimerize or multimerize in the presence of certain light wavelengths, and/or naturally reconstituting split proteins.

In some embodiments, the cargo comprises a fusion to a dimerization domain or protein-protein binding domain that may or may not require a molecule to trigger dimerization or protein-protein binding.

In some embodiments, the producer cells are FDA-approved cells lines, allogenic cells, and/or autologous cells derived from a donor. In some embodiments, the full or active peptide domains of human CD47 may be incorporated in the heVLP surface to reduce immunogenicity. Examples of AAV proteins included here are AAV REP 52, REP 78, and VP 1-3. The capsid site where proteins can be inserted is T138 starting from the VP1 amino acid counting. Dimerization domains could be inserted at this point in the capsid, for instance. Examples of dimerization domains included here that may or may not need a small molecule inducer are dDZF1, dDZF2, DmrA (Takara Bio), DmrB (Takara Bio), DmrC (Takara Bio), FKBP, FRB, GCN4 scFv, 10×/24× GCN4, GFP nanobody and GFP. Examples of split inteins included here are Npu DnaE, Cfa, Vma, and Ssp DnaE. Examples of other split proteins included here that make a covalent bond together are Spy Tag and Spy Catcher. Examples of RNA binding proteins included here are MS2, Com, and PP7. Examples of synthetic DNA-binding zinc fingers included here are ZF6/10, ZF8/7, ZF9, MK10, Zinc Finger 268, and Zinc Finger 268/NRE. Examples of proteins that multimerize as a result of quaternary structure included here are E. coli ferritin, and the other chimeric forms of ferritin. Examples of optogenetic “light-inducible proteins” included here are Cry2, CIBN, and Lov2-Ja. Examples of peptides the enhance transduction included here are L17E, Vectofusin-1 (Miltenyi Biotec), KALA, and the various forms of nisin.

In another embodiment, T1-T4 heVLPs that are produced and isolated can be loaded with biomolecule or chemical molecule cargo by utilizing nucleofection, lipid, polymer, or CaCh transfection, sonication, freeze thaw, incubation at various temperatures, and/or heat shock of purified particles mixed with cargo. These techniques are adapted from techniques employed to load cargo into exosomes for therapeutic or research applications. For example, 100 μg of heVLPs or humanized VLPs can be resuspended in 200-450 ul of 50 mM trehalose in PBS, mixed with cargo at a desired concentration, and electroporated (GenePulser II Electroporation System with capacitance extender, Bio-Rad, Hercules, CA, USA) in a 0.4 cm cuvette at 0.200 kV and 125 uF.

Preferably heVLPs or humanized VLPs are harvested from cell culture medium supernatant 36-48 hours post-transfection, or when heVLPs or humanized VLPs are at the maximum concentration in the medium of the producer cells (the producer cells are expelling particles into the media and at some point in time, the particle concentration in the media will be optimal for harvesting the particles). Supernatant can be purified by any known methods in the art, such as centrifugation, ultracentrifugation, precipitation, ultrafiltration, and/or chromatography. In some embodiments, the supernatant is first filtered, e.g., to remove particles larger than 1 μm, e.g., through 0.45 pore size polyvinylidene fluoride hydrophilic membrane (Millipore Millex-HV) or 0.8 pm pore size mixed cellulose esters hydrophilic membrane (Millipore Millex-AA). After filtration, the supernatant can be further purified and concentrated, e.g., using ultracentrifugation, e.g., at a speed of 80,000 to 100,000×g at a temperature between 1° C. and 5° C. for 1 to 2 hours, or at a speed of 8,000 to 15,000 g at a temperature between 1° C. and 5° C. for 10 to 16 hours. After this centrifugation step, the heVLPs or humanized VLPs are concentrated in the form of a centrifugate (pellet), which can be resuspended to a desired concentration, mixed with transduction-enhancing reagents, subjected to a buffer exchange, or used as is. In some embodiments, heVLP-containing supernatant or humanized VLP-containing supernatant can be filtered, precipitated, centrifuged and resuspended to a concentrated solution. For example, polyethylene glycol (PEG), e.g., PEG 8000, or antibody-bead conjugates that bind to heVLP or humanized VLP surface proteins or membrane components can be used to precipitate particles.

Purified particles are stable and can be stored at 4° C. for up to a week or −80° C. for years without losing appreciable activity.

Preferably, heVLPs or humanized VLPs are resuspended or undergo buffer exchange so that particles are suspended in an appropriate carrier. In some embodiments, buffer exchange can be performed by ultrafiltration (Sartorius Vivaspin 500 MWCO100,000).

### Exosomes

In some aspects, the delivery vehicles disclosed herein are exosomes. In aspects, disclosed herein are compositions, methods, and systems related to exosomes that can be utilized to deliver cargo into a cell. The term “exosome,” as used herein, can refer to small membrane-bound vesicle (30-100 nm) of endosomal origin. In some cases, exosomes are released into the extracellular environment following fusion of multivesicular bodies with the plasma membrane. In some cases, exosomes described herein are derived from B lymphocytes, dendritic cells (DCs), mesenchymal stromal cells (MSCs), amnion epithelial (AE) cells, and/or placenta-derived cells.

The source cells per the present invention can be select from a wide range of cells, for instance mesenchymal stem or stromal cells or fibroblasts (obtainable from, e.g., bone marrow, adipose tissue, Wharton's jelly, perinatal tissue, tooth buds, umbilical cord blood, skin tissue, etc.), amnion cells and more specifically amnion epithelial cells, myeloid suppressor cells. Generally, both primary cells and cell lines are suitable sources of exosomes. Non-limiting examples include for instance the following: human embryonic kidney (HEK) cells, pericytes, endothelial cells, lymphocytes, endothelial cells and epithelial cells from different organs such as from trachea, lung, GI-tract, urinary tract, etc., dendritic cells (DCs) or other cells from the hematopoietic system such as macrophages, monocytes, B- or T-cells, NK cells, neutrophils, eosinophils, mast cells or basophils, erythrocytes or erythrocyte progenitor cells, thrombocytes and megakaryocytes, etc., cells from different origins such as placenta-derived cells (e.g. decidual placenta cells), syncytiotrophoblasts and amniotic epithelial cells, etc., and cells from CNS and PNS such as microglia, astrocytes, oligodendrocytes and Schwann cells, ependymal cells and nerve cells etc., adipocyte cells from brown or white fat, muscle cells of both smooth muscle and skeletal muscle origin as well as heart muscle cells, to name a few. Generally, exosomes can be derived from essentially any cell source, be it a primary cell source or cell line. The exosome source cells can be any embryonic, fetal, and adult somatic stem cell types, including induced pluripotent stem cells (iPSCs) and other stem or progenitor cells derived by any method. When treating neurological diseases, one may contemplate to utilize as source cells, e.g., primary neurons, astrocytes, oligodendrocytes, microglia, and neural progenitor cells. The source cell can be either allogeneic, autologous, or even xenogeneic in nature to the patient to be treated, i.e., the cells may be from the patient himself or from an unrelated, matched or unmatched donor. In certain contexts, allogeneic cells can be preferable from a medical standpoint, as they could provide immuno-modulatory effects that may not be obtainable from autologous cells of a patient suffering from a certain indication.

In some cases, exosomes are produced by many different types of cells including immune cells such as B lymphocytes, T lymphocytes, dendritic cells (DCs) and most cells. In some cases, exosomes are also produced, for example, by glioma cells, platelets, reticulocytes, neurons, intestinal epithelial cells and tumor cells. In some cases, exosomes for use in accordance with the present application can be derived from any suitable cell, including the cells identified above. Exosomes have also been isolated from physiological fluids, such as plasma, urine, amniotic fluid and malignant effusions.

In some cases, exosomes are derived from immature DCs. In some cases, exosomes produced from immature DCs do not express MHC-II, MHC-I or CD86. As such, such exosomes do not stimulate naïve T cells to a significant extent and are unable to induce a response in a mixed lymphocyte reaction. Thus, exosomes produced from immature dendritic cells can be ideal candidates for use in delivery of a cargo, e.g., a therapeutic cargo.

In some cases, exosomes are obtained from any autologous patient-derived, heterologous haplotype-matched or heterologous stem cells so to reduce or avoid the generation of an immune response in a patient to whom the exosomes are delivered. Any exosome-producing cell can be utilized for this specific purpose.

In some cases, exosomes are produced by many different types of cell and have also been isolated from physiological fluids. Thus, in accordance with the present disclosure, exosomes can be obtained from any suitable cell type as discussed above, or by isolation from physiological fluids. Typically, the methods of the present invention comprise isolation of the exosomes from cell culture medium or tissue supernatant.

Exosomes produced from cells can be collected from the culture medium by any suitable method. Typically, a preparation of exosomes can be prepared from cell culture or tissue supernatant by centrifugation, filtration or combinations of these methods. For example, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 μm filter), gradient ultracentrifugation (for example, with sucrose gradient) or a combination of these methods.

In some cases, the exosomes are loaded with a cargo, e.g., a therapeutic cargo, e.g., a protein, nucleic acid molecule, or small molecule. In some cases, exosomes are prepared and then loaded with the desired therapeutic cargo for delivery.

In some aspects, the exosomes disclosed herein are engineered to target a desired cell type or tissue. This targeting can be achieved by expressing on the surface of the exosome a targeting moiety which binds to a cell surface moiety expressed on the surface of the cell to be targeted. In some cases, the targeting moiety is a peptide which is expressed as a fusion protein with a transmembrane protein typically expressed on the surface of the exosome.

In some cases, the exosomes are targeted to particular cell types or tissues by expressing on their surface a targeting moiety such as a peptide. Suitable peptides are those which bind to cell surface moieties such as receptors or their ligands found on the cell surface of the cell to be targeted. Non-limiting examples of suitable targeting moieties are short peptides, scFv and complete proteins, so long as the targeting moiety can be expressed on the surface of the exosome and does not interfere with insertion of the membrane protein into the exosome.

Typically, the targeting peptide is heterologous to the transmembrane exosomal protein. Peptide targeting moieties may typically be less than 100 amino acids in length, for example less than 50 amino acids in length, less than 30 amino acids in length, to a minimum length of 10, 5 or 3 amino acids.

Targeting moieties can be selected to target particular tissue types such as muscle, brain, liver, pancreas and lung for example, or to target a diseased tissue such as a tumor. In a particularly preferred embodiment of the present invention, the exosomes are targeted to brain tissue.

Specific non-limiting examples of targeting moieties include muscle specific peptide, discovered by phage display, to target skeletal muscle, a 29 amino acid fragment of Rabies virus glycoprotein that binds to the acetylcholine receptor or a fragment of neural growth factor that targets its receptor to target neurons and secretin peptide that binds to the secretin receptor can be used to target biliary and pancreatic epithelia. As an alternative, immunoglobulins and their derivatives, including scFv antibody fragments can also be expressed as a fusion protein to target specific antigens, such as VEGFR for cancer gene therapy. As an alternative, natural ligands for receptors can be expressed as fusion proteins to confer specificity, such as NGF which binds NGFR and confers neuron-specific targeting.

The peptide targeting moiety can be expressed on the surface of the exosome by expressing it as a fusion protein with an exosomal transmembrane protein. A number of proteins are known to be associated with exosomes; that is, they are incorporated into the exosome as it is formed. In some cases, the targeting moiety include or is derived from those which are transmembrane proteins. Examples include but are not limited to Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta and tetraspanins. In particularly preferred embodiments of the present invention, the transmembrane protein is selected from Lamp-1, Lamp-2, CD13, CD86, Flotillin, Syntaxin-3. In some cases, the targeting moiety includes or is derived from variations, alterations, modifications or derivatizations of amino acid sequence of the proteins discussed above. It will be understood that such variations, alterations, modifications or derivatizations of polypeptides as are described herein are subject to the requirement that the polypeptides retain any further required activity or characteristic as may be specified subsequent sections of this disclosure.

A targeting moiety can include, e.g., an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Cell fusion proteins may be re-targeted by non-covalently conjugating a targeting moiety to the fusion protein or targeting protein (e.g., the hemagglutinin protein). For example, the fusion protein can be engineered to bind the Fc region of an antibody that targets an antigen on a target cell, redirecting the fusion activity towards cells that display the antibody's target.

A targeting moiety can comprise, e.g., a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE® s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR® s.

In embodiments, the targeting moiety linked to the membrane protein binds a cell surface marker on the target cell, e.g., a protein, glycoprotein, receptor, cell surface ligand, agonist, lipid, sugar, class I transmembrane protein, class II transmembrane protein, or class III transmembrane protein.

In some cases, the targeting moiety is introduced into the exosome by expressing the fusion protein comprising the targeting moiety and exosomal transmembrane protein within a cell used to produce the exosomes. Expression of this fusion protein in the cell, allows for the fusion protein to be incorporated into the exosome as it is produced from the cell.

In some cases, the targeting moiety disclosed herein that is applicable for exosomes can also be used for other lipid-containing particles disclosed herein, e.g., viral-like particles, lipid nanoparticles, and proteo-lipid vehicles.

For example, a polynucleotide construct such as a DNA plasmid, which expressed the fusion protein is transfected into the cell. Any suitable method can be used for introduction of the polynucleotide construct into the cell. The polynucleotide construct includes suitable promoter sequences so that the encoded fusion protein is expressed in the cell. Signal peptide sequences are also included so that the protein is incorporated into the membrane of the endoplasmic reticulum as it is produced. The membrane protein is then subsequently exported to the exosomal/lysosomal compartment before incorporation into the exosome. The signal sequence is typically a signal peptide sequence for an exosomal transmembrane protein.

In some cases, exosomes produced from cells can be collected from the culture medium by any suitable method. Typically, a preparation of exosomes can be prepared from cell culture or tissue supernatant by centrifugation, filtration or combinations of these methods. For example, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 μm filter), gradient ultracentrifugation (for example, with sucrose gradient) or a combination of these methods.

In some cases, a specific targeting moiety does not need to be included in the exosome. For example, exosomes may be administered directly to the site where therapy is required. Alternatively, for example, where exosomes contain genetic material encoding immunogens, direct targeting to a specific site may not be required and delivery, for example, intradermal or muscular delivery may be sufficient to generate the desired immune response without targeting exosomes to any specific cell type. In some cases, no targeting moiety is included on the surface of the exosomes. However, the exosomes are selected such that they are more likely to target a specific tissue type. For example, exosomes derived from different cells may have natural affinities for specific cell subtypes as required by their physiological function such as the well-established affinity of mature dendritic cell-derived exosomes to T-cells. This affinity may be utilized to specifically deliver above-mentioned cargo to a tissue.

In some cases, exosomes are produced from a cell that is modified to express chimeric polypeptide receptor, e.g., a chimeric antigen receptor (CAR). In some cases, exosomes are produced from a cell genetically modified to produce a chimeric polypeptide receptor comprising (i) an extracellular recognition domain, (ii) at least one protease cleavage site, and (iii) an intracellular transcription factor, wherein binding of the extracellular recognition domain to its target induces proteolytic cleavage of the at least one protease cleavage site and endogenous transcription by the intracellular transcription factor of at least one polynucleotide encoding a gene product comprising at least one exosomal polypeptide. In some cases, the gene product further comprises a protein of interest, for instance, an antibody, a single-chain antibody or any other antibody derivative, a bispecific T cell engager (BiTE), a receptor, a cytokine such as an interleukin, an enzyme such as caspase, granzyme, Cas, Cas9, a checkpoint inhibitor, a costimulation inhibitor, an RNA-binding protein, a membrane transporter such as NPC-1, a splicing factor, a protein associated with cellular organelles, a lysosomal enzyme, a transcription factor, a mitochondrial proteins, an intracellular protein, an antiviral protein, an antibacterial protein. In some cases, when the protein of interest is an RNA-binding protein, the cell, from which exosomes are produced, is further genetically modified to comprise an RNA cargo molecule selected from the group consisting of mRNA, sgRNA, shRNA, miRNA, shRNA, siRNA, lncRNA, ncRNA, piRNA, piwiRNA, circRNA, tRNA, rRNA, crRNA and any combination thereof. In some cases, the genetic modification is an in vitro or ex vivo genetic modification. In some cases, the cell, from which exosomes are produced, is an effector immune cell, such as a T cell, a cytotoxic CD8+ T cell, a CD4+ T cell, a regulatory T cell, a natural killer (NK) cell, a B cell, a plasma cell, a dendritic cell (DC), a macrophage, a monocyte, a neutrophil, an epithelial cell, an endothelial cell, a microglia, an astrocyte, a neuron, a stem cell, a bone marrow derived mesenchymal stromal cell, a Wharton's jelly derived MSC, or any other cell type. In some cases, the extracellular recognition domain of the chimeric polypeptide receptor is an antibody, an antibody derivative, a single-chain fragment, a single-chain antibody, a nanobody, a peptide, a ligand for a receptor, an adhesion molecule, a receptor, an interleukin receptor, an extracellular matrix component, or any combination thereof. In some cases, the at least one protease cleavage site is at least one of an S1, an S2 and/or an S3 cleavage site. In some cases, the fusion polypeptide is a chimeric Notch polypeptide comprising from N-terminus to C-terminus and in covalent linkage: (i) an extracellular recognition domain that is not naturally present in a Notch receptor polypeptide; (ii) a Notch regulatory region which comprises a Lin 12-Notch repeat, an S2 proteolytic cleavage site, and a transmembrane domain comprising an S3 proteolytic cleavage site; (iii) an intracellular transcription factor that is heterologous to the Notch regulatory region, wherein binding of the extracellular recognition domain to its target induces cleavage at the S2 and S3 protease cleavage sites, thereby releasing the intracellular transcription factor which activates transcription of the polynucleotide. In some cases, the Notch regulatory region further comprises a heterodimerization domain comprising the S2 proteolytic cleavage site. In some cases, the S1 proteolytic cleavage site is a furin-like protease cleavage site comprising the amino acid sequence Arg-X-(Arg/Lys)-Arg, where X is any amino acid. In some cases, the fusion polypeptide comprises at least one linker. In some cases, the polynucleotide further comprises a transcriptional control element, responsive to the transcription factor, operably linked to a coding sequence. In some cases, the cell is genetically modified to produce at least two types of fusion polypeptides, wherein at least one of the (i) extracellular recognition domain, the (ii) protease cleavage site, and the (iii) intracellular transcription factor differ between the fusion polypeptides. In some cases, the extracellular recognition domains of the fusion polypeptides are different from one another.

In some cases, loading of the exosomes disclosed herein with a protein cargo is achieved by expressing a tri-domain polypeptide construct in a source cell from which exosomes are produced. In some cases, such polypeptide constructs comprise (i) at least one protein of interest (POI), (ii) at least one multimerization domain, and (iii) at least one exosomal sorting domain. The design of the tri-domain polypeptide construct can enable highly efficient loading of a POI into an exosome, and also drives increased production of exosomes from source cells.

The multimerization polypeptide domain is an important component to achieve this increasing loading of the resultant exosomes, and such multimerization domains may interestingly be selected from a large variety of different species and may also display relatively different mechanisms of action (e.g., it may be a hetero-dimerization domain, or it may be a homo-trimerization domain, or a homopentameric domain, etc.). In some cases, the multimerization domains are homo-multimerization domains, as these enable a simple design of the fusion proteins and importantly supports controlled loading of one single type of fusion polypeptide constructs into exosomes (as opposed to multiple fusion constructs). The multimerization domains can be either dimerization domains, trimerization domains, tetramerization domains, or essentially any higher order of multimerization domains, as long as the domain is capable of facilitating interaction of at least two domains (and the polypeptides of which they form part). For instance, a non-limiting list of multimerization domains comprises the following domains: leucine zipper homodimerization domain of GCN4 from S. cerevisiae, Retro-Leucine zipper homodimerization domain of GCN4 from S. cerevisiae, Fold-on homodimerization domain of Fibritin (from the T4 bacteriophage), Fragment X heteromerization domain of Phosphoprotein (from human respiratory syncytial virus A), human alpha helical coiled coil oligomerization domain of collagen superfamily, leucine zipper heterodimerization domain of Fos and Jun (human), transmembrane homopentameric domain of Cardiac phospholamban (human), homodimerization domain of parathyroid hormone (human), transmembrane homodimeric domain of Glycophorin A (human), trimerization domain of Gp41 (from HIV), C-terminal Homodimeric domain of oncoprotein E7 (from HPV 45), and EVH2 homotetramer domain of Vasodilator-stimulated phosphoprotein (human), mitochondrial antiviral-signaling protein CARD filament and/or any combination thereof.

The multimerization domain can be placed in several different locations in the polypeptide construct. For instance, the multimerization domain can be placed between the POI sequence and exosomal sorting domain sequence, within or adjacent to the exosomal sorting domain sequence, and/or within or adjacent to the POI sequence. Overall, the design of the tri-domain polypeptide construct (with regard to both the choice of multimerization domain and its location in the construct, and with regard to the choice of exosomal sorting domain and its location in the construct) is important for determining where in the exosomes that the polypeptide ends up after production in an exosome source cell. By selecting, e.g., a tetraspanin exosomal sorting protein (e.g., CD9, CD63 or CD81) or any other exosome membrane protein (such as Lamp2b) it is possible to enrich for the POI on the exosome surface. Conversely, selecting an exosomal sorting protein typically present in the exosome lumen, such as ALIX or syntenin, enables enriching for the polypeptide construct (and thereby the POI) essentially inside the exosome interior. Naturally, the polypeptide constructs may be present simultaneously on the outside and on the inside of the exosomes, as well as in the exosome membrane. Furthermore, in preferred embodiments, the fusion polypeptide constructs may comprise various types of linkers between the different domains, i.e., between the at least one POI, the at least one multimerization domain, and the at least one exosomal sorting domain. The linker may for instance be a GS (i.e., glycine-serine) linker, i.e., a linker comprising the amino acids glycine and serine, or any other type of suitable linker domain that ensures that the activity of the different domains is not restricted when they are present in a fusion polypeptide construct.

A typical tri-domain fusion polypeptide construct as per the present invention can be described schematically as follows (the below notation is not to be construed as illustrating any C and/or N terminal direction, it is merely meant for illustrative purposes): POI-Multimerization Domain-Exosomal Sorting Domain

The exosomal sorting domains of the present disclosure can be selected from any one of the following proteins: CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLLA, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc Receptors, Interleukin receptors, Immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, LICAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, and any combinations thereof.

In some cases, the exosome is loaded with aid of cell penetrating peptides, such as those described in U.S. Patent Publication No. US20190388347, which is incorporated herein by reference in its entirety.

Non-limiting examples of exosomes, source cells from which exosomes are produced, cargos that can be delivered in an exosome, methods of loading exosomes with cargos, and methods of producing the exosomes include those described in U.S. Patent Publication Nos. US20070298118, US20180177727, US20200062813, US20200206360, US20200023012, US20160137716, US20170173113, US20130053426, US20190167810, US20190388347, US20190224331, US20160137716, US20210188903, US20210069254, and US20200407418, each of which is incorporated herein by reference in its entirety.

### Lipid Nanoparticles or Proteo-Lipid Vehicles

In some aspects, disclosed herein are compositions, methods, and systems related to lipid nanoparticles that can be utilized to deliver cargo into a cell. In some aspects, disclosed herein are compositions, methods, and systems related to proteo-lipid vehicles that can be utilized to deliver cargo into a cell.

Lipid nanoparticles can provide a biocompatible and biodegradable delivery system for therapeutic cargos disclosed herein. In some cases, the lipid nanoparticles disclosed herein comprise nanostructured lipid carriers (NLCs), polymer nanoparticles (PNPs), or lipid-polymer nanoparticles (PLNs). NLCs are modified solid lipid nanoparticles (SLNs) that retain the characteristics of the SLN, improve drug stability and loading capacity, and prevent drug leakage. Polymer nanoparticles (PNPs) are an important component of therapeutic delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs.

Non-limiting examples of lipid nanoparticles disclosed herein include those described in JA Zuris et al., Nat Biotechnol. 2014 Oct. 30; 33(1):73-80; Hou et al. Lipid nanoparticles for mRNA delivery. Nat Rev Mater (2021); WO2019/067992, WO2017/173054, WO2015/095340, WO2014/136086, and WO2019/217941, each of which is incorporated herein by reference in its entirety.

### Cargo

“Cargo” as used herein can refer to a one or more of chemicals, e.g., small molecule compounds, combination of DNA, RNA, and protein, a combination of RNA and protein, a combination of DNA and protein, or protein, e.g., for therapeutic or diagnostic use, or for the applications of genome editing, epigenome modulation, and/or transcriptome modulation. In addition, endogenous RNA and protein from a producer cells can get packaged and/or incorporated into delivery vehicles (e.g., VLPs, e.g., heVLPs or humanized VLPs).

In some embodiments, the cargo contained in and to be delivered by the delivery vehicles disclosed herein comprises a polypeptide, e.g., a nuclear transport polypeptide, a nucleic acid binding polypeptide, a reprogramming polypeptide, a DNA editing polypeptide, a DNA repair polypeptide, a DNA recombination polypeptide, a transposase polypeptide, a DNA integration polypeptide, a targeted endonuclease (e.g., a Zinc-finger nuclease (ZFN), a transcription-activator-like nuclease (TALENs), Cas9 or a homolog thereof), a recombinase, an enzyme, a structural polypeptide, a signaling polypeptide, a regulatory polypeptide, a transport polypeptide, a sensory polypeptide, a motor polypeptide, a defense polypeptide, a storage polypeptide, a transcription factor, an antibody, a cytokine, a hormone, a catabolic polypeptide, an anabolic polypeptide, a proteolytic polypeptide, a metabolic polypeptide, a kinase, a transferase, a hydrolase, a lyase, an isomerase, a ligase, an enzyme modulator polypeptide, a protein binding polypeptide, a lipid binding polypeptide, a membrane fusion polypeptide, a cell differentiation polypeptide, an epigenetic polypeptide, a cell death polypeptide, or any combination thereof. In some embodiments, the cargo contained in the delivery vehicles disclosed herein comprises a protein that targets a protein in the cell for degradation. In some cases, the cargo contained in the delivery vehicles disclosed herein comprises a chimeric antigen receptor (CAR), an antibody, a T cell receptor, or a functional fragment thereof, or any combination thereof.

In some embodiments, the cargo contained in and to be delivered by the delivery vehicles disclosed herein comprises a polynucleotide, e.g., a deoxyribonucleic acid (DNA) or a ribonucleic acid (RNA) molecule. In some cases, the polynucleotide encodes a polypeptide such as those described in the paragraph above. In some cases, the polynucleotide comprises a napR/DNAbp-programming nucleic acid molecule described below.

In some embodiments, the cargo contained in and to be delivered by the delivery vehicles disclosed herein comprises a ribonucleoprotein (RNP) complex that is formed between one or more proteins and one or more polynucleotides. For instance, the cargo can comprise a RNP complex formed by a nucleic acid programmable R/DNA binding protein (napR/DNAbp) described below and a napR/DNAbp-programming nucleic acid molecule, e.g., a Cas protein and a guide RNA.

In some embodiments, the cargo contained in and to be delivered by the delivery vehicles disclosed herein comprises other therapeutic molecules, such as ribozymes, aptamers, aptazymes, peptides, oligonucleotides, antibody mimetics, peptide mimetics, antibody-drug conjugates, antibiotics, carbohydrate, ribosomes, mitochondria, and small molecule compounds.

In some embodiments, the cargo contained in and to be delivered by the delivery vehicles disclosed herein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein. In some embodiments, the protein is a mutant protein. In some embodiments the protein is a fusion or chimeric protein.

In some cases, the cargo contained in and to be delivered by the delivery vehicles disclosed herein comprises decoy proteins for binding to dis-ease-causing target proteins; peptides or proteins for inducing endosomal escape, such as HA2; peptides or proteins for targeting the exosome to a tissue or organ or cell type of interest; antibodies, intrabodies, single chain variable fragments (scFv), affibodies, bispecific or multispecific antibodies or binders, receptors, etc.; enzymes such as alpha-glucosidase and/or glucocerebrosidase for enzyme re-placement therapy; transport proteins such as NPC1 or cystinosin; peptides or proteins for optimizing the in vivo behavior of exosomes (e.g. their circulation time or immune system recognition), e.g. CD47 and/or CD55 or parts of these proteins; cytokines or chemokines; a targeting peptide or protein, such as an RVG peptide, a VSV-G peptide, a p-selectin binding peptide, or an e-selectin binding peptide; a cell-penetrating peptide (CPP) (e.g., Tat, penetratin, TP10, CADY); or tumor suppressors.

In some cases, the cargo contained in and to be delivered by the delivery vehicles disclosed herein comprises an immunogenic molecule, such as a vaccine. The vaccine can be a peptide antigen, RNA (e.g., mRNA or circRNA), DNA (e.g., a DNA molecule encoding an antigen). The cargo can also include an adjuvant that enhances immunogenicity of the vaccine composition.

In some cases, a cargo protein loaded in the delivery vehicle functions to bind to another cargo molecule (e.g., nucleic acid molecule, protein, RNP, etc.) to be delivered by the delivery vehicle.

In some embodiments, the cargo contained in and to be delivered by the delivery vehicles disclosed herein includes a small molecule, e.g., ions (e.g., Ca²⁺, Cl⁻, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical agent that interacts with a target in the cell. In some embodiments, the small molecule targets a protein in the cell for degradation. In some embodiments, the small molecule targets a protein in the cell for degradation by localizing the protein to the proteasome. In some embodiments, the small molecule is a proteolysis targeting chimera molecule (PROTAC).

In some embodiments, the cargo contained in and to be delivered by the delivery vehicles disclosed herein includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the cargo contained in and to be delivered by the delivery vehicles disclosed herein includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, networks of organelles, and any combination thereof.

In some cases, the cargo contained in and to be delivered by the delivery vehicles disclosed herein comprises one or more of RNA (viral or heterologous), DNA (single-stranded, double-stranded), Green fluorescent protein, Nuclease, Iron oxide NP (IONP), Taxol, Alexa Fluor® 488, Porphyrin, Doxorubicin, Fluorescein, DOTA chelators, RNA (messenger, micro, small-interfering), Ricin toxin A-chain, HIV-1 Tat peptide, Alkaline phosphatase, Green fluorescent protein, Quantum dot 585, Methacrylate (monomers, polymers), CpG DNA, Fluorescent proteins, Luciferase, Nickel, Biotin, Fluorescein polymethacrylate, Gadopentetic acid polymethacrylate, CRISPR (Cas9 and guide RNA), Green fluorescent protein or mCherry, CellB protein, [NiFe] hydrogenase, Ziconotide peptide, Three enzyme cascade (genetically linked), Alcohol dehydrogenase, Polystyrene sulfonate, RNA, Green or teal fluorescent Protein, Pseudozyma antarctica lipase B, Horseradish peroxidase, DOTAC10 micelles with Gd(III) or Zn(II), Gd(DOTA), Fluorescent probes, Doxorubicin, DAPI, Acridine orange, Propidium iodide, Proflavin, Iron oxide NP, Gd(III), or Tb(III). In some cases, the cargo contained in the delivery vehicles disclosed herein comprises those described in Rohovie, M. J., et al., Bioengineering & Translational Medicine, 2:43-57, which is incorporated herein by reference in its entirety.

In some cases, the cargo contained in and to be delivered by the delivery vehicles of the present disclosure comprises a polypeptide that has a length of at least 10 amino acids (aa), at least 20 aa, at least 30 aa, at least 50 aa, at least 80 aa, at least 100 aa, at least 150 aa, at least 200 aa, at least 250 aa, at least 300 aa, at least 350 aa, at least 400 aa, at least 500 aa, at least 600 aa, at least 700 aa, at least 800 aa, at least 900 aa, at least 1000 aa, at least 1200 aa, at least 1400 aa, at least 1500 aa, at least 1800 aa, at least 2000 aa, at least 2500 aa, at least 3000 aa, at least 4000 aa, or at least 5000 aa. In some cases, the cargo contained in the delivery vehicles of the present disclosure comprises a polypeptide that has a length of about 20 aa, about 30 aa, about 50 aa, about 80 aa, about 100 aa, about 150 aa, about 200 aa, about 250 aa, about 300 aa, about 350 aa, about 400 aa, about 500 aa, about 600 aa, about 700 aa, about 800 aa, about 900 aa, about 1000 aa, about 1200 aa, about 1400 aa, about 1500 aa, about 1800 aa, about 2000 aa, about 2500 aa, about 3000 aa, about 4000 aa, or about 5000 aa.

In some cases, the cargo contained in and to be delivered by the delivery vehicles disclosed herein comprises a polynucleotide encoding a polypeptide that has a length of at least 20 aa, at least 30 aa, at least 50 aa, at least 80 aa, at least 100 aa, at least 150 aa, at least 200 aa, at least 250 aa, at least 300 aa, at least 350 aa, at least 400 aa, at least 500 aa, at least 600 aa, at least 700 aa, at least 800 aa, at least 900 aa, at least 1000 aa, at least 1200 aa, at least 1400 aa, at least 1500 aa, at least 1800 aa, at least 2000 aa, at least 2500 aa, at least 3000 aa, at least 4000 aa, or at least 5000 aa. In some cases, the cargo contained in the delivery vehicles of the present disclosure comprises a polynucleotide encoding a polypeptide that has a length of about 20 aa, about 30 aa, about 50 aa, about 80 aa, about 100 aa, about 150 aa, about 200 aa, about 250 aa, about 300 aa, about 350 aa, about 400 aa, about 500 aa, about 600 aa, about 700 aa, about 800 aa, about 900 aa, about 1000 aa, about 1200 aa, about 1400 aa, about 1500 aa, about 1800 aa, about 2000 aa, about 2500 aa, about 3000 aa, about 4000 aa, or about 5000 aa.

In some cases, the polypeptide contained in and to be delivered by the delivery vehicles disclosed herein forms a protein that is at least 1 kDa, at least 2 kDa, at least 5 kDa, at least 10 kDa, at least 15 kDa, at least 20 kDa, at least 25 kDa, at least 30 kDa, at least 35 kDa, at least 40 kDa, at least 50 kDa, at least 60 kDa, at least 70 kDa, at least 80 kDa, at least 100 kDa, at least 120 kDa, at least 150 kDa, at least 180 kDa, at least 200 kDa, at least 220 kDa, at least 250 kDa, at least 280 kDa, at least 300 kDa, at least 320 kDa, at least 350 kDa, at least 400 kDa, at least 500 kDa, at least 600 kDa, at least 700 kDa, at least 800 kDa, at least 900 kDa, or at least 1000 kDa. In some cases, the polypeptide contained in and to be delivered by the delivery vehicles disclosed herein forms a protein that is about 1 kDa, about 2 kDa, about 5 kDa, about 10 kDa, about 15 kDa, about 20 kDa, about 25 kDa, about 30 kDa, about 35 kDa, about 40 kDa, about 50 kDa, about 60 kDa, about 70 kDa, about 80 kDa, about 100 kDa, about 120 kDa, about 150 kDa, about 180 kDa, about 200 kDa, about 220 kDa, about 250 kDa, about 280 kDa, about 300 kDa, about 320 kDa, about 350 kDa, about 400 kDa, about 500 kDa, about 600 kDa, about 700 kDa, about 800 kDa, about 900 kDa, or about 1000 kDa.

In some cases, the cargo contained in and to be delivered by the delivery vehicles disclosed herein comprises a single-stranded polynucleotide that has a length of at least 50 nucleotides, at least 80 nucleotides, at least 100 nucleotides, at least 150 nucleotides, at least 200 nucleotides, at least 250 nucleotides, at least 300 nucleotides, at least 350 nucleotides, at least 400 nucleotides, at least 500 nucleotides, at least 600 nucleotides, at least 700 nucleotides, at least 800 nucleotides, at least 900 nucleotides, at least 1000 nucleotides, at least 1200 nucleotides, at least 1400 nucleotides, at least 1500 nucleotides, at least 1800 nucleotides, at least 2000 nucleotides, at least 2500 nucleotides, at least 3000 nucleotides, at least 4000 nucleotides, at least 5000 nucleotides, at least 6000 nucleotides, at least 8000 nucleotides, at least 10000 nucleotides, at least 12000 nucleotides, at least 14000 nucleotides, or at least 15000 nucleotides. In some cases, the cargo contained in the delivery vehicles of the present disclosure comprises a single-stranded polynucleotide encoding a polypeptide that has a length of about 20 nucleotides, about 30 nucleotides, about 50 nucleotides, about 70 nucleotides, about 80 nucleotides, about 100 nucleotides, about 120 nucleotides, about 150 nucleotides, about 200 nucleotides, about 250 nucleotides, about 300 nucleotides, about 350 nucleotides, about 400 nucleotides, about 500 nucleotides, about 600 nucleotides, about 700 nucleotides, about 800 nucleotides, about 900 nucleotides, about 1000 nucleotides, about 1200 nucleotides, about 1400 nucleotides, about 1500 nucleotides, about 1800 nucleotides, about 2000 nucleotides, about 2500 nucleotides, about 3000 nucleotides, about 4000 nucleotides, about 5000 nucleotides, about 6000 nucleotides, about 8000 nucleotides, about 10000 nucleotides, about 12000 nucleotides, about 14000 nucleotides, or about 15000 nucleotides.

In some cases, the cargo contained in and to be delivered by the delivery vehicles disclosed herein comprises a double-stranded polynucleotide that has a length of at least 50 nucleotides, at least 80 nucleotides, at least 100 base pairs (bp), at least 150 bp, at least 200 bp, at least 250 bp, at least 300 bp, at least 350 bp, at least 400 bp, at least 500 bp, at least 600 bp, at least 700 bp, at least 800 bp, at least 900 bp, at least 1000 bp, at least 1200 bp, at least 1400 bp, at least 1500 bp, at least 1800 bp, at least 2000 bp, at least 2500 bp, at least 3000 bp, at least 4000 bp, at least 5000 bp, at least 6000 bp, at least 8000 bp, at least 10000 bp, at least 12000 bp, at least 14000 bp, or at least 15000 bp. In some cases, the cargo contained in the delivery vehicles of the present disclosure comprises a double-stranded polynucleotide encoding a polypeptide that has a length of about 20 bp, about 30 bp, about 50 bp, about 70 bp, about 80 bp, about 100 bp, about 120 bp, about 150 bp, about 200 bp, about 250 bp, about 300 bp, about 350 bp, about 400 bp, about 500 bp, about 600 bp, about 700 bp, about 800 bp, about 900 bp, about 1000 bp, about 1200 bp, about 1400 bp, about 1500 bp, about 1800 bp, about 2000 bp, about 2500 bp, about 3000 bp, about 4000 bp, about 5000 bp, about 6000 bp, about 8000 bp, about 10000 bp, about 12000 bp, about 14000 bp, or about 15000 bp.

In some cases, the cargo contained in and to be delivered by the delivery vehicles disclosed herein does not comprise a nuclease, a reverse trancriptase, a base editor, or a prime editor.

### Nucleases

Any suitable nuclease can be delivered by the delivery vehicles disclosed herein that either contain the nuclease or a polynucleotide encoding the nuclease. In some embodiments, a nuclease delivered by the delivery vehicles disclosed herein (e.g., lipid-containing particles) is not a nucleic acid-programmable DNA-binding protein (napDNAbp) (e.g., a Cas protein programmable by a guide RNA, such as a Cas9 protein). Suitable nucleases include, but are not limited to, CRISPR-associated (Cas) proteins or Cas nucleases including type I CRISPR-associated (Cas) polypeptides, type II CRISPR-associated (Cas) polypeptides (e.g., Cas9 or Cas14), type III CRISPR-associated (Cas) polypeptides, type IV CRISPR-associated (Cas) polypeptides, type V CRISPR-associated (Cas) polypeptides (e.g., Cpf1/Cas12a, C2c1, or c2c3), and type VI CRISPR-associated (Cas) polypeptides (e.g., C2c2/Cas13a, Cas13b, Cas13c, Cas13d); zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof; any variant thereof; and any fragment thereof.

In some embodiments, the cargo in the delivery vehicles disclosed herein comprises or encodes a CRISPR-associated (Cas) protein or a Cas nuclease which functions in a non-naturally occurring CRISPR (Clustered Regularly Interspaced Short Palindromic Repeats)/Cas (CRISPR-associated) system. In bacteria, this system can provide adaptive immunity against foreign DNA (Barrangou, R., et al, “CRISPR provides acquired resistance against viruses in prokaryotes,” Science (2007) 315:1709-1712; Makarova, K. S., et al, “Evolution and classification of the CRISPR-Cas systems,” Nat Rev Microbiol (2011) 9:467-477; Garneau, J. E., et al, “The CRISPR/Cas bacterial immune system cleaves bacteriophage and plasmid DNA,” Nature (2010) 468:67-71; Sapranauskas, R., et al, “The Streptococcus thermophilus CRISPR/Cas system provides immunity in Escherichia coli,” Nucleic Acids Res (2011) 39:9275-9282).

One or more components of a CRISPR/Cas system (e.g., modified and/or unmodified) delivered by the delivery vehicles disclosed herein can be utilized as a genome engineering tool in a wide variety of organisms including diverse mammals, animals, plants, and yeast. A CRISPR/Cas system can comprise a guide nucleic acid such as a guide RNA (gRNA) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al, “Cas9-crRNA ribonucleoprotein complex mediates specific DNA cleavage for adaptive immunity in bacteria,” Proc Natl Acad Sci USA (2012) 109: E2579-E2 86; Jinek, M., et al, “A programmable dual-RNA-guided DNA endonuclease in adaptive bacterial immunity,” Science (2012) 337:816-821; Sternberg, S. H., et al, “DNA interrogation by the CRISPR RNA-guided endonuclease Cas9,” Nature (2014) 507:62; Deltcheva, E., et al, “CRISPR RNA maturation by trans-encoded small RNA and host factor RNase III,” Nature (2011) 471:602-607), and has been widely used for programmable genome editing in a variety of organisms and model systems (Cong, L., et al, “Multiplex genome engineering using CRISPR Cas systems,” Science (2013) 339:819-823; Jiang, W., et al, “RNA-guided editing of bacterial genomes using CRISPR-Cas systems,” Nat. Biotechnol. (2013) 31:233-239; Sander, J. D. & Joung, J. K, “CRISPR-Cas systems for editing, regulating and targeting genomes,” Nature Biotechnol. (2014) 32:347-355).

In some cases, the Cas protein delivered by the delivery vehicles of the present disclosure is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity. A cargo protein or a protein encoded by a cargo nucleic acid molecule comprising a Cas nuclease (e.g., retaining wild-type nuclease activity, having reduced nuclease activity, and/or lacking nuclease activity) can function in a CRISPR/Cas system to regulate the level and/or activity of a target gene or protein (e.g., decrease, increase, or elimination). The Cas protein can bind to a target polynucleotide and prevent transcription by physical obstruction or edit a nucleic acid sequence to yield non-functional gene products.

In some embodiments, the cargo in the delivery vehicles disclosed herein comprises or encodes a Cas protein that forms a complex with a guide nucleic acid, such as a guide RNA (gRNA). In some embodiments, the cargo in the delivery vehicles disclosed herein comprises or encodes a Cas protein that forms a complex with a single guide nucleic acid, such as a single guide RNA (sgRNA). In some embodiments, the cargo in the delivery vehicles disclosed herein comprises or encodes an RNA-binding protein (RBP) optionally complexed with a guide nucleic acid, such as a guide RNA (e.g., sgRNA), which is able to form a complex with a Cas protein.

One or more components of any suitable CRISPR/Cas system can be delivered by the delivery vehicles of the present disclosure. A CRISPR/Cas system can be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3, or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

A cargo delivered by the delivery vehicles of the present disclosure can comprise or encode a Class 1 or a Class 2 Cas protein. A Cas protein can be a type I, type II, type III, type IV, type V, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

Non-limiting examples of Cas proteins that can be delivered by the delivery vehicles of the present disclosure include c2c1, Cas13a (formerly C2c2), Cas13b, Cas13c, Cas13d, c2c3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Cas9 (Csn1 or Csx12), Cas10, Cas10d, Cas14, Cas10, Cas10d, CasF, CasG, CasH, Cas12a (formerly Cpf1), Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof. Non-limiting examples of mutant Cas9 proteins or Cas9 variants include SpG, SpRY, eSpCas9 (1.1), SpCas9-HF1, nSpCas9, SpCas9 (H840A), dSpCas9, SpCas9 (N863A), SpCas9 (D839A), SpCas9 (H983A), as well as others described in Chuang C K et al., Int J Mol Sci. 2021 Sep. 13; 22(18):9872, which is incorporated herein by reference in its entirety.

Another example of a Cas protein that can be delivered by a delivery vehicle of the present disclosure includes Cas14. A Cas14 protein or polypeptide (also termed as “CasZ” protein or polypeptide) can bind and/or modify (e.g., cleave, nick, methylate, demethylate, etc.) a target nucleic acid and/or a polypeptide associated with target nucleic acid (e.g., methylation or acetylation of a histone tail) (e.g., in some cases the CasZ protein includes a fusion partner with an activity, and in some cases the CasZ protein provides nuclease activity). In some cases, the Cas14 protein or polypeptide is a naturally-occurring protein (e.g., naturally occurs in prokaryotic cells) (e.g., a CasZ protein). In other cases, the Cas14 protein or polypeptide not a naturally-occurring polypeptide (e.g., the Cas14 protein is a variant Cas14 protein, a chimeric protein, and the like). A Cas14 protein includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A naturally occurring Cas14 protein functions as an endonuclease that catalyzes cleavage at a specific sequence in a targeted nucleic acid (e.g., a double stranded DNA (dsDNA)). The sequence specificity is provided by the associated guide RNA, which hybridizes to a target sequence within the target DNA. The naturally occurring Cas14 guide RNA is a crRNA, where the crRNA includes (i) a guide sequence that hybridizes to a target sequence in the target DNA and (ii) a protein binding segment that binds to the Cas14 protein. Non-limiting examples of Cas14 proteins include those described U.S. Patent Publication Nos. US20200172886 and US20210214697, Harrington L B et al., Science. 2018 Nov. 16; 362(6416):839-842; Aquino-Jarquin G. Nanomedicine. 2019 June; 18:428-431; each of which is incorporated herein by reference in its entirety. In some cases, the cargo disclosed herein comprises Cas14 polypeptide or a nucleic acid molecule encoding Cas 14 polypeptide. In some cases, the cargo disclosed herein comprises Cas14a polypeptide or a nucleic acid molecule encoding Cas 14a polypeptide. In some cases, the cargo disclosed herein comprises Cas14b polypeptide or a nucleic acid molecule encoding Cas14b polypeptide. In some cases, the cargo disclosed herein comprises Cas14c polypeptide or a nucleic acid molecule encoding Cas14c polypeptide.

A Cas protein can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, Leptotrichia wadeii, Leptotrichia wadeii F0279, Rhodobacter capsulatus SB1003, Rhodobacter capsulatus R121, Rhodobacter capsulatus DE442, Lachnospiraceae bacterium NK4A179, Lachnospiraceae bacterium MA2020, Clostridium aminophilum DSM 10710, Paludibacter propionicigenes WB4, Carnobacterium gallinarum DMS4847, Carnobacterium gallinarum DSM4847, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Listeria seeligeri, Listeria weihenstephanensis FSL R90317, Listeria weihenstephanensis FSL M60635, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as disclosed herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like nuclease domain. The RuvC and HNH domains can each cut a different strand of double-stranded DNA to make a double-stranded break in the DNA. A Cas protein can comprise only one nuclease domain (e.g., Cpf1 comprises RuvC domain but lacks HNH domain).

A Cas protein can comprise an amino acid sequence having at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a nuclease domain (e.g., RuvC domain, HNH domain) of a wild-type Cas protein.

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

In some embodiments, the cargo delivered by the delivery vehicles of the present disclosure comprises a nuclease-null DNA binding protein derived from a DNA nuclease that can induce transcriptional activation or repression of a target DNA sequence. In some embodiments, the cargo comprises or encodes a nuclease-null RNA binding protein derived from an RNA nuclease that can induce transcriptional activation or repression of a target RNA sequence. For example, a cargo can comprise or encode a Cas protein which lacks cleavage activity.

A Cas protein can be a fusion protein. For example, a Cas protein can be fused to a heterologous functional domain. A heterologous functional domain can comprise a cleavage domain, an epigenetic modification domain, a transcriptional activation domain, or a transcriptional repressor domain. A Cas protein can also be fused to a heterologous polypeptide providing increased or decreased stability. The fused domain or heterologous polypeptide can be located at the N-terminus, the C-terminus, or internally within the Cas protein.

The regulation of genes can be of any gene of interest. It is contemplated that genetic homologues of a gene described herein are covered. For example, a gene can exhibit a certain identity and/or homology to genes disclosed herein. Therefore, it is contemplated that a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level) can be modified. It is also contemplated that a gene that exhibits or exhibits about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be modified.

A Cas protein can be provided in any form. For example, a Cas protein can be provided in the form of a protein, such as a Cas protein alone or complexed with a guide nucleic acid. A Cas protein can be provided in the form of a nucleic acid encoding the Cas protein, such as an RNA (e.g., messenger RNA (mRNA)) or DNA.

The nucleic acid encoding the Cas protein that is delivered by the delivery vehicles of the present disclosure can be codon optimized for efficient translation into protein in a particular cell or organism.

In some embodiments, a Cas protein is a dead Cas protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

A dCas9 polypeptide can associate with a single guide RNA (sgRNA) to activate or repress transcription of target DNA. sgRNAs can be introduced into cells expressing the engineered chimeric receptor polypeptide. In some cases, such cells contain one or more different sgRNAs that target the same nucleic acid. In other cases, the sgRNAs target different nucleic acids in the cell. The nucleic acids targeted by the guide RNA can be any that are expressed in a cell such as an immune cell. The nucleic acids targeted can be a gene involved in immune cell regulation. In some embodiments, the nucleic acid is associated with cancer. The nucleic acid associated with cancer can be a cell cycle gene, cell response gene, apoptosis gene, or phagocytosis gene. The recombinant guide RNA can be recognized by a CRISPR protein, a nuclease-null CRISPR protein, variants thereof, derivatives thereof, or fragments thereof.

Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g., nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type Cas9 activity).

One or a plurality of the nuclease domains (e.g., RuvC, HNH) of a Cas protein can be deleted or mutated so that they are no longer functional or comprise reduced nuclease activity (e.g., deactivated or dead Cas, i.e., “dCas”). For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A dead Cas protein can comprise one or more mutations relative to a wild-type version of the protein. The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

As non-limiting examples, residues D10, G12, G17, E762, H840, N854, N863, H982, H983, A984, D986, and/or A987 (or the corresponding mutations of any of the Cas proteins) can be mutated. For example, e.g., D10A, G12A, G17A, E762A, H840A, N854A, N863A, H982A, H983A, A984A, and/or D986A. Mutations other than alanine substitutions can be suitable.

A D10A mutation can be combined with one or more of H840A, N854A, or N856A mutations to produce a Cas9 protein substantially lacking DNA cleavage activity (e.g., a dead Cas9 protein). A H840A mutation can be combined with one or more of D10A, N854A, or N856A mutations to produce a site-directed polypeptide substantially lacking DNA cleavage activity. A N854A mutation can be combined with one or more of H840A, D10A, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking cleavage activity. In some embodiments, the Cas9 protein is a Cas9 protein from S. pyogenes (e.g., SwissProt accession number Q99ZW2). In some embodiments, the Cas9 protein is a Cas9 from S. aureus (e.g., SwissProt accession number J7RUA5). In some embodiments, the Cas9 protein is a modified version of a Cas9 protein from S. pyogenes or S. Aureus. In some embodiments, the Cas9 protein is derived from a Cas9 protein from S. pyogenes or S. Aureus. For example, a S. pyogenes or S. Aureus Cas9 protein lacking cleavage activity.

Cas9 can generally refer to a polypeptide with at least about 5%, 10%, 20%, 30%, 40%, 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most about 5%, 10%, 20%, 30%, 40%, 50%, 60%, 70%, 80%, 90%, 100% sequence identity and/or sequence similarity to a wild type exemplary Cas9 polypeptide (e.g., from S. pyogenes). Cas9 can refer to the wildtype or a modified form of the Cas9 protein that can comprise an amino acid change such as a deletion, insertion, substitution, variant, mutation, fusion, chimera, or any combination thereof.

In some embodiments, the cargo comprises or encodes a “zinc finger nuclease” or “ZFN.” ZFNs refer to a fusion between a cleavage domain, such as a cleavage domain of FokI, and at least one zinc finger motif (e.g., at least 2, 3, 4, or 5 zinc finger motifs) which can bind polynucleotides such as DNA and RNA. The heterodimerization at a certain position in a polynucleotide of two individual ZFNs in certain orientation and spacing can lead to cleavage of the polynucleotide. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain. Exemplary ZFNs include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

In some embodiments, a cargo protein or a protein encoded by a cargo nucleic acid molecule, which comprises a ZFN, can generate a double-strand break in a target polynucleotide, such as DNA. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template that contains homology arms flanking sites of the target DNA can be provided. In some embodiments, a ZFN is a zinc finger nickase which induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33. In some embodiments, a ZFN binds a polynucleotide (e.g., DNA and/or RNA) but is unable to cleave the polynucleotide.

In some embodiments, the cleavage domain of cargo protein or a protein encoded by a cargo nucleic acid molecule, which comprises a ZFN, comprises a modified form of a wild type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type cleavage domain. The modified form of the cleavage domain can have no substantial nucleic acid-cleaving activity. In some embodiments, the cleavage domain is enzymatically inactive.

In some embodiments, a cargo protein or a protein encoded by a cargo nucleic acid molecule comprises a “TALEN” or “TAL-effector nuclease.” TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HDR. In some cases, a single-stranded donor DNA repair template is provided to promote HDR. Detailed descriptions of TALENs and their uses for gene editing are found, e.g., in U.S. Pat. Nos. 8,440,431; 8,440,432; 8,450,471; 8,586,363; and 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

In some embodiments, a TALEN is engineered for reduced nuclease activity. In some embodiments, the nuclease domain of a TALEN comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type nuclease domain. The modified form of the nuclease domain can have no substantial nucleic acid-cleaving activity. In some embodiments, the nuclease domain is enzymatically inactive.

In some embodiments, the transcription activator-like effector (TALE) protein is fused to a domain that can modulate transcription and does not comprise a nuclease. In some embodiments, the transcription activator-like effector (TALE) protein is designed to function as a transcriptional activator. In some embodiments, the transcription activator-like effector (TALE) protein is designed to function as a transcriptional repressor. For example, the DNA-binding domain of the transcription activator-like effector (TALE) protein can be fused (e.g., linked) to one or more transcriptional activation domains, or to one or more transcriptional repression domains. Non-limiting examples of a transcriptional activation domain include a herpes simplex VP16 activation domain and a tetrameric repeat of the VP16 activation domain, e.g., a VP64 activation domain. Other examples include VP16, VP32, VP64, VPR, p65, RTA, KRAB, or P65HSF1. A non-limiting example of a transcriptional repression domain includes a Krüppel-associated box domain.

In some embodiments, a cargo protein or a protein encoded by a cargo nucleic acid molecule comprises a meganuclease. Meganucleases generally refer to rare-cutting endonucleases or homing endonucleases that can be highly specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-MsoI meganuclease, I-SceI meganuclease, variants thereof, derivatives thereof, and fragments thereof. Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

In some embodiments, the nuclease domain of a meganuclease comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have less than 90%, less than 80%, less than 70%, less than 60%, less than 50%, less than 40%, less than 30%, less than 20%, less than 10%, less than 5%, or less than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA.

### Targetable 3′-Overhang Nuclease

In some cases, the cargo to be delivered by the delivery vehicles of the present disclosure comprises a nuclease that generates a 3′-overhang double strand breaks in DNA, e.g., a Type IIS restriction enzyme or a functional domain of a Type IIS restriction enzyme. The term “Type IIS restriction enzyme,” as used here in, is a restriction enzyme that recognizes asymmetric DNA sequences and cleaves outside of their recognition sequence. In one embodiment, the restriction enzyme is Acul.

In some cases, the cargo comprises a targetable nuclease fusion protein that comprises a dimerization-dependent nuclease domain, e.g., Type IIS restriction enzyme domain. For instance, the targetable nuclease fusion protein comprises a dimerization-dependent nuclease domain, wherein the domain generates 3′ overhang double strand breaks in DNA; and a DNA-binding domain (DBD). In some cases, the dimerization-dependent nuclease domain is a Type IIS restriction enzyme nuclease domain, e.g., an Acul nuclease domain.

In some cases, the DBD is a protein or a protein domain that binds to its target nucleic acid in a sequence-dependent manner. In some cases, the DBD disclosed herein is either a zinc finger array or a dCas9.

In some cases, the nuclease fusion protein is a zinc finger nuclease fusion protein. The zinc finger nuclease fusion proteins described herein comprise a nuclease domain that generates a 3′ overhang double strand break in DNA upon dimerization (i.e., the nuclease activity is “dimerization-dependent”); an optional amino acid linker; and a zinc finger domain comprising one or more carboxy-terminal or amino-terminal zinc finger(s). Zinc finger nuclease fusion proteins in the monomer form, comprising one or more carboxy-terminal or amino-terminal zinc finger(s), can join together to form a dimer either upon or prior to binding to a target site, thereby activating the nuclease cleavage. The zinc finger nuclease fusion proteins described herein can be used to create insertion/deletion mutations (indels) with high frequency via repair of nuclease-induced DNA breaks by non-homologous end-joining.

Zinc finger nuclease fusion proteins can also be used to copy, incorporate, or insert an exogenous nucleic acid sequence of interest into a target site of a genomic locus of a cell. In some embodiments, the methods provided herein comprise providing to the nucleus of a cell an exogenous nucleic acid “donor template” sequence and the zinc finger nuclease fusion protein or another nucleic acid sequence encoding the zinc finger nuclease fusion protein self. In some cases, both the exogenous nucleic acid “donor template” sequence and the zinc finger nuclease fusion protein are delivered by a delivery vehicle provided herein. The exogenous nucleic acid donor template sequence comprises end sequences homologous to sequences within the target site of the genomic locus. Zinc fingers can be designed to recognize and bind to the genomic target site with specificity. Upon binding to the target site, the dimerized nuclease domains of the fusion protein(s) can generate a 3′ overhang double strand break within the target site to induce homology-directed repair between sequences surrounding the break and the exogenous nucleic acid sequence, thereby copying, incorporating and/or inserting the exogenous nucleic acid sequence into the target site of the genomic locus of the cell.

Zinc finger nuclease fusion proteins can comprise any nuclease domain capable of generating a 3′ overhang double strand break in DNA upon dimerization.

The nuclease domain can be, for example, a Type IIS restriction enzyme nuclease domain including, but not limited to a Acul, Alol, Bpml, Bael, or Mmel nuclease domain. In some instances, the Acul nuclease domain can have an amino acid sequence.

Exemplary nucleotide and amino acid sequences encoding Acul are known in the art and can be located, for example, at GenBank accession number HQ327692.1.

In some embodiments, the Type IIS restriction enzyme nuclease domain includes isoschizomers of Acul, e.g., Eco57I. The nucleotide and amino acid sequences encoding Eco57I can be located, for example at UniProt database reference number P25239.

Exemplary nucleotide and amino acid sequences encoding Alol are known in the art and can be located, for example, at GenBank accession number AJ312389.1.

Exemplary nucleotide and amino acid sequences encoding Bpml are known in the art and can be located, for example, at GenBank accession number ADK30556.1. Exemplary nucleotide and amino acid sequences encoding Bael are known in the art and can be located, for example, at GenBank accession number ABS74060.1.

Exemplary nucleotide and amino acid sequences encoding Mmel are known in the art and can be located, for example, at GenBank accession number EU616582.1.

Any Type IIS restriction enzyme nuclease domain having dimerization-dependent nuclease activity could be fused to a zinc finger domain and used to conduct the methods described herein. In some embodiments, the nuclease domain is attached to the C-terminus of the zinc finger domain. In other embodiments, the nuclease domain is attached to the N-terminus of the zinc finger domain.

The dimerization-dependent nuclease domain and the zinc finger domain of the zinc finger nuclease fusion protein can be joined together by an amino acid linker. The terms linked, joined and fused are used interchangeably herein to refer to the means by which two domains of a fusion protein are joined. The amino acid linker can comprise any sequence of at least one amino acid and up to a sequence of 10 amino acids. In specific embodiments, the linker can comprise Leucine, Arginine, Glycine and Serine (LRGS (SEQ ID NO: 50)); glycine, glycine, glycine, glycine and serine (GGGGS (SEQ ID NO: 51)); or a non-standard amino acid, threonine, glutamic acid and asparagine (XTEN) as described by Shellenberger, et al. Nat Biotechnol. 2009 December; 27(12): 1186-90.

In some embodiments, the dimerization-dependent nuclease domain, the zinc finger domain, the TALE, and/or the dCas9 domain can have an amino acid sequences that have at least 80%, at least 85%, at least 90%, at least 95%, at least 96%, at least 97%, at least 98%, or at least 99% sequence identity to the amino acid sequence of the exemplary amino acid sequences of the dimerization-dependent nuclease domain, the zinc finger domain, the TALE, and/or the dCas9, described herein.

Upon binding to the target site and forming a dimer complex, the nuclease domain of the zinc finger nuclease fusion protein can generate a 3′ overhang double strand break within the target site to induce homology-directed repair, with resulting copying, incorporating, and/or integrating of the exogenous nucleic acid sequence, or a portion thereof, within the target site. Where there is nucleotide sequence homology, a donor template oligonucleotide sequence (either single- or double-stranded) can act as a template to repair a target DNA sequence that experienced the double-strand break, leading to the transfer of genetic information from the donor to the target. Such transfer can involve mismatch correction of heteroduplex DNA that forms between the broken target and the donor, and/or synthesis-dependent strand annealing, in which the donor is used to re-synthesize genetic information that will become part of the target, and/or related processes. Homology-directed repair often results in an alteration of the sequence of the target nucleotide such that part or all of the sequence of the donor nucleotide sequence is copied and/or incorporated into the target nucleotide.

The zinc finger nuclease fusion protein can create a double-stranded break in the target sequence at a predetermined site, and an exogenous nucleic acid sequence acting as a donor template, having homology to the nucleotide sequence in the region of the break, can be copied, incorporated, and/or introduced into the genomic locus. The presence of the double-stranded break has been shown to greatly enhance the efficiencies of these different repair outcomes. The donor sequence may be physically integrated or, alternatively, the donor nucleotide is used as a template for repair of the break via homologous recombination, resulting in the introduction of all or part of the nucleotide sequence as in the donor into the genomic locus. Thus, a sequence in the genomic locus can be altered and, in certain embodiments, can be converted into a sequence present in a donor nucleotide.

Also described herein are dCas9 nuclease fusion proteins and methods of using the same for enhancing homology-directed repair frequencies at the site of a nuclease-induced double strand breaks. dCas9 nuclease fusion proteins comprise a catalytically inactive Cas9 carboxy-terminal or amino-terminal domain linked to a dimerization-dependent nuclease domain that generates 3′ overhang double strand breaks in DNA. A catalytically inactive Cas9 domain contains mutations (e.g., D10A and/or H841 A) which results in the loss of native endonuclease activity (Qi et ah, Cell (2013)). The endonuclease activity is instead provided by the linked dimerization-dependent nuclease domain to which it is fused. dCas9 nuclease fusion proteins in the monomer form join together to form a dimer either prior to or upon binding to a dCas9 target site, thereby activating the nuclease cleavage. Clustered regularly interspaced short palindromic repeats (CRISPR) and associated Cas proteins constitute the CRISPR-Cas system. The RNA-guided Cas9 endonuclease specifically targets and cleaves DNA in a sequence-dependent manner (Gasiunas, G., et al, Proc Natl Acad Sci USA 109, E2579-E2586 (2012); Jinek, M., et al, Science 337, 816-821 (2012); Sternberg, S. H., et al, Nature 507, 62 (2014); Deltcheva, E., et al, Nature 471, 602-607 (2011)), and has been widely used for programmable genome editing in a variety of organisms and model systems (Cong, L., et al, Science 339, 819-823 (2013); Jiang, W., et al, Nat. Biotechnol 31, 233-239 (2013); Sander, J. D. & Joung, J. K., Nature Biotechnol. 32, 347-355. (2014)). Cas9 requires a guide RNA composed of two RNAs that associate or are covalently linked together to make a guide RNA; the CRISPR RNA (crRNA), and the trans-activating RNA (tracrRNA). If the nucleotide sequence of a genomic locus of interest is complementary to the guide RNA, Cas9 recognizes and cleaves the site. A ternary complex of Cas9 with crRNA and tracrRNA or a binary complex of Cas9 with a guide RNA can bind to and cleave dsDNA protospacer sequences that match the crRNA spacer and that are also adjoined to a short protospacer-adjacent motif. dCas9 can still associate with a crRNA/tracrRNA complex or with a guide RNA and then recognize and bind to a target site even though its native catalytic activity is inactivated. The nucleotide and amino acid sequences encoding Cas9 are known in the art and can be located, for example, at GenBank accession number NC_002737.2.

dCas9 nuclease fusion proteins described herein can be used to induce homology-directed repair events at a target site of a genomic locus of a cell. This method comprises providing an exogenous nucleic acid sequence, a nucleic acid sequence encoding the dCas9 nuclease fusion protein and one or more (e.g., at least two) guide RNAs to the nucleus of a cell. The exogenous nucleic acid sequence comprises end sequences homologous to sequences within the target site of the genomic locus. The guide RNA is designed to direct two dCas9 nuclease fusions to a predetermined target site in which each dCas9/gRNA complex binds to one of two “half-sites”. The dCas9 domains will recognize and bind to their target sites with complementary to the guide RNA and an adjoining PAM sequence with specificity. Upon binding to the target site, the linked nuclease domain of the fusion protein functions as a dimer to generate a 3′ overhang double strand break within the target site to induce homology-directed repair between sequences surrounding the break and the exogenous nucleic acid sequence, thereby copying, incorporating, and/or inserting the exogenous nucleic acid sequence into the target site of the genomic locus of the cell. The nucleotide and amino acid sequences encoding dCas9 are known in the art and can be located, for example, at GenBank accession number KR011748.1. dCas9 is also described by Zetsche et al, Nature Biotechnology 33, 139-142 (2015).

dCas9 nuclease fusion proteins can comprise any nuclease domain capable of generating a 3′ overhang double strand break in DNA upon dimerization. The nuclease domain can be, for example, a Type IIS restriction enzyme nuclease domain including, but not limited to a Acul, Alol, Bpml, Bael, or Mmel nuclease domain. The dimerization-dependent nuclease domain and the dCas9 domain of the dCas9 nuclease fusion proteins are joined together by an optional amino acid linker. The amino acid linker can comprise any sequence of at least one amino acid and up to a sequence of 10 amino acids. In specific embodiments, the amino acid linker can comprise, for example glycine, glycine, glycine, glycine and serine (GGGGS (SEQ ID NO: 51)) or a non-standard amino acid, threonine, glutamic acid and asparagine (XTEN).

In any of the methods and compositions described herein, the exogenous nucleotide sequence acting as a donor can contain sequences that are homologous, but not identical, to genomic sequences in the target site, thereby stimulating homology-directed repair to copy, incorporate, and/or insert a non-identical sequence within the target site. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the target site, such that new sequences are introduced into the region of interest. In these instances, the non-homologous sequence is generally flanked by sequences of 50-1,000 base pairs (or any integral value there between) or any number of base pairs greater than 1,000, that are homologous or identical to sequences in the target site.

In some embodiments, an entire donor template sequence or a portion of the donor template sequence is integrated at the target site. Any of the methods described herein can be used for partial or complete inactivation of one or more genomic loci in a cell by targeted integration of donor sequence that disrupts expression of the gene(s) of interest. Any of the methods described herein can be used to replace mutated sequences within the target site, thereby correcting a mutated gene or inducing formerly inactive gene expression. The nature of the exogenous nucleic acid sequence to be incorporated will depend on the therapeutic goal to be achieved and can range from inducing or inhibiting gene transcription, to replacing mutated sequences of a defective gene or adding or deleting sequences within a gene.

In other embodiments, the DBD (e.g., zinc finger or dCas9) nuclease fusion protein introduces a variable-length insertion or deletion mutation that overlaps, partially or completely, with a nuclease target site of a genomic locus of a cell through non-homologous end-joining or microhomology-mediated end joining. In these embodiments, no exogenous donor sequence is provided. Rather, a nucleic acid sequence encoding a zinc finger nuclease fusion protein or an isolated zinc finger nuclease fusion protein is provided to the nucleus of a cell, and the zinc finger nuclease fusion protein binds to the nuclease target site to generate a 3′ overhang double strand break within the nuclease target site, followed by repair of the break by non-homologous end-joining or microhomology-mediated end joining. Both non-homologous end-joining or microhomology-mediated end joining can produce insertions or deletions that interfere with, or inhibit, gene transcription at the nuclease target site.

Non-limiting examples of the targetable 3′-overhang nuclease (e.g., the Type IIS restriction enzymes, e.g., DBD nuclease fusion protein), sequences encoding the nuclease, compositions, methods of use, and systems include those described in international publication no. WO2020160481, which is incorporated herein by reference in its entirety.

### Base Editing

In some cases, the cargo to be delivered by the delivery vehicles of the present disclosure comprises a nucleobase editor (also termed as “base editor”) or one or more components of a nucleobase editing (also termed as “base editing”) complex.

The term “base editor (BE),” or “nucleobase editor (NBE),” as used herein, can refer to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating an adenosine (A) in DNA. In some embodiments, the base editor is capable of deaminating a cytosine (C) in DNA.

In some cases, the base editor disclosed herein comprises a deaminase or a functional domain thereof (“deaminase domain”) that catalyzes deamination reaction.

The term “deaminase” or “deaminase domain,” as used herein, refers to a protein or enzyme that catalyzes a deamination reaction. In some embodiments, the deaminase or deaminase domain is an adenosine deaminase, catalyzing the deamination of adenosine, converting it to the nucleoside hypoxanthine. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

As used herein, an “adenosine deaminase” is an enzyme that catalyzes the deamination of adenosine, converting it to the nucleoside hypoxanthine. Under standard Watson-Crick hydrogen bond pairing, an adenosine base hydrogen bonds to a thymine base (or a uracil in case of RNA). When adenine is converted to hypoxanthine, the hypoxanthine undergoes hydrogen bond pairing with cytosine. Thus, a conversion of “A” to hypoxanthine by adenosine deaminase will cause the insertion of “C” instead of a “T” during cellular repair and/or replication processes. Since the cytosine “C” pairs with guanine “G”, the adenosine deaminase in coordination with DNA replication causes the conversion of an A·T pairing to a C·G pairing in the double-stranded DNA molecule.

In some embodiments, the base editor is a fusion protein comprising a nucleic acid programmable R/DNA binding protein (napR/DNAbp) fused to a deaminase (e.g., cytidine deaminase or adenosine deaminase) domain. The term “nucleic acid programmable D/RNA binding protein (napR/DNAbp)” refers to any protein that may associate (e.g., form a complex) with one or more nucleic acid molecules (i.e., which may broadly be referred to as a “napR/DNAbp-programming nucleic acid molecule” and includes, for example, guide RNA in the case of Cas systems) which direct or otherwise program the protein to localize to a specific target nucleotide sequence (e.g., a gene locus of a genome, or a RNA molecule) that is complementary to the one or more nucleic acid molecules (or a portion or region thereof) associated with the protein, thereby causing the protein to bind to the nucleotide sequence at the specific target site. This term napR/DNAbp embraces CRISPR Cas 9 proteins, as well as Cas9 equivalents, homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. However, the nucleic acid programmable R/DNA binding protein (napR/DNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao F, Shen X Z, Jiang F, Wu Y, Han C. DNA-guided genome editing using the Natronobacterium gregoryi Argonaute. Nat Biotechnol 2016; 34(7):768-73, which is incorporated herein by reference.

In some cases, the napR/DNAbp is derived from a nuclease disclosed herein, such as, Cas9 (e.g., dCas9 and nCas9), CasX, CasY, Cas14, Cpf1, C2c1, C2c2, C2c3, Argonaute protein, or a variant thereof. In some embodiments, the base editor comprises a Cas9 (e.g., dCas9 and nCas9), CasX, CasY, Cpf1, C2c1, C2c2, C2c3, or Argonaute protein fused to a deaminase (e.g., cytidine deaminase or adenosine deaminase). In some embodiments, the base editor comprises a Cas9 nickase (nCas9) fused to a deaminase (e.g., cytidine deaminase or adenosine deaminase). In some embodiments, the base editor comprises a CasX protein fused to a deaminase (e.g., cytidine deaminase or adenosine deaminase). In some embodiments, the base editor comprises a nuclease-inactive Cas9 (dCas9) fused to a deaminase (e.g., cytidine deaminase or adenosine deaminase). In some embodiments, the base editor comprises a CasY protein fused to a deaminase (e.g., cytidine deaminase or adenosine deaminase). In some embodiments, the base editor comprises a Cas14 protein fused to a deaminase (e.g., cytidine deaminase or adenosine deaminase). In some embodiments, the base editor comprises a Cpf1 protein fused to a deaminase (e.g., cytidine deaminase or adenosine deaminase). In some embodiments, the base editor comprises a C2c1 protein fused to a deaminase (e.g., cytidine deaminase or adenosine deaminase). In some embodiments, the base editor comprises a C2c2 protein fused to a deaminase (e.g., cytidine deaminase or adenosine deaminase). In some embodiments, the base editor comprises a C2c3 protein fused to a deaminase (e.g., cytidine deaminase or adenosine deaminase). In some embodiments, the base editor comprises an Argonaute protein fused to a deaminase (e.g., cytidine deaminase or adenosine deaminase).

In some embodiments, the adenosine deaminases provided herein are capable of deaminating adenosine. In some embodiments, the adenosine deaminases provided herein are capable of deaminating adenosine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some cases, the deaminase domain of the base editor disclosed herein is derived from a cytidine deaminase. In some cases, the cytidine deaminase domain is derived from the apolipoprotein B mRNA-editing complex (APOBEC) family deaminase, such as APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, or APOBEC3H deaminase.

In some embodiments, the base editor is fused to, or further comprises as part of a fusion protein, an inhibitor of base excision repair, for example, a uracil glycosylase inhibitor (UGI) domain.

In some cases, the base editor disclosed herein is a fusion protein that comprises a structure such as, NH₂-[deaminase domain]-[napR/DNAbp]-[UGI domain]-COOH; NH₂-[deaminase domain]-[napR/DNAbp]-[UGI]-[UGI]-COOH; NH₂-[deaminase domain]-[napR/DNAbp]-[UGI]-COOH; NH₂-[UGI]-[deaminase domain]-[napR/DNAbp]-COOH; NH₂-[deaminase domain]-[UGI]-[napR/DNAbp]-COOH; NH₂-[napR/DNAbp]-[UGI]-[deaminase domain]-COOH; or NH₂-[napR/DNAbp]-[deaminase domain]-[UGI]-COOH; wherein each instance of “-” comprises an optional linker.

In some cases, the base editor is fused to, or further comprises as part of a fusion protein, a uracil binding protein (UBP). The term “uracil binding protein” or “UBP,” as used herein, refers to a protein that is capable of binding to uracil. In some embodiments, the uracil binding protein is a uracil modifying enzyme. In some embodiments, the uracil binding protein is a uracil base excision enzyme. In some embodiments, the uracil binding protein is a uracil DNA glycosylase (UDG). In some embodiments, a uracil binding protein binds uracil with an affinity that is at least 1%, 2%, 3%, 5%, 10%, 15%, 20%, 30%, 40%, 50%, 60%, 70%, 80%, 90%, or at least 95% of the affinity that a wild type UDG (e.g., a human UDG) binds to uracil. The term “base excision enzyme” or “BEE,” as used herein, refers to a protein that is capable of removing a base (e.g., A, T, C, G, or U) from a nucleic acid molecule (e.g., DNA or RNA). In some embodiments, a BEE is capable of removing a cytosine from DNA. In some embodiments, a BEE is capable of removing a thymine from DNA. Exemplary BEEs include, without limitation UDG Tyr147Ala, and UDG Asn204Asp as described in Sang et al., “A Unique Uracil-DNA binding protein of the uracil DNA glycosylase superfamily,” Nucleic Acids Research, Vol. 43, No. 17 2015; the entire contents of which are hereby incorporated by reference.

In some embodiments, the UBP is a uracil modifying enzyme. In some embodiments, the UBP is a uracil base excision enzyme. In some embodiments, the UBP is a uracil DNA glycosylase. In some embodiments, the UBP is any of the uracil binding proteins provided herein. For example, the UBP may be a UDG, a UdgX, a UdgX*, a UdgX_On, or a SMUG1. In some embodiments, the UBP comprises an amino acid sequence that is at least 75%, 80%, 85%, 90%, 95%, 96%, 97%, 98%, 99%, or 99.5% identical to a uracil binding protein, a uracil base excision enzyme or a uracil DNA glycosylase (UDG) enzyme.

In some cases, the base editor is fused to, or comprises as a part of the fusion protein, a nucleic acid polymerase domain (NAP). For instance, the nucleic acid polymerase domain is a eukaryotic nucleic acid polymerase domain. In some cases, the nucleic acid polymerase domain is a DNA polymerase domain. In some cases, the nucleic acid polymerase domain has translesion polymerase activity. In some cases, the nucleic acid polymerase domain is a translesion DNA polymerase. In some cases, the nucleic acid polymerase domain is from Rev7, Rev1 complex, polymerase iota, polymerase kappa, and polymerase eta. In some cases, the nucleic acid polymerase domain is selected from the group of eukaryotic polymerases consisting of alpha, beta, gamma, delta, epsilon, gamma, eta, iota, kappa, lambda, mu, and nu.

In some cases, the base editor disclosed herein is a fusion protein that comprises a structure such as, NH₂-[deaminase domain]-[napR/DNAbp domain]-[UBP]-[NAP]-COOH; NH₂-[deaminase domain]-[napR/DNAbp]-[NAP]-[UBP]-COOH; NH₂-[deaminase domain]-[NAP]-[napR/DNAbp]-[UBP]-COOH; or NH₂-[NAP]-[deaminase domain]-[napR/DNAbp]-[UBP]-COOH; wherein each instance of “-” comprises an optional linker.

In some cases, the base editor disclosed herein is complexed with a napR/DNAbp-programming nucleic acid molecule. In some cases, the base editing system disclose herein comprises a base editor and a napR/DNAbp-programming nucleic acid molecule, e.g., the base editor complexed with the napR/DNAbp-programming nucleic acid molecule. In some cases, the delivery vehicles of the present disclosure deliver a base editing system that comprises both a base editor and a napR/DNAbp-programming nucleic acid molecule, e.g., the base editor complexed with the napR/DNAbp-programming nucleic acid molecule. In some cases, a base editor is delivered separately from the napR/DNAbp-programming nucleic acid molecule through delivery vehicles disclosed herein, or together with other delivery methods, into a cell.

The term “napR/DNAbp-programming nucleic acid molecule” or equivalently “guide sequence” refers the one or more nucleic acid molecules which associate with and direct or otherwise program a napR/DNAbp protein to localize to a specific target nucleotide sequence (e.g., a gene locus of a genome) that is complementary to the one or more nucleic acid molecules (or a portion or region thereof) associated with the protein, thereby causing the napR/DNAbp protein to bind to the nucleotide sequence at the specific target site. A non-limiting example is a guide RNA of a Cas protein of a CRISPR-Cas genome editing system.

Exemplary configurations, sequences, and mutations thereof for deaminase domains, napR/DNAbp domains, UGI domains, and whole base editor proteins, and exemplary configurations of a base editing system (e.g., comprising both a base editor and a napR/DNAbp-programming nucleic acid molecule) that can be delivered by a delivery vehicle disclosed herein include those described in U.S. Patent Publication Nos. US20170121693, US20180073012, US20180312828, US20210230577, US20210198330, US20210277379, US2020399626, US2021371858, US2021380955, US2021277379, US2021301274; international patent publication nos. WO20051562, WO21041885, WO21050512, and WO21113494, each of which is incorporated herein by reference in its entirety. Exemplary configurations, sequences, and mutations thereof for deaminase domains, napR/DNAbp domains, UGI domains, and whole base editor proteins, that can be delivered by a delivery vehicle disclosed herein also include those described in Komor A C et al. Nature. 2016 May 19; 533(7603):420-4; Kim Y B et al. Nat Biotechnol. 2017 April; 35(4):371-376; Rees H A et al. Nat Commun. 2017 Jun. 6; 8:15790; Newby G A et al. Mol Ther. 2021 Nov. 3; 29(11):3107-3124; Huang T P et al. Nat Protoc. 2021 February; 16(2):1089-1128; Lapinaite A et al. Science. 2020 Jul. 31; 369(6503):566-571; Anzalone A V et al. Nat Biotechnol. 2020 July; 38(7):824-844; Rees H A et al. Nat Rev Genet. 2018 December; 19(12):770-788; Koblan L W et al. Nat Biotechnol. 2018 October; 36(9):843-846; and Gaudelli N M et al. Nature. 2017 Nov. 23; 551(7681):464-471; each of which is incorporated herein by reference in its entirety.

### Prime Editing

In some cases, the cargo to be delivered by the delivery vehicles of the present disclosure comprises one or more components of a prime editing system.

Prime editing is a ‘search-and-replace’ genome editing technology by which the genome of living organisms may be modified. In some cases, the priming editing system delivered by the delivery vehicles of the present disclosure uses a fusion protein, comprising a nucleic acid-programmable RNA or DNA binding protein (napR/DNAbp) and a nucleic acid polymerase (e.g., a reverse transcriptase or an RNA-dependent RNA polymerase), and a napR/DNAbp-programming nucleic acid molecule. In some cases, the fusion protein comprises a catalytically impaired Cas9 endonuclease fused to an engineered reverse transcriptase enzyme. In some cases, the napR/DNAbp-programming nucleic acid molecule comprises a prime editing guide RNA (pegRNA), capable of identifying the target site and providing the new genetic information to replace the target DNA nucleotides. The prime editing system disclosed herein can mediate targeted insertions, deletions, and/or base-to-base conversions without the need for double strand breaks (DSBs) or donor DNA templates.

In some cases, a napR/DNAbp-programming nucleic acid molecule for a prime editing system, e.g., a prime editing guide RNA (pegRNA), is capable of (i) identifying the target nucleotide sequence to be edited, and (ii) encoding new genetic information that replaces the targeted sequence. In some cases, the pegRNA comprises an extended single guide RNA (sgRNA) containing a primer binding site (PBS) and a template sequence for nucleic acid polymerase (e.g., reverse transcriptase or RNA polymerase). In some cases, during genome editing, the primer binding site allows the 3′ end of the nicked DNA strand to hybridize to the pegRNA, while the reverse transcriptase template serves as a template for the synthesis of edited genetic information.

One or more components of a prime editing system that can be delivered by the delivery vehicles of the present disclosure include those described in International Patent Publication Nos. WO2020191242, WO2020191234, WO2020086908, WO2021072328, WO2021226558, and WO2020191248, and Anzalone A V, et al. Nature. 2019 December; 576(7785):149-157; Anzalone A V, et al. Nat Biotechnol. 2021 Dec. 9; Hsu J Y, et al. Nat Commun. 2021 Feb. 15; 12(1):1034; Nelson J W, et al. Nat Biotechnol. 2021 Oct. 4; Chen P J, et al. Cell. 2021 Oct. 28; 184(22):5635-5652.e29; Scholefield J, et al. Gene Ther. 2021 August; 28(7-8):396-401; Newby G A, et al. Mol Ther. 2021 Nov. 3; 29(11):3107-3124, each of which is incorporated herein by reference in its entirety.

### Epigenetic Editing

In some cases, the cargo to be delivered by the delivery vehicles of the present disclosure comprises an epigenetic editor or one or more components of an epigenetic editing complex (e.g., comprising an epigenetic editor and a nucleic acid molecule that guides the epigenetic editor to bind and/or modify one or more specific target sequences).

In some cases, the epigenetic editor or epigenetic editing complex disclosed herein has epigenetic activities, such as, methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, or demyristoylation activity. In some cases, the epigenetic editor or epigenetic editing complex disclosed herein has a chromosome modification enzyme, or a functional domain that has the functional activity equivalent to a chromosome modification enzyme, such as a methylase, demethylase, acetylase, deacetylase, deaminase, phosphorylase, dephosphorylase, histone modifying enzyme, or nucleotide modifying enzyme. In some cases, the epigenetic editor or epigenetic editing complex disclosed herein has a histone modifying enzyme, or a functional domain that has the functional activity equivalent to a histone modifying enzyme. In some cases, the epigenetic editor or epigenetic editing complex disclosed herein has a nucleotide modifying enzyme, or a functional domain that has the functional activity equivalent to a nucleotide modifying enzyme.

In some cases, the epigenetic editor or epigenetic editing system provides the effect of modulating expression of a target gene without altering the DNA sequence of the target gene. For example, in some embodiments, the epigenetic editor or epigenetic editing system results in repression or silencing of expression of a target gene. In some embodiments, the epigenetic editor or epigenetic editing system results in activation or increased expression of a target gene.

In an aspect, the epigenetic editor or epigenetic editing system is not sequence specific, e.g., the epigenetic modification effectuated by the epigenetic editor or epigenetic editing system is not specific to one or more target sequences. In another aspect, the epigenetic editor or epigenetic editing system described herein is sequence specific, or allele specific. For example, an epigenetic editor may specifically target a DNA sequence recognized by a DNA binding domain of the epigenetic editor. In some embodiments, the target DNA sequence is specific to one copy of a target gene. In some embodiments, the target gene sequence is specific to one allele of a target gene. Accordingly, the epigenetic modification and modulation of expression thereof may be specific to one copy or one allele of the target gene.

In some embodiments, the epigenetic editor or epigenetic editing system comprises a histone methyltransferase domain. In some embodiments, the histone methyltransferase domain is a DOT1L domain, a SET domain, a SUV39H1 domain, a G9a/EHMT2 protein domain, a EZH1 domain, a EZH2 domain, a SETDB1 domain, or any combination thereof.

In some embodiments, the epigenetic editor or epigenetic editing system comprises a DNA methyltransferase domain or a histone methyltransferase domain. DNA methyltransferase domains may mediate methylation at DNA nucleotides, for example at any of an A, T, G or C nucleotide. In some embodiments, the methylated nucleotide is a N6-methyladenosine (m6A). In some embodiments, the methylated nucleotide is a 5-methylcytosine (5mC). In some embodiments, the methylation is at a CG (or CpG) dinucleotide sequence. In some embodiments, the methylation is at a CHG or CHH sequence, where H is any one of A, T, or C.

In some embodiments, the epigenetic editor or epigenetic editing system comprises a DNA methyltransferase DNMT domain that catalyzes transfer of a methyl group to cytosine, thereby repressing expression of the target gene through the recruitment of repressive regulatory proteins. In some embodiments, the epigenetic editor or epigenetic editing system comprises a DNA methyltransferase (DNMT) family protein domain. In some embodiments, the epigenetic editor or epigenetic editing system comprises a DNMT1 domain, TRDMT1 domain, DNMT3 domain, DNMT3A domain, DNMT3B domain, DNMT3C domain, DNMT3L domain, TRDMT1 (DNMT2) domain, M.MpeI domain, M.SssI domain, M.HpaII domain, M.AluI domain, M.HaeIII domain, M.HhaI domain, M.MspI domain, Masc1 domain, MET1 domain, Masc2 domain, Dim-2 domain, dDnmt2 domain, Pmt1 domain, DRM1 domain, DRM2 domain, CMT1 domain, CMT2 domain, CMT3 domain, Rid domain, hsdM gene domain, hsdS gene domain, M.TaqI domain, M.EcoDam domain, M.CcrMI domain, CamA domain, or any combination thereof (e.g., a fusion protein comprising any combination thereof).

In some embodiments, the epigenetic editor or epigenetic editing system recruits one or more protein domains that repress expression of the target gene. In some embodiments, the epigenetic editor or epigenetic editing system interacts with a scaffold protein domain that recruits one or more protein domains that repress expression of the target gene. For example, the epigenetic editor or epigenetic editing system may recruit or interact with a scaffold protein domain that recruits a PRMT protein, a HDAC protein, a SETDB1 protein, or a NuRD protein domain. In some embodiments, the epigenetic editor or epigenetic editing system comprises a Krüppel associated box (KRAB) repression domain; a Repressor Element Silencing Transcription Factor (REST) repression domain, KRAB-associated protein 1 (KAP1) domain, a MAD domain, a FKHR (forkhead in rhabdosarcoma gene) repressor domain, aEGR-1 (early growth response gene product-1) repressor domain, a ets2 repressor factor repressor domain (ERD), a MAD smSIN3 interaction domain (SID), a WRPW motif of the hairy-related basic helix-loop-helix (bHLH) repressor proteins; an HP1 alpha chromo-shadow repression domain, or any combination thereof. In some embodiments, the epigenetic editor or epigenetic editing system comprises a KRAB domain. In some embodiments, the epigenetic editor or epigenetic editing system comprises a Tripartite motif containing 28 (TRIM28, TIF1-beta, or KAP1) protein.

In some embodiments, the epigenetic editor or epigenetic editing system comprises a protein domain that represses expression of the target gene. For example, the epigenetic editor or epigenetic editing system may comprise a functional domain derived from a zinc finger repressor protein. In some embodiments, the epigenetic editor or epigenetic editing system comprises a functional repression domain derived from a KOX1/ZNF10 domain, a KOX8/ZNF708 domain, a ZNF43 domain, a ZNF184 domain, a ZNF91 KRAB domain, a HPF4 domain, a HTF10 domain or a HTF34 domain or any combination thereof. In some embodiments, the epigenetic editor or epigenetic editing system comprises a functional repression domain derived from a ZIM3 protein domain, a ZNF436 domain, a ZNF257 domain, a ZNF675 domain, a ZNF490 domain, a ZNF320 domain, a ZNF331 domain, a ZNF816 domain, a ZNF680 domain, a ZNF41 domain, a ZNF189 domain, a ZNF528 domain, a ZNF543 domain, a ZNF554 domain, a ZNF140 domain, a ZNF610 domain, a ZNF264 domain, a ZNF350 domain, a ZNF8 domain, a ZNF582 domain, a ZNF30 domain, a ZNF324 domain, a ZNF98 domain, a ZNF669 domain, a ZNF677 domain, a ZNF596 domain, a ZNF214 domain, a ZNF37A domain, a ZNF34 domain, a ZNF250 domain, a ZNF547 domain, a ZNF273 domain, a ZNF354A domain, a ZFP82 domain, a ZNF224 domain, a ZNF33A domain, a ZNF45 domain, a ZNF175 domain, a ZNF595 domain, a ZNF184 domain, a ZNF419 domain, a ZFP28-1 domain, a ZFP28-2 domain, a ZNF18 domain, a ZNF213 domain, a ZNF394 domain, a ZFP1 domain, a ZFP14 domain, a ZNF416 domain, a ZNF557 domain, a ZNF566 domain, a ZNF729 domain, a ZIM2 domain, a ZNF254 domain, a ZNF764 domain, a ZNF785 domain or any combination thereof. In some embodiments, the domain is a ZIM3 domain, a ZNF554 domain, a ZNF264 domain, a ZNF324 domain, a ZNF354A domain, a ZNF189 domain, a ZNF543 domain, a ZFP82 domain, a ZNF669 domain, or a ZNF582 domain or any combination thereof. In some embodiments, the domain is a ZIM3 domain, a ZNF554 domain, a ZNF264 domain, a ZNF324 domain, or a ZNF354A domain or any combination thereof.

Sequences of exemplary functional domains of an epigenetic editor or epigenetic editing system that may reduce or silence target gene expression are provided can be found in PCT/US2021/030643 and Tycko et al. (Tycko J, DelRosso N, Hess G T, Aradhana, Banerjee A, Mukund A, Van M V, Ego B K, Yao D, Spees K, Suzuki P, Marinov G K, Kundaje A, Bassik M C, Bintu L. High-Throughput Discovery and Characterization of Human Transcriptional Effectors. Cell. 2020 Dec. 23; 183(7):2020-2035.e16. doi: 10.1016/j.cell.2020.11.024. Epub 2020 Dec. 15. PMID: 33326746; PMCID: PMC8178797.), each of which is incorporated here by reference in its entirety.

In some embodiments, the epigenetic editor or epigenetic editing system comprises a functional repression domain derived from ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37A, ZNF34, ZNF250, ZNF547, ZNF273, ZNF354A, ZFP82, ZNF224, ZNF33A, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, ZNF10 (KOX1), CBX5 (chromoshadow domain), RYBP (YAF2_RYBP component of PRC1), YAF2 (YAF2_RYBP component of PRC1), MGA (component of PRC1.6), CBX1 (chromoshadow), SCMH1 (SAM_1/SPM), MPP8 (Chromodomain), SUMO3 (Rad60-SLD), HERC2 (Cyt-b5), BIN1 (SH3_9), PCGF2 (RING finger protein domain), TOX (HMG box), FOXA1 (HNF3A C-terminal domain), FOXA2 (HNF3B C-terminal domain), IRF2BP1 (IRF-2BP1_2 N-terminal domain), IRF2BP2 (IRF-2BP1_2 N-terminal domain), IRF2BPL IRF-2BP1_2 N-terminal domain, HOXA13 (homeodomain), HOXB13 (homeodomain), HOXC13 (homeodomain), HOXA11 (homeodomain), HOXC11 (homeodomain), HOXC10 (homeodomain), HOXA10 (homeodomain), HOXB9 (homeodomain), HOXA9 (homeodomain), or any combination thereof.

In some embodiments, the epigenetic editor or epigenetic editing system comprises a histone deacetylase protein domain. In some embodiments, the epigenetic editor or epigenetic editing system comprises a HDAC family protein domain, for example, a HDAC1, HDAC3, HDAC5, HDAC7, or HDAC9 protein domain. In some embodiments, the epigenetic editor or epigenetic editing system removes the acetyl group from histones. In some embodiments, the epigenetic editor or epigenetic editing system comprises a nucleosome remodeling domain. In some embodiments, the epigenetic editor or epigenetic editing system comprises a nucleosome remodeling and deacetylase complex (NURD), which removes acetyl groups from histones.

In some embodiments, the epigenetic editor or epigenetic editing system comprises a Tripartite motif containing 28 (TRIM28, TIF1-beta, or KAP1) protein. In some embodiments, the epigenetic editor or epigenetic editing system comprises one or more KAP1 protein. The KAP1 protein in an epigenetic editor may form a complex with one or more other effector domains of the epigenetic editor or one or more proteins involved in modulation of gene expression in a cellular environment. For example, KAP1 may be recruited by a KRAB domain of a transcriptional repressor. In some embodiments, KAP1 interacts with or recruits a histone deacetylase protein, a histone-lysine methyltransferase protein (e.g., depositing methyl groups on lysine 9 [K9] of a histone H3 tail [H3K9]), a chromatin remodeling protein, and/or a heterochromatin protein. In some embodiments, a KAP1 protein interacts with or recruits one or more protein complexes that reduces or silences gene expression. In some embodiments, a KAP1 protein interacts with or recruits a heterochromatin protein 1 (HP1) protein (e.g., via a chromoshadow domain of the HP1 protein), a SETDB1 protein, a HDAC protein, and/or a NuRD protein complex component. In some embodiments, a KAP1 protein recruits a CHD3 subunit of the nucleosome remodeling and deacetylation (NuRD) complex, thereby decreasing or silencing expression of a target gene. In some embodiments, a KAP1 protein recruits a SETDB1 protein (e.g., to a promoter region of a target gene), thereby decreasing or silencing expression of the target gene via H3K9 methylation associated with, e.g., the promoter region of the target gene. In some embodiments, recruitment of the SETDB1 protein results in heterochromatinization of a chromosome region harboring the target gene, thereby reducing or silencing expression of the target gene. In some embodiments, a KAP1 protein interacts with or recruits a HP1 protein, thereby decreasing or silencing expression of a target gene via reduced acetylation of H3K9 or H3K14 on histone tails associated with the target gene. Recruitment of SETDB1 induces heterochromatinization. In some embodiments, a KAP1 protein interacts with or recruits a ZFP90 protein (e.g., isoform 2 of ZFP90), and/or a FOXP3 protein.

In some embodiments, the epigenetic editor or epigenetic editing system comprises a protein domain that interacts with or is recruited by one or more DNA epigenetic marks. For example, the epigenetic editor or epigenetic editing system may comprise a methyl CpG binding protein 2 (MECP2) protein that interacts with methylated DNA nucleotides in the target gene. In some embodiments, the MECP2 protein interacts with methylated DNA nucleotides in a CpG island of the target gene. In some embodiments, the MECP2 protein interacts with methylated DNA nucleotides not in a CpG island of the target gene. In some embodiments, the MECP2 protein in an epigenetic editor results in condensed chromatin structure, thereby reducing or silencing expression of the target gene. In some embodiments, the MECP2 protein in an epigenetic editor interacts with a histone deacetylase (e.g., HDAC), thereby repressing or silencing expression of the target gene. In some embodiments, the MECP2 protein in an epigenetic editor blocks access of a transcription factor or transcriptional activator to the target gene, thereby repressing or silencing expression of the target gene.

In some embodiments, the epigenetic editor or epigenetic editing system comprises a chromoshadow domain, a ubiquitin-2 like Rad60 SUMO-like (Rad60-SLD/SUMO) domain, a chromatin organization modifier domain (Chromo) domain, a Yaf2/RYBP C-terminal binding motif domain (YAF2_RYBP), a CBX family C-terminal motif domain (CBX7_C), a Zinc finger C3HC4 type (RING finger) domain (zf-C3HC4_2), a Cytochrome b5 domain (Cyt-b5), a helix-loop-helix domain (HLH), a high mobility group box domain (HMG-box), a Sterile alpha motif domain (SAM_1), basic leucine zipper domain (bZIP_1), a Myb_DNA-binding domain, a Homeodomain, a MYM-type Zinc finger with FCS sequence domain (zf-FCS), a interferon regulatory factor 2-binding protein zinc finger domain (IRF-2BP1_2), a SSX repression domain (SSXRD), a B-box-type zinc finger domain (zf-B_box), a sterile alpha motif domain (SAM_2), a CXXC zinc finger domain (zf-CXXC), a regulator of chromosome condensation 1 domain (RCC1), a SRC homology 3 domain (SH3_9), a sterile alpha motif/Pointed domain (SAM_PNT), a Vestigial/Tondu family domain (Vg_Tdu), a LIM domain, a RNA recognition motif domain (RRM_1), a basic leucine zipper domain (bZIP_2), a paired amphipathic helix domain (PAH), a proteasomal ATPase OB C-terminal domain (Prot_ATP_ID_OB), a nervy homology 2 domain (NHR2), a helix-hairpin-helix motif domain (HHH_3), a hinge domain of cleavage stimulation factor subunit 2 (CSTF2_hinge), a PPAR gamma N-terminal region domain (PPARgamma_N), a CDC48 N-terminal domain (CDC48_2), a WD40 repeat domain (WD40), a Fip1 motif domain (Fip1), a PDZ domain (PDZ_6), a Von Willebrand factor type C domain (VWC), a NAB conserved region 1 domain (NCD1), a S1 RNA-binding domain (S1), a HNF3 C-terminal domain (HNF_C), a Tudor domain (Tudor_2), a histone-like transcription factor (CBF/NF-Y) and archaeal histone domain (CBFD_NFYB_HMF), a Zinc finger protein domain (DUF3669), a EGF-like domain (cEGF), a GATA zinc finger domain (GATA), a TEA/ATTS domain (TEA), a phorbol esters/diacylglycerol binding domain (C1-1), polycomb-like MTF2 factor 2 domain (Mtf2_C), a transactivation domain of FOXO protein family (FOXO-TAD), a Homeobox KN domain (Homeobox_KN), a BED zinc finger domain (zf-BED), a zinc finger of C3HC4-type RING domain (zf-C3HC4_4), a RAD51 interacting motif domain (RAD51_interact), a p55-binding region of a Methyl-CpG-binding domain protein MBD (MBDa), Notch domain, a Raf-like Ras-binding domain (RBD), a Spin/Ssty family domain (Spin-Ssty), a PHD finger domain (PHD_3), a Low-density lipoprotein receptor domain class A (Ldl_recept_a), a CS domain, a DM DNA binding domain, or a QLQ domain. In some embodiments, the epigenetic editor or epigenetic editing system comprises a protein domain comprising a YAF2_RYBP domain, or homeodomain or any combination thereof. In some embodiments, the homeodomain of the YAF2_RYBP domain is a PRD domain, a NKL domain, a HOXL domain, or a LIM domain. In some embodiments, the epigenetic editor or epigenetic editing system comprises a protein domain selected from a group consisting of SUMO3 domain, Chromo domain from M phase phosphoprotein 8 (MPP8), chromoshadow domain from Chromobox 1 (CBX1), and SAM_1/SPM domain from Scm Polycomb Group Protein Homolog 1 (SCMH1). In some embodiments, the epigenetic editor or epigenetic editing system comprises a HNF3 C-terminal domain (HNF_C). In some embodiments, the HNF_C domain is from FOXA1 or FOXA2. In some embodiments, the HNF_C domain comprises an EHI (engrailed homology 1) motif. In some embodiments, the epigenetic editor or epigenetic editing system comprises an interferon regulatory factor 2-binding protein zinc finger domain (IRF-2BP1_2). In some embodiments, the epigenetic editor or epigenetic editing system comprises a Cyt-b5 domain from DNA repair factor HERC2 E3 ligase. In some embodiments, the epigenetic editor or epigenetic editing system comprises a variant SH3 domain (SH3_9) from Bridging Integrator 1 (BIN1). In some embodiments, the epigenetic editor or epigenetic editing system comprises HMG-box domain from transcription factor TOX or zf-C3HC4-2 RING finger domain from the polycomb component PCGF2. In some embodiment, the epigenetic editor or epigenetic editing system comprises a Chromodomain-helicase-DNA-binding protein 3 (CHD3). In some embodiments, the epigenetic editor or epigenetic editing system comprises a ZNF783 domain. In some embodiments, the epigenetic editor or epigenetic editing system comprises a YAF2_RYBP domain. In some embodiment, the YAF2_RYBP domain comprises a 32 amino acid Yaf2/RYBP C-terminal binding motif domain (32 AA RYBP).

In some embodiments, the epigenetic editor or epigenetic editing system makes an epigenetic modification at a target gene that activates expression of the target gene. In some embodiments, the epigenetic editor or epigenetic editing system modifies the chemical modification of DNA or histone residues associated with the DNA at a target gene harboring the target sequence, thereby activating or increasing expression of the target gene. In some embodiments, the epigenetic editor or epigenetic editing system comprises a DNA demethylase, a DNA dioxygenase, a DNA hydroxylase, or a histone demethylase domain.

In some embodiments, the epigenetic editor or epigenetic editing system comprises a DNA demethylase domain that removes a methyl group from DNA nucleotides, thereby increasing or activating expression of the target gene. For example, the epigenetic editor or epigenetic editing system can comprise a TET (ten-eleven translocation methylcytosine dioxygenase) family protein domain that demethylates cytosine in methylated form and thereby increases expression of a target gene, such as a TET1, TET2, or TET3 protein domain or any combination thereof.

In some embodiments, the epigenetic editor or epigenetic editing system comprises a KDM family protein domain that demethylates lysines in DNA-associated histones, thereby increasing expression of the target gene.

In some cases, the epigenetic editor or epigenetic editing system comprises a functional domain derived from TET1, TET2, TET3, TDG, ROS1, DME, DML2, DML3, or any combination thereof.

In some embodiments, the epigenetic editor or epigenetic editing system comprises a protein domain that recruits one or more transcription activator domains. In some embodiments, the epigenetic editor or epigenetic editing system comprises a protein domain that recruits one or more transcription factors. In some embodiments, the epigenetic editor or epigenetic editing system comprises a transcription activator or a transcription factor domain. In some embodiments, the epigenetic editor or epigenetic editing system comprises a Herpes Simplex Virus Protein 16 (VP16) activation domain. In some embodiments, the epigenetic editor or epigenetic editing system comprises an activation domain comprising a tandem repeat of multiple VP16 activation domains. In some embodiments, the epigenetic editor or epigenetic editing system comprises a p65 activation domain of NFκB; an Epstein-Barr virus R transactivator (Rta) activation domain. In some embodiments, the epigenetic editor or epigenetic editing system comprises a fusion of multiple activators, e.g., a tripartite activator of the VP64, the p65, and the Rta activation domains, (a VPR activation domain).

In some embodiments, the epigenetic editor or epigenetic editing system comprises a transactivation domain of FOXO protein family (FOXO-TAD), a LMSTEN motif domain (LMSTEN), a Transducer of regulated CREB activity C terminus domain (TORC_C), a QLQ domain, a Nuclear receptor coactivator domain (Nuc_rec_co-act), an Autophagy receptor zinc finger-C2H2 domain (Zn-C2H2-12), an Anaphase-promoting complex subunit 16 (ANAPC16), a Dpy-30 domain, a ANCI homology domain (AHD), a Signal transducer and activator of transcription 2 C terminal (STAT2_C), a I-kappa-kinase-beta NEMO binding domain (IKKbetaNEMObind), an Early growth response N-terminal domain (DUF3446), a TFIIE beta subunit core domain (TFIIE_beta), a N-terminal domain of DPF2/REQ (Requiem_N), a LNR domain (Notch), an Atypical Arm repeat (Arm_3), a Protein kinase C terminal domain (PKinase_C), WW domain, a SH3 domain (SH3_1), a Myb-like DNA-binding domain, a WD domain G-beta repeat (WD40), a PHD-finger (PHD), a RNA recognition motif domain (RRM_1), a GATA zinc finger domain (GATA), a Vps4 C terminal oligomerization domain (Vps4_C), or in any combination thereof. In some embodiments, the epigenetic editor or epigenetic editing system comprises a KRAB domain that activates expression of a target gene. For example, the KRAB domain may be a ZNF473 KRAB domain, a ZFP28 KRAB domain, a ZNF496 KRAB domain, or a ZNF597 KRAB domain or any combination thereof.

Exemplary domains that can activate or increase target gene expression can include, but not limited to, VP16, VP64, VP160, HIFIalpha, CITED2, Stat3, p65, p53, ZNF473, FOXO1, Myb, CRTC1, Med9, EGR3, SMARCA2, Dpy-30, NCOA3, ZFP28, ZNF496, ZNF597, HSF1, RTA, or any combination thereof. Other exemplary domains that can activate or increase target gene expression can include, but not limited to, ABL1, AF9, ANM2, APBB1, APC16, BTK, CACO1, CRTC2, CRTC3, CXXC1, DPF1, DPY30, EGR3, ENL, FIGN, FOXO1, FOXO3, IKKA, IMA5, ITCH, KIBRA, KPCI, KS6B2, MTA3, MYB, MYBA, NCOA2, NCOA3, NOTC1, NOTC1, NOTC2, PRP19, PYGO1, PYGO2, SAVI, SMCA2, SMRC2, STAT2, T2EB, U2AF4, WBP4, WWP1, WWP2, WWTR1, ZFP28, ZN473, ZN496 ZN597, or any combination thereof.

In some embodiments, the epigenetic editor or epigenetic editing system regulates acetylation of a histone associated with the target gene. In some embodiments, the epigenetic editor or epigenetic editing system comprises a histone acetyltransferase domain. In some embodiments, the epigenetic editor or epigenetic editing system comprises a protein domain that interacts with a histone acetyltransferase domain to effect histone acetylation. In some embodiments, the epigenetic editor or epigenetic editing system comprises a histone acetyltransferase 1 (HAT1) domain. In some embodiments, the epigenetic editor or epigenetic editing system comprises a histone acetyltransferase (HAT) core domain of the human E1A-associated protein p300. In some embodiments, the epigenetic editor or epigenetic editing system comprises a CBP/p300 histone acetyltransferase or a catalytic domain thereof. In some embodiments, the epigenetic editor or epigenetic editing system comprises a CREBBP, GCN4, GCN5, SAGA, SALSA, HAP2, HAP3, HAP4, PCAF, KMT2A, or any combination thereof.

In some cases, the epigenetic editor disclosed herein can be, or epigenetic editing system disclosed herein can comprise, any agent that binds a target polynucleotide and has epigenetic modulation activity.

In some embodiments, the epigenetic editor disclosed herein binds the polynucleotide at a specific target sequence using a DNA binding domain. In some cases, the epigenetic editing system disclosed herein comprises a nucleic acid that guides DNA binding of the epigenetic editor. In some embodiments, the epigenetic editor comprises an effector domain capable of modulating epigenetic state of a nucleic acid sequence at or adjacent to the target polynucleotide.

### Transcription Factors

In some embodiments, the cargo to be delivered by the delivery vehicles of the present disclosure comprises a transcription factor. The transcription factor can be fused to a DNA binding domain described herein.

Non-limiting examples of transcription factor can include a transcription activator or a transcription repressor domain (e.g., the Kruppel associated box (KRAB or SKD); the Mad mSIN3 interaction domain (SID); the ERF repressor domain (ERD), etc.); zinc-finger-based artificial transcription factors (see, e.g., Sera (2009) Adv. Drug Deliv. 61:513); TALE-based artificial transcription factors (see, e.g., Liu et al. (2013) Nat. Rev. Genetics 14:781); CRISPR/Cas-based artificial transcription factors (see, e.g., Pandelakis M, et al. Cell Syst. 2020 Jan. 22; 10(1):1-14; Martinez-Escobar, et al. Frontiers in oncology vol. 10 604948. 3 Feb. 2021), and the like.

In some cases, the transcription factor comprises a VP64 polypeptide (transcriptional activation). In some cases, the transcription factor comprises a Kriippel-associated box (KRAB) polypeptide (transcriptional repression). In some cases, the transcription factor comprises a Mad mSIN3 interaction domain (SID) polypeptide (transcriptional repression). In some cases, the transcription factor comprises an ERF repressor domain (ERD) polypeptide (transcriptional repression). For example, in some cases, the transcription factor is a transcriptional activator, where the transcriptional activator is GALA-VP16.

### Antibodies

In some embodiments, the cargo to be delivered by the delivery vehicles of the present disclosure comprises or encodes an antibody or a functional fragment thereof, or a fusion protein that comprises an antigen-binding domain.

In some embodiments, the antibody or a functional fragment thereof disclosed herein, or antigen-binding domain disclosed herein binds to an antigen associated with a disease such as a viral, bacterial, and/or parasitic infection; inflammatory and/or autoimmune disease; or neoplasm such as a cancer and/or tumor. In some embodiments, the antibody or a functional fragment thereof disclosed herein, or antigen-binding domain disclosed herein binds a tumor associated antigen (e.g., protein or polypeptide). In some embodiments, the antibody or a functional fragment thereof disclosed herein, or antigen-binding domain disclosed herein is a monoclonal antibody, a polyclonal antibody, a recombinant antibody, a human antibody, a humanized antibody, or a functional derivative, variant or fragment thereof, including, but not limited to, a Fab, a Fab′, a F(ab′)₂, an Fc, an Fv, a scFv, minibody, a diabody, and a single-domain antibody such as a heavy chain variable domain (VH), a light chain variable domain (VL) and a variable domain (VHH) of camelid derived Nanobody.

In some embodiments, the antibody or a functional fragment thereof disclosed herein, or antigen-binding domain disclosed herein comprises, or is derived from, or is functional equivalent to an antibody selected from the group consisting of: 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, ABP 494 (cetuximab biosimilar), abrilumab, ABT-700, ABT-806, Actimab-A (actinium Ac-225 lintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine I-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imciromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDI0680, MEDI-3617, MEDI-551 (inebilizumab), MEDI-565, MEDI6469, mepolizumab, metelimumab, MGB453, MGD006/S80880, MGD007, MGD009, MGD011, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, RO7009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIVIA, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox. In certain embodiments, the ligand interacting domain binds an Fc domain of an aforementioned antibody.

In some embodiments, the antibody or a functional fragment thereof disclosed herein, or antigen-binding domain disclosed herein binds an antibody selected from the group consisting of: 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, ABP 494 (cetuximab biosimilar), abrilumab, ABT-700, ABT-806, Actimab-A (actinium Ac-225 lintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine I-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imciromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDI0680, MEDI-3617, MEDI-551 (inebilizumab), MEDI-565, MEDI6469, mepolizumab, metelimumab, MGB453, MGD006/S80880, MGD007, MGD009, MGD011, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, RO7009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIVIA, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox. In certain embodiments, the ligand interacting domain binds an Fc domain of an aforementioned antibody.

In some embodiments, the antibody or a functional fragment thereof disclosed herein, or antigen-binding domain disclosed herein binds an antigen selected from the group consisting of: 1-40-β-amyloid, 4-1BB, 5AC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD23 (IgE receptor), CD25 (α chain of IL-2receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSFIR, CSF2, CTLA-4, C—X—C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor α-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-α, IFN-γ, IgE, IgE Fc region, IGF-1 receptor, IGF-1, IGHE, IL 17A, IL 17F, IL 20, IL-12, IL-13, IL-17, IL-1β, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin α4β7, integrin α4, integrin α5β1, integrin α7 β7, integrin αIIbβ3, integrin αvβ3, interferon α/β receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-R a, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-β 1, TGF-β 2, TGF-β, TNF-α, TRAIL-R1, TRAIL-R2, tumor antigen CTAA16.88, tumor specific glycosylation of MUC1, tumor-associated calcium signal transducer 2, TWEAK receptor, TYRPI (glycoprotein 75), VEGFA, VEGFR1, VEGFR2, vimentin, and VWF.

### Nucleic Acid Molecules

In some cases, the cargo delivered by the delivery vehicles of the present disclosure comprises a nucleic acid molecule. The nucleic acid molecule can have a coding sequence that encodes a protein or polypeptide described herein. For instance, the nucleic acid molecule can be delivered by the delivery vehicle disclosed herein for the purpose of delivering a protein encoded by the nucleic acid molecule. In some cases, the nucleic acid molecule is a functional nucleic acid molecule, for instance, that nucleic acid molecule can have a non-coding sequence or a coding sequence that has biological functions other than being used as a template for protein synthesis. For instance, the nucleic acid molecule can regulate RNA splicing, regulate translation of mRNA, target genomic DNA for transcriptional regulation, or bind to a protein or organelle.

In some cases, the nucleic acid molecules are loaded into the delivery vehicle (e.g., VLP, e.g., heVLP or humanized VLP) by direct loading, such as electroporation of the delivery vehicle in vitro. In some cases, the nucleic acid molecules are loaded into the delivery vehicle (e.g., VLP, e.g., heVLP or humanized VLP) by binding to a nucleic acid binding protein (e.g., Cas protein) that is part of the delivery vehicle or is already loaded into the delivery vehicle.

Non-limiting examples of the nucleic acid molecules include DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), IRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the nucleic acid is a mutant nucleic acid. In some embodiments, the nucleic acid is a fusion or chimera of multiple nucleic acid sequences.

In some embodiments, the nucleic acid delivered by the delivery vehicles of the present disclosure, e.g., a DNA sequence, is edited to correct a genetic mutation. For instance, the edited nucleic acid has been edited using a gene editing technology, e.g., a guide RNA and CRISPR-Cas9/Cpf1, or using a different targeted endonuclease (e.g., Zinc-finger nucleases, transcription-activator-like nucleases (TALENs)). In some cases, the nucleic acid is synthesized in vitro. In some embodiments, the genetic mutation is linked to a disease in a subject. Examples of edits to DNA include small insertions/deletions, large deletions, gene corrections with template DNA, or large insertions of DNA. In some embodiments, gene editing is accomplished with non-homologous end joining (NHEJ) or homology directed repair (HDR). In some embodiments, the edit is a knockout. In some embodiments, the edit is a knock-in. In some embodiments, both alleles of DNA are edited. In some embodiments, a single allele is edited. In some embodiments, multiple edits are made.

In some embodiments, the cargo may include a nucleic acid. For example, the cargo may comprise RNA to enhance expression of an endogenous protein, or a siRNA or miRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the cargo is a nucleic acid that targets a transcriptional activator that modulate structure or function in the target cells.

### Examples of Delivery Vehicle and Cargo Configurations

Non-limiting examples of delivery vehicle and cargo configurations include the following:
(1) A delivery vehicle (e.g., VLP, e.g., heVLP or humanized VLP) is created by producer cells expressing an envelope protein. Cargo is packaged inside the particle either by producer cells expressing cargo or particles being loaded by various particle loading methods described herein, such as electroporation.
(2) A delivery vehicle (e.g., VLP, e.g., heVLP or humanized VLP) is created by producer cells expressing an envelope protein. Cargo is packaged inside the particle either by producer cells expressing cargo-gag fusion or particles being loaded by various particle loading methods described herein, such as electroporation.
(3) A delivery vehicle (e.g., VLP, e.g., heVLP or humanized VLP) is created by producer cells expressing an envelope protein. Cargo is packaged inside the particle either by producer cells expressing cargo-PH fusion or particles being loaded by various particle loading methods described herein, such as electroporation.
(4) A delivery vehicle (e.g., VLP, e.g., heVLP or humanized VLP) is created by producer cells expressing an envelope protein. Cargo is packaged inside the particle either by producer cells expressing cargo-gag/PH fusion or particles being loaded by various particle loading methods described herein, such as electroporation.
(5) A delivery vehicle (e.g., VLP, e.g., heVLP or humanized VLP) is created by producer cells expressing an envelope protein. Cargo is packaged inside the particle in the presence of a dimerization molecule (A/C heterodimerizer) either by producer cells expressing cargo and gag fused to DmrA or DmrC or particles being loaded by various particle loading methods described herein, such as electroporation.
(6) A delivery vehicle (e.g., VLP, e.g., heVLP or humanized VLP) is created by producer cells expressing an envelope protein. Cargo is packaged inside the particle in the presence of a dimerization molecule (A/C heterodimerizer) either by producer cells expressing cargo and PH fused to DmrA or DmrC or particles being loaded by various particle loading methods described herein, such as electroporation.
(7) A delivery vehicle (e.g., VLP, e.g., heVLP or humanized VLP) is created by producer cells expressing an envelope protein. Cargo is packaged inside the particle in the presence of a dimerization molecule (A/C heterodimerizer) either by producer cells expressing cargo and gag/PH fused to DmrA or DmrC or particles being loaded by various particle loading methods described herein, such as electroporation.
(8) A delivery vehicle (e.g., VLP, e.g., heVLP or humanized VLP) is created by producer cells expressing an envelope protein. Cargo is packaged inside the particle either by producer cells expressing cargo and gag fused to an RNA binding protein (RBP), MS2, that binds to its MS2 RNA stem loop (MS2 SL) that is complexed with cargo or particles being loaded by various particle loading methods described herein, such as electroporation.
(9) A delivery vehicle (e.g., VLP, e.g., heVLP or humanized VLP) is created by producer cells expressing an envelope protein. Cargo is packaged inside the particle either by producer cells expressing cargo and PH fused to an RNA binding protein (RBP), MS2, that binds to its RNA stem loop (MS2 SL) that is complexed with cargo or particles being loaded by various particle loading methods described herein, such as electroporation.
(10) A delivery vehicle (e.g., VLP, e.g., heVLP or humanized VLP) is created by producer cells expressing an envelope protein. Cargo is packaged inside the particle either by producer cells expressing cargo and gag/PH fused to an RNA binding protein (RBP), MS2, that binds to its RNA stem loop (MS2 SL) that is complexed with cargo or particles being loaded by various particle loading methods described herein, such as electroporation.
(11) A delivery vehicle (e.g., VLP, e.g., heVLP or humanized VLP) is created by producer cells expressing an envelope protein. Cargo is packaged inside the particle in the presence of dimerization molecule (A/C Heterodimerizer) either by producer cells expressing cargo and gag and an RNA binding protein (RBP), MS2, fused to DmrA or DmrC that binds to its RNA stem loop (MS2 SL) that is complexed with cargo or particles being loaded by various particle loading methods described herein, such as electroporation.
(12) A delivery vehicle (e.g., VLP, e.g., heVLP or humanized VLP) is created by producer cells expressing an envelope protein. Cargo is packaged inside the particle in the presence of dimerization molecule (A/C Heterodimerizer) either by producer cells expressing cargo and PH and an RNA binding protein (RBP), MS2, fused to DmrA or DmrC that binds to its RNA stem loop (MS2 SL) that is complexed with cargo or particles being loaded by various particle loading methods described herein, such as electroporation.
(13) A delivery vehicle (e.g., VLP, e.g., heVLP or humanized VLP) is created by producer cells expressing an envelope protein. Cargo is packaged inside the particle in the presence of dimerization molecule (A/C Heterodimerizer) either by producer cells expressing cargo and gag/PH and an RNA binding protein (RBP), MS2, fused to DmrA or DmrC that binds to its RNA stem loop (MS2 SL) that is complexed with cargo or particles being loaded by various particle loading methods described herein, such as electroporation.
(14) A delivery vehicle (e.g., VLP, e.g., heVLP or humanized VLP) is created by producer cells expressing an envelope protein. Cargo is packaged inside the particle either by producer cells expressing cargo and gag fused to a repetitive GCN4 domain that is bound by an scFv that is fused with cargo or particles being loaded by various particle loading methods described herein, such as electroporation.
(15) A delivery vehicle (e.g., VLP, e.g., heVLP or humanized VLP) is created by producer cells expressing an envelope protein. Cargo is packaged inside the particle either by producer cells expressing cargo and PH fused to a repetitive GCN4 domain that is bound by an scFv that is fused with cargo or particles being loaded by various particle loading methods described herein, such as electroporation.
(16) A delivery vehicle (e.g., VLP, e.g., heVLP or humanized VLP) is created by producer cells expressing an envelope protein. Cargo is packaged inside the particle either by producer cells expressing cargo and gag/PH fused to a repetitive GCN4 domain that is bound by an scFv that is fused with cargo or particles being loaded by various particle loading methods described herein, such as electroporation.
(17) A delivery vehicle (e.g., VLP, e.g., heVLP or humanized VLP) is created by producer cells expressing an envelope protein. Cargo is packaged inside the particle in the presence of a dimerization molecule (A/C Heterodimerizer) by producer cells expressing gag and a repetitive GCN4 domain that are fused to DmrA or DmrC. GCN4 is bound by an scFv that is fused with cargo that is also being expressed in producer cells. Particles could also be loaded by various particle loading methods described herein, such as electroporation.
(18) A delivery vehicle (e.g., VLP, e.g., heVLP or humanized VLP) is created by producer cells expressing an envelope protein. Cargo is packaged inside the particle in the presence of a dimerization molecule (A/C Heterodimerizer) by producer cells expressing PH and a repetitive GCN4 domain that are fused to DmrA or DmrC. GCN4 is bound by an scFv that is fused with cargo that is also being expressed in producer cells. Particles could also be loaded by various particle loading methods described herein, such as electroporation.
(19) A delivery vehicle (e.g., VLP, e.g., heVLP or humanized VLP) is created by producer cells expressing an envelope protein. Cargo is packaged inside the particle in the presence of a dimerization molecule (A/C Heterodimerizer) by producer cells expressing gag/PH and a repetitive GCN4 domain that are fused to DmrA or DmrC. GCN4 is bound by an scFv that is fused with cargo that is also being expressed in producer cells. Particles could also be loaded by various particle loading methods described herein, such as electroporation.
(20) A delivery vehicle (e.g., VLP, e.g., heVLP or humanized VLP) is created by producer cells expressing an envelope protein. Cargo (AAV particles) is packaged inside the particle either by producer cells expressing cargo or particles being loaded by various particle loading methods described herein, such as electroporation.
(21) A delivery vehicle (e.g., VLP, e.g., heVLP or humanized VLP) is created by producer cells expressing an envelope protein. Cargo (AAV particles) is packaged inside the particle either by producer cells expressing cargo and gag or particles being loaded by various particle loading methods described herein, such as electroporation.
(22) A delivery vehicle (e.g., VLP, e.g., heVLP or humanized VLP) is created by producer cells expressing an envelope protein. Cargo (AAV particles) is packaged inside the particle either by producer cells expressing cargo and PH or particles being loaded by various particle loading methods described herein, such as electroporation.
(23) A delivery vehicle (e.g., VLP, e.g., heVLP or humanized VLP) is created by producer cells expressing an envelope protein. Cargo (AAV particles) is packaged inside the particle either by producer cells expressing cargo and gag/PH or particles being loaded by various particle loading methods described herein, such as electroporation.
(24) A delivery vehicle (e.g., VLP, e.g., heVLP or humanized VLP) is created by producer cells expressing an envelope protein. Cargo (AAV particles with DmrB inserted in the Capsid protein, VP2) is packaged inside the particle in the presence of DmrB dimerizer molecule either by producer cells expressing cargo and gag fused to DmrB or particles being loaded by various particle loading methods described herein, such as electroporation.
(25) A delivery vehicle (e.g., VLP, e.g., heVLP or humanized VLP) is created by producer cells expressing an envelope protein. Cargo (AAV particles with DmrB inserted in the Capsid protein, VP2) is packaged inside the particle in the presence of DmrB dimerizer molecule either by producer cells expressing cargo and PH fused to DmrB or particles being loaded by various particle loading methods described herein, such as electroporation.
(26) A delivery vehicle (e.g., VLP, e.g., heVLP or humanized VLP) is created by producer cells expressing an envelope protein. Cargo (AAV particles with DmrB inserted in the Capsid protein, VP2) is packaged inside the particle in the presence of DmrB dimerizer molecule either by producer cells expressing cargo and gag/PH fused to DmrB or particles being loaded by various particle loading methods described herein, such as electroporation.
(27) A delivery vehicle (e.g., VLP, e.g., heVLP or humanized VLP) is created by producer cells expressing an envelope protein. Cargo (AAV particles with DmrB inserted in the Capsid protein, VP2) is packaged inside the particle in the presence of DmrB dimerizer and A/C Heterodimerizer molecules either by producer cells expressing cargo and gag fused to DmrA, DmrB, or DmrC, or particles being loaded by various particle loading methods described herein, such as electroporation.
(28) A delivery vehicle (e.g., VLP, e.g., heVLP or humanized VLP) is created by producer cells expressing an envelope protein. Cargo (AAV particles with DmrB inserted in the Capsid protein, VP2) is packaged inside the particle in the presence of DmrB dimerizer and A/C Heterodimerizer molecules either by producer cells expressing cargo and PH fused to DmrA, DmrB, or DmrC, or particles being loaded by various particle loading methods described herein, such as electroporation.
(29) A delivery vehicle (e.g., VLP, e.g., heVLP or humanized VLP) is created by producer cells expressing an envelope protein. Cargo (AAV particles with DmrB inserted in the Capsid protein, VP2) is packaged inside the particle in the presence of DmrB dimerizer and A/C Heterodimerizer molecules either by producer cells expressing cargo and gag/PH fused to DmrA, DmrB, or DmrC, or particles being loaded by various particle loading methods described herein, such as electroporation.
(30) A delivery vehicle (e.g., VLP, e.g., heVLP or humanized VLP) is created by producer cells expressing an envelope protein. Cargo (single-stranded DNA) can be packaged inside the particle by various particle loading methods described herein, such as electroporation.
(31) A delivery vehicle (e.g., VLP, e.g., heVLP or humanized VLP) is created by producer cells expressing an envelope protein and gag. Cargo (single-stranded DNA) can be packaged inside the particle by various particle loading methods described herein, such as electroporation.
(32) A delivery vehicle (e.g., VLP, e.g., heVLP or humanized VLP) is created by producer cells expressing an envelope protein and PH. Cargo (single-stranded DNA) can be packaged inside the particle by various particle loading methods described herein, such as electroporation.
(33) A delivery vehicle (e.g., VLP, e.g., heVLP or humanized VLP) is created by producer cells expressing an envelope protein and gag/PH. Cargo (single-stranded DNA) can be packaged inside the particle by various particle loading methods described herein, such as electroporation.
(34) A delivery vehicle (e.g., VLP, e.g., heVLP or humanized VLP) is created by producer cells expressing an envelope protein. Cargo (double-stranded DNA) can be packaged inside the particle by various particle loading methods described herein, such as electroporation.
(35) A delivery vehicle (e.g., VLP, e.g., heVLP or humanized VLP) is created by producer cells expressing an envelope protein and gag. Cargo (double-stranded DNA) can be packaged inside the particle by various particle loading methods described herein, such as electroporation.
(36) A delivery vehicle (e.g., VLP, e.g., heVLP or humanized VLP) is created by producer cells expressing an envelope protein and PH. Cargo (double-stranded DNA) can be packaged inside the particle by various particle loading methods described herein, such as electroporation.
(37) A delivery vehicle (e.g., VLP, e.g., heVLP or humanized VLP) is created by producer cells expressing an envelope protein and gag/PH. Cargo (double-stranded DNA) can be packaged inside the particle by various particle loading methods described herein, such as electroporation.
(38) A delivery vehicle (e.g., VLP, e.g., heVLP or humanized VLP) is created by producer cells expressing an envelope protein and gag fused to a zinc finger protein (ZFP) that will bind a specific sequence in the cargo. Cargo (double-stranded DNA) can be packaged inside the particle by various particle loading methods described herein, such as electroporation.
(39) A delivery vehicle (e.g., VLP, e.g., heVLP or humanized VLP) is created by producer cells expressing an envelope protein and PH fused to a zinc finger protein (ZFP) that will bind a specific sequence in the cargo. Cargo (double-stranded DNA) can be packaged inside the particle by various particle loading methods described herein, such as electroporation.
(40) A delivery vehicle (e.g., VLP, e.g., heVLP or humanized VLP) is created by producer cells expressing an envelope protein and gag/PH fused to a zinc finger protein (ZFP) that will bind a specific sequence in the cargo. Cargo (double-stranded DNA) can be packaged inside the particle by various particle loading methods described herein, such as electroporation.
(41) A delivery vehicle (e.g., VLP, e.g., heVLP or humanized VLP) is created by producer cells expressing an envelope protein and gag and a zinc finger protein (ZFP) that will bind a specific sequence in the cargo fused to DmrA or DmrC in the presence of A/C Heterodimerizer molecule. Cargo (double-stranded DNA) can be packaged inside the particle by various particle loading methods described herein, such as electroporation.
(42) A delivery vehicle (e.g., VLP, e.g., heVLP or humanized VLP) is created by producer cells expressing an envelope protein and PH and a zinc finger protein (ZFP) that will bind a specific sequence in the cargo fused to DmrA or DmrC in the presence of A/C Heterodimerizer molecule. Cargo (double-stranded DNA) can be packaged inside the particle by various particle loading methods described herein, such as electroporation.
(43) A delivery vehicle (e.g., VLP, e.g., heVLP or humanized VLP) is created by producer cells expressing an envelope protein and gag/PH and a zinc finger protein (ZFP) that will bind a specific sequence in the cargo fused to DmrA or DmrC in the presence of A/C Heterodimerizer molecule. Cargo (double-stranded DNA) can be packaged inside the particle by various particle loading methods described herein, such as electroporation.
(44) A delivery vehicle (e.g., VLP, e.g., heVLP or humanized VLP) is created by producer cells expressing an envelope protein and gag fused to a zinc finger protein (ZFP) that will bind a specific sequence in the cargo. Cargo (double-stranded DNA bound by Cas9 RNP-ZFP fusion) can be packaged inside the particle by various particle loading methods described herein, such as electroporation. Alternatively, the Cas9 RNP-ZFP fusion could be expressed by the producer cells and the particles could be loaded by various particle loading methods described herein, such as electroporation.
(45) A delivery vehicle (e.g., VLP, e.g., heVLP or humanized VLP) is created by producer cells expressing an envelope protein and PH fused to a zinc finger protein (ZFP) that will bind a specific sequence in the cargo. Cargo (double-stranded DNA bound by Cas9 RNP-ZFP fusion) can be packaged inside the particle by various particle loading methods described herein, such as electroporation. Alternatively, the Cas9 RNP-ZFP fusion could be expressed by the producer cells and the particles could be loaded by various particle loading methods described herein, such as electroporation.
(46) A delivery vehicle (e.g., VLP, e.g., heVLP or humanized VLP) is created by producer cells expressing an envelope protein and gag/PH fused to a zinc finger protein (ZFP) that will bind a specific sequence in the cargo. Cargo (double-stranded DNA bound by Cas9 RNP-ZFP fusion) can be packaged inside the particle by various particle loading methods described herein, such as electroporation. Alternatively, the Cas9 RNP-ZFP fusion could be expressed by the producer cells and the particles could be loaded by various particle loading methods described herein, such as electroporation.
(47) A delivery vehicle (e.g., VLP, e.g., heVLP or humanized VLP) is created by producer cells expressing an envelope protein and gag fused to a zinc finger protein (ZFP) fused to DmrA or DmrC that will bind a specific sequence in the cargo in the presence of A/C Heterodimerizer molecule. Cargo (double-stranded DNA bound by Cas9 RNP-ZFP fusion) can be packaged inside the particle by various particle loading methods described herein, such as electroporation. Alternatively, the Cas9 RNP-ZFP fusion could be expressed by the producer cells and the particles could be loaded by various particle loading methods described herein, such as electroporation.
(48) A delivery vehicle (e.g., VLP, e.g., heVLP or humanized VLP) is created by producer cells expressing an envelope protein and PH fused to a zinc finger protein (ZFP) fused to DmrA or DmrC that will bind a specific sequence in the cargo in the presence of A/C Heterodimerizer molecule. Cargo (double-stranded DNA bound by Cas9 RNP-ZFP fusion) can be packaged inside the particle by various particle loading methods described herein, such as electroporation. Alternatively, the Cas9 RNP-ZFP fusion could be expressed by the producer cells and the particles could be loaded by various particle loading methods described herein, such as electroporation.
(49) A delivery vehicle (e.g., VLP, e.g., heVLP or humanized VLP) is created by producer cells expressing an envelope protein and gag/PH fused to a zinc finger protein (ZFP) fused to DmrA or DmrC that will bind a specific sequence in the cargo in the presence of A/C Heterodimerizer molecule. Cargo (double-stranded DNA bound by Cas9 RNP-ZFP fusion) can be packaged inside the particle by various particle loading methods described herein, such as electroporation. Alternatively, the Cas9 RNP-ZFP fusion could be expressed by the producer cells and the particles could be loaded by various particle loading methods described herein, such as electroporation.
(50) A delivery vehicle (e.g., VLP, e.g., heVLP or humanized VLP) is created by producer cells expressing an envelope protein. Cargo (RNA) is packaged inside the particle either by producer cells expressing cargo or particles being loaded by various particle loading methods described herein, such as electroporation.
(51) A delivery vehicle (e.g., VLP, e.g., heVLP or humanized VLP) is created by producer cells expressing an envelope protein. Cargo (RNA) is packaged inside the particle either by producer cells expressing cargo and gag or particles being loaded by various particle loading methods described herein, such as electroporation.
(52) A delivery vehicle (e.g., VLP, e.g., heVLP or humanized VLP) is created by producer cells expressing an envelope protein. Cargo (RNA) is packaged inside the particle either by producer cells expressing cargo and PH or particles being loaded by various particle loading methods described herein, such as electroporation.
(53) A delivery vehicle (e.g., VLP, e.g., heVLP or humanized VLP) is created by producer cells expressing an envelope protein. Cargo (RNA) is packaged inside the particle either by producer cells expressing cargo and gag/PH or particles being loaded by various particle loading methods described herein, such as electroporation.
(54) A delivery vehicle (e.g., VLP, e.g., heVLP or humanized VLP) is created by producer cells expressing an envelope protein. Cargo (RNA with MS2 stem loop(s)) is packaged inside the particle either by producer cells expressing cargo and gag fused to MS2 or particles being loaded by various particle loading methods described herein, such as electroporation.
(55) A delivery vehicle (e.g., VLP, e.g., heVLP or humanized VLP) is created by producer cells expressing an envelope protein. Cargo (RNA with MS2 stem loop(s)) is packaged inside the particle either by producer cells expressing cargo and PH fused to MS2 or particles being loaded by various particle loading methods described herein, such as electroporation.
(56) A delivery vehicle (e.g., VLP, e.g., heVLP or humanized VLP) is created by producer cells expressing an envelope protein. Cargo (RNA with MS2 stem loop(s)) is packaged inside the particle either by producer cells expressing cargo and gag/PH fused to MS2 or particles being loaded by various particle loading methods described herein, such as electroporation.
(57) A delivery vehicle (e.g., VLP, e.g., heVLP or humanized VLP) is created by producer cells expressing an envelope protein. Cargo (RNA with MS2 stem loop(s)) is packaged inside the particle either by producer cells expressing cargo and gag and MS2 fused to DmrA or DmrC in the presence of A/C heterodimerizer, or particles being loaded by various particle loading methods described herein, such as electroporation.
(58) A delivery vehicle (e.g., VLP, e.g., heVLP or humanized VLP) is created by producer cells expressing an envelope protein. Cargo (RNA with MS2 stem loop(s)) is packaged inside the particle either by producer cells expressing cargo and PH and MS2 fused to DmrA or DmrC in the presence of A/C heterodimerizer, or particles being loaded by various particle loading methods described herein, such as electroporation.
(59) A delivery vehicle (e.g., VLP, e.g., heVLP or humanized VLP) is created by producer cells expressing an envelope protein. Cargo (RNA with MS2 stem loop(s)) is packaged inside the particle either by producer cells expressing cargo and gag/PH and MS2 fused to DmrA or DmrC in the presence of A/C heterodimerizer, or particles being loaded by various particle loading methods described herein, such as electroporation.
(60) A delivery vehicle (e.g., VLP, e.g., heVLP or humanized VLP) is created by producer cells expressing an envelope protein. Cargo (RNA with RBP stem loop(s)) is packaged inside the particle either by producer cells expressing cargo fused to an RBP and gag fused to another RBP or particles being loaded by various particle loading methods described herein, such as electroporation.
(61) A delivery vehicle (e.g., VLP, e.g., heVLP or humanized VLP) is created by producer cells expressing an envelope protein. Cargo (RNA with RBP stem loop(s)) is packaged inside the particle either by producer cells expressing cargo fused to an RBP and PH fused to another RBP or particles being loaded by various particle loading methods described herein, such as electroporation.
(62) A delivery vehicle (e.g., VLP, e.g., heVLP or humanized VLP) is created by producer cells expressing an envelope protein. Cargo (RNA with RBP stem loop(s)) is packaged inside the particle either by producer cells expressing cargo fused to an RBP and gag/PH fused to another RBP or particles being loaded by various particle loading methods described herein, such as electroporation.
(63) A delivery vehicle (e.g., VLP, e.g., heVLP or humanized VLP) is created by producer cells expressing an envelope protein. Cargo (RNA with RBP stem loop(s)) is packaged inside the particle either by producer cells expressing cargo fused to an RBP and gag and another RBP fused to DmrA or DmrC in the presence of A/C Heterodimerizer molecule, or particles being loaded by various particle loading methods described herein, such as electroporation.
(64) A delivery vehicle (e.g., VLP, e.g., heVLP or humanized VLP) is created by producer cells expressing an envelope protein. Cargo (RNA with RBP stem loop(s)) is packaged inside the particle either by producer cells expressing cargo fused to an RBP and PH and another RBP fused to DmrA or DmrC in the presence of A/C Heterodimerizer molecule, or particles being loaded by various particle loading methods described herein, such as electroporation.
(65) A delivery vehicle (e.g., VLP, e.g., heVLP or humanized VLP) is created by producer cells expressing an envelope protein. Cargo (RNA with RBP stem loop(s)) is packaged inside the particle either by producer cells expressing cargo fused to an RBP and gag/PH and another RBP fused to DmrA or DmrC in the presence of A/C Heterodimerizer molecule, or particles being loaded by various particle loading methods described herein, such as electroporation.

### Systems

In aspects, also provided herein are nucleic acid molecules that encode one or more of the components of the delivery vehicles of the present disclosure. For instance, a nucleic acid molecule encoding the fusion protein is provided. A nucleic acid molecule encoding the cell fusion protein is also provided. In aspects, provided herein are compositions or systems that include nucleic acid molecules that encode one or more of the components of the delivery vehicles of the present disclosure. The compositions or systems can be used for producing a delivery vehicle of the present disclosure, for instance, by transfecting or otherwise delivering the nucleic acid molecules in the compositions or systems into a producer cell. The nucleic acid molecules can be expressed in the producer cell, the result of which assemble, package, and subsequently cause the producer cell to release the delivery vehicle.

In some cases, provided herein are producer cell lines that have been genetically modified to produce the delivery vehicles of the present disclosure. For instance, the producer cell can have one or more nucleic acid molecules that encode one or more of the components of the delivery vehicles of the present disclosure. The producer cells can be a stable cell line, or temporarily genetically modified. In some cases, provided herein are systems comprising the producer cells from which the delivery vehicles of the present disclosure are produced. In some cases, the systems further comprise the produced delivery vehicles. In some cases, the producer cell is a suitable cell line, e.g., a human cell line, such as VERO, WI38, MRC5, A549, HEK293, HEK293T, B-50 or any other HeLa cells, HepG2, Saos-2, HuH7, Chinese Hamster Ovary (CHO) cells, and HT1080 cell lines.

In some cases, a delivery vehicle of the present disclosure facilitates gene editing efficiency greater than 70%. In some cases, a delivery vehicle of the present disclosure facilitates gene editing efficiency comprising 8-fold increase of base editing efficiency when compared to conventional VLP (e.g., the VLPs described in Mangeot, P. E. et al. Genome editing in primary cells and in vivo using viral-derived Nanoblades loaded with Cas9-sgRNA ribonucleoproteins. Nat. Commun. 10, 45 (2019).). In some cases, a delivery vehicle of the present disclosure exhibits reduced immunogenicity in transduced target cells. In some cases, a delivery vehicle of the present disclosure produces reduced off-target genome editing in target cells when delivering genome editing system into the target cells. In some cases, a delivery vehicle of the present disclosure leads to more than 100-fold reduction in Cas-independent off-target editing. In some cases, a delivery vehicle of the present disclosure leads to at least 10-fold, such as 12- to 900-fold, lower Cas-dependent off-target editing.

### Pharmaceutical Composition and Method of Treatment

A delivery vehicle provided herein can find use in a variety of fields and methods. In some cases, the delivery vehicle of the present disclosure can be used to deliver one or more therapeutic cargos, such as therapeutic polypeptides, therapeutic nucleic acid molecules, or therapeutic ribonucleoprotein complexes, to a cell.

In some cases, the target cells to which the delivery vehicles are delivered are in vitro cells, ex vivo cells, or in vivo cells. The delivery vehicles of the present disclosure can be applicable for delivery of cargos into a variety of cell types, such as, animal cells, plant cells, bacteria cells, algal cells, or fungal cells.

In some cases, the target cells include animal cells, such as a cell derived from or present in an invertebrate animal (e.g., fruit fly, cnidarian, echinoderm, nematode), a vertebrate animal (e.g., fish, amphibians, reptiles, birds, mammals), a mammal (e.g., an ungulate (e.g., a pig, a cow, a goat, a sheep, a camel); a rodent (e.g., a rat, a mouse); a non-human primate; a human; a feline (e.g., a cat); a canine (e.g., a dog); etc.), and the like. In some embodiments, the target cell is a cell from an aquaculture animal (fish, crabs, shrimp, oysters etc.), a mammal, e.g., from a pet or zoo animal (cats, dogs, lizards, birds, lions, tigers and bears etc.), from a farm or working animal (horses, cows, pigs, chickens etc.), or a human. In some cases, the target cell comprises cultured cells, primary cells or cell lines, stem cells, progenitor cells, differentiated cells, germ cells, cancer cells (e.g., tumorigenic, metastatic), non-tumorigenic cells (normal cells), fetal cells, embryonic cells, adult cells, mitotic cells, non-mitotic cells, or any combination thereof.

In some cases, the target cells include cancer cells, and the cargo delivered by the delivery vehicle of the present disclosure includes agents that can lead to oncolysis (e.g., cell death of the cancer cells that the delivery vehicle enters). For instance, the cargo can include oncolytic polypeptides, e.g., polypeptides that can specifically kill cancer cells. Additionally or alternatively, the cargo can include polynucleotide encoding an oncolytic polypeptide. In some cases, the delivery vehicle (e.g., VLPs, heVLPs, or humanized VLPs) delivers polypeptides, polynucleotides, or small molecule agents, or any combination thereof, which can lead to oncolysis. In some cases, the delivery vehicle is pseudotyped with cell fusion proteins or carries one or more targeting moieties that mediate tropism toward cancer cells.

In some embodiments, a target cell is from an organ, a tissue, or an organism. The target cell can be removed from a subject prior to use in the methods disclosed herein, e.g., excised surgically, by venipuncture, etc. The target cell can be from a cell culture.

The delivery vehicles of the present disclosure can be applicable for delivery of cargos into a variety of different cell types, such as, but not limited to, a stem cell (e.g. an embryonic stem(ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, an immune cell, an epithelial cell, or an endothelial cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg). Other non-limiting examples of target cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells. The target cells can be adult stem cells that are resident in differentiated tissue, but retain the properties of self-renewal and ability to give rise to multiple cell types. In some cases, the target cells are somatic stem cells, such as, muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

In some cases, the target cell is a plant cell. For example, the target cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the target cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, Chinese artichoke (crosnes), Chinese cabbage, Chinese celery, Chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (Chinese mustard), gallon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf-green), lettuce (oak leaf-red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

In some cases, the target cell that the compositions and methods of the present disclosure are applicable for is an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Derimaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota, Holometabola, Hymenoptera, Coleoptera, Strepsiptera, Raphidioiptera, Megaloptera, Neuroptera, Mecoptera, Siphonaptera, Diptera, Trichoptera, or Lepidoptera.

In some cases, the target cell that the compositions and methods of the present disclosure are applicable for is an insect cell. For example, in some cases, the cell is a cell of a mosquito, a grasshopper, a true bug, a fly, a flea, a bee, a wasp, an ant, a louse, a moth, or a beetle.

### Pharmaceutical Composition

In one aspect, provided herein is a pharmaceutical composition comprising delivery vehicles according to some embodiments of the present disclosure. For instance, the pharmaceutical compositions provided herein can comprise at least one delivery vehicles according to some embodiments formulated with at least one pharmaceutically acceptable excipient.

For instance, the delivery vehicles disclosed herein can be formulated with at least one pharmaceutically acceptable excipient for parenteral administration by injection, e.g., subcutaneous injection, intramuscular injection, intravenous injection, or intrathecal injection. In some cases, the pharmaceutical composition is injected by bolus injection or continuous infusion. The method of administration of the pharmaceutical compositions include, but are not limited to, oral administration, rectal administration, parenteral, intravenous administration, intravitreal administration, intramuscular administration, inhalation, intranasal administration, topical administration, transdermal administration, ophthalmic administration or otic administration.

The pharmaceutical compositions can be presented in unit dosage form, e.g., in ampoules or in multi-dose containers. The pharmaceutical compositions can take such forms as suspensions, solutions or emulsions in oily or aqueous vehicles, and can contain formulation agents such as suspending, stabilizing and/or dispersing agents. Liquid preparations of the pharmaceutical compositions can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts for pH adjustment and/or osmolarity adjustment. Alternatively, the pharmaceutical compositions can be in powder form for constitution with a suitable vehicle, e.g., sterile pyrogen-free water.

The pharmaceutically acceptable excipient that can be used in the pharmaceutical compositions disclosed herein, in some cases, comprises any pharmaceutically acceptable material, composition or vehicle, for instance a solid or liquid filler, a diluent, an excipient, a carrier, a solvent or an encapsulating material, which can be involved in e.g., suspending, maintaining the activity of or carrying or transporting the therapeutic delivery vesicles from one organ, or portion of the body, to another organ, or portion of the body (e.g., from the blood to any tissue and/or organ and/or body part of interest).

A pharmaceutically acceptable excipient can be a non-carrier excipient. A non-carrier excipient serves as a vehicle or medium for a pharmaceutical composition described herein. A non-carrier excipient serves as a vehicle or medium for a pharmaceutical composition described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions may optionally comprise one or more additional active substances, e.g., therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Pharmaceutical compositions described herein can be used in therapeutic and veterinary applications. In some embodiments, pharmaceutical composition provided herein are suitable for administration to a subject, wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, goats, cats, dogs, mice, and/or rats; and/or birds, including commercially relevant birds such as parrots, poultry, chickens, ducks, geese, hens or roosters and/or turkeys; zoo animals, e.g., a feline; non-mammal animals, e.g., reptiles, fish, amphibians, etc.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the pharmaceutically acceptable carrier or excipient is a sugar (e.g., sucrose, lactose, mannitol, maltose, sorbitol or fructose), a neutral salt (e.g., sodium chloride, magnesium sulfate, magnesium chloride, potassium sulfate, sodium carbonate, sodium sulfite, potassium acid phosphate, or sodium acetate), an acidic component (e.g., fumaric acid, maleic acid, adipic acid, citric acid or ascorbic acid), an alkaline component (e.g., tris(hydroxymethyl) aminomethane (TRIS), meglumine, tribasic or dibasic phosphates of sodium or potassium), or an amino acid (e.g., glycine or arginine).

In some embodiments, a pharmaceutical composition can comprise a diluent (e.g., a parenterally acceptable diluent). A diluent may be a liquid diluent or a solid diluent. In some embodiments, a diluent may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl) propan-2-yl]amino]ethanesulfonic acid (TES), 3-(N-morpholino) propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Method of Treatment

In aspects, also provided herein are methods of treating a subject by administering a delivery vehicle or pharmaceutical composition according to some embodiments of the present disclosure.

The subject in the method of present disclosure can be an animal. In some embodiments, the subject is an animal cell. In some embodiments, the subject is a mammal. In some embodiments, the subject is a human. In some embodiments, the subject is an aquaculture animal (fish, crabs, shrimp, oysters etc.), a mammal, e.g., from a pet or zoo animal (cats, dogs, lizards, birds (e.g., parrots), lions, tigers and bears etc.), from a farm or working animal (horses, cows (e.g., dairy and beef cattle) pigs, chickens, turkeys, hens or roosters, goats, sheep, etc.), or a human. In some embodiments, the target cell as disclosed herein is in a subject to whom the method of the present disclosure is applicable.

The methods described herein can be therapeutic or veterinary methods for treating a subject. In some embodiments, the methods described herein are used to treat a disease resulting from a non-functional, poorly functional, or poorly expressed protein or gene product, for instance, resulting from a genetic mutation in one or more cells of the subject. In some embodiments, the methods described herein are used to treat a genetic disease (e.g., a mutation, a substitution, a deletion, an expansion, or a recombination), a monogenic disease, an inherited metabolic disease, a cancer, a neurodegenerative disease, a cardiovascular disease, a pulmonary disease, a renal disease, a liver disease, a genetic disease, a vascular disease, ophthalmic disease, musculoskeletal disease, lymphatic disease, auditory and inner ear disease, a metabolic disease, an inflammatory disease, an autoimmune disease, or an infectious disease. In some cases, provided herein are pharmaceutical compositions and methods for treating a retinal disease, e.g., Leber congenital amaurosis, by administering a pharmaceutical composition formulated for subretinal injection.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Materials and Methods

This example describes materials and methods that were used in the experiments described in Examples 2 and 3 below.

### Cell Culture Conditions

HEK293T cells (ATCC; CRL-3216), Gesicle Producer 293T cells (Takara; 632617), 3T3 cells (ATCC; CRL-1658), and Neuro-2a cells (ATCC; CCL-131) were maintained in DMEM+GlutaMAX (Life Technologies) supplemented with 10% (v/v) fetal bovine serum. Primary human and mouse fibroblasts were maintained in MEM alpha media (Thermo Fisher; 12571063) containing 20% (v/v) FBS, 2 mM GlutaMAX (Thermo Fisher; 35050061), 1% penicillin and streptomycin (Thermo Fisher; 15070063), 1× Nonessential amino acids (Thermo Fisher; 11140050), 1× Antioxidant Supplement (Sigma Aldrich; A1345), 10 ng/mL Epidermal Growth Factor from murine submaxillary gland (Sigma Aldrich; E4127) and 0.5 ng/mL Fibroblast Growth Factor (Sigma Aldrich; F3133). Cells were cultured at 37° C. with 5% carbon dioxide and were confirmed to be negative for mycoplasma by testing with MycoAlert (Lonza Biologics).

### Isolation of Primary Human T Cells

Primary human T cells were isolated as described previously (Chen et al., 2021). Buffy coats were obtained from Memorial Blood Centers (St. Paul, MN) and peripheral blood mononuclear cells were isolated using SepMate tubes (STEMCELL Technologies; 85450). The EasySep Human T-cell Isolation Kit was used to enrich for T-cells that were then frozen for long-term storage.

### Cloning

All plasmids used in this study were cloned using either USER cloning or KLD cloning as described previously (Doman et al., 2020). DNA was PCR-amplified using PhusionU Green Multiplex PCR Master Mix (Thermo Fisher Scientific). Mach1 (Thermo Fisher Scientific) chemically competent E. coli were used for plasmid propagation.

### BE-eVLP Production and Purification

BE-eVLPs were produced by transient transfection of Gesicle Producer 293T cells. Gesicle cells were seeded in T-75 flasks (Corning) at a density of 5′106 cells per flask. After 20-24 h, cells were transfected using the jetPRIME transfection reagent (Polyplus) according to the manufacturer's protocols. For producing v1-v3 BE-eVLPs, a mixture of plasmids expressing VSV-G (400 ng), MLVgag-pro-pol (2,800 ng), MLVgag-ABE8e (1,700 ng), and an sgRNA (4,400 ng) were co-transfected per T-75 flask. For MLVgag-ABE8e:MLVgag-pro-pol stoichiometry optimization, the total amount of plasmid DNA for these two components was fixed at 4,500 ng and the relative amounts of each were varied. For producing v4 BE-eVLPs, a mixture of plasmids expressing VSV-G (400 ng), MMLVgag-pro-pol (3,375 ng), MMLVgag-3×NES-ABE8e (1,125 ng), and an sgRNA (4,400 ng) were co-transfected per T-75 flask. BE-eVLP construct protein sequences are provided in Table 3.

40-48 h post-transfection, producer cell supernatant was harvested and centrifuged for 5 min at 500 g to remove cell debris. The clarified eVLP-containing supernatant was filtered through a 0.45-μm PVDF filter. For BE-eVLPs that were used in cell culture, unless otherwise stated, the filtered supernatant was concentrated 100-fold using PEG-it Virus Precipitation Solution (System Biosciences; LV825A-1) according to the manufacturer's protocols. For BE-eVLPs that were injected into mice, the filtered supernatant was concentrated 1000-3000-fold by ultracentrifugation using a cushion of 20% (w/v) sucrose in PBS. Ultracentrifugation was performed at 26,000 rpm for 2 h (4° C.) using either an SW28 rotor in an Optima XPN Ultracentrifuge (Beckman Coulter) or an AH-629 rotor in a Sorvall WX+ Ultracentrifuge (Thermo Fisher Scientific). Following ultracentrifugation, BE-eVLP pellets were resuspended in cold PBS (pH 7.4) and centrifuged at 1,000 g for 5 min to remove debris. BE-eVLPs were frozen at a rate of −1° C./min and stored at −80° C. BE-VLPs were thawed on ice immediately prior to use.

### BE-eVLP Transduction in Cell Culture and Genomic DNA Isolation

Cells were plated for transduction in 48-well plates (Corning) at a density of 30,000-40,000 cells per well. After 20-24 h, BE-eVLPs were added directly to the culture media in each well. 48-72 h post-transduction, cellular genomic DNA was isolated as previously reported (Doman et al., 2020). Briefly, cells were washed once with PBS and lysed in 150 μL of lysis buffer (10 mM Tris-HCl pH 8.0, 0.05% SDS, 25 μg mL-1 Proteinase K (Thermo Fisher Scientific)) at 37° C. for 1 h followed by heat inactivation at 80° C. for 30 min.

### High-Throughput Sequencing of Genomic DNA

Genomic DNA was isolated as described above. Following genomic DNA isolation, 1 μL of the isolated DNA (1-10 ng) was used as input for the first of two PCR reactions.

Genomic loci were amplified in PCR1 using PhusionU polymerase (Thermo Fisher Scientific). PCR1 primers for genomic loci are listed in Table 3. PCRI was performed as follows: 95° C. for 3 min; 30-35 cycles of 95° C. for 15 s, 61° C. for 20 s, and 72° C. for 30 s; 72° C. for 1 min. PCR1 products were confirmed on a 1% agarose gel. 1 μL of PCRI was used as an input for PCR2 to install Illumina barcodes. PCR2 was conducted for nine cycles of amplification using a Phusion HS II kit (Life Technologies). Following PCR2, samples were pooled and gel purified in a 1% agarose gel using a Qiaquick Gel Extraction Kit (Qiagen). Library concentration was quantified using the Qubit High-Sensitivity Assay Kit (Thermo Fisher Scientific). Samples were sequenced on an Illumina MiSeq instrument (paired-end read, read 1:200-280 cycles, read 2:0 cycles) using an Illumina MiSeq 300 v2 Kit (Illumina).

### High-Throughput Sequencing Data Analysis

Sequencing reads were demultiplexed using the MiSeq Reporter software (Illumina) and were analyzed using CRISPResso2 (Clement et al., 2019) as previously described (Doman et al., 2020). Batch analysis mode (one batch for each unique amplicon and sgRNA combination analyzed) was used in all cases. Reads were filtered by minimum average quality score (Q>30) prior to analysis. The following quantification window parameters were used: -w 20-wc-10. Base editing efficiencies are reported as the percentage of sequencing reads containing a given base conversion at a specific position. Prism 9 (GraphPad) was used to generate dot plots and bar plots.

### Immunoblot Analysis of BE-eVLP Protein Content

BE-eVLPs were lysed in Laemmli sample buffer (50 mM Tris-HCl pH 7.0, 2% sodium dodecyl sulfate (SDS), 10% (v/v) glycerol, 2 mM dithiothreitol (DTT)) by heating at 95° C. for 15 min. Lysed BE-eVLPs were spotted onto a dry nitrocellulose membrane (Thermo Fisher Scientific) and dried for 30 min. The membrane was blocked for 1 h at room temperature with rocking in blocking buffer: 1% bovine serum albumin (BSA) in TBST (150 mM NaCl, 0.5% Tween-20, and 50 mM Tris-HCl). After blocking, the membrane was incubated overnight at 4° C. with rocking with one of the following primary antibodies diluted in blocking buffer: mouse anti-Cas9 (Thermo Fisher; MA5-23519, 1:1000 dilution), mouse anti-MLV p30 (Abcam; ab130757, 1:1500 dilution), or mouse anti-VSV-G (Sigma Aldrich; V5507, 1:50000 dilution). The membrane was washed three times with 1×TBST (Tris-buffered saline+0.5% Tween-20) for 10 min each time at room temperature, then incubated with goat anti-mouse antibody (LI-COR IRDye 680RD; 926-68070, 1:10000 dilution in blocking buffer) for 1 h at room temperature with rocking. The membrane was washed as before and imaged using an Odyssey Imaging System (LI-COR).

### Western Blot Analysis of BE-eVLP Protein Content

BE-eVLPs were lysed as described above. Protein extracts were separated by electrophoresis at 150 V for 45 min on a NuPAGE 3-8% Tris-Acetate gel (Thermo Fisher Scientific) in NuPAGE Tris-Acetate SDS running buffer (Thermo Fisher Scientific). Transfer to a PVDF membrane was performed using an iBlot 2 Gel Transfer Device (Thermo Fisher Scientific) at 20 V for 7 min. The membrane was blocked for 1 h at room temperature with rocking in blocking buffer: 1% bovine serum albumin (BSA) in TBST (150 mM NaCl, 0.5% Tween-20, and 50 mM Tris-HCl). After blocking, the membrane was incubated overnight at 4° C. with rocking with mouse anti-Cas9 (Cell Signaling Technology; 14697, 1:1000 dilution). The membrane was washed three times with 1×TBST for 10 min each time at room temperature, then incubated with goat anti-mouse antibody (LI-COR IRDye 680RD; 926-68070, 1:10000 dilution in blocking buffer) for 1 h at room temperature with rocking. The membrane was washed as before and imaged using an Odyssey Imaging System (LI-COR). The relative amounts of cleaved ABE and full-length gag-ABE were quantified by densitometry using ImageJ, and the fraction of cleaved ABE relative to total (cleaved+full-length) ABE was calculated.

### Immunofluorescence Microscopy of Producer Cells

Gesicle Producer 293T cells were seeded at a density of 15,000 cells per well in PhenoPlate™ 96-well microplates coated with poly-D-lysine (PerkinElmer). After 24 h, cells were co-transfected with 1 ng of v2.4 or v3.4 BE-eVLP plasmids, 40 ng of mouse Dnmt1-targeting sgRNA plasmid, and 40 ng of pUC19 plasmid using the jetPRIME transfection reagent (Polyplus) according to the manufacturer's protocols. After 40 h, 32% aqueous paraformaldehyde (Electron Microscopy Sciences) was added dropwise directly into the cellular media to a final concentration of 4% paraformaldehyde. Cells were subsequently fixed for 20 min at room temperature. After fixation, cells were washed three times with PBS and then permeabilized with 1×PBST (PBS+0.1% Triton X-100) for 30 min at room temperature. Cells were then blocked in blocking buffer (3% w/v BSA in 1×PBST) for 30 min at room temperature. After blocking, cells were incubated overnight at 4° C. with mouse anti-Cas9 (Cell Signaling Technology; 14697, 1:250 dilution) and rabbit anti-tubulin (abcam; 52866, 1:400 dilution) diluted in blocking buffer. Cells were washed four times with 1×PBST, then incubated for 1 h at room temperature with goat anti-mouse AlexaFluor® 647-conjugated antibody (abcam; 150115, 1:500 dilution), goat anti-rabbit AlexaFluor® 488-conjugated antibody (abcam; 150077, 1:500 dilution), and 1 μM DAPI diluted in blocking buffer. Cells were washed three times with 1×PBST and two times with PBS before imaging using an Opera Phenix High-Content Screening System (PerkinElmer). Images were acquired using a 20× water immersion objective in a confocal mode. Automated image analysis was performed using the Harmony software (PerkinElmer). The normalized cytoplasmic intensity was determined by calculating the ratio of the mean cytoplasmic intensity of Cas9 signal per cell to the mean cytoplasmic intensity of tubulin signal per cell.

### Negative-Stain Transmission Electron Microscopy

Negative-stain TEM was performed at the Koch Nanotechnology Materials Core Facility of MIT. v4 BE-eVLPs were centrifuged for 5 min at 15,000 g to remove debris. From the clarified supernatant, 10 μL of sample and buffer containing solution was added to 200 mesh copper grid coated with a continuous carbon film. The sample was allowed to adsorb for 60 seconds after which excess solution was removed with kimwipes. 10 μL of negative staining solution containing 1% aqueous phosphotungstic acid was added to the TEM grid and the stain was immediately blotted off with kimwipes. The grid was then air-dried at room temperature in the chemical hood. The grid was then mounted on a JEOL single tilt holder equipped within the TEM column. The specimen was cooled down by liquid-nitrogen and then observed using JEOL 2100 FEG microscope at 200 kV with a magnification of 10,000-60,000. Images were taken using Gatan 2k×2k UltraScan CCD camera.

### BE-eVLP Protein Content Quantification

For protein quantification, BE-eVLPs were lysed in Laemmli sample buffer as described above. The concentration of BE protein in purified BE-eVLPs was quantified using the FastScan™ Cas9 (S. pyogenes) ELISA kit (Cell Signaling Technology; 29666C) according to the manufacturer's protocols. Recombinant Cas9 (S. pyogenes) nuclease protein (New England Biolabs; M0386) was used to generate the standard curve for quantification. The concentration of MLV p30 protein in purified BE-eVLPs was quantified using the MuLV Core Antigen ELISA kit (Cell Biolabs; VPK-156) according to the manufacturer's protocols. The concentration of VLP-associated p30 protein was calculated with the assumption that 20% of the observed p30 in solution was associated with VLPs, as was previously reported for MLV particles (Renner et al., 2020). The number of BE protein molecules per e VLP was calculated by assuming a copy number of 1800 molecules of p30 per eVLP, as was previously reported for MLV particles (Renner et al., 2020). The same analysis was used to determine eVLP titers for all therapeutic application experiments.

BE-eVLP sgRNA Extraction and Quantification

RNA was extracted from BE-eVLPs using the QIAmp Viral RNA Mini Kit (Qiagen; 52904) according to the manufacturer's protocols. Extracted RNA was reverse transcribed using SuperScript™ III First-Strand Synthesis SuperMix (Thermo Fisher Scientific; 18080400) and an sgRNA-specific DNA primer (Table 3) according to the manufacturer's protocols. qPCR was performed using a CFX96 Touch Real-Time PCR Detection System (Bio-Rad) with SYBR green dye (Lonza; 50512). The amount of cDNA input was normalized to MLV p30 content, and the sgRNA abundance per eVLP was calculated as log 2 [fold change] (DCq) relative to v1 BE-VLPs.

### Cell Viability Assays

Cell viability was quantified using a Promega CellTiter-Glo luminescent cell viability kit (Promega; G7570). 4×104 cells (for HEK293T and NIH 3T3) and 2.5×104 cells (for RDEB patient fibroblasts) were seeded in 250 μL of media per well. The cells were allowed to adhere for 16-18 h before treatment with BE-eVLPs. After 48 h of transduction, 100 μL of CellTiter-Glo reagent was added to each well in the dark. Cells were incubated for 10 min at room temperature and the 80 μL of solution was transferred into black 96-well flat bottom plates (Greiner Bio-one; 655096), and the luminescence was measured on a M1000 Pro microplate reader (Tecan) with a 1-second integration time. Cells treated with Opti-MEM were defined as 100% viable. The percentage of viable cells in BE-eVLP treated wells was calculated by normalizing the luminescence reading from each treatment well to the luminescence of PBS treated cells.

### Plasmid Transfections

Plasmid transfections were performed as described previously (Doman et al., 2020). Plasmids were prepared for transfection using a PlasmidPlus Midi Kit (Qiagen) with endotoxin removal. HEK293T cells were plated for transfection in 48-well plates (Corning) at a density of 40,000 cells per well. After 20-24 h, cells were transfected with 1 μg total DNA using 1.5 μL of Lipofectamine 2000 (Thermo Fisher Scientific) per well according to the manufacturer's protocols. Unless otherwise specified, 750 ng of base editor plasmid and 250 ng of guide RNA plasmid were co-transfected per well. Genomic DNA was isolated from transfected cells at 72 h post-transfection as described above.

### Assessment of Off-Target DNA Base Editing in HEK293T Cells

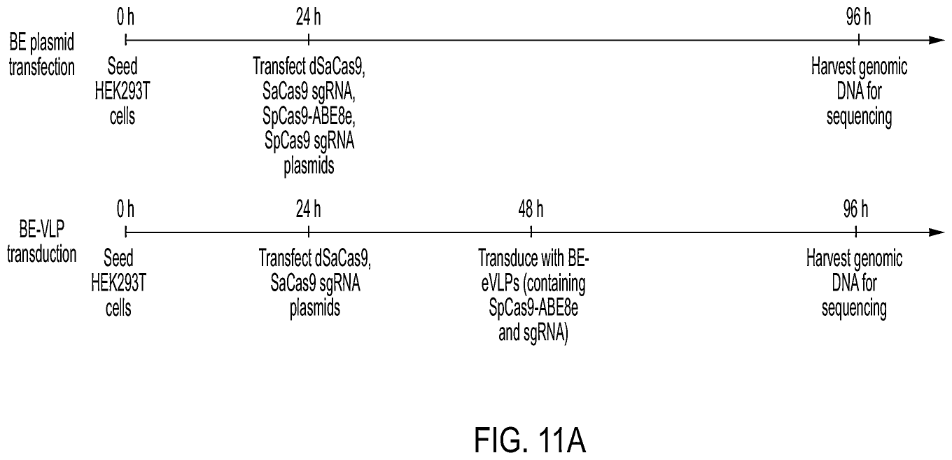

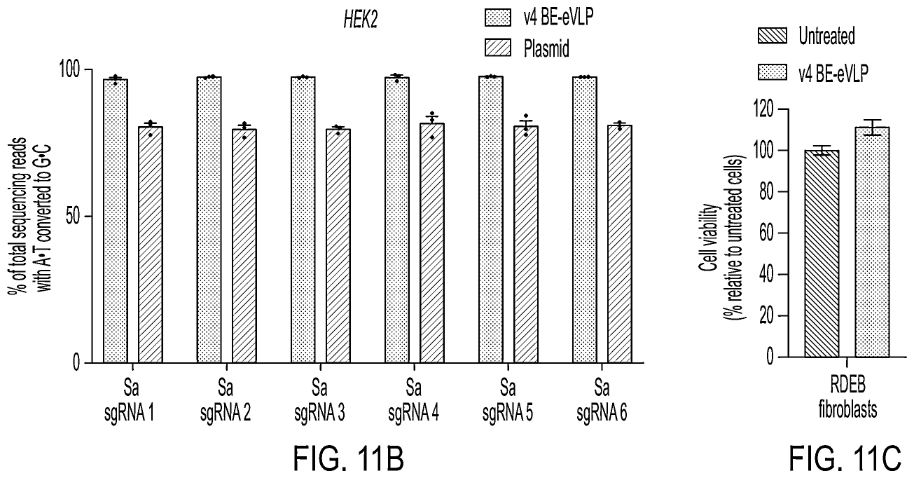

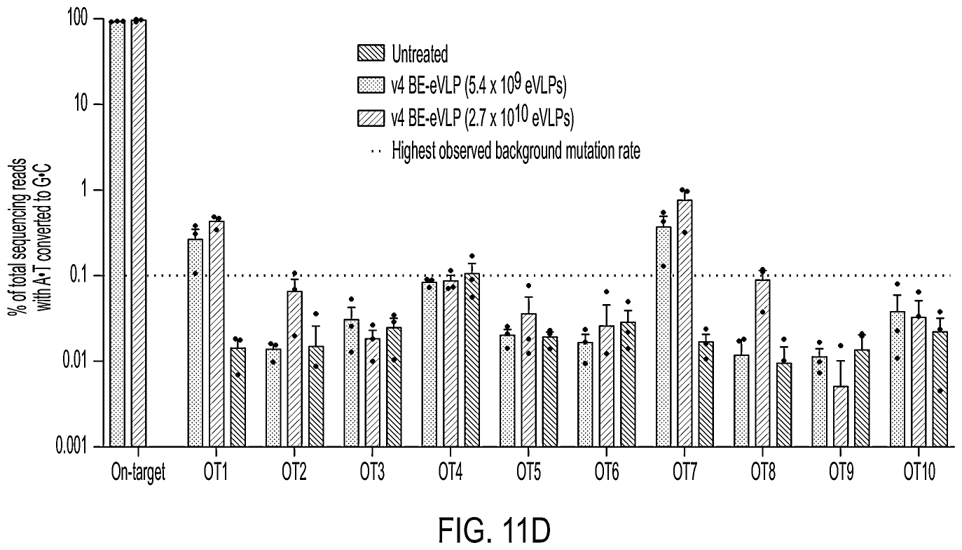

HEK293T cells were transduced with v4 BE-eVLPs or transfected with BE-encoding plasmid as described above. To assess Cas-dependent off-target editing, cells were transfected or transduced with 1 μL of v4 BE-eVLPs on the same day and genomic DNA was isolated 72 h post treatment in both cases. On-target and off-target loci were amplified and sequenced as described above. Orthogonal R-loop assays were performed as described previously (Doman et al., 2020) to assess Cas-independent off-target editing. To allow time for expression of SaCas9 and formation of the off-target R-loops following plasmid transfection, cells were transduced with 1 μL of PEG-concentrated v4 BE-eVLPs at 24 h post-transfection with dSaCas9- and orthogonal sgRNA-encoding plasmids. Genomic DNA was isolated 72 h post-transfection (48 h post-transduction) and sequenced as described above. See also FIG. 11A for an experimental schematic.

### Quantification of BE-Encoding DNA

For quantifying the amount of BE-encoding DNA in BE-eVLP preparations, v4 BE-eVLPs were lysed as described above, and the lysate was used as input into a qPCR reaction with BE-specific primers (Table 3). For quantifying the amount of BE-encoding DNA in eVLP-transduced vs. plasmid-transfected HEK293T cells, DNA was isolated from cell lysate as described above and used as input into a qPCR reaction with BE-specific primers (Table 3). In both cases, a standard curve was generated with BE-encoding plasmid standards of known concentration and was used to infer the amount of BE-encoding DNA present in the original samples.

### Transduction of T Cells and Genomic DNA Preparation

Thawed cells (day 0) were rested for 24 h in basal T-cell media comprised of X-VIVO™ 15 Serum-free Hematopoietic Cell Medium (Lonza; BE02-0606F) with 10% AB human serum (Valley Biomedical; HP1022), 2 mg/mL N-acetyl-cysteine (Sigma Aldrich; A7250), 300 IU/mL recombinant human IL-2 (Peprotech; 200-02) and 5 ng/mL recombinant human IL-7 (Peprotech; 200-07) and 5 ng/mL IL-15 (Peprotech; 500-P15).

On day 1, 50,000 cells in 50 μL of T-cell media were plated in 96-well-plates coated with 10 μg/cm2 RectroNectin® (Clontech/Takara; catalog number T100A/B). 5 μL (3.0×1010 eVLPs) of ultracentrifuge-purified v4 BE-eVLPs were used to transduce the cells on day 1 and on day 2 the cells were stimulated with Dynabeads™ Human T-Expander CD3/CD28 beads (Thermo Fisher; 11161D). Beads were added at a bead to cell ratio of 3:1 in a volume of 50 μL. On day 3, the cells were transduced for a second time with 5 μL (3.0×1010 eVLPs) of v4 BE-eVLPs in a total media volume of 200 μL.

Twenty-four hours later (day 4) the cells were resuspended in 1 mL of fresh T-cell media and re-plated in wells of a 48 well plate. On day 6 the cells were harvested and genomic DNA was isolated using the QuickExtract™ DNA Extraction Solution (Lucigen; QE09050).

### Lentiviral Vector Cloning and Production

Lentiviral vectors were constructed via USER cloning into the lentiCRISPRv2 backbone (Addgene #135955). Lentiviral transfer vectors were propagated in NEB Stable Competent E. coli (New England Biolabs). HEK293T/17 (ATCC CRL-11268) cells were maintained in antibiotic-free DMEM supplemented with 10% fetal bovine serum (v/v).

On day 1, 5′106 cells were plated in 10 mL of media in T75 flasks. The following day, cells were transfected with 6 μg of VSV-G envelope plasmid, 9 μg of psPAX2 (plasmid encoding viral packaging proteins) and 9 μg of transfer vector plasmid (plasmid encoding the gene of interest) diluted in 1,500 μL Opti-MEM with 70 μL of FuGENE. Two days after transfection, media was centrifuged at 500 g for 5 min to remove cell debris following filtration using 0.45-μm PVDF vacuum filter. The lentiviruses were further concentrated by ultracentrifugation with a 20% (w/v) sucrose cushion as described above for eVLP production.

### AAV Production

AAV production was performed as previously described (Deverman et al., 2016; Levy et al., 2020) with some alterations. HEK293T/17 cells were maintained in DMEM with 10% fetal bovine serum without antibiotics in 150-mm dishes (Thermo Fisher Scientific; 157150) and passaged every 2-3 days. Cells for production were split 1:3 one day before polyethylenimine transfection. Then, 5.7 μg AAV genome, 11.4 μg pHelper (Clontech) and 22.8 μg AAV8 rep-cap plasmid were transfected per plate. The day after transfection, media was exchanged for DMEM with 5% fetal bovine serum. Three days after transfection, cells were scraped with a rubber cell scraper (Corning), pelleted by centrifugation for 10 min at 2,000 g, resuspended in 500 μl hypertonic lysis buffer per plate (40 mM Tris base, 500 mM NaCl, 2 mM MgCl₂ and 100 U mL⁻¹ salt active nuclease

(ArcticZymes; 70910-202)) and incubated at 37° C. for 1 h to lyse the cells. The media was decanted, combined with a 5× solution of 40% poly(ethylene glycol) (PEG) in 2.5 M NaCl (final concentration: 8% PEG/500 mM NaCl), incubated on ice for 2 h to facilitate PEG precipitation, and centrifuged at 3,200 g for 30 min. The supernatant was discarded and the pellet was resuspended in 500 μL lysis buffer per plate and added to the cell lysate. Crude lysates were either incubated at 4° C. overnight or directly used for ultracentrifugation.

Cell lysates were clarified by centrifugation at 2,000 g for 10 min and added to Beckman Quick-Seal tubes via 16-gauge 5″ disposable needles (Air-Tite N165). A discontinuous iodixanol gradient was formed by sequentially floating layers: 9 mL 15% iodixanol in 500 mM NaCl and 1×PBS-MK (1×PBS plus 1 mM MgCl₂ and 2.5 mM KCl), 6 mL 25% iodixanol in 1×PBS-MK, and 5 mL each of 40 and 60% iodixanol in 1×PBS-MK. Phenol red at a final concentration of 1 μg mL-1 was added to the 15, 25 and 60% layers to facilitate identification. Ultracentrifugation was performed using a Ti 70 rotor in a Optima XPN-100 Ultracentrifuge (Beckman Coulter) at 58,600 rpm for 2 h 15 min at 18° C.

Following ultracentrifugation, 3 mL of solution was withdrawn from the 40-60% iodixanol interface via an 18-gauge needle, dialyzed with PBS containing 0.001% F-68 using 100-KD MWCO columns (EMD Millipore). The concentrated viral solution was sterile filtered using a 0.22-μm filter. The final AAV preparation was quantified via qPCR (AAVpro Titration Kit version 2; Clontech), and stored at 4° C. until use.

### Animals

All mice experiments were approved by the Broad Institute, the University of California, Irvine, and the University of Pennsylvania institutional animal care and use committees. Timed pregnant C57BL/6J mice for P0 studies were purchased from Charles River Laboratories (027). Wild-type adult C57BL/6J mice (000664) and pigmented rd12 mice (005379) were purchased from the Jackson Laboratory. All mice were housed in a room maintained on a 12 h light and dark cycle with ad libitum access to standard rodent diet and water. Animals were randomly assigned to various experimental groups.

### P0 Ventricle Injections

P0 ventricle injections were performed as described previously (Levy et al., 2020). Drummond PCR pipettes (5-000-1001-X₁₀) were pulled at the ramp test value on a Sutter P1000 micropipette puller and passed through a Kimwipe three times, resulting in a tip size of ˜100 μm. A small amount of Fast Green was added to the BE-eVLP injection solution to assess ventricle targeting. The injection solution was loaded via front filling using the included Drummond plungers. P0 pups were anaesthetized by placement on ice for 2-3 min until they were immobile and unresponsive to a toe pinch. Then, 2 μL of injection mix (containing 2.6×10¹⁰ eVLPs encapsulating a total of 3.2 pmol of BE protein) was injected freehand into each ventricle. Ventricle targeting was assessed by the spread of Fast Green throughout the ventricles via transillumination of the head.

### Nuclear Isolation and Sorting

Nuclei were isolated from the cortex and the mid-brain as previously described (Levy et al., 2020). Briefly, dissected cortex and mid-brain were homogenized using a glass Dounce homogenizer (Sigma-Aldrich; D8938) with 20 strokes using pestle A followed by 20 strokes from pestle B in 2 mL of ice-cold EZ-PREP buffer (Sigma-Aldrich; NUC-101). Samples were then decanted into a new tube containing an additional 2 mL of EZ-PREP buffer on ice. After 5 min, homogenized tissues were centrifuged for 5 min at 500 g at 4° C. The nuclei pellet was resuspended in 4 mL of ice-cold Nuclei Suspension Buffer (NSB) consisting of 100 μg/mL BSA (NEB; B9000S) and 3.33 μM Vybrant DyeCycle Ruby (Thermo Fisher; V10309) in PBS followed by centrifugation at 500 g for 5 min at 4° C. After centrifugation, the supernatant was removed, and nuclei were resuspended in 1-2 mL of NSB, passed through 35-μm cell strainer, followed by flow sorting using the Sony MA900 Cell Sorter (Sony Biotechnology) at the Broad Institute flow cytometry core. See Figure S5A for example FACS gating. Nuclei were sorted into DNAdvance lysis buffer, and the genomic DNA was purified according to the manufacturer's protocol (Beckman Coulter; A48705).

### Retro-Orbital Injections

50 μL of VLPs (containing 4×1011 or 7×1011 VLPs) were centrifuged for 10 min at 15,000 g to remove debris. The clarified supernatant was diluted to 120 μL in 0.9% NaCl (Fresenius Kabi; 918610) right before injection. 1×1011 viral genomes (vg) of total AAV was diluted to 120 μL in 0.9% NaCl (Fresenius Kabi; 918610) right before injection.

Anesthesia was induced with 4% isoflurane. Following induction, as measured by unresponsiveness to bilateral toe pinch, the right eye was protruded by gentle pressure on the skin, and an insulin syringe was advanced, with the bevel facing away from the eye, into the retrobulbar sinus where VLP or AAV mix was slowly injected. One drop of Proparacaine Hydrochloride Ophthalmic Solution (Patterson Veterinary; 07-885-9765) was then applied to the eye as an analgesic. Genomic DNA was purified from various tissue using Agencourt DNAdvance kits (Beckman Coulter; A48705) following the manufacturer's instructions.

### Histology and Staining

Liver tissue was fixed in 4% PFA overnight at 4° C. The next day, fixed liver was transferred into 1×PBS with 10 mM glycine to quench free aldehyde for at least 24 h followed by paraffinization at the Rodent Histopathology Core of Harvard Medical School. Liver paraffin block was then cut into 5 μm sections followed by hematoxylin and eosin staining for histopathological examination.

### Alanine Aminotransferase (ALT) and Aspartate Aminotransferase (AST) Assay

Blood was collected 7 days after injection via submandibular bleeding and allowed to clot at room temperature for 1 h. The serum was then separated by centrifugation at 2000 g for 15 min and sent to IDEXX Bioanalytics, MA, for analysis.

### Serum Pcsk9 Measurements

To track serum levels of Pcsk9 blood was collected using a submandibular bleed in a serum separator tube. Serum was separated by centrifugation at 2000 g for 15 min and stored at −80° C. Pcsk9 levels were determined by ELISA using the Mouse Proprotein Convertase 9/PCSK9 Quantikine ELISA Kit (R&D Systems; MPC900) following the manufacturer's instructions.

### CIRCLE-seq

Circularization for In vitro Reporting of Cleavage Effects by sequencing (CIRCLE-seq) was performed and analyzed as described previously (Tsai et al., 2017) save for the following modifications: For the Cas9 cleavage step, guide denaturation, incubation, and proteinase K treatment was conducted using the more efficient method described in the CHANGE-seq protocol (Lazzarotto et al., 2020). Specifically, the sgRNA with the guide sequence “GCCCATACCTTGGAGCAACGG” (SEQ ID NO: 52) was ordered from Synthego with their standard chemical modifications, 2′O-Methyl for the first three and last three bases, and phosphorothioate bonds between the first three and last two bases. A 5′ “G” nucleotide was included with the 20-nucleotide specific guide sequence to recapitulate the sequence expressed and packaged into VLPs. The sgRNA was diluted to 9 UM in nuclease-free water and re-folded by incubation at 90° C. for 5 min followed by a slow annealing down to 25° C. at a ramp rate of 0.1° C./second. The sgRNA was complexed with Cas9 nuclease (NEB; M0386T) via a 10 min room temperature incubation after mixing 5 μL of 10× Cas9 Nuclease Reaction Buffer provided with the nuclease, 4.5 μL of 1 μM Cas9 nuclease (diluted from the 20 μM stock in 1× Cas9 Nuclease Reaction Buffer), and 1.5 μL of 9 UM annealed sgRNA. Circular DNA from mouse N2A cells was added to a total mass of 125 ng and diluted to a final volume of 50 μL. Following 1 h of incubation at 37° C., Proteinase K (NEB; P8107S) was diluted 4-fold in water and 5 μL of the diluted mixture was added to the cleavage reaction. Following a 15 min Proteinase K treatment at 37° C., DNA was A-tailed, adapter ligated, and USER-treated, and PCR-amplified as described in the CIRCLE-seq protocol (Tsai et al., 2017). Following PCR, samples were loaded on a preparative 1% agarose gel and DNA was extracted between the 300 bp and 1 kb range to eliminate primer dimers before sequencing on an Illumina MiSeq. Data was processed using the CIRCLE-seq analysis pipeline and aligned to the human genome Hg19 (GRCh37) with parameters: “read_threshold: 4; window_size: 3; mapq_threshold: 50; start_threshold: 1; gap_threshold: 3; mismatch_threshold: 6; merged_analysis: True”.

### Amplicon Sequencing of Off-Target Sites Nominated by CIRCLE-Seq

In prior work, it was observed for exhaustively assessed ABE8e off-target sites nominated by CIRCLE-seq that off-target editing efficiency did not track well with the CIRCLE-seq read count (Newby et al., 2021). However, nominated off-target sites where editing was observed shared some striking similarities. Namely, over 90.7% of the 54 off-target sites with validated off-target editing had zero mismatches or one mismatch to the guide in the 9 nucleotides proximal to the PAM. The few sites with more than 1 mismatch in this region were all edited with low efficiency (the bottom half of sites, when ranked by editing efficiency). Based on this knowledge, 14 off-target sites in the CIRCLE-seq list that showed one or fewer mismatches in the 9 nucleotides of the protospacer proximal to the PAM were chosen to be assessed to increase the chance that a true off-target site would be sequenced (Table 3).

### Mouse Subretinal Injection

Mice were anesthetized by intraperitoneal injection of a cocktail consisting of 20 mg/mL ketamine and 1.75 mg/mL xylazine in phosphate-buffered saline at a dose of 0.1 mL per 20 g body weight, and their pupils were dilated with topical administration of 1% tropicamide ophthalmic solution (Akorn; 17478-102-12). Subretinal injections were performed under an ophthalmic surgical microscope (Zeiss). An incision was made through the cornea adjacent to the limbus at the nasal side using a 25-gauge needle. A 34-gauge blunt-end needle (World Precision Instruments; NF34BL-2) connected to an RPE-KIT (World Precision Instruments, no. RPE-KIT) by SilFlex tubing (World Precision Instruments; SILFLEX-2) was inserted through the corneal incision while avoiding the lens and advanced through the retina. Each mouse was injected with 1 μL of experimental reagent (lentivirus or eVLPs) per eye. Lentivirus titer was >1×109 TU/mL as measured by the QuickTiter™ Lentivirus Titer Kit (Cell Biolabs; VPK-107-5). BE-eVLPs were normalized to a titer of 4×1010 eVLPs/μL, corresponding to an encapsulated BE protein content of 3 pmol/μL. After injections, pupils were hydrated with the application of GenTeal Severe Lubricant Eye Gel (0.3% Hypromellose, Alcon) and kept for recovery.

### RPE Dissociation and Genomic DNA and RNA Preparation

Under a light microscope, mouse eyes were dissected to separate the posterior eyecup (containing RPE, choroid and sclera) from the retina and anterior segments. Each posterior eyecup was immediately immersed in 350 μl of RLT Plus tissue lysis buffer provided with AllPrep DNA/RNA Mini Kit (Qiagen; 80284). After 1 min incubation, RPE cells were detached in the lysis buffer from the posterior eyecup by gentle pipetting, followed by a removal of the remaining posterior eyecup. The lysis buffer containing RPE cells was further processed for DNA and RNA extraction using the AllPrep DNA/RNA Mini Kit protocol. The final DNA and RNA were eluted in 30 μL and 15 μL water, respectively. cDNA synthesis was performed using the SuperScript™ III First-Strand Synthesis SuperMix (Thermo Fisher; 18080400).

### Western Blot Analysis of Mouse RPE Tissue Extracts

To prepare the protein lysate from the mouse RPE tissue, the dissected mouse eyecup, consisting of RPE, choroid, and sclera, was transferred to a microcentrifuge tube containing 30 μL of RIPA buffer with protease inhibitors and homogenized with a motor tissue grinder (Fisher Scientific; K749540-0000) and centrifuged for 30 min at 20,000 g at 4° C. The resulting supernatant was pre-cleared with Dynabeads Protein G (Thermo Fisher; 10003D) to remove contaminants from blood prior to gel loading. Twenty μL of RPE lysates pre-mixed with NuPAGE LDS Sample Buffer (Thermo Fisher; NP0007) and NuPAGE Sample Reducing Agent (Thermo Fisher; NP0004) was loaded into each well of a NuPAGE 4-12% Bis-Tris gel (Thermo Fisher; NP0321BOX), separated for 1 h at 130 V and transferred onto a PVDF membrane (Millipore; IPVH00010). After 1 h blocking in 5% (w/v) non-fat milk in PBS containing 0.1% (v/v) Tween-20 (PBS-T), the membrane was incubated with primary antibody, mouse anti-RPE65 monoclonal antibody (1:1,000; in-house production) (Golczak et al., 2010), diluted in 1% (w/v) non-fat milk in PBS-T overnight at 4° C. After overnight incubation, membranes were washed three times with PBS-T for 5 min each and then incubated with goat anti-mouse IgG-HRP antibody (1:5,000; Cell Signaling Technology; 7076S) for 1 h at room temperature.

After washing the membrane three times with PBS-T for 5 min each, protein bands were visualized after exposure to SuperSignal West Pico Chemiluminescent substrate (Thermo Fisher; 34580). Membranes were stripped and reprobed for ABE and β-actin expression using mouse anti-Cas9 monoclonal antibody (1:1,000; Invitrogen; MA523519) and rabbit anti-β-actin polyclonal antibody (1:1,000; Cell Signaling Technology; 4970S), following the same protocol. Corresponding secondary antibodies were goat anti-mouse IgG-HRP antibody (1:5,000; Cell Signaling Technology; 7076S) and goat anti-rabbit IgG-HRP antibody (1:5,000; Cell Signaling Technology; 7074S).

### Electroretinography

Prior to recording, mice were dark adapted for 24 h overnight. Under a safety light, mice were anesthetized by intraperitoneal injection of a cocktail consisting of 20 mg/mL ketamine and 1.75 mg/mL xylazine in phosphate-buffered saline at a dose of 0.1 mL per 20 g body weight, and their pupils were dilated with topical administration of 1% tropicamide ophthalmic solution (Akorn; 17478-102-12) followed by 2.5% hypromellose (Akorn; 9050-1) for hydration. The mouse was placed on a heated Diagnosys Celeris rodent ERG device (Diagnosys LCC). Ocular electrodes were placed on the corneas, and the reference electrode was positioned subdermally between the ears. The eyes were stimulated with a green light (peak emission 544 nm, bandwidth ˜160 nm) stimulus of −0.3 log (cd·s/m2). The responses for 10 stimuli with an inter-stimulus interval of 10 s were averaged together, and the a- and b-wave amplitudes were acquired from the averaged ERG waveform. The ERGs were recorded with the Celeris rodent electrophysiology system (Diagnosys LLC) and analyzed with Espion V6 software (Diagnosys LLC).

### Quantification And Statistical Analysis

Data are presented as mean and standard error of the mean (s.e.m.). No statistical methods were used to predetermine sample size. Statistical analysis was performed using GraphPad Prism software. Sample size and the statistical tests used are described in the figure legends.

### Example 2: Development and Testing of Viral-like Particles for Base Editing

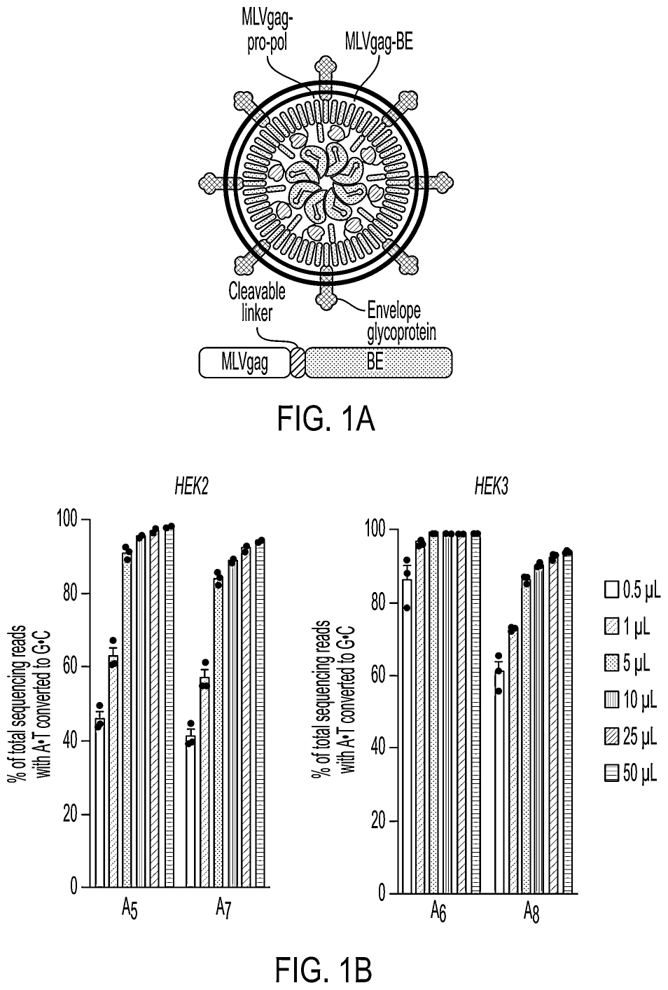

As an initial (v1) BE-VLP design, ABE8e, a highly active adenine base editor (Richter et al., 2020), was fused to the C-terminus of the Friend murine leukemia virus (FMLV) gag polyprotein via a linker peptide that would be cleaved by the FMLV protease upon particle maturation (FIG. 1A). FMLV-based VLPs were previously used successfully to package and deliver Cas9 RNPs (Mangeot et al., 2019). BE-VLPs were produced by transfecting Gesicle 293T producer cells with plasmids expressing this FMLV gag-ABE8e fusion construct, wild-type FMLV gag-pro-pol polyprotein, the VSV-G envelope glycoprotein, and an sgRNA targeting HEK293T cell genomic site 2 or site 3, hereafter referred to as HEK2 or HEK3.

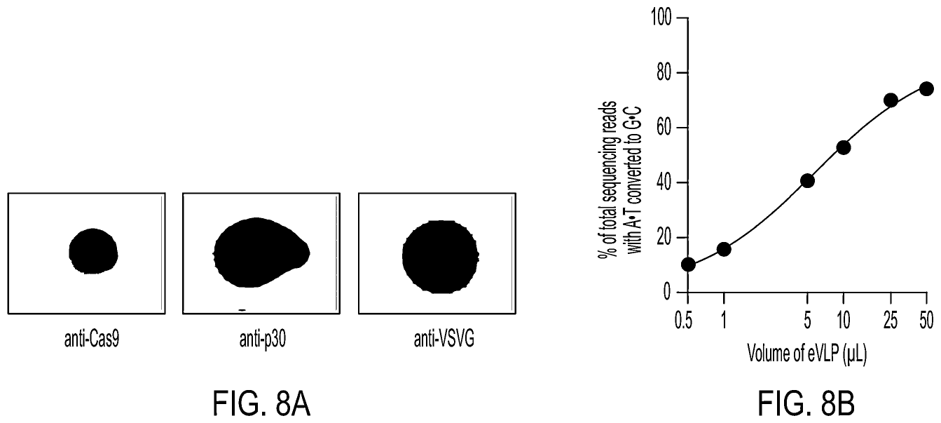

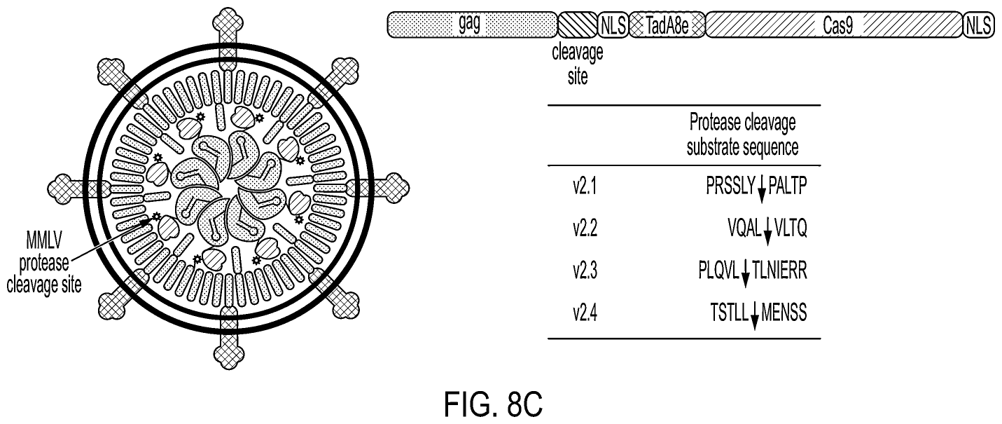

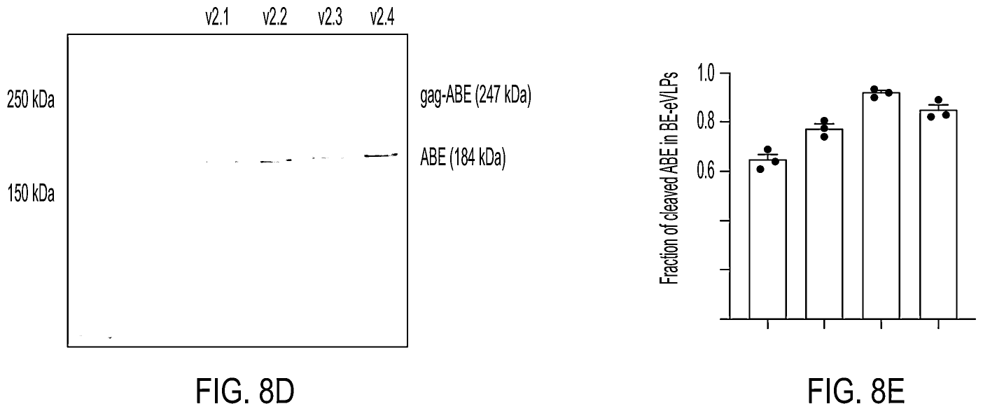

After harvesting BE-VLPs from producer cell supernatant, HEK293T cells were transduced in vitro with concentrated BE-VLPs. Encouragingly, v1 BE-VLPs robustly edited the HEK2 and HEK3 genomic loci with efficiencies >97% at the highest doses in unsorted cells (FIG. 1B). It was confirmed via immunoblotting that these BE-VLPs contained Cas9, the MLV capsid, and VSV-G proteins (FIG. 8A). These observations indicated that the FMLV retroviral scaffold supports BE-VLP formation and that v1 BE-VLPs can efficiently transduce and edit HEK293T cells in vitro.

Improving Cargo Release after VLP Maturation

While v1 BE-VLPs robustly edited the HEK2 and HEK3 loci in HEK293T cells, these commonly used test loci are especially amenable to gene editing and lack therapeutic relevance (Anzalone et al., 2020). To evaluate the therapeutic potential of BE-VLPs, the ability of the BE-VLPs were assessed to install mutations in the BCL11A erythroid-specific enhancer that upregulate the expression of fetal hemoglobin in erythrocytes, an established base editing strategy for the treatment of β-hemoglobinopathies (Richter et al., 2020; Zeng et al., 2020). It was observed that v1 BE-VLPs achieved 73% editing at the BCL11A enhancer locus in HEK293T cells at high doses, but editing levels dropped steeply with decreasing doses (FIG. 8B). These results indicated that v1 BE-VLP activity could be improved.

Cleavage of the gag-ABE8e linker by the MLV protease after particle maturation is required to liberate free ABE8e RNP. A series of second-generation (v2) engineered BE-eVLPs were constructed that contain a variety of protease-cleavable linker sequences between the MLV gag and ABE8e (FIG. 8C).

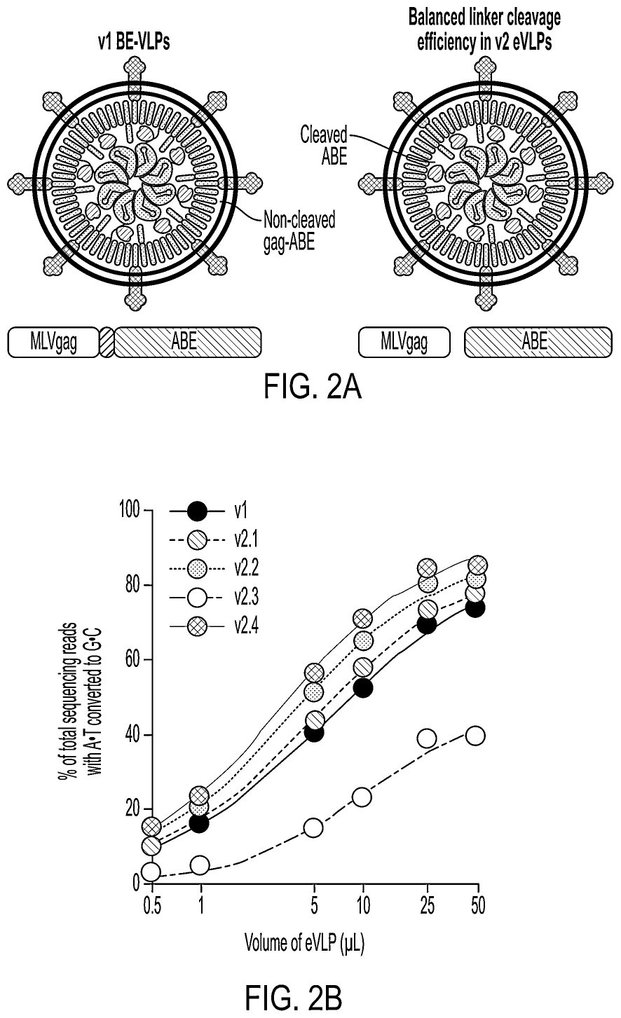

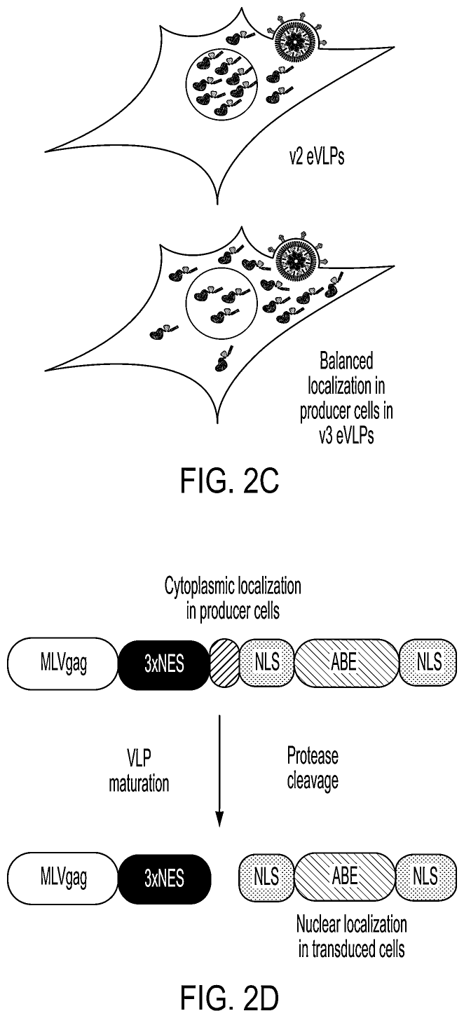

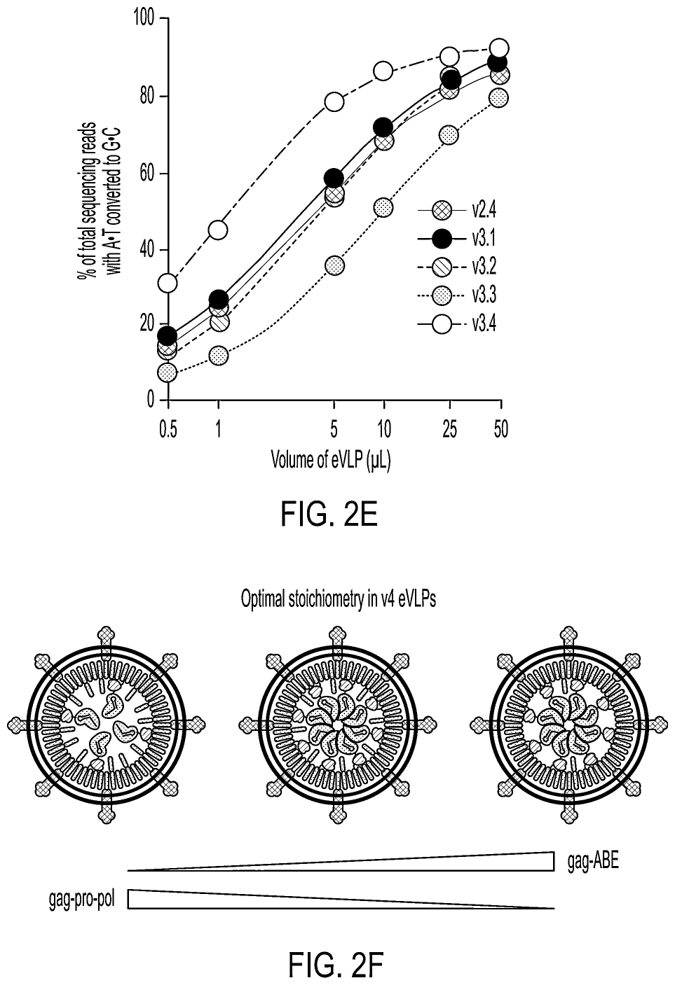

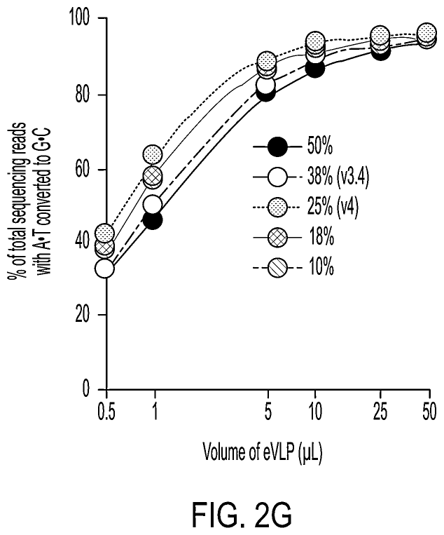

First, the retroviral scaffold was switched from Friend MLV to Moloney MLV (MMLV), a similar MLV strain whose protease substrate specificity has been extensively characterized (Feher et al., 2006). Then four different linker sequences were tested, which were known to be cleaved with varying efficiencies by the MMLV protease and identified several new gag-ABE8e linkers that improved editing efficiencies compared to v1 BE-VLPs (FIG. 2B). Specifically, v2.4 BE-eVLPs exhibited 1.2-1.5-fold higher editing efficiencies at all doses tested relative to v1 BE-VLPs (FIG. 2B). To investigate the cleavage efficiencies of the linker sequences in v2.1-v2.4 BE-eVLPs, western blots were performed to determine the fraction of cleaved ABE8e versus full-length gag-ABE8e present in purified BE-VLPs. This analysis revealed that the v2.4 linker is cleaved more efficiently than the v2.1 and v2.2 linkers, but less efficiently than the v2.3 linker (FIGS. 8D and 8E).

These results support a model in which the linker sequence in v2.4 BE-eVLPs is cleaved at an optimal rate that supports efficient release of ABE8e RNP after VLP maturation but precludes premature release of ABE8e RNP prior to its incorporation into VLPs. The findings demonstrate that the gag-cargo protein linker sequence is an important parameter of VLP architectures and that optimizing this sequence to balance the linker cleavage kinetics between these two constraints can improve eVLP activity.

Improving Cargo Localization and Loading into eVLPs

Previously optimized BEs are fused at their N- and C-termini to bipartite nuclear localization signals (NLSs), which promotes nuclear import of BEs and enhances their access to genomic DNA (Koblan et al., 2018). However, gag-BE fusions must be localized to the cytoplasm and outer membrane of producer cells in order to be incorporated into VLPs as they form (FIG. 2C). Without wishing to be bound by a certain theory, the presence of two NLSs within the gag-BE fusion can hamper gag-BE localization to the outer membrane and impede BE incorporation into VLPs.

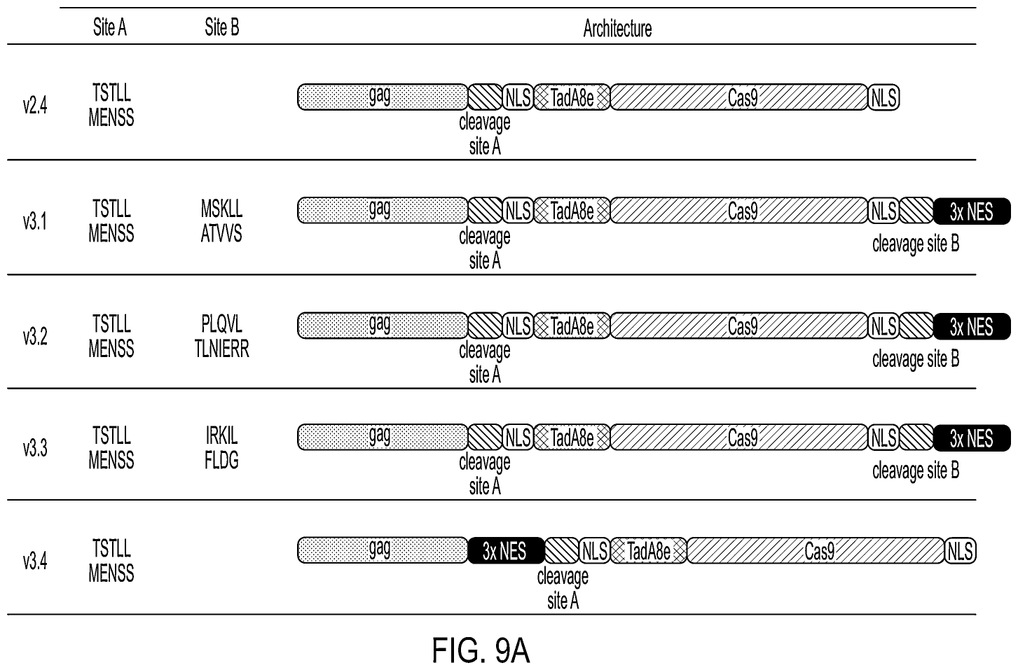

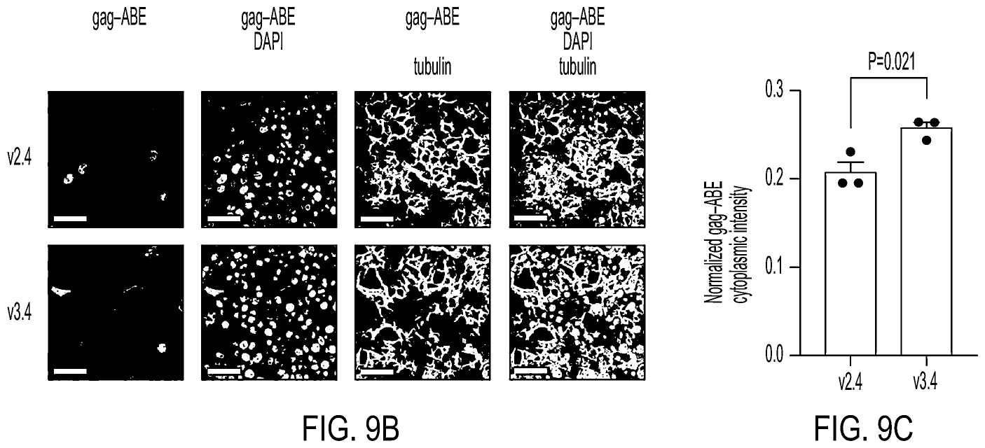

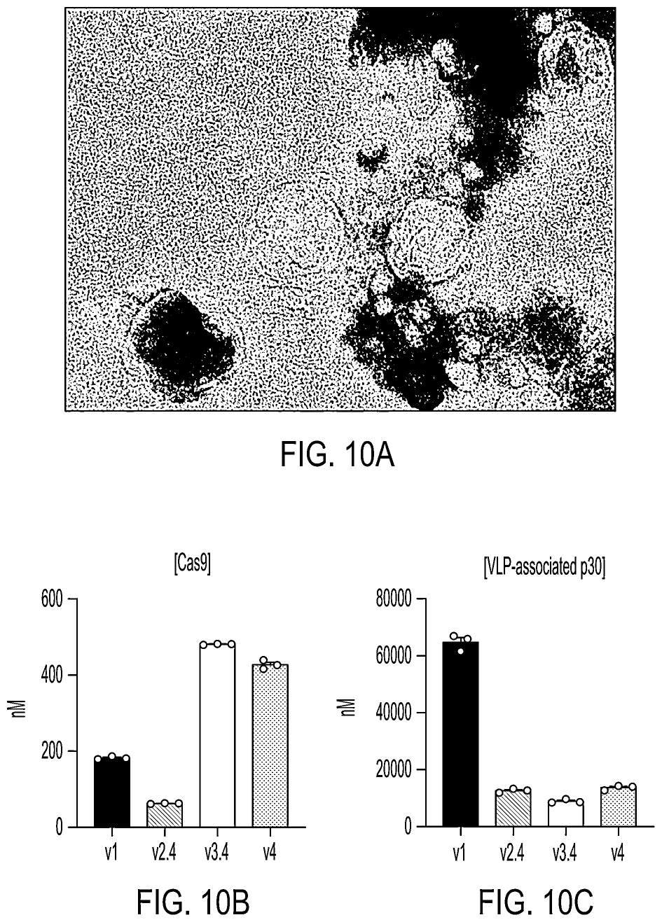

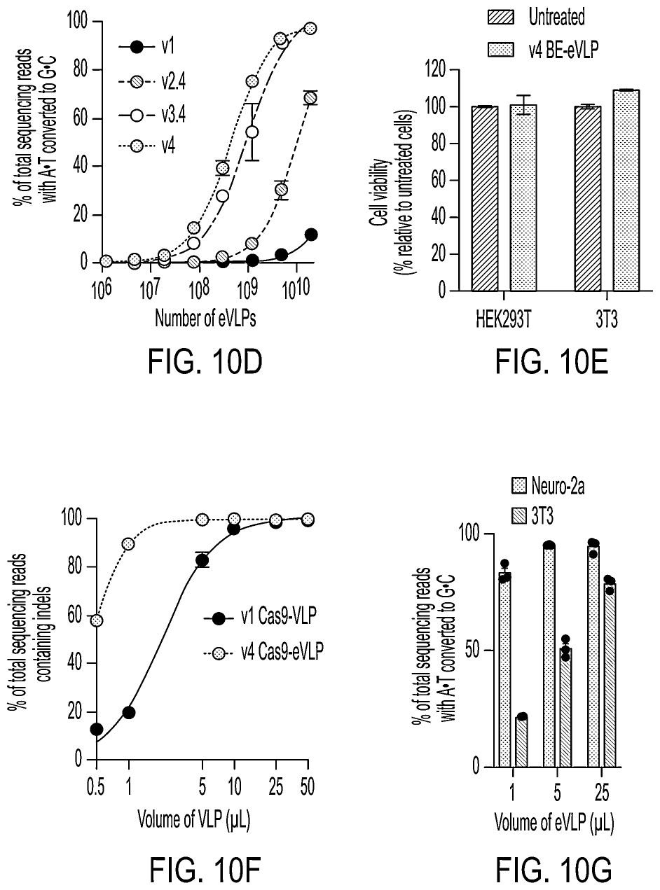

To encourage cytosolic gag-cargo localization in producer cells, third-generation (v3) eVLP architectures were designed that contain nuclear export signals (NESs) in addition to NLSs. Previous work demonstrated that MLV-based VLPs can tolerate the addition of NESs at multiple locations within the gag protein (Wu and Roth, 2014). In the v3 designs, MMLV protease-cleavable linker sequences were placed at locations next to NESs to ensure that the NESs would be cleaved from the cargo following VLP maturation (FIGS. 2D and 9A), thereby liberating NLS-flanked cargo proteins that could be efficiently imported into the nucleus of the transduced cells. All v3 BE-eVLP architectures contained the optimal gag-ABE8e linker sequence from v2.4 BE-eVLPs. BE-eVLPs v3.1, v3.2, and v3.3 harbor a 3×NES motif fused at the C-terminus of ABE8e via an additional MMLV protease-cleavable linker and exhibited comparable or lower efficiencies relative to v2.4 BE-eVLPs (FIG. 2E). However, v3.4 BE-eVLPs, which contain a 3×NES motif at the C-terminus of MMLV gag immediately before the v2.4 optimized cleavable linker sequence, exhibited 1.1-2.1-fold improvements in editing efficiencies at the BCL11A enhancer locus at all doses tested relative to v2.4 BE-eVLPs (FIG. 2E). Notably, v3.4 BE-eVLPs require only a single viral protease cleavage event to liberate NLS-flanked, NES-free BEs (FIGS. 2D and 9A), compared to the two distinct cleavage events required in v3.1, v3.2, and v3.3 BE-eVLPs, which might explain their superior efficiency. To further investigate the effect of NES addition on gag-ABE localization, immunofluorescence microscopy of producer cells transfected with the v3.4 gag-3×NES-ABE construct or the v2.4 gag-ABE construct was performed. This analysis revealed a 1.3-fold increase in cytoplasmic localization of ABE protein detected in v3.4-transfected producer cells relative to v2.4-transfected producer cells (FIGS. 10B and 10C).

These results demonstrate that BE-eVLP activity can be improved by promoting the extranuclear localization of the gag-BE fusion in producer cells while maintaining the nuclear localization of the BEs released into transduced cells.

### Example 3. Improving Component Stoichiometry of eVLPs

This example demonstrates the adjustment of gag-cargo:gag-pro-pol stoichiometry of v3.4 eVLPs and its effect on delivery efficiency.

It was hypothesized that an optimal gag-cargo:gag-pro-pol stoichiometry would balance the amount of gag-cargo available to be packaged into VLPs with the amount of MMLV protease (“pro” in gag-pro-pol) required for VLP maturation (FIG. 2F).

To modulate this stoichiometry, the ratio of gag-3×NES-ABE8e to wild-type MMLV gag-pro-pol plasmids transfected for VLP production were varied. It was found that increasing the amount of gag-BE plasmid beyond the original proportion used for producing v3.4 BE-eVLPs (38% gag-BE plasmid and 62% gag-pro-pol plasmid) did not improve editing efficiencies (FIG. 2G). Decreasing the proportion of gag-BE plasmid from 38% to 25% modestly improved editing efficiencies (FIG. 2G). However, further decreasing the proportion of gag-BE plasmid below 25% reduced editing efficiencies (FIG. 2G). The results of this round of VLP engineering revealed a fourth-generation (v4) BE-eVLP formulation (FIG. 2G) which combines the optimal gag-BE:gag-pro-pol stoichiometry (25% gag-BE) with the v3.4 BE-eVLP architecture. v4 BE-eVLPs was visualized by transmission electron microscopy and confirmed their spherical morphology and approximate particle diameter of 100-150 nm (FIG. 10A).

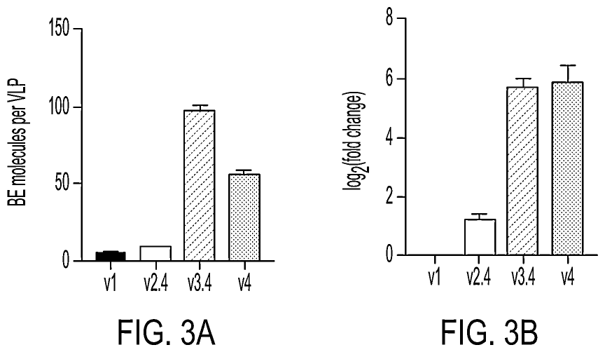

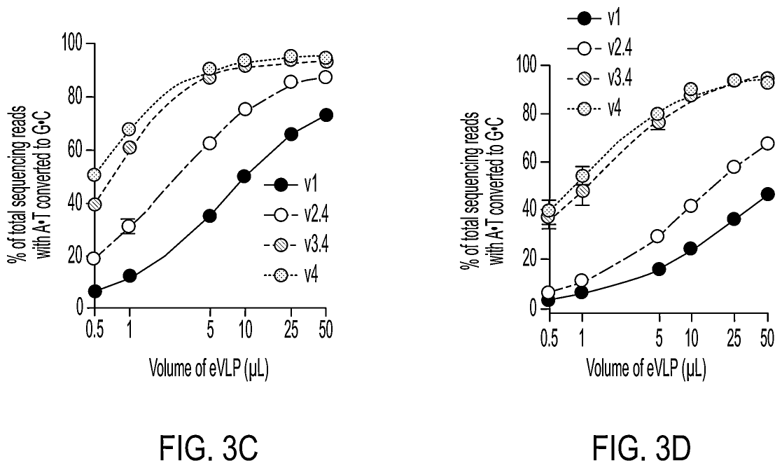

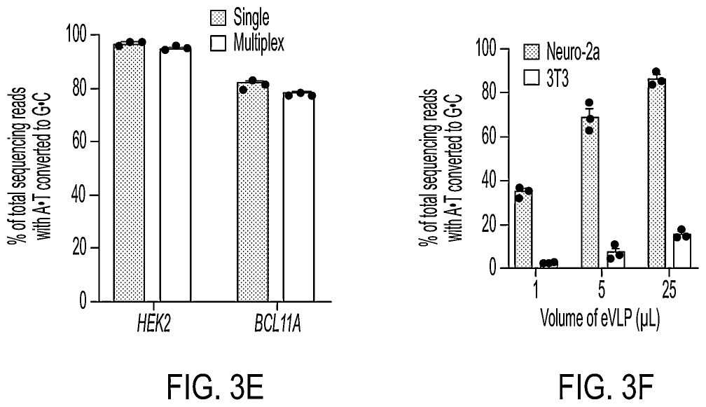

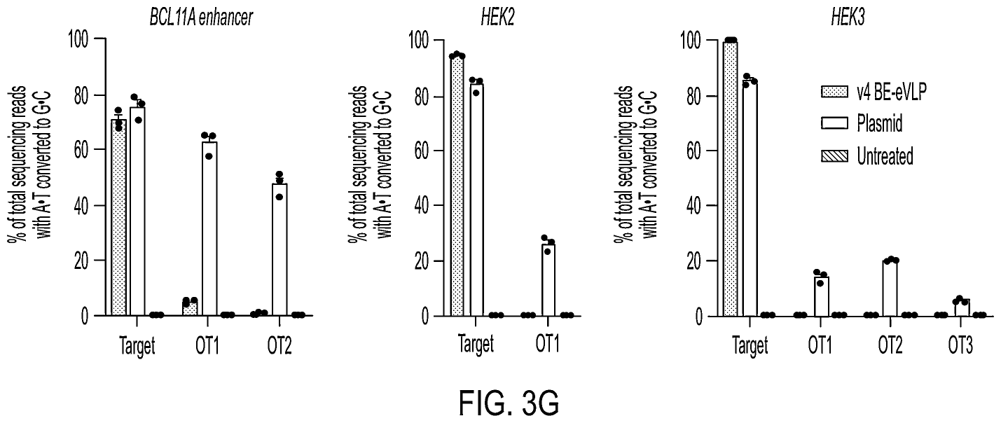

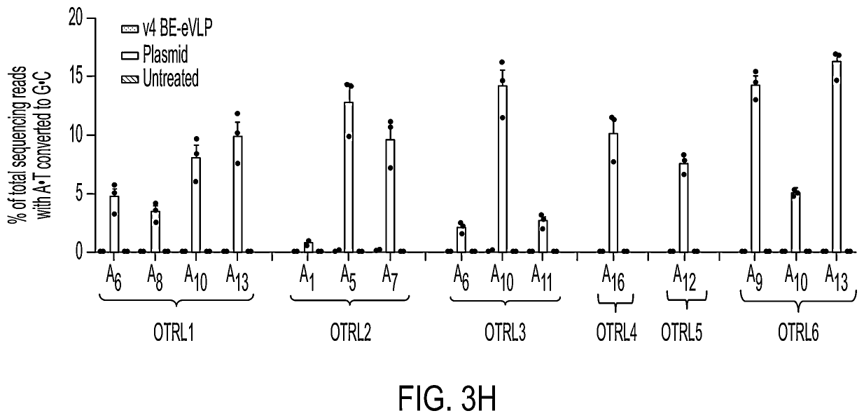

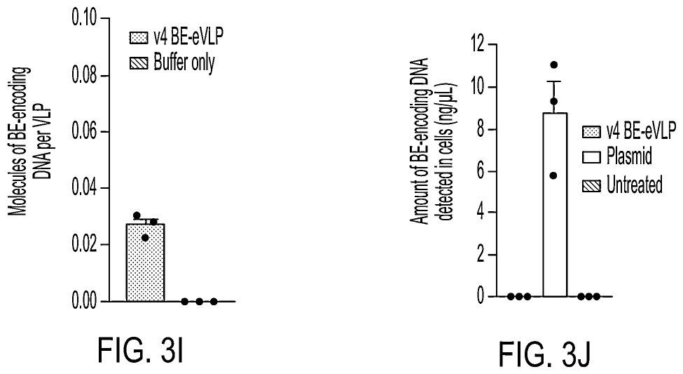

Next, the effects of the architecture engineering on the protein content of BE-eVLPs were determined. ELISAs with anti-Cas9 and anti-MLV (p30) antibodies were performed to quantify the number of BE molecules and p30 (MLV capsid) molecules present in v1 through v4 BE-eVLPs (FIGS. 10B and 10C). These experiments revealed that v2.4, v3.4, and v4 BE-eVLPs contain 1.8-, 19.2-, and 11-fold more BE cargo protein molecules per particle respectively compared to v1 BE-VLPs (FIG. 3A). This increase in BE protein content per particle correlates with an increase in the relative amount of sgRNAs per particle as measured by targeted RT-qPCR of lysed VLPs (FIG. 3B). Interestingly, v4 BE-eVLPs contain fewer BE protein molecules per particle than v3.4 BE-eVLPs but the same amount of sgRNA molecules, which suggests that v3.4 and v4 BE-eVLPs may contain similar amounts of active BE RNPs per particle. Additionally, v4 BE-eVLPs are produced at higher titer than v3.4 BE-eVLPs (FIG. 10C).

These results support a model in which increasing the number of active BE RNP molecules per particle can improve BE-eVLP editing efficiencies. However, increasing the number of BE molecules per particle beyond a certain threshold can be harmful, since these additional BE molecules do not appear to be complexed with sgRNAs, and there is an apparent trade-off between the number of cargo molecules incorporated per VLP and overall VLP titers.

The successive VLP engineering efforts described above substantially improved editing efficiencies of v4 BE-eVLPs at the BCL11A enhancer locus in HEK293T cells to 95% at the maximal dose (FIG. 3C). v4 BE-eVLPs exhibit a 5.6-fold improvement in editing efficiency per unit volume compared to v1 BE-VLPs and a 2.2-fold improvement compared to v2.4 BE-eVLPs (FIG. 3C). It was also observed that v4 BE-eVLPs exhibit 8.5-fold improvements in base editing activity per viral particle in HEK293T cells (FIG. 10D). To confirm that v4 VLP engineering supported general base editing improvements that were not restricted to one particular genomic locus or target cell line, v1, v2.4, v3.4, and v4 BE-eVLPs targeting the Dnmt1 locus were tested in 3T3 mouse fibroblasts. It was observed a very similar trend in the editing efficiencies of the four eVLP architectures, with an 8.6-fold improvement in editing efficiency per unit volume of v4 BE-eVLPs compared to v1 BE-VLPs in 3T3 cells (FIG. 3D). Additionally, treatment with v4 BE-eVLPs had no negative impact on the viability of HEK293T or 3T3 cells (FIG. 10E). v4 BE-eVLPs also supported robust multiplex editing of the BCL11A enhancer and HEK2 genomic loci in HEK293T cells (FIG. 3E). These results show that v4 BE-eVLPs mediate high-efficiency base editing while being minimally perturbative to the treated cells.

v1 and v4 VLPs that packaged Cas9 nuclease (Cas9-VLPs) and an sgRNA targeting the EMX1 genomic locus were constructed. A 4.7-fold improvement was observed in indel frequencies per unit volume generated by v4 Cas9-eVLPs compared to v1 Cas9-VLPs in HEK293T cells (FIG. 10F). This observation suggests that the engineered v4 eVLP architecture offers improvements to VLP-mediated delivery of proteins that are not limited to BEs.

An attractive feature of e VLPs is that their cellular tropism in principle can be modulated by producing them with different envelope glycoproteins. v4 BE-eVLPs pseudotyped with the FuG-B2 envelope glycoprotein were constructed (Kato et al., 2011). FuG-B2 is an engineered envelope glycoprotein that contains the extracellular and transmembrane domains of the rabies virus envelope glycoprotein and the cytoplasmic domain of VSV-G, and can be used to pseudotype lentiviruses for neuron-specific transduction (Kato et al., 2011). Indeed, it was observed that FuG-B2-pseudotyped v4 BE-eVLPs efficiently transduce and edit Neuro-2a cells (a mouse neuroblastoma cell line) but not mouse 3T3 fibroblasts (FIGS. 3F and 10G). These results validate that the tissue specificity of eVLPs can be targeted by swapping in other glycoproteins such as those used to pseudotype lentiviruses to transduce specific cell populations.

BEs can mediate Cas-dependent off-target editing at a subset of Cas9 off-target binding sites, as well as Cas-independent off-target editing at a low level throughout the genome (Anzalone et al., 2020). To evaluate Cas-dependent off-target editing by v4 BE-eVLPs relative to ABESe plasmid transfection in HEK293T cells, targeted amplicon sequencing was performed of known Cas9 off-target sites associated with three different sgRNAs targeting the HEK2, HEK3, and BCL11A enhancer loci. Comparable or higher on-target editing efficiency was observed from v4 BE-eVLPs compared to plasmid transfection at these three genomic loci, but 12- to 900-fold lower Cas-dependent off-target editing from v4 BE-eVLPs (FIG. 3G).

To evaluate Cas-independent off-target DNA editing, an orthogonal R-loop assay was performed, which multiple labs previously validated as a strategy for assessing the ability of a base editor to deaminate DNA in an unguided manner without requiring whole-genome sequencing (Doman et al., 2020; Yu et al., 2020). Compared with transfection of DNA plasmid encoding the same BE, v4 BE-eVLPs exhibited a >100-fold reduction in Cas-independent off-target editing, down to virtually undetected levels (FIGS. 3H and 11B). These results confirm and extend previous findings that off-target editing by highly active BEs can be substantially minimized with RNP delivery (Doman et al., 2020; Jang et al., 2021; Lyu et al., 2021; Newby et al., 2021; Rees and Liu, 2018; Richter et al., 2020; Yeh et al., 2018) and highlight the ability of eVLPs to support highly efficient on-target base editing with minimal off-target editing.

The DNA-free nature of eVLPs in principle avoids the possibility of DNA integration into the genomes of transduced cells, an important safety advantage over existing viral delivery modalities (David and Doherty, 2017; Milone and O'Doherty, 2018). It was verified by qPCR that purified v4 BE-eVLPs contain <0.03 molecules of BE-encoding DNA per VLP (FIG. 3I). Additionally, while substantial amounts (8.7 ng/μL) of BE-encoding DNA were detected in cellular lysate from HEK293T cells that were transfected with BE-encoding plasmids, BE-encoding DNA was not detected in cellular lysate from v4 BE-eVLP-treated HEK293T cells above background levels in samples from untreated cells (<0.02 ng/μL) (FIG. 3J). These results demonstrate that BE-eVLPs do not expose transduced cells to detected levels of DNA encoding BEs, thereby minimizing the possibility of genomic integration of cargo DNA.

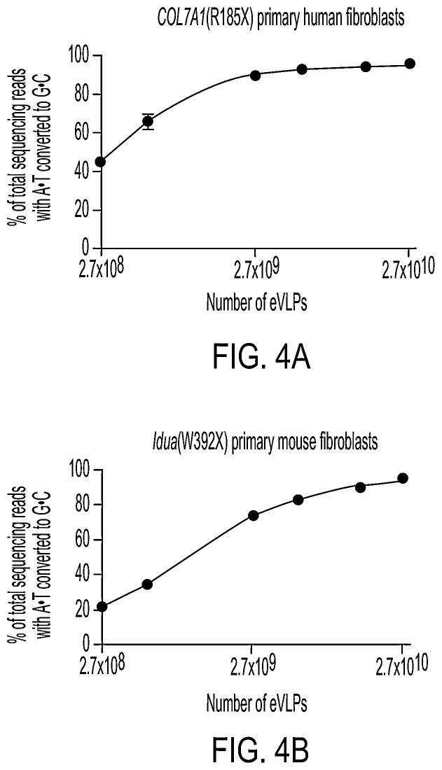

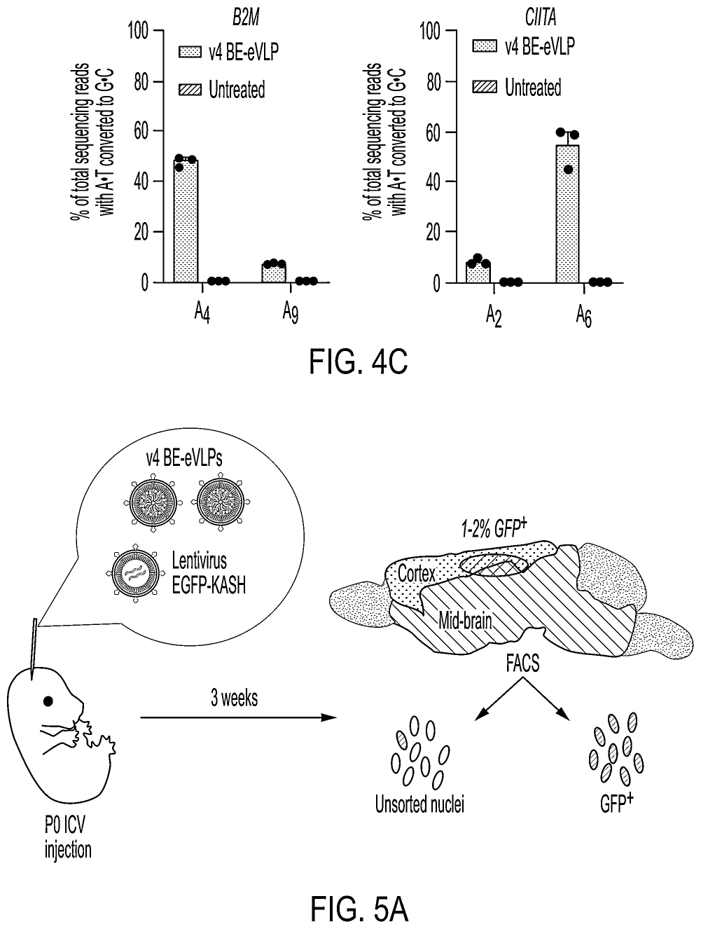

v4 BE-eVLPs efficiently edit primary human and mouse cells To further explore the utility of v4 BE-eVLPs, their ability to target and edit a variety of primary human or mouse cells ex vivo was tested. There has been previous report of ABE-mediated correction of nonsense mutations in COL7A1 that cause recessive dystrophic epidermolysis bullosa (RDEB) in primary human patient-derived fibroblasts (Osborn et al., 2020). After transducing primary fibroblasts harboring a homozygous COL7AI (R185X) mutation with v4 BE-eVLPs, it was observed >95% editing at the target adenine base with no difference in the cellular viability between eVLP-treated and untreated cells (FIGS. 4A and 11C). Additionally, it was observed minimal Cas-dependent off-target editing at ten previously identified off-target sites (Osborn et al., 2020) (FIG. 11D).

The ability of v4 BE-eVLPs to correct a nonsense mutation in primary fibroblasts derived from a mouse model of Mucopolysaccharidosis type IH (Wang et al., 2010) was also assessed. Over 95% correction of the Idua (W392X) mutation following v4 BE-eVLP transduction was observed (FIG. 4B). These results validate that BE-eVLP activity is not restricted to immortalized cell lines and demonstrate that v4 BE-eVLPs can achieve levels of base editing in primary human and mouse fibroblasts approaching 100%.

Next, BE-eVLP-mediated editing in primary human T cells was investigated. Gene editing strategies that reduce the expression of immunomodulatory proteins on the surface of T cells, including MHC class I and MHC class II, could advance T-cell therapies by enabling “off-the-shelf” allogeneic chimeric antigen receptor (CAR) T cells. Previous reports have shown that disrupting splice sites in the B2M and CIITA genes reduces expression of MHC class I and MHC class II in primary human T cells (Gaudelli et al., 2020; LeibundGut-Landmann et al., 2004; Serreze et al., 1994). Treating primary human T cells with v4 BE-eVLPs led to 45-60% disruption of B2M and CIITA splice sites (FIG. 4C). Collectively, these results confirm that BE-VLPs can efficiently edit clinically relevant primary human cell types ex vivo and lay a foundation for the further optimization of BE-VLP editing efficiencies in primary human T cells.

In Vivo Base Editing in the CNS with eVLPs

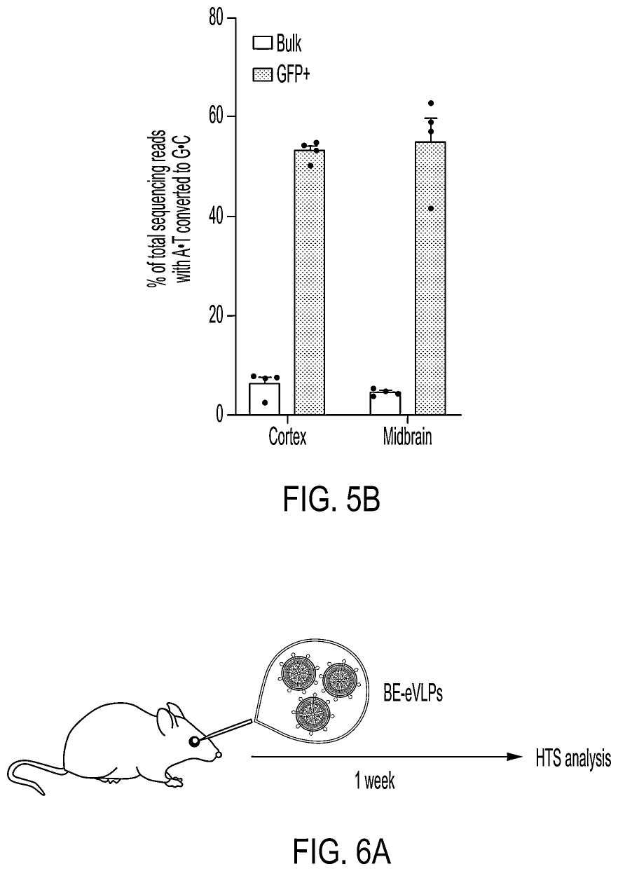

The robust activity of eVLPs ex vivo suggested that they might be promising vehicles for delivering BE RNPs in vivo. To begin to assess their in vivo efficacy, the ability of eVLPs to enable base editing within the mouse central nervous system (CNS) was investigated. v4 BE-eVLPs were produced that install a silent mutation in mouse Dnmt1 at a genomic locus known to be amenable to nuclease-mediated indel formation and adenine base editing in vivo (Levy et al., 2020; Swiech et al., 2015). To deliver BE-eVLPs to the CNS, neonatal cerebroventricular (P0 ICV) injections were performed, which are direct injections into cerebrospinal fluid that bypass the blood-brain barrier, similar to the intrathecal injections currently used to deliver nusinersen in patients with spinal muscular atrophy (Mercuri et al., 2018). v4 BE-eVLPs were co-injected into each hemisphere together with a VSV-G-pseudotyped lentivirus encoding EGFP fused to a nuclear membrane-localized Klarsicht/ANC-1/Syne-1 homology (KASH) domain (FIG. 5A). It was reasoned that this strategy would enable the isolation of GFP-positive nuclei as a way to enrich cells that were exposed to eVLPs. This approach is particularly useful to determine editing efficiencies following injection in the brain, where many cells may not be accessible. Three weeks post-injection, bulk unsorted and GFP-positive nuclei from cortical and mid-brain tissues were analyzed, and base editing was assessed by high-throughput sequencing (FIG. 5A).

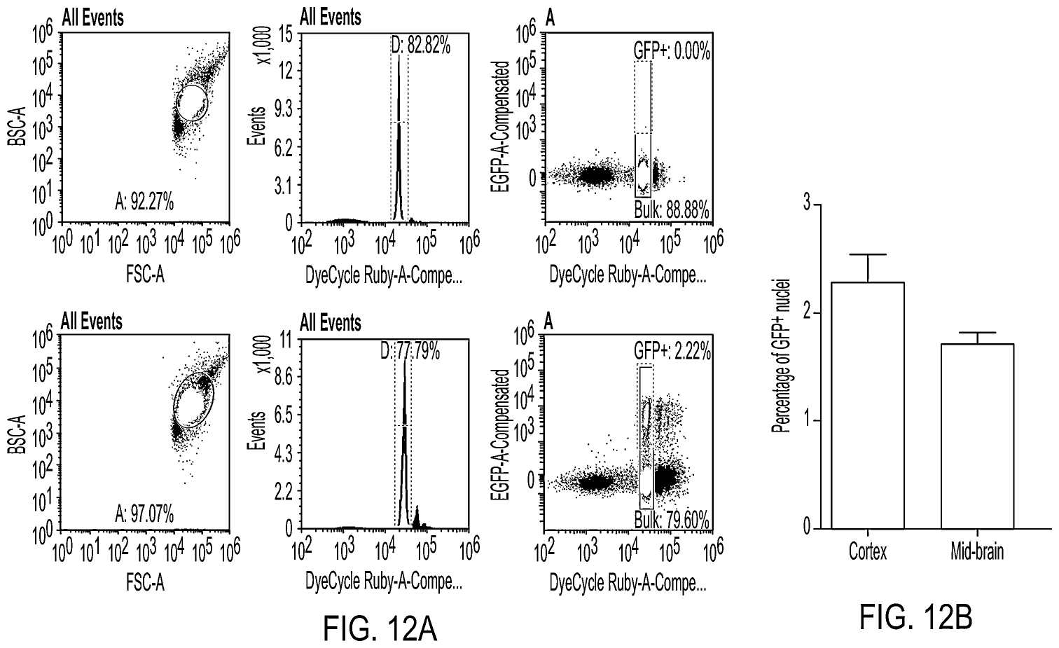

The frequencies of GFP-positive nuclei in both cortical and mid-brain tissues were low (FIG. 12B), consistent with previous reports that the cells transduced by VSV-G-pseudotyped lentiviruses injected into the mouse brain are localized near the injection site (Humbel et al., 2021; Parr-Brownlie et al., 2015), possibly because the size of the viral particles, which have an average diameter ˜3-fold larger than the width of the brain extracellular space (Thorne and Nicholson, 2006), may hinder diffusion through bulk brain tissue. Encouragingly, 53% and 55% editing in GFP-positive cortex and mid-brain cells, respectively, was observed, corresponding to 6.1% and 4.4% editing of bulk cortex and mid-brain (FIG. 5B). These data establish BE-eVLPs as a new non-viral delivery system for CNS base editing applications that deliver robust levels of active BE RNP per transduction event, although improvements in transduction efficiency are needed to achieve high levels of editing in bulk brain tissue. In vivo liver base editing with eVLPs leads to efficient knockdown of Pcsk9.

To further explore the utility of BE-eVLPs in vivo, their ability to mediate therapeutic base editing in adult animals was investigated. First, proprotein convertase subtilisin/kexin type 9 (Pcsk9) was targeted, which is a therapeutically relevant gene involved in cholesterol homeostasis (Abifadel et al., 2003; Fitzgerald et al., 2014). Loss-of-function PCSK9 mutations occur naturally without apparent adverse health consequences (Abifadel et al., 2003; Cohen et al., 2005; Cohen et al., 2006; Hooper et al., 2007; Rao et al., 2018). These individuals have lower levels of low-density lipoprotein (LDL) cholesterol in the blood and a reduced risk of atherosclerotic cardiovascular disease, suggesting that disrupting the PCSK9 gene could be a promising strategy for the treatment of familial hypercholesterolemia (Musunuru et al., 2021; Rothgangl et al., 2021).

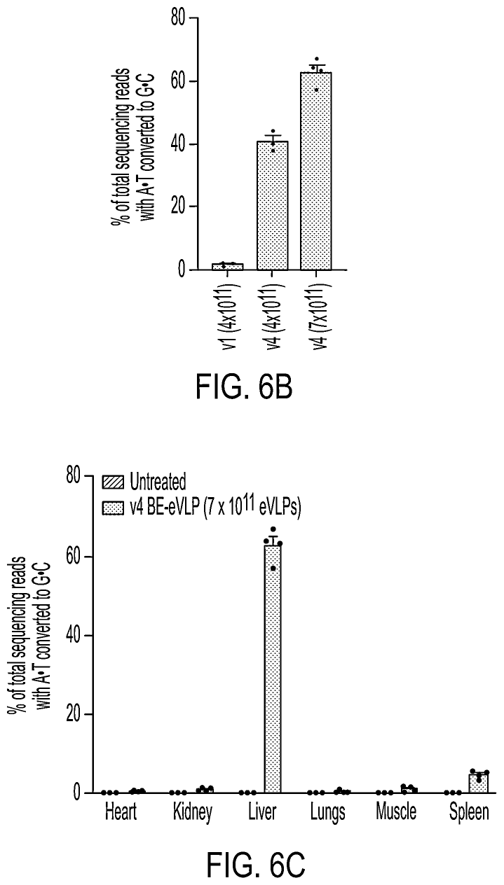

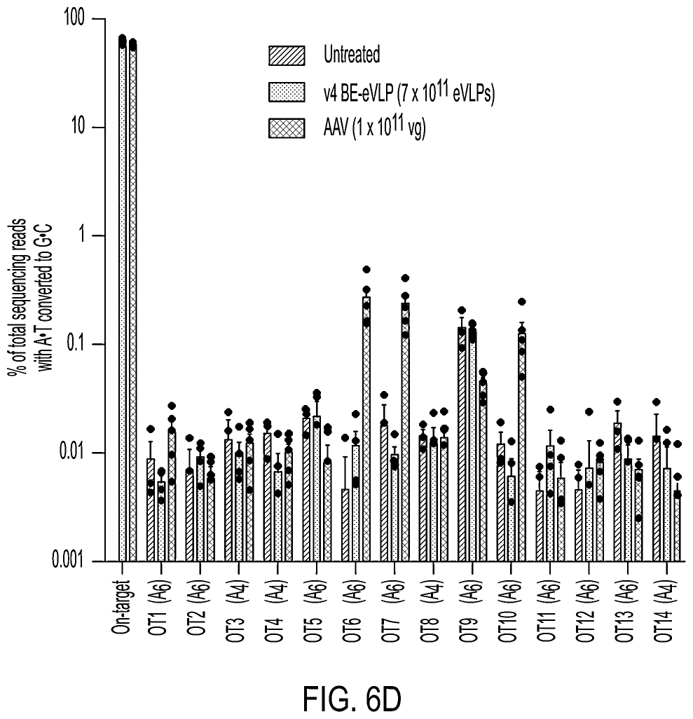

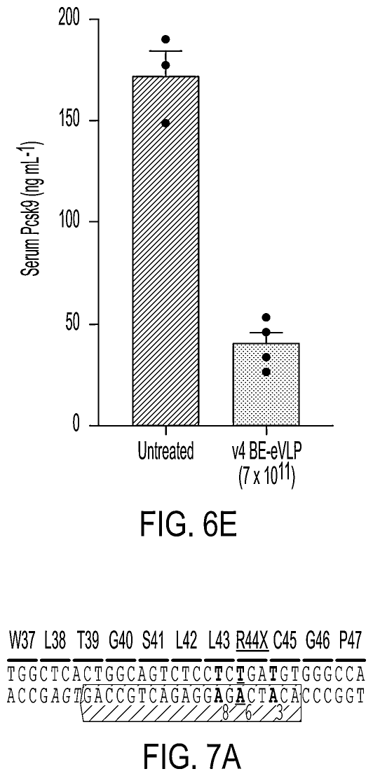

v4 BE-eVLPs were produced that target and disrupt the splice donor at the boundary of Pcsk9 exon 1 and intron 1, a previously established base editing strategy for Pcsk9 knockdown in the mouse liver (Musunuru et al., 2021; Rothgangl et al., 2021). Systemic (retro-orbital) injections of the eVLPs into 6- to 7-week-old adult C57BL/6 mice were performed and base editing in the liver was measured one week after injection (FIG. 6A). 63% editing was observed in bulk liver following treatment with the highest dose (7×1011 eVLPs) of v4 BE-eVLPs (FIG. 6B), comparable to editing efficiencies typically achieved at this site with optimized, state-of-the-art AAV-based delivery modalities and lipid nanoparticle (LNP)-based mRNA delivery systems (Musunuru et al., 2021; Rothgangl et al., 2021). The engineered v4 BE-eVLP architecture supported 26-fold higher editing levels in the liver than the VLP architecture based on a previously reported design (v1 BE-VLP) at the same dose (FIG. 6B). These results establish efficient base editing by RNPs at a therapeutically relevant locus in the mouse liver.

In mice treated with the highest dose of v4 BE-eVLPs, base editing efficiencies were also assessed in non-liver tissues, including the heart, skeletal muscle, lungs, kidney, and spleen. 4.3% base editing was observed in the spleen, and no editing above background levels in the lungs, kidneys, heart, and muscle (FIG. 6C). This pattern of editing across tissues is consistent with the previously characterized tissue tropism of intravenously administered VSV-G-pseudotyped particles (Pan et al., 2002).

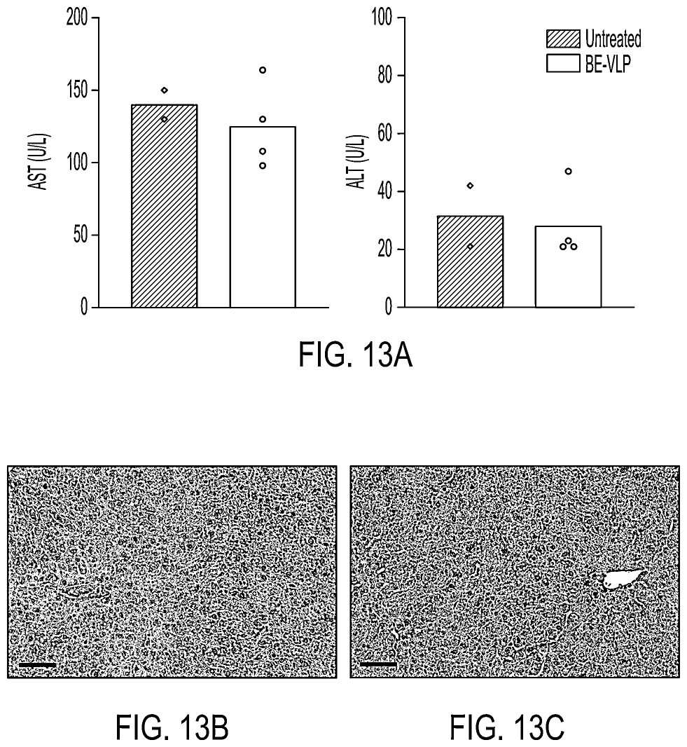

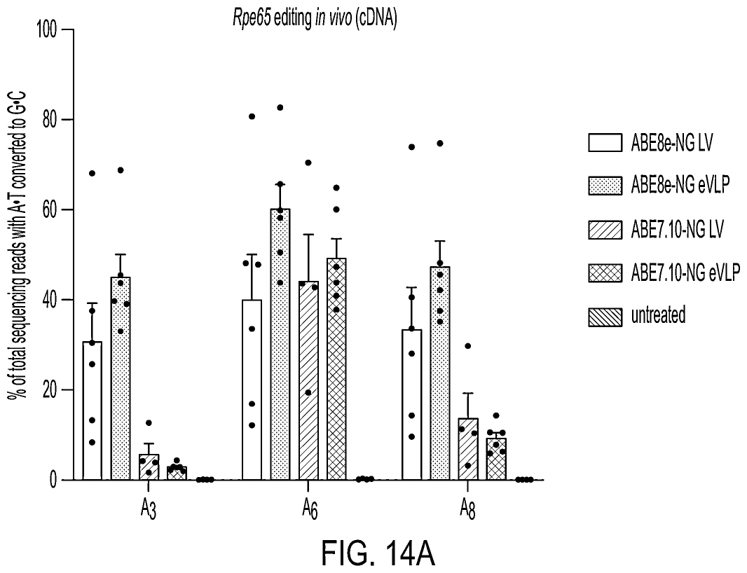

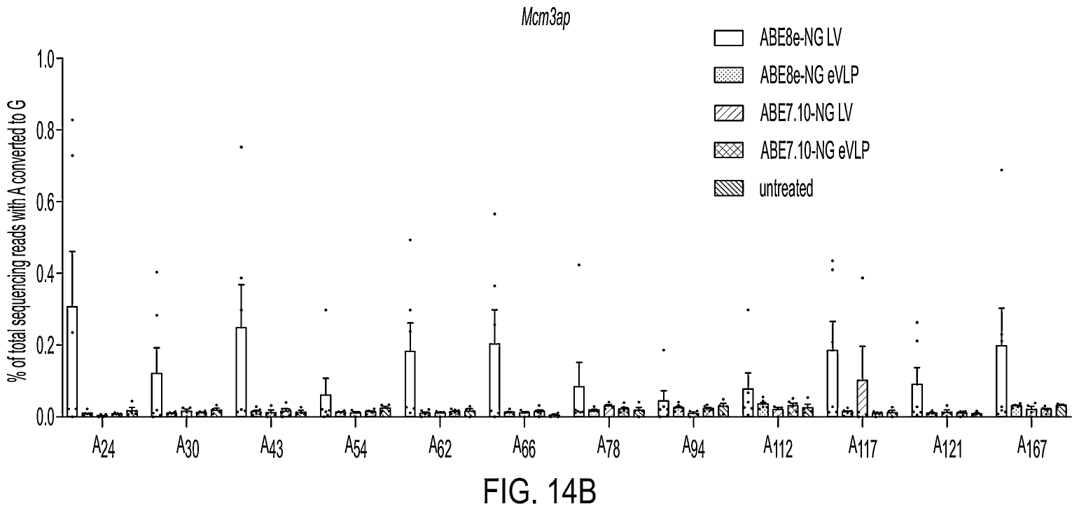

To assess whether treatment with BE-eVLPs resulted in Cas-dependent off-target editing in liver tissue, CIRCLE-seq was performed (Tsai et al., 2017) to nominate potential off-target loci. From the nominated loci, 14 candidate off-target sites were selected to examine by targeted high-throughput sequencing based on homology near the PAM-proximal region of the protospacer. No detectable off-target editing was detected above background levels at any of these loci in genomic DNA isolated from livers of mice treated with 7×1011 v4 BE-eVLPs (FIG. 6D). In contrast, low but detectable (0.1-0.3%) levels of off-target editing were detected at three of these loci in genomic DNA isolated from livers of mice treated with dual AAV8 vectors (1×1011 viral genomes) encoding ABE8e and the same Pcsk9-targeting sgRNA (FIG. 6D). These results demonstrate that v4 BE-eVLPs can offer comparable on-target editing but minimal off-target editing in vivo, an improvement compared to existing viral delivery approaches. Phenotypic analyses performed one-week post-injection revealed a 78% reduction in serum Pcsk9 protein level in mice treated with 7×1011 v4 BE-eVLPs compared to untreated mice (FIG. 6E). To assess the potential toxicity of systemically administered eVLPs, serum alanine aminotransferase (ALT) and aspartate transaminase (AST) levels were examined, which are important biomarkers of hepatocellular injury (Meunier and Larrey, 2019), one-week after injection of 7×1011 v4 BE-eVLPs. All mice exhibited AST and ALT levels within the normal range and there were no discernible differences between the untreated mice and the e VLP-treated mice (FIG. 13A). Additionally, liver histology was performed on samples from eVLP-treated and untreated mice and found no evident morphological differences due to eVLP treatment (FIGS. 13B and 13C). Together, these results demonstrate that v4 BE-eVLPs can mediate efficient, therapeutically relevant base editing in the mouse liver with no apparent adverse consequences and no detected off-target editing. v4 BE-eVLPs restore visual function in a mouse model of genetic blindness.

Finally, BE-eVLPs were applied to correct a disease-causing point mutation in an adult mouse model of a genetic retinal disorder. Loss-of-function mutations in multiple genes are associated with various forms of Leber congenital amaurosis (LCA), a family of monogenic retinal disorders that involve retinal degeneration, early-onset visual impairment, and eventual blindness (Cideciyan, 2010; den Hollander et al., 2008). Gene editing approaches hold promise to treat and cure congenital blindness; an ongoing clinical trial (NCT03872479) uses AAV-delivered Cas9 nucleases to disrupt an aberrant splice site in CEP290 that is associated with rare Leber congenital amaurosis 10 (LCA10). Loss-of-function mutations in other genes, including the retinoid isomerohydrolase RPE65, are also candidates for in vivo correction using precision gene editing agents (Sodi et al., 2021; Suh et al., 2021).

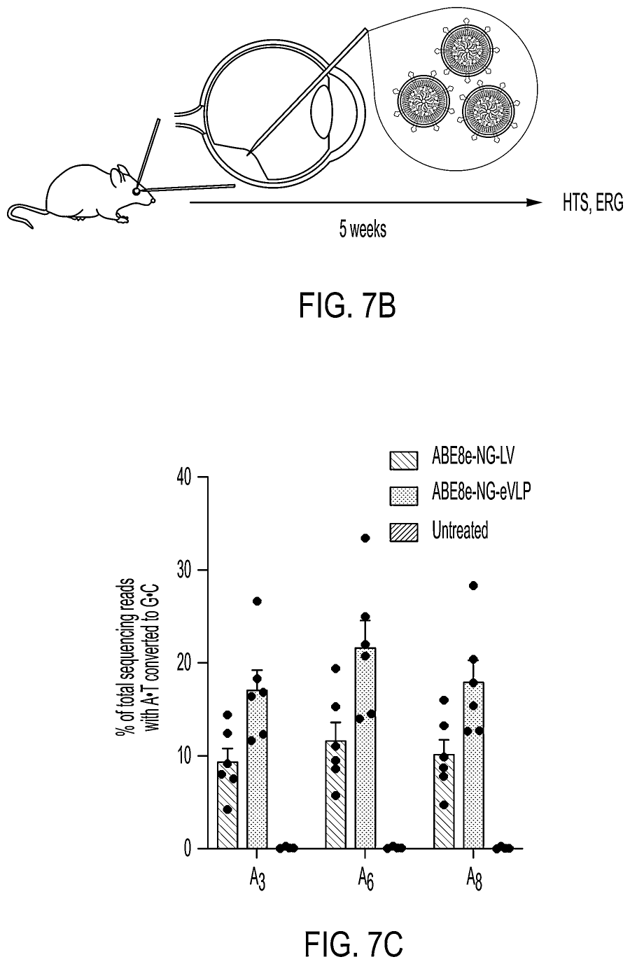

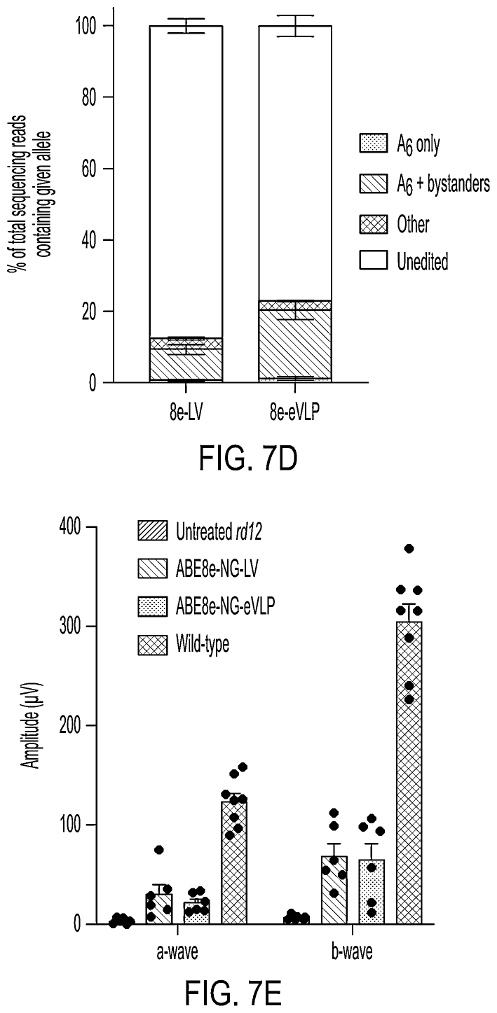

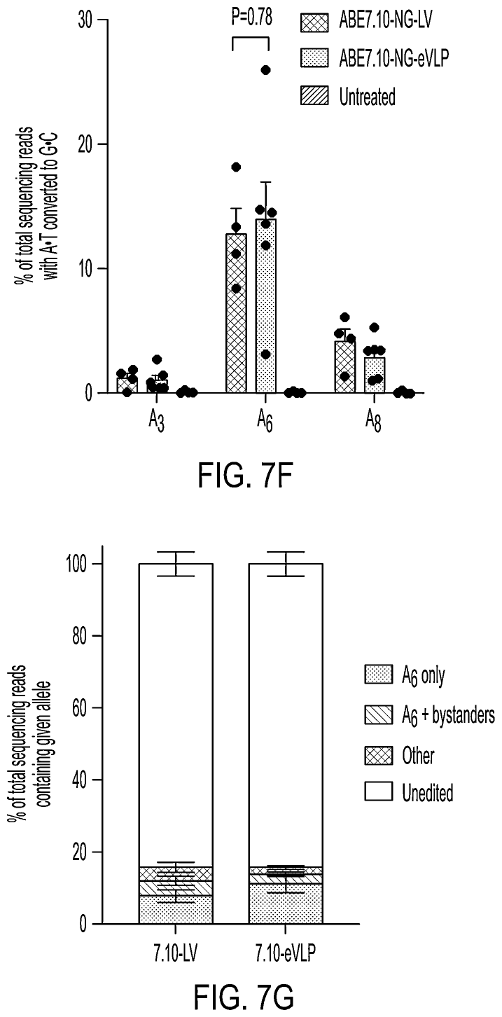

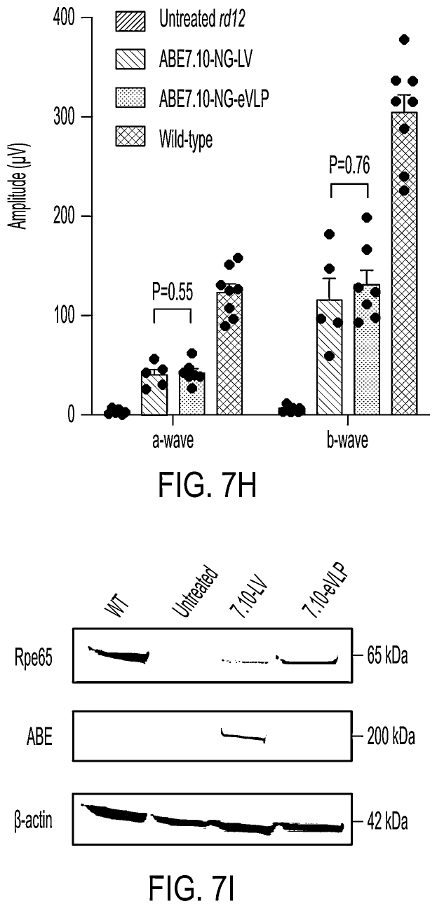

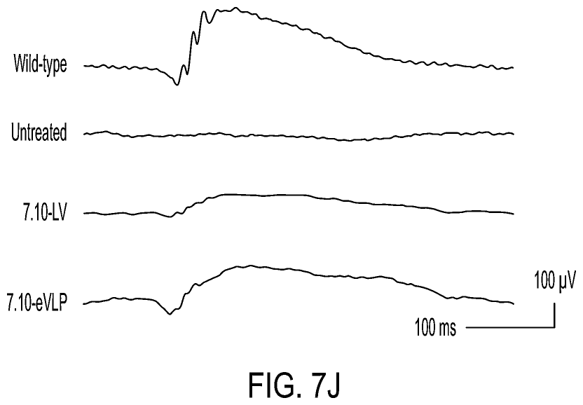

It was investigated whether v4 BE-eVLPs can restore visual function in a mouse model of LCA. rd12 mice harbor a nonsense mutation in exon 3 of Rpe65 (c.130C>T; p.R44X) that causes a near-complete loss of visual function (Pang et al., 2005; Suh et al., 2021). A homologous mutation responsible for LCA has recently been identified in people (Zhong et al., 2019), highlighting the clinical relevance of the rd12 model. v4 BE-eVLPs were designed and produced, which encapsulate ABE8e-NG RNPs and an sgRNA (FIG. 7A) that targets the Rpe65 (R44X) mutation (hereafter referred to as ABE8e-NG-eVLPs). ABE8e-NG-eVLPs were pseudotyped with VSV-G to enable them to efficiently transduce retinal pigment epithelium (RPE) cells (Puppo et al., 2014; Suh et al., 2021). ABE8e-NG-eVLPs were injected subretinally into 4-week-old rd12 mice. In a separate cohort, replication-incompetent lentivirus encoding the identical ABE8e-NG and sgRNA constructs (ABE8e-NG-LV) were subretinally injected. It has been reported previously that lentiviral delivery of ABEs can successfully restore visual function in rd12 mice (Suh et al., 2021).

Five weeks post-injection, RPE tissue was harvested and high-throughput sequencing of RPE genomic DNA was performed (FIG. 7B). Encouragingly, sequencing analysis revealed that ABE8e-NG-eVLPs and ABE8e-NG-LV successfully mediated 21% and 11.5% correction, respectively, of the R44× mutation at position A6 of the protospacer (FIG. 7C). Notably, ABE8e-NG-eVLPs achieved 1.8-fold higher editing at the target base compared to ABE8e-NG-LV, even though BE-eVLP delivery is transient. These results demonstrate that v4 BE-eVLPs enable highly efficient correction of a pathogenic mutation in the mouse RPE.

While highly efficient correction of the target mutation was observed, it was also observed that both ABE8e-NG-eVLP and ABE8e-NG-LV induced substantial levels of bystander editing (FIG. 7C) due to the wide editing window of ABE8e-NG (Richter et al., 2020), such that the majority of edited alleles contained conversions at A3, A6, and/or A8 as opposed to A6 alone (FIG. 7D). The bystander edits at positions A3 and A8 lead to Rpe65 missense mutations C45R and L43P respectively. It has been shown previously that the L43P mutation renders the Rpe65 enzyme inactive (Suh et al., 2021). Indeed, after performing scotopic electroretinography (ERG) to assess retinal cell response, minimal rescue was observed of visual function in both ABE8e-NG-eVLP-injected and ABE8e-NG-LV-injected eyes (FIG. 7E). These results suggested that the wide base editing window of ABE8e-NG is not well-suited to precisely correct the Rpe65 (R44X) mutation.

To address this limitation, v4 BE-eVLPs, which encapsulate ABE7.10-NG, which exhibits a narrower editing window compared to ABE8e-NG, were designed and produced (Huang et al., 2019; Richter et al., 2020). Subretinal injection of ABE7.10-NG-eVLPs into adult rd12 mice led to 12% correction of the R44× mutation in RPE genomic DNA with virtually no bystander editing (FIG. 7F). Specifically, it was observed that ABE7.10-NG-eVLP treatment resulted in 11% perfect R44× correction without bystander edits, a 9-fold improvement in perfect correction relative to ABE8e-NG-eVLP treatment (FIG. 7G). Furthermore, treatment with ABE7.10-NG-eVLPs resulted in a 1.4-fold improvement in bystander-free correction relative to treatment with ABE7.10-NG-LV, a lentivirus encoding the identical ABE7.10-NG and sgRNA constructs, an additional demonstration that v4 BE-eVLP transient delivery can achieve comparable or higher editing efficiencies compared to lentiviral BE delivery (FIG. 7G). It was confirmed via western blot that ABE7.10-NG-eVLP treatment restored the expression of Rpe65 protein. Notably, ABE7.10-NG-LV-treated eyes still expressed BE protein 5-weeks post-injection, while ABE7.10-NG-eVLP-treated eyes did not (FIG. 7I), demonstrating the transient exposure of cells in vivo to BEs delivered using e VLPs. Importantly, ABE7.10-NG-eVLPs successfully rescued visual function to similar levels relative to ABE7.10-NG-LV as measured by ERG of the treated eyes (FIGS. 7H, 7J).

It has been shown previously that this level of ERG rescue corresponds to other improvements in visual function, including restoration of the visual chromophore and recovery of visual cortical responses (Suh et al., 2021). These results demonstrate that eVLPs can mediate efficient correction of a pathogenic mutation in the mouse RPE with amelioration of the disease phenotype.

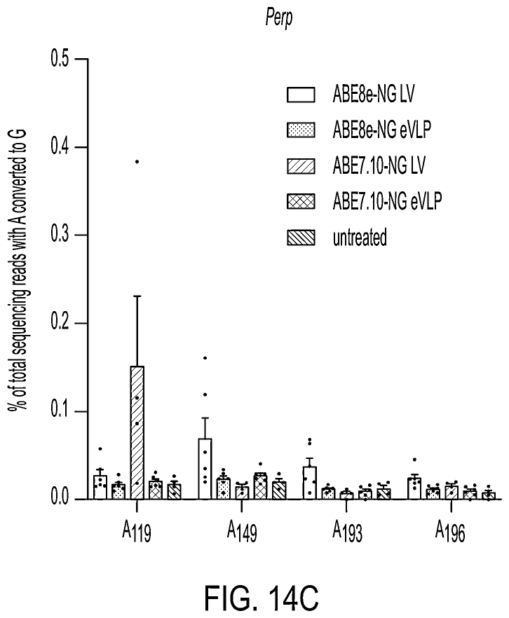

To further analyze editing outcomes, RNA was extracted from treated eyes and performed targeted high-throughput sequencing of specific cDNAs. As expected, in the eVLP treated eyes, up to 64% of A·T-to-G·C conversion of the target adenine (A6) was observed in the on-target Rpe65 transcript (FIG. 14A). The higher proportion of corrected Rpe65 transcripts compared to Rpe65 genomic loci potentially reflects nonsense-mediated decay of uncorrected mRNAs. BEs are known to exhibit low-level transcriptome-wide Cas-independent off-target RNA editing (Anzalone et al., 2020). To investigate this possibility, off-target RNA editing by ABE-eVLPs and ABE-LVs was assessed by sequencing the Mcm3ap and Perp transcripts from treated eyes, two transcripts that were previously identified as potential candidates for off-target RNA editing based on their sequence similarity to the native TadA deaminase substrate (Jo et al., 2021). RNA off-target editing by ABE8e-NG-LV was observed in both transcripts and low but detectable RNA off-target editing was detected by ABE7.10-NG-LV at one adenine in Perp (FIGS. 14B and 14C). In contrast, not any RNA off-target editing above background was detected in these two transcripts by ABE8e-NG-eVLPs or ABE7.10-NG-eVLPs (FIGS. 14B and 14C). Collectively, these findings highlight the therapeutic utility of eVLPs as a DNA-free method for transiently delivering BE RNPs in vivo with high on-target editing and minimal off-target editing.

TABLE 3
Example sequences

| | | SEQ ID |
|---|---|---|
| Sequence Name | Sequence | NO: |
| v1 BE-VLP | MGQAVTTPLSLTLDHWKDVERTAHNLSVEVRKRRWVTFCSAEW | 53 |
| | PTFNVGWPRDGTFNPDIITQVKIKVFSPGPHGHPDQVPYIVTWEAI | |
| | AVDPPPWVRPFVHPKPPLSLPPSAPSLPPEPPLSTPPQSSLYPALTSP | |
| | LNTKPRPQVLPDSGGPLIDLLTEDPPPYRDPGPPSPDGNGDSGEVA | |
| | PTEGAPDPSPMVSRLRGRKEPPVADSTTSQAFPLRLGGNGQYQYW | |
| | PFSSSDLYNWKNNNPSFSEDPAKLTALIESVLLTHQPTWDDCQQL | |
| | LGTLLTGEEKQRVLLEARKAVRGEDGRPTQLPNDINDAFPLERPD | |
| | WDYNTQRGRNHLVHYRQLLLAGLQNAGRSPTNLAKVKGITQGP | |
| | NESPSAFLERLKEAYRRYTPYDPEDPGQETNVAMSFIWQSAPDIGR | |
| | KLERLEDLKSKTLGDLVREAEKIFNKRETPEEREERIRRETEEKEER | |
| | RRAEDVQREKERDRRRHREMSKLLATVVSGQRQDRQGGERRRPQ | |
| | LDHDQCAYCKEKGHWARDCPKKPRGPRGPRPQASLLTRSSLYPA | |
| | LTPTGDYKDDDDKKRTADGSEFESPKKKRKVSEVEFSHEYWMRH | |
| | ALTLAKRARDEREVPVGAVLVLNNRVIGEGWNRAIGLHDPTAHA | |
| | EIMALRQGGLVMQNYRLIDATLYVTFEPCVMCAGAMIHSRIGRV | |
| | VFGVRNSKRGAAGSLMNVLNYPGMNHRVEITEGILADECAALLC | |
| | DFYRMPRQVFNAQKKAQSSINSGGSSGGSSGSETPGTSESATPESS | |
| | GGSSGGSDKKYSIGLAIGTNSVGWAVITDEYKVPSKKFKVLGNTD | |
| | RHSIKKNLIGALLFDSGETAEATRLKRTARRRYTRRKNRICYLQEIF | |
| | SNEMAKVDDSFFHRLEESFLVEEDKKHERHPIFGNIVDEVAYHEK | |
| | YPTIYHLRKKLVDSTDKADLRLIYLALAHMIKFRGHFLIEGDLNPD | |
| | NSDVDKLFIQLVQTYNQLFEENPINASGVDAKAILSARLSKSRRLE | |
| | NLIAQLPGEKKNGLFGNLIALSLGLTPNFKSNFDLAEDAKLQLSKD | |
| | TYDDDLDNLLAQIGDQYADLFLAAKNLSDAILLSDILRVNTEITKA | |
| | PLSASMIKRYDEHHQDLTLLKALVRQQLPEKYKEIFFDQSKNGYA | |
| | GYIDGGASQEEFYKFIKPILEKMDGTEELLVKLNREDLLRKQRTFD | |
| | NGSIPHQIHLGELHAILRRQEDFYPFLKDNREKIEKILTFRIPYYVGP | |
| | LARGNSRFAWMTRKSEETITPWNFEEVVDKGASAQSFIERMTNFD | |
| | KNLPNEKVLPKHSLLYEYFTVYNELTKVKYVTEGMRKPAFLSGE | |
| | QKKAIVDLLFKTNRKVTVKQLKEDYFKKIECFDSVEISGVEDREN | |
| | ASLGTYHDLLKIIKDKDFLDNEENEDILEDIVLTLTLFEDREMIEER | |
| | LKTYAHLFDDKVMKQLKRRRYTGWGRLSRKLINGIRDKQSGKTI | |
| | LDFLKSDGFANRNFMQLIHDDSLTFKEDIQKAQVSGQGDSLHEHI | |
| | ANLAGSPAIKKGILQTVKVVDELVKVMGRHKPENIVIEMARENQT | |
| | TQKGQKNSRERMKRIEEGIKELGSQILKEHPVENTQLQNEKLYLY | |
| | YLQNGRDMYVDQELDINRLSDYDVDHIVPQSFLKDDSIDNKVLTR | |
| | SDKNRGKSDNVPSEEVVKKMKNYWRQLLNAKLITQRKFDNLTK | |
| | AERGGLSELDKAGFIKRQLVETRQITKHVAQILDSRMNTKYDEND | |
| | KLIREVKVITLKSKLVSDFRKDFQFYKVREINNYHHAHDAYLNAV | |
| | VGTALIKKYPKLESEFVYGDYKVYDVRKMIAKSEQEIGKATAKYF | |
| | FYSNIMNFFKTEITLANGEIRKRPLIETNGETGEIVWDKGRDFATVR | |
| | KVLSMPQVNIVKKTEVQTGGFSKESILPKRNSDKLIARKKDWDPK | |
| | KYGGFDSPTVAYSVLVVAKVEKGKSKKLKSVKELLGITIMERSSF | |
| | EKNPIDFLEAKGYKEVKKDLIIKLPKYSLFELENGRKRMLASAGEL | |
| | QKGNELALPSKYVNFLYLASHYEKLKGSPEDNEQKQLFVEQHKH | |
| | YLDEIIEQISEFSKRVILADANLDKVLSAYNKHRDKPIREQAENIIHL | |
| | FTLTNLGAPAAFKYFDTTIDRKRYTSTKEVLDATLIHQSITGLYETR | |
| | IDLSQLGGDSGGSKRTADGSEFEPKKKRKV | |
| v2.1 BE-VLP | MGQTVTTPLSLTLGHWKDVERIAHNQSVDVKKRRWVTFCSAEW | 54 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDPRS | |
| | SLYPALTPGDYKDDDDKKRTADGSEFESPKKKRKVSEVEFSHEY | |
| | WMRHALTLAKRARDEREVPVGAVLVLNNRVIGEGWNRAIGLHD | |
| | PTAHAEIMALRQGGLVMQNYRLIDATLYVTFEPCVMCAGAMIHS | |
| | RIGRVVFGVRNSKRGAAGSLMNVLNYPGMNHRVEITEGILADEC | |
| | AALLCDFYRMPRQVFNAQKKAQSSINSGGSSGGSSGSETPGTSESA | |
| | TPESSGGSSGGSDKKYSIGLAIGTNSVGWAVITDEYKVPSKKFKVL | |
| | GNTDRHSIKKNLIGALLFDSGETAEATRLKRTARRRYTRRKNRICY | |
| | LQEIFSNEMAKVDDSFFHRLEESFLVEEDKKHERHPIFGNIVDEVA | |
| | YHEKYPTIYHLRKKLVDSTDKADLRLIYLALAHMIKFRGHFLIEGD | |
| | LNPDNSDVDKLFIQLVQTYNQLFEENPINASGVDAKAILSARLSKS | |
| | RRLENLIAQLPGEKKNGLFGNLIALSLGLTPNFKSNFDLAEDAKLQ | |
| | LSKDTYDDDLDNLLAQIGDQYADLFLAAKNLSDAILLSDILRVNT | |
| | EITKAPLSASMIKRYDEHHQDLTLLKALVRQQLPEKYKEIFFDQSK | |
| | NGYAGYIDGGASQEEFYKFIKPILEKMDGTEELLVKLNREDLLRK | |
| | QRTFDNGSIPHQIHLGELHAILRRQEDFYPFLKDNREKIEKILTFRIP | |
| | YYVGPLARGNSRFAWMTRKSEETITPWNFEEVVDKGASAQSFIER | |
| | MTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVE | |
| | DRFNASLGTYHDLLKIIKDKDFLDNEENEDILEDIVLTLTLFEDRE | |
| | MIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSL | |
| | HEHIANLAGSPAIKKGILQTVKVVDELVKVMGRHKPENIVIEMAR | |
| | ENQTTQKGQKNSRERMKRIEEGIKELGSQILKEHPVENTQLQNEK | |
| | LYLYYLQNGRDMYVDQELDINRLSDYDVDHIVPQSFLKDDSIDNK | |
| | VLTRSDKNRGKSDNVPSEEVVKKMKNYWRQLLNAKLITQRKFDN | |
| | LTKAERGGLSELDKAGFIKRQLVETRQITKHVAQILDSRMNTKYD | |
| | ENDKLIREVKVITLKSKLVSDFRKDFQFYKVREINNYHHAHDAYL | |
| | NAVVGTALIKKYPKLESEFVYGDYKVYDVRKMIAKSEQEIGKAT | |
| | AKYFFYSNIMNFFKTEITLANGEIRKRPLIETNGETGEIVWDKGRDF | |
| | ATVRKVLSMPQVNIVKKTEVQTGGFSKESILPKRNSDKLIARKKD | |
| | WDPKKYGGFDSPTVAYSVLVVAKVEKGKSKKLKSVKELLGITIM | |
| | ERSSFEKNPIDFLEAKGYKEVKKDLIIKLPKYSLFELENGRKRMLA | |
| | SAGELQKGNELALPSKYVNFLYLASHYEKLKGSPEDNEQKQLFVE | |
| | QHKHYLDEIIEQISEFSKRVILADANLDKVLSAYNKHRDKPIREQA | |
| | ENIIHLFTLTNLGAPAAFKYFDTTIDRKRYTSTKEVLDATLIHQSIT | |
| | GLYETRIDLSQLGGDSGGSKRTADGSEFEPKKKRKV | |
| v2.2 BE-VLP | MGQTVTTPLSLTLGHWKDVERIAHNQSVDVKKRRWVTFCSAEW | 55 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDVQA | |
| | LVLTQGDYKDDDDKKRTADGSEFESPKKKRKVSEVEFSHEYWMR | |
| | HALTLAKRARDEREVPVGAVLVLNNRVIGEGWNRAIGLHDPTAH | |
| | AEIMALRQGGLVMQNYRLIDATLYVTFEPCVMCAGAMIHSRIGR | |
| | VVFGVRNSKRGAAGSLMNVLNYPGMNHRVEITEGILADECAALL | |
| | CDFYRMPRQVFNAQKKAQSSINSGGSSGGSSGSETPGTSESATPES | |
| | SGGSSGGSDKKYSIGLAIGTNSVGWAVITDEYKVPSKKFKVLGNT | |
| | DRHSIKKNLIGALLFDSGETAEATRLKRTARRRYTRRKNRICYLQE | |
| | IFSNEMAKVDDSFFHRLEESFLVEEDKKHERHPIFGNIVDEVAYHE | |
| | KYPTIYHLRKKLVDSTDKADLRLIYLALAHMIKFRGHFLIEGDLNP | |
| | DNSDVDKLFIQLVQTYNQLFEENPINASGVDAKAILSARLSKSRRL | |
| | ENLIAQLPGEKKNGLFGNLIALSLGLTPNFKSNFDLAEDAKLQLSK | |
| | DTYDDDLDNLLAQIGDQYADLFLAAKNLSDAILLSDILRVNTEITK | |
| | APLSASMIKRYDEHHQDLTLLKALVRQQLPEKYKEIFFDQSKNGY | |
| | AGYIDGGASQEEFYKFIKPILEKMDGTEELLVKLNREDLLRKQRTF | |
| | DNGSIPHQIHLGELHAILRRQEDFYPFLKDNREKIEKILTFRIPYYVG | |
| | PLARGNSRFAWMTRKSEETITPWNFEEVVDKGASAQSFIERMTNF | |
| | DKNLPNEKVLPKHSLLYEYFTVYNELTKVKYVTEGMRKPAFLSG | |
| | EQKKAIVDLLFKTNRKVTVKQLKEDYFKKIECFDSVEISGVEDREN | |
| | ASLGTYHDLLKIIKDKDFLDNEENEDILEDIVLTLTLFEDREMIEER | |
| | LKTYAHLFDDKVMKQLKRRRYTGWGRLSRKLINGIRDKQSGKTI | |
| | LDFLKSDGFANRNFMQLIHDDSLTFKEDIQKAQVSGQGDSLHEHI | |
| | ANLAGSPAIKKGILQTVKVVDELVKVMGRHKPENIVIEMARENQT | |
| | TQKGQKNSRERMKRIEEGIKELGSQILKEHPVENTQLQNEKLYLY | |
| | YLQNGRDMYVDQELDINRLSDYDVDHIVPQSFLKDDSIDNKVLTR | |
| | SDKNRGKSDNVPSEEVVKKMKNYWRQLLNAKLITQRKFDNLTK | |
| | AERGGLSELDKAGFIKRQLVETRQITKHVAQILDSRMNTKYDEND | |
| | KLIREVKVITLKSKLVSDFRKDFQFYKVREINNYHHAHDAYLNAV | |
| | VGTALIKKYPKLESEFVYGDYKVYDVRKMIAKSEQEIGKATAKYF | |
| | FYSNIMNFFKTEITLANGEIRKRPLIETNGETGEIVWDKGRDFATVR | |
| | KVLSMPQVNIVKKTEVQTGGFSKESILPKRNSDKLIARKKDWDPK | |
| | KYGGFDSPTVAYSVLVVAKVEKGKSKKLKSVKELLGITIMERSSF | |
| | EKNPIDFLEAKGYKEVKKDLIIKLPKYSLFELENGRKRMLASAGEL | |
| | QKGNELALPSKYVNFLYLASHYEKLKGSPEDNEQKQLFVEQHKH | |
| | YLDEIIEQISEFSKRVILADANLDKVLSAYNKHRDKPIREQAENIIHL | |
| | FTLTNLGAPAAFKYFDTTIDRKRYTSTKEVLDATLIHQSITGLYETR | |
| | IDLSQLGGDSGGSKRTADGSEFEPKKKRKV | |
| v2.3 BE-VLP | MGQTVTTPLSLTLGHWKDVERIAHNQSVDVKKRRWVTFCSAEW | 56 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDPLQ | |
| | VLTLNIERRGDYKDDDDKKRTADGSEFESPKKKRKVSEVEFSHEY | |
| | WMRHALTLAKRARDEREVPVGAVLVLNNRVIGEGWNRAIGLHD | |
| | PTAHAEIMALRQGGLVMQNYRLIDATLYVTFEPCVMCAGAMIHS | |
| | RIGRVVFGVRNSKRGAAGSLMNVLNYPGMNHRVEITEGILADEC | |
| | AALLCDFYRMPRQVFNAQKKAQSSINSGGSSGGSSGSETPGTSESA | |
| | TPESSGGSSGGSDKKYSIGLAIGTNSVGWAVITDEYKVPSKKFKVL | |
| | GNTDRHSIKKNLIGALLFDSGETAEATRLKRTARRRYTRRKNRICY | |
| | LQEIFSNEMAKVDDSFFHRLEESFLVEEDKKHERHPIFGNIVDEVA | |
| | YHEKYPTIYHLRKKLVDSTDKADLRLIYLALAHMIKFRGHFLIEGD | |
| | LNPDNSDVDKLFIQLVQTYNQLFEENPINASGVDAKAILSARLSKS | |
| | RRLENLIAQLPGEKKNGLFGNLIALSLGLTPNFKSNFDLAEDAKLQ | |
| | LSKDTYDDDLDNLLAQIGDQYADLFLAAKNLSDAILLSDILRVNT | |
| | EITKAPLSASMIKRYDEHHQDLTLLKALVRQQLPEKYKEIFFDQSK | |
| | NGYAGYIDGGASQEEFYKFIKPILEKMDGTEELLVKLNREDLLRK | |
| | QRTFDNGSIPHQIHLGELHAILRRQEDFYPFLKDNREKIEKILTFRIP | |
| | YYVGPLARGNSRFAWMTRKSEETITPWNFEEVVDKGASAQSFIER | |
| | MTNFDKNLPNEKVLPKHSLLYEYFTVYNELTKVKYVTEGMRKPA | |
| | FLSGEQKKAIVDLLFKTNRKVTVKQLKEDYFKKIECFDSVEISGVE | |
| | DRFNASLGTYHDLLKIIKDKDFLDNEENEDILEDIVLTLTLFEDRE | |
| | MIEERLKTYAHLFDDKVMKQLKRRRYTGWGRLSRKLINGIRDKQ | |
| | SGKTILDFLKSDGFANRNFMQLIHDDSLTFKEDIQKAQVSGQGDSL | |
| | HEHIANLAGSPAIKKGILQTVKVVDELVKVMGRHKPENIVIEMAR | |
| | ENQTTQKGQKNSRERMKRIEEGIKELGSQILKEHPVENTQLQNEK | |
| | LYLYYLQNGRDMYVDQELDINRLSDYDVDHIVPQSFLKDDSIDNK | |
| | VLTRSDKNRGKSDNVPSEEVVKKMKNYWRQLLNAKLITQRKFDN | |
| | LTKAERGGLSELDKAGFIKRQLVETRQITKHVAQILDSRMNTKYD | |
| | ENDKLIREVKVITLKSKLVSDFRKDFQFYKVREINNYHHAHDAYL | |
| | NAVVGTALIKKYPKLESEFVYGDYKVYDVRKMIAKSEQEIGKAT | |
| | AKYFFYSNIMNFFKTEITLANGEIRKRPLIETNGETGEIVWDKGRDF | |
| | ATVRKVLSMPQVNIVKKTEVQTGGFSKESILPKRNSDKLIARKKD | |
| | WDPKKYGGFDSPTVAYSVLVVAKVEKGKSKKLKSVKELLGITIM | |
| | ERSSFEKNPIDFLEAKGYKEVKKDLIIKLPKYSLFELENGRKRMLA | |
| | SAGELQKGNELALPSKYVNFLYLASHYEKLKGSPEDNEQKQLFVE | |
| | QHKHYLDEIIEQISEFSKRVILADANLDKVLSAYNKHRDKPIREQA | |
| | ENIIHLFTLTNLGAPAAFKYFDTTIDRKRYTSTKEVLDATLIHQSIT | |
| | GLYETRIDLSQLGGDSGGSKRTADGSEFEPKKKRKV | |
| v2.4 BE-VLP | MGQTVTTPLSLTLGHWKDVERIAHNQSVDVKKRRWVTFCSAEW | 57 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDTST | |
| | LLMENSSGDYKDDDDKKRTADGSEFESPKKKRKVSEVEFSHEYW | |
| | MRHALTLAKRARDEREVPVGAVLVLNNRVIGEGWNRAIGLHDPT | |
| | AHAEIMALRQGGLVMQNYRLIDATLYVTFEPCVMCAGAMIHSRI | |
| | GRVVFGVRNSKRGAAGSLMNVLNYPGMNHRVEITEGILADECAA | |
| | LLCDFYRMPRQVFNAQKKAQSSINSGGSSGGSSGSETPGTSESATP | |
| | ESSGGSSGGSDKKYSIGLAIGTNSVGWAVITDEYKVPSKKFKVLG | |
| | NTDRHSIKKNLIGALLFDSGETAEATRLKRTARRRYTRRKNRICYL | |
| | QEIFSNEMAKVDDSFFHRLEESFLVEEDKKHERHPIFGNIVDEVAY | |
| | HEKYPTIYHLRKKLVDSTDKADLRLIYLALAHMIKFRGHFLIEGDL | |
| | NPDNSDVDKLFIQLVQTYNQLFEENPINASGVDAKAILSARLSKSR | |
| | RLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEI | |
| | TKAPLSASMIKRYDEHHQDLTLLKALVRQQLPEKYKEIFFDQSKN | |
| | GYAGYIDGGASQEEFYKFIKPILEKMDGTEELLVKLNREDLLRKQ | |
| | RTFDNGSIPHQIHLGELHAILRRQEDFYPFLKDNREKIEKILTFRIPY | |
| | YVGPLARGNSRFAWMTRKSEETITPWNFEEVVDKGASAQSFIERM | |
| | TNFDKNLPNEKVLPKHSLLYEYFTVYNELTKVKYVTEGMRKPAF | |
| | LSGEQKKAIVDLLFKTNRKVTVKQLKEDYFKKIECFDSVEISGVED | |
| | RFNASLGTYHDLLKIIKDKDFLDNEENEDILEDIVLTLTLFEDREMI | |
| | EERLKTYAHLFDDKVMKQLKRRRYTGWGRLSRKLINGIRDKQSG | |
| | KTILDFLKSDGFANRNFMQLIHDDSLTFKEDIQKAQVSGQGDSLHE | |
| | HIANLAGSPAIKKGILQTVKVVDELVKVMGRHKPENIVIEMAREN | |
| | QTTQKGQKNSRERMKRIEEGIKELGSQILKEHPVENTQLQNEKLY | |
| | LYYLQNGRDMYVDQELDINRLSDYDVDHIVPQSFLKDDSIDNKVL | |
| | TRSDKNRGKSDNVPSEEVVKKMKNYWRQLLNAKLITQRKFDNLT | |
| | KAERGGLSELDKAGFIKRQLVETRQITKHVAQILDSRMNTKYDEN | |
| | DKLIREVKVITLKSKLVSDFRKDFQFYKVREINNYHHAHDAYLNA | |
| | VVGTALIKKYPKLESEFVYGDYKVYDVRKMIAKSEQEIGKATAK | |
| | YFFYSNIMNFFKTEITLANGEIRKRPLIETNGETGEIVWDKGRDFAT | |
| | VRKVLSMPQVNIVKKTEVQTGGFSKESILPKRNSDKLIARKKDWD | |
| | PKKYGGFDSPTVAYSVLVVAKVEKGKSKKLKSVKELLGITIMERS | |
| | SFEKNPIDFLEAKGYKEVKKDLIIKLPKYSLFELENGRKRMLASAG | |
| | ELQKGNELALPSKYVNFLYLASHYEKLKGSPEDNEQKQLFVEQH | |
| | KHYLDEIIEQISEFSKRVILADANLDKVLSAYNKHRDKPIREQAENI | |
| | IHLFTLTNLGAPAAFKYFDTTIDRKRYTSTKEVLDATLIHQSITGLY | |
| | ETRIDLSQLGGDSGGSKRTADGSEFEPKKKRKV | |
| v3.1 BE-VLP | MGQTVTTPLSLTLGHWKDVERIAHNQSVDVKKRRWVTFCSAEW | 58 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDTST | |
| | LLMENSSGDYKDDDDKKRTADGSEFESPKKKRKVSEVEFSHEYW | |
| | MRHALTLAKRARDEREVPVGAVLVLNNRVIGEGWNRAIGLHDPT | |
| | AHAEIMALRQGGLVMQNYRLIDATLYVTFEPCVMCAGAMIHSRI | |
| | GRVVFGVRNSKRGAAGSLMNVLNYPGMNHRVEITEGILADECAA | |
| | LLCDFYRMPRQVFNAQKKAQSSINSGGSSGGSSGSETPGTSESATP | |
| | ESSGGSSGGSDKKYSIGLAIGTNSVGWAVITDEYKVPSKKFKVLG | |
| | NTDRHSIKKNLIGALLFDSGETAEATRLKRTARRRYTRRKNRICYL | |
| | QEIFSNEMAKVDDSFFHRLEESFLVEEDKKHERHPIFGNIVDEVAY | |
| | HEKYPTIYHLRKKLVDSTDKADLRLIYLALAHMIKFRGHFLIEGDL | |
| | NPDNSDVDKLFIQLVQTYNQLFEENPINASGVDAKAILSARLSKSR | |
| | RLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEI | |
| | TKAPLSASMIKRYDEHHQDLTLLKALVRQQLPEKYKEIFFDQSKN | |
| | GYAGYIDGGASQEEFYKFIKPILEKMDGTEELLVKLNREDLLRKQ | |
| | RTFDNGSIPHQIHLGELHAILRRQEDFYPFLKDNREKIEKILTFRIPY | |
| | YVGPLARGNSRFAWMTRKSEETITPWNFEEVVDKGASAQSFIERM | |
| | TNFDKNLPNEKVLPKHSLLYEYFTVYNELTKVKYVTEGMRKPAF | |
| | LSGEQKKAIVDLLFKTNRKVTVKQLKEDYFKKIECFDSVEISGVED | |
| | RFNASLGTYHDLLKIIKDKDFLDNEENEDILEDIVLTLTLFEDREMI | |
| | EERLKTYAHLFDDKVMKQLKRRRYTGWGRLSRKLINGIRDKQSG | |
| | KTILDFLKSDGFANRNFMQLIHDDSLTFKEDIQKAQVSGQGDSLHE | |
| | HIANLAGSPAIKKGILQTVKVVDELVKVMGRHKPENIVIEMAREN | |
| | QTTQKGQKNSRERMKRIEEGIKELGSQILKEHPVENTQLQNEKLY | |
| | LYYLQNGRDMYVDQELDINRLSDYDVDHIVPQSFLKDDSIDNKVL | |
| | TRSDKNRGKSDNVPSEEVVKKMKNYWRQLLNAKLITQRKFDNLT | |
| | KAERGGLSELDKAGFIKRQLVETRQITKHVAQILDSRMNTKYDEN | |
| | DKLIREVKVITLKSKLVSDFRKDFQFYKVREINNYHHAHDAYLNA | |
| | VVGTALIKKYPKLESEFVYGDYKVYDVRKMIAKSEQEIGKATAK | |
| | YFFYSNIMNFFKTEITLANGEIRKRPLIETNGETGEIVWDKGRDFAT | |
| | VRKVLSMPQVNIVKKTEVQTGGFSKESILPKRNSDKLIARKKDWD | |
| | PKKYGGFDSPTVAYSVLVVAKVEKGKSKKLKSVKELLGITIMERS | |
| | SFEKNPIDFLEAKGYKEVKKDLIIKLPKYSLFELENGRKRMLASAG | |
| | ELQKGNELALPSKYVNFLYLASHYEKLKGSPEDNEQKQLFVEQH | |
| | KHYLDEIIEQISEFSKRVILADANLDKVLSAYNKHRDKPIREQAENI | |
| | IHLFTLTNLGAPAAFKYFDTTIDRKRYTSTKEVLDATLIHQSITGLY | |
| | ETRIDLSQLGGDSGGSKRTADGSEFEPKKKRKVSGGSMSKLLATV | |
| | VSSGGSLQLPPLERLTLGSLQLPPLERLTLGSLQLPPLERLTL | |
| v3.2 BE-VLP | MGQTVTTPLSLTLGHWKDVERIAHNQSVDVKKRRWVTFCSAEW | 59 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDTST | |
| | LLMENSSGDYKDDDDKKRTADGSEFESPKKKRKVSEVEFSHEYW | |
| | MRHALTLAKRARDEREVPVGAVLVLNNRVIGEGWNRAIGLHDPT | |
| | AHAEIMALRQGGLVMQNYRLIDATLYVTFEPCVMCAGAMIHSRI | |
| | GRVVFGVRNSKRGAAGSLMNVLNYPGMNHRVEITEGILADECAA | |
| | LLCDFYRMPRQVFNAQKKAQSSINSGGSSGGSSGSETPGTSESATP | |
| | ESSGGSSGGSDKKYSIGLAIGTNSVGWAVITDEYKVPSKKFKVLG | |
| | NTDRHSIKKNLIGALLFDSGETAEATRLKRTARRRYTRRKNRICYL | |
| | QEIFSNEMAKVDDSFFHRLEESFLVEEDKKHERHPIFGNIVDEVAY | |
| | HEKYPTIYHLRKKLVDSTDKADLRLIYLALAHMIKFRGHFLIEGDL | |
| | NPDNSDVDKLFIQLVQTYNQLFEENPINASGVDAKAILSARLSKSR | |
| | RLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEI | |
| | TKAPLSASMIKRYDEHHQDLTLLKALVRQQLPEKYKEIFFDQSKN | |
| | GYAGYIDGGASQEEFYKFIKPILEKMDGTEELLVKLNREDLLRKQ | |
| | RTFDNGSIPHQIHLGELHAILRRQEDFYPFLKDNREKIEKILTFRIPY | |
| | YVGPLARGNSRFAWMTRKSEETITPWNFEEVVDKGASAQSFIERM | |
| | TNFDKNLPNEKVLPKHSLLYEYFTVYNELTKVKYVTEGMRKPAF | |
| | LSGEQKKAIVDLLFKTNRKVTVKQLKEDYFKKIECFDSVEISGVED | |
| | RFNASLGTYHDLLKIIKDKDFLDNEENEDILEDIVLTLTLFEDREMI | |
| | EERLKTYAHLFDDKVMKQLKRRRYTGWGRLSRKLINGIRDKQSG | |
| | KTILDFLKSDGFANRNFMQLIHDDSLTFKEDIQKAQVSGQGDSLHE | |
| | HIANLAGSPAIKKGILQTVKVVDELVKVMGRHKPENIVIEMAREN | |
| | QTTQKGQKNSRERMKRIEEGIKELGSQILKEHPVENTQLQNEKLY | |
| | LYYLQNGRDMYVDQELDINRLSDYDVDHIVPQSFLKDDSIDNKVL | |
| | TRSDKNRGKSDNVPSEEVVKKMKNYWRQLLNAKLITQRKFDNLT | |
| | KAERGGLSELDKAGFIKRQLVETRQITKHVAQILDSRMNTKYDEN | |
| | DKLIREVKVITLKSKLVSDFRKDFQFYKVREINNYHHAHDAYLNA | |
| | VVGTALIKKYPKLESEFVYGDYKVYDVRKMIAKSEQEIGKATAK | |
| | YFFYSNIMNFFKTEITLANGEIRKRPLIETNGETGEIVWDKGRDFAT | |
| | VRKVLSMPQVNIVKKTEVQTGGFSKESILPKRNSDKLIARKKDWD | |
| | PKKYGGFDSPTVAYSVLVVAKVEKGKSKKLKSVKELLGITIMERS | |
| | SFEKNPIDFLEAKGYKEVKKDLIIKLPKYSLFELENGRKRMLASAG | |
| | ELQKGNELALPSKYVNFLYLASHYEKLKGSPEDNEQKQLFVEQH | |
| | KHYLDEIIEQISEFSKRVILADANLDKVLSAYNKHRDKPIREQAENI | |
| | IHLFTLTNLGAPAAFKYFDTTIDRKRYTSTKEVLDATLIHQSITGLY | |
| | ETRIDLSQLGGDSGGSKRTADGSEFEPKKKRKVSGGSPLQVLTNIE | |
| | RRSGGSLQLPPLERLTLGSLQLPPLERLTLGSLQLPPLERLTL | |
| v3.3 BE-VLP | MGQTVTTPLSLTLGHWKDVERIAHNQSVDVKKRRWVTFCSAEW | 60 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDTST | |
| | LLMENSSGDYKDDDDKKRTADGSEFESPKKKRKVSEVEFSHEYW | |
| | MRHALTLAKRARDEREVPVGAVLVLNNRVIGEGWNRAIGLHDPT | |
| | AHAEIMALRQGGLVMQNYRLIDATLYVTFEPCVMCAGAMIHSRI | |
| | GRVVFGVRNSKRGAAGSLMNVLNYPGMNHRVEITEGILADECAA | |
| | LLCDFYRMPRQVFNAQKKAQSSINSGGSSGGSSGSETPGTSESATP | |
| | ESSGGSSGGSDKKYSIGLAIGTNSVGWAVITDEYKVPSKKFKVLG | |
| | NTDRHSIKKNLIGALLFDSGETAEATRLKRTARRRYTRRKNRICYL | |
| | QEIFSNEMAKVDDSFFHRLEESFLVEEDKKHERHPIFGNIVDEVAY | |
| | HEKYPTIYHLRKKLVDSTDKADLRLIYLALAHMIKFRGHFLIEGDL | |
| | NPDNSDVDKLFIQLVQTYNQLFEENPINASGVDAKAILSARLSKSR | |
| | RLENLIAQLPGEKKNGLFGNLIALSLGLTPNFKSNFDLAEDAKLQL | |
| | SKDTYDDDLDNLLAQIGDQYADLFLAAKNLSDAILLSDILRVNTEI | |
| | TKAPLSASMIKRYDEHHQDLTLLKALVRQQLPEKYKEIFFDQSKN | |
| | GYAGYIDGGASQEEFYKFIKPILEKMDGTEELLVKLNREDLLRKQ | |
| | RTFDNGSIPHQIHLGELHAILRRQEDFYPFLKDNREKIEKILTFRIPY | |
| | YVGPLARGNSRFAWMTRKSEETITPWNFEEVVDKGASAQSFIERM | |
| | TNFDKNLPNEKVLPKHSLLYEYFTVYNELTKVKYVTEGMRKPAF | |
| | LSGEQKKAIVDLLFKTNRKVTVKQLKEDYFKKIECFDSVEISGVED | |
| | RFNASLGTYHDLLKIIKDKDFLDNEENEDILEDIVLTLTLFEDREMI | |
| | EERLKTYAHLFDDKVMKQLKRRRYTGWGRLSRKLINGIRDKQSG | |
| | KTILDFLKSDGFANRNFMQLIHDDSLTFKEDIQKAQVSGQGDSLHE | |
| | HIANLAGSPAIKKGILQTVKVVDELVKVMGRHKPENIVIEMAREN | |
| | QTTQKGQKNSRERMKRIEEGIKELGSQILKEHPVENTQLQNEKLY | |
| | LYYLQNGRDMYVDQELDINRLSDYDVDHIVPQSFLKDDSIDNKVL | |
| | TRSDKNRGKSDNVPSEEVVKKMKNYWRQLLNAKLITQRKFDNLT | |
| | KAERGGLSELDKAGFIKRQLVETRQITKHVAQILDSRMNTKYDEN | |
| | DKLIREVKVITLKSKLVSDFRKDFQFYKVREINNYHHAHDAYLNA | |
| | VVGTALIKKYPKLESEFVYGDYKVYDVRKMIAKSEQEIGKATAK | |
| | YFFYSNIMNFFKTEITLANGEIRKRPLIETNGETGEIVWDKGRDFAT | |
| | VRKVLSMPQVNIVKKTEVQTGGFSKESILPKRNSDKLIARKKDWD | |
| | PKKYGGFDSPTVAYSVLVVAKVEKGKSKKLKSVKELLGITIMERS | |
| | SFEKNPIDFLEAKGYKEVKKDLIIKLPKYSLFELENGRKRMLASAG | |
| | ELQKGNELALPSKYVNFLYLASHYEKLKGSPEDNEQKQLFVEQH | |
| | KHYLDEIIEQISEFSKRVILADANLDKVLSAYNKHRDKPIREQAENI | |
| | IHLFTLTNLGAPAAFKYFDTTIDRKRYTSTKEVLDATLIHQSITGLY | |
| | ETRIDLSQLGGDSGGSKRTADGSEFEPKKKRKVSGGSIRKIFLDGS | |
| | GGSLQLPPLERLTLGSLQLPPLERLTLGSLQLPPLERLTL | |
| v3.4/v4 BE-VLP | MGQTVTTPLSLTLGHWKDVERIAHNQSVDVKKRRWVTFCSAEW | 61 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDSGG | |
| | SLQLPPLERLTLGSLQLPPLERLTLGSLQLPPLERLTLTSTLLMENSS | |
| | GDYKDDDDKKRTADGSEFESPKKKRKVSEVEFSHEYWMRHALTL | |
| | AKRARDEREVPVGAVLVLNNRVIGEGWNRAIGLHDPTAHAEIMA | |
| | LRQGGLVMQNYRLIDATLYVTFEPCVMCAGAMIHSRIGRVVFGV | |
| | RNSKRGAAGSLMNVLNYPGMNHRVEITEGILADECAALLCDFYR | |
| | MPRQVFNAQKKAQSSINSGGSSGGSSGSETPGTSESATPESSGGSS | |
| | GGSDKKYSIGLAIGTNSVGWAVITDEYKVPSKKFKVLGNTDRHSI | |
| | KKNLIGALLFDSGETAEATRLKRTARRRYTRRKNRICYLQEIFSNE | |
| | MAKVDDSFFHRLEESFLVEEDKKHERHPIFGNIVDEVAYHEKYPTI | |
| | YHLRKKLVDSTDKADLRLIYLALAHMIKFRGHFLIEGDLNPDNSD | |
| | VDKLFIQLVQTYNQLFEENPINASGVDAKAILSARLSKSRRLENLIA | |
| | QLPGEKKNGLFGNLIALSLGLTPNFKSNFDLAEDAKLQLSKDTYD | |
| | DDLDNLLAQIGDQYADLFLAAKNLSDAILLSDILRVNTEITKAPLS | |
| | ASMIKRYDEHHQDLTLLKALVRQQLPEKYKEIFFDQSKNGYAGYI | |
| | DGGASQEEFYKFIKPILEKMDGTEELLVKLNREDLLRKQRTFDNG | |
| | SIPHQIHLGELHAILRRQEDFYPFLKDNREKIEKILTFRIPYYVGPLA | |
| | RGNSRFAWMTRKSEETITPWNFEEVVDKGASAQSFIERMTNFDKN | |
| | LPNEKVLPKHSLLYEYFTVYNELTKVKYVTEGMRKPAFLSGEQK | |
| | KAIVDLLFKTNRKVTVKQLKEDYFKKIECFDSVEISGVEDRFNASL | |
| | GTYHDLLKIIKDKDFLDNEENEDILEDIVLTLTLFEDREMIEERLKT | |
| | YAHLFDDKVMKQLKRRRYTGWGRLSRKLINGIRDKQSGKTILDFL | |
| | KSDGFANRNFMQLIHDDSLTFKEDIQKAQVSGQGDSLHEHIANLA | |
| | GSPAIKKGILQTVKVVDELVKVMGRHKPENIVIEMARENQTTQKG | |
| | QKNSRERMKRIEEGIKELGSQILKEHPVENTQLQNEKLYLYYLQN | |
| | GRDMYVDQELDINRLSDYDVDHIVPQSFLKDDSIDNKVLTRSDKN | |
| | RGKSDNVPSEEVVKKMKNYWRQLLNAKLITQRKFDNLTKAERG | |
| | GLSELDKAGFIKRQLVETRQITKHVAQILDSRMNTKYDENDKLIRE | |
| | VKVITLKSKLVSDFRKDFQFYKVREINNYHHAHDAYLNAVVGTA | |
| | LIKKYPKLESEFVYGDYKVYDVRKMIAKSEQEIGKATAKYFFYSNI | |
| | MNFFKTEITLANGEIRKRPLIETNGETGEIVWDKGRDFATVRKVLS | |
| | MPQVNIVKKTEVQTGGFSKESILPKRNSDKLIARKKDWDPKKYGG | |
| | FDSPTVAYSVLVVAKVEKGKSKKLKSVKELLGITIMERSSFEKNPI | |
| | DFLEAKGYKEVKKDLIIKLPKYSLFELENGRKRMLASAGELQKGN | |
| | ELALPSKYVNFLYLASHYEKLKGSPEDNEQKQLFVEQHKHYLDEII | |
| | EQISEFSKRVILADANLDKVLSAYNKHRDKPIREQAENIIHLFTLTN | |
| | LGAPAAFKYFDTTIDRKRYTSTKEVLDATLIHQSITGLYETRIDLSQ | |
| | LGGDSGGSKRTADGSEFEPKKKRKV | |
| v4 BE-VLP | MGQTVTTPLSLTLGHWKDVERIAHNQSVDVKKRRWVTFCSAEW | 62 |
| (ABE8e-NG) | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDSGG | |
| | SLQLPPLERLTLGSLQLPPLERLTLGSLQLPPLERLTLTSTLLMENSS | |
| | GDYKDDDDKKRTADGSEFESPKKKRKVSEVEFSHEYWMRHALTL | |
| | AKRARDEREVPVGAVLVLNNRVIGEGWNRAIGLHDPTAHAEIMA | |
| | LRQGGLVMQNYRLIDATLYVTFEPCVMCAGAMIHSRIGRVVFGV | |
| | RNSKRGAAGSLMNVLNYPGMNHRVEITEGILADECAALLCDFYR | |
| | MPRQVFNAQKKAQSSINSGGSSGGSSGSETPGTSESATPESSGGSS | |
| | GGSDKKYSIGLAIGTNSVGWAVITDEYKVPSKKFKVLGNTDRHSI | |
| | KKNLIGALLFDSGETAEATRLKRTARRRYTRRKNRICYLQEIFSNE | |
| | MAKVDDSFFHRLEESFLVEEDKKHERHPIFGNIVDEVAYHEKYPTI | |
| | YHLRKKLVDSTDKADLRLIYLALAHMIKFRGHFLIEGDLNPDNSD | |
| | VDKLFIQLVQTYNQLFEENPINASGVDAKAILSARLSKSRRLENLIA | |
| | QLPGEKKNGLFGNLIALSLGLTPNFKSNFDLAEDAKLQLSKDTYD | |
| | DDLDNLLAQIGDQYADLFLAAKNLSDAILLSDILRVNTEITKAPLS | |
| | ASMIKRYDEHHQDLTLLKALVRQQLPEKYKEIFFDQSKNGYAGYI | |
| | DGGASQEEFYKFIKPILEKMDGTEELLVKLNREDLLRKQRTFDNG | |
| | SIPHQIHLGELHAILRRQEDFYPFLKDNREKIEKILTFRIPYYVGPLA | |
| | RGNSRFAWMTRKSEETITPWNFEEVVDKGASAQSFIERMTNFDKN | |
| | LPNEKVLPKHSLLYEYFTVYNELTKVKYVTEGMRKPAFLSGEQK | |
| | KAIVDLLFKTNRKVTVKQLKEDYFKKIECFDSVEISGVEDRFNASL | |
| | GTYHDLLKIIKDKDFLDNEENEDILEDIVLTLTLFEDREMIEERLKT | |
| | YAHLFDDKVMKQLKRRRYTGWGRLSRKLINGIRDKQSGKTILDFL | |
| | KSDGFANRNFMQLIHDDSLTFKEDIQKAQVSGQGDSLHEHIANLA | |
| | GSPAIKKGILQTVKVVDELVKVMGRHKPENIVIEMARENQTTQKG | |
| | QKNSRERMKRIEEGIKELGSQILKEHPVENTQLQNEKLYLYYLQN | |
| | GRDMYVDQELDINRLSDYDVDHIVPQSFLKDDSIDNKVLTRSDKN | |
| | RGKSDNVPSEEVVKKMKNYWRQLLNAKLITQRKFDNLTKAERG | |
| | GLSELDKAGFIKRQLVETRQITKHVAQILDSRMNTKYDENDKLIRE | |
| | VKVITLKSKLVSDFRKDFQFYKVREINNYHHAHDAYLNAVVGTA | |
| | LIKKYPKLESEFVYGDYKVYDVRKMIAKSEQEIGKATAKYFFYSNI | |
| | MNFFKTEITLANGEIRKRPLIETNGETGEIVWDKGRDFATVRKVLS | |
| | MPQVNIVKKTEVQTGGFSKESIRPKRNSDKLIARKKDWDPKKYGG | |
| | FVSPTVAYSVLVVAKVEKGKSKKLKSVKELLGITIMERSSFEKNPI | |
| | DFLEAKGYKEVKKDLIIKLPKYSLFELENGRKRMLASARFLQKGN | |
| | ELALPSKYVNFLYLASHYEKLKGSPEDNEQKQLFVEQHKHYLDEII | |
| | EQISEFSKRVILADANLDKVLSAYNKHRDKPIREQAENIIHLFTLTN | |
| | LGAPRAFKYFDTTIDRKVYRSTKEVLDATLIHQSITGLYETRIDLSQ | |
| | LGGDSGGSKRTADGSEFEPKKKRKV | |
| v4 BE-VLP | MGQTVTTPLSLTLGHWKDVERIAHNQSVDVKKRRWVTFCSAEW | 63 |
| (ABE7.10-NG) | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDDSGG | |
| | SLQLPPLERLTLGSLQLPPLERLTLGSLQLPPLERLTLTSTLLMENSS | |
| | GDYKDDDDKKRTADGSEFESPKKKRKVSEVEFSHEYWMRHALTL | |
| | AKRAWDEREVPVGAVLVHNNRVIGEGWNRPIGRHDPTAHAEIMA | |
| | LRQGGLVMQNYRLIDATLYVTLEPCVMCAGAMIHSRIGRVVFGA | |
| | RDAKTGAAGSLMDVLHHPGMNHRVEITEGILADECAALLSDFFR | |
| | MRRQEIKAQKKAQSSTDSGGSSGGSSGSETPGTSESATPESSGGSS | |
| | GGSSEVEFSHEYWMRHALTLAKRARDEREVPVGAVLVLNNRVIG | |
| | EGWNRAIGLHDPTAHAEIMALRQGGLVMQNYRLIDATLYVTFEP | |
| | CVMCAGAMIHSRIGRVVFGVRNAKTGAAGSLMDVLHYPGMNHR | |
| | VEITEGILADECAALLCYFFRMPRQVFNAQKKAQSSTDSGGSSGGS | |
| | SGSETPGTSESATPESSGGSSGGSDKKYSIGLAIGTNSVGWAVITDE | |
| | YKVPSKKFKVLGNTDRHSIKKNLIGALLFDSGETAEATRLKRTAR | |
| | RRYTRRKNRICYLQEIFSNEMAKVDDSFFHRLEESFLVEEDKKHER | |
| | HPIFGNIVDEVAYHEKYPTIYHLRKKLVDSTDKADLRLIYLALAH | |
| | MIKFRGHFLIEGDLNPDNSDVDKLFIQLVQTYNQLFEENPINASGV | |
| | DAKAILSARLSKSRRLENLIAQLPGEKKNGLFGNLIALSLGLTPNFK | |
| | SNFDLAEDAKLQLSKDTYDDDLDNLLAQIGDQYADLFLAAKNLS | |
| | DAILLSDILRVNTEITKAPLSASMIKRYDEHHQDLTLLKALVRQQL | |
| | PEKYKEIFFDQSKNGYAGYIDGGASQEEFYKFIKPILEKMDGTEEL | |
| | LVKLNREDLLRKQRTFDNGSIPHQIHLGELHAILRRQEDFYPFLKD | |
| | NREKIEKILTFRIPYYVGPLARGNSRFAWMTRKSEETITPWNFEEV | |
| | VDKGASAQSFIERMTNFDKNLPNEKVLPKHSLLYEYFTVYNELTK | |
| | VKYVTEGMRKPAFLSGEQKKAIVDLLFKTNRKVTVKQLKEDYFK | |
| | KIECFDSVEISGVEDRFNASLGTYHDLLKIIKDKDFLDNEENEDILE | |
| | DIVLTLTLFEDREMIEERLKTYAHLFDDKVMKQLKRRRYTGWGR | |
| | LSRKLINGIRDKQSGKTILDFLKSDGFANRNFMQLIHDDSLTFKEDI | |
| | QKAQVSGQGDSLHEHIANLAGSPAIKKGILQTVKVVDELVKVMG | |
| | RHKPENIVIEMARENQTTQKGQKNSRERMKRIEEGIKELGSQILKE | |
| | HPVENTQLQNEKLYLYYLQNGRDMYVDQELDINRLSDYDVDHIV | |
| | PQSFLKDDSIDNKVLTRSDKNRGKSDNVPSEEVVKKMKNYWRQL | |
| | LNAKLITQRKFDNLTKAERGGLSELDKAGFIKRQLVETRQITKHV | |
| | AQILDSRMNTKYDENDKLIREVKVITLKSKLVSDFRKDFQFYKVR | |
| | EINNYHHAHDAYLNAVVGTALIKKYPKLESEFVYGDYKVYDVRK | |
| | MIAKSEQEIGKATAKYFFYSNIMNFFKTEITLANGEIRKRPLIETNG | |
| | ETGEIVWDKGRDFATVRKVLSMPQVNIVKKTEVQTGGFSKESIRP | |
| | KRNSDKLIARKKDWDPKKYGGFVSPTVAYSVLVVAKVEKGKSK | |
| | KLKSVKELLGITIMERSSFEKNPIDFLEAKGYKEVKKDLIIKLPKYS | |
| | LFELENGRKRMLASARFLQKGNELALPSKYVNFLYLASHYEKLK | |
| | GSPEDNEQKQLFVEQHKHYLDEIIEQISEFSKRVILADANLDKVLS | |
| | AYNKHRDKPIREQAENIIHLFTLTNLGAPRAFKYFDTTIDRKVYRS | |
| | TKEVLDATLIHQSITGLYETRIDLSQLGGDSGGSKRTADGSEFEPK | |
| | KKRKV | |
| MMLV gag | MGQTVTTPLSLTLGHWKDVERIAHNQSVDVKKRRWVTFCSAEW | 64 |
| | PTFNVGWPRDGTFNRDLITQVKIKVFSPGPHGHPDQVPYIVTWEA | |
| | LAFDPPPWVKPFVHPKPPPPLPPSAPSLPLEPPRSTPPRSSLYPALTP | |
| | SLGAKPKPQVLSDSGGPLIDLLTEDPPPYRDPRPPPSDRDGNGGEA | |
| | TPAGEAPDPSPMASRLRGRREPPVADSTTSQAFPLRAGGNGQLQY | |
| | WPFSSSDLYNWKNNNPSFSEDPGKLTALIESVLITHQPTWDDCQQ | |
| | LLGTLLTGEEKQRVLLEARKAVRGDDGRPTQLPNEVDAAFPLERP | |
| | DWDYTTQAGRNHLVHYRQLLLAGLQNAGRSPTNLAKVKGITQGP | |
| | NESPSAFLERLKEAYRRYTPYDPEDPGQETNVSMSFIWQSAPDIGR | |
| | KLERLEDLKNKTLGDLVREAEKIFNKRETPEEREERIRRETEEKEE | |
| | RRRTEDEQKEKERDRRRHREMSKLLATVVSGQKQDRQGGERRRS | |
| | QLDRDQCAYCKEKGHWAKDCPKKPRGPRGPRPQTSLLTLDD | |
| NES | LQLPPLERLTL | 43 |
| Cleavage site A | TSTLLMENSS | 1 |
| (cleavable linker | | |
| sequence) | | |
| NLS | PKKKRKV | 19 |
| TadA8e | SEVEFSHEYWMRHALTLAKRARDEREVPVGAVLVLNNRVIGEGW | 65 |
| | NRAIGLHDPTAHAEIMALRQGGLVMQNYRLIDATLYVTFEPCVM | |
| | CAGAMIHSRIGRVVFGVRNSKRGAAGSLMNVLNYPGMNHRVEIT | |
| | EGILADECAALLCDFYRMPRQVFNAQKKAQSSIN | |
| Cas9 | DKKYSIGLAIGTNSVGWAVITDEYKVPSKKFKVLGNTDRHSIKKN | 66 |
| | LIGALLFDSGETAEATRLKRTARRRYTRRKNRICYLQEIFSNEMAK | |
| | VDDSFFHRLEESFLVEEDKKHERHPIFGNIVDEVAYHEKYPTIYHL | |
| | RKKLVDSTDKADLRLIYLALAHMIKFRGHFLIEGDLNPDNSDVDK | |
| | LFIQLVQTYNQLFEENPINASGVDAKAILSARLSKSRRLENLIAQLP | |
| | GEKKNGLFGNLIALSLGLTPNFKSNFDLAEDAKLQLSKDTYDDDL | |
| | DNLLAQIGDQYADLFLAAKNLSDAILLSDILRVNTEITKAPLSASMI | |
| | KRYDEHHQDLTLLKALVRQQLPEKYKEIFFDQSKNGYAGYIDGG | |
| | ASQEEFYKFIKPILEKMDGTEELLVKLNREDLLRKQRTFDNGSIPH | |
| | QIHLGELHAILRRQEDFYPFLKDNREKIEKILTFRIPYYVGPLARGN | |
| | SRFAWMTRKSEETITPWNFEEVVDKGASAQSFIERMTNFDKNLPN | |
| | EKVLPKHSLLYEYFTVYNELTKVKYVTEGMRKPAFLSGEQKKAIV | |
| | DLLFKTNRKVTVKQLKEDYFKKIECFDSVEISGVEDRFNASLGTY | |
| | HDLLKIIKDKDFLDNEENEDILEDIVLTLTLFEDREMIEERLKTYAH | |
| | LFDDKVMKQLKRRRYTGWGRLSRKLINGIRDKQSGKTILDFLKSD | |
| | GFANRNFMQLIHDDSLTFKEDIQKAQVSGQGDSLHEHIANLAGSP | |
| | AIKKGILQTVKVVDELVKVMGRHKPENIVIEMARENQTTQKGQK | |
| | NSRERMKRIEEGIKELGSQILKEHPVENTQLQNEKLYLYYLQNGR | |
| | DMYVDQELDINRLSDYDVDHIVPQSFLKDDSIDNKVLTRSDKNRG | |
| | KSDNVPSEEVVKKMKNYWRQLLNAKLITQRKFDNLTKAERGGLS | |
| | ELDKAGFIKRQLVETRQITKHVAQILDSRMNTKYDENDKLIREVK | |
| | VITLKSKLVSDFRKDFQFYKVREINNYHHAHDAYLNAVVGTALIK | |
| | KYPKLESEFVYGDYKVYDVRKMIAKSEQEIGKATAKYFFYSNIMN | |
| | FFKTEITLANGEIRKRPLIETNGETGEIVWDKGRDFATVRKVLSMP | |
| | QVNIVKKTEVQTGGFSKESILPKRNSDKLIARKKDWDPKKYGGFD | |
| | SPTVAYSVLVVAKVEKGKSKKLKSVKELLGITIMERSSFEKNPIDF | |
| | LEAKGYKEVKKDLIIKLPKYSLFELENGRKRMLASAGELQKGNEL | |
| | ALPSKYVNFLYLASHYEKLKGSPEDNEQKQLFVEQHKHYLDEIIE | |
| | QISEFSKRVILADANLDKVLSAYNKHRDKPIREQAENIIHLFTLTNL | |
| | GAPAAFKYFDTTIDRKRYTSTKEVLDATLIHQSITGLYETRIDLSQL | |
| | GGD | |
| HEK2 Protospacer | GAACACAAAGCATAGACTGC | 67 |
| HEK2 HTS_fwd | ACACTCTTTCCCTACACGACGCTCTTCCGATCTNNNNCCAGCCC | 68 |
| | CATCTGTCAAACT | |
| HEK2 HTS_rev | TGGAGTTCAGACGTGTGCTCTTCCGATCTTGAATGGATTCCTTG | 69 |
| | GAAACAATGA | |
| HEK2 amplicon | TGAATGGATTCCTTGGAAACAATGATAACAAGACCTGGCTGAG | 70 |
| | CTAACTGTGACAGCATGTGGTAATTTTCCAGCCCGCTGGCCCTG | |
| | TAAAGGAAACTGGAACACAAAGCATAGACTGCGGGGGGGGCC | |
| | AGCCTGAATAGCTGCAAACAAGTGCAGAATATCTGATGATGTC | |
| | ATACGCACAGTTTGACAGATGGGGCTGG | |
| HEK3 Protospacer | GGCCCAGACTGAGCACGTGA | 71 |
| HEK3 HTS_fwd | ACACTCTTTCCCTACACGACGCTCTTCCGATCTNNNNATGTGGG | 72 |
| | CTGCCTAGAAAGG | |
| HEK3 HTS_rev | TGGAGTTCAGACGTGTGCTCTTCCGATCTCCCAGCCAAACTTGT | 73 |
| | CAACC | |
| HEK3 amplicon | ATGTGGGCTGCCTAGAAAGGCATGGATGAGAGAAGCCTGGAG | 74 |
| | ACAGGGATCCCAGGGAAACGCCCATGCAATTAGTCTATTTCTG | |
| | CTGCAAGTAAGCATGCATTTGTAGGCTTGATGCTTTTTTTCTGC | |
| | TTCTCCAGCCCTGGCCTGGGTCAATCCTTGGGGCCCAGACTGAG | |
| | CACGTGATGGCAGAGGAAAGGAAGCCCTGCTTCCTCCAGAGGG | |
| | CGTCGCAGGACAGCTTTTCCTAGACAGGGGCTAGTATGTGCAG | |
| | CTCCTGCACCGGGATACTGGTTGACAAGTTTGGCTGGG | |
| BCL11A enhancer | TTTATCACAGGCTCCAGGAA | 75 |
| protospacer | | |
| BCL11A HTS_fwd | ACACTCTTTCCCTACACGACGCTCTTCCGATCTNNNNGCCAGAA | 76 |
| | AAGAGATATGGCATC | |
| BCL11A HTS_rev | TGGAGTTCAGACGTGTGCTCTTCCGATCTAGAGAGCCTTCCGAA | 77 |
| | AGAGG | |
| BCL11A amplicon | GCCAGAAAAGAGATATGGCATCTACTCTTAGACATAACACACC | 78 |
| | AGGGTCAATACAACTTTGAAGCTAGTCTAGTGCAAGCTAACAG | |
| | TTGCTTTTATCACAGGCTCCAGGAAGGGTTTGGCCTCTGATTAG | |
| | GGTGGGGGCGTGGGTGGGGTAGAAGAGGACTGGCAGACCTCT | |
| | CCATCGGTGGCCGTTTGCCCAGGGGGGCCTCTTTCGGAAGGCT | |
| | CTCT | |
| mDnmt1 protospace | AACAGCTCTGAACGAGACCC | 79 |
| mDnmt1 HTS_fwd | ACACTCTTTCCCTACACGACGCTCTTCCGATCTNNNNCCTTCGG | 80 |
| | GCATAGCATGGTC | |
| mDnmt1 HTS_rev | TGGAGTTCAGACGTGTGCTCTTCCGATCTTATATGCCTCGGCAT | 81 |
| | CGGTCC | |
| mDnmt1 amplicon | TATATGCCTCGGCATCGGTCCCGCCCCTCACCCCCACCCTGCGT | 82 |
| | GGCACCTACCGCCTGCGGACATGGTCCGGGAGCGAGCCTGCCG | |
| | GGGAGGCAAGCGCAGGCACTCGGGCTGGAGCTGTTCGCGCTGG | |
| | CATCTTGCAGGTTGCAGACGACAGAACAGCTCTGAACGAGACC | |
| | CCGGCTTTTTCGCGCGCGCGGAAACCAATTGGGAGGGGGCGGC | |
| | GCAAGCGGAAGCAGCATGTACCACACAGGGCAAGAGAGTGGG | |
| | GGAAGACCATGCTATGCCCGAAGG | |
| BCL11A off-target | CCTATCACTGGCTCCAGGAA | 83 |
| 1 protospacer | | |
| BCL11A off-target | ACACTCTTTCCCTACACGACGCTCTTCCGATCTNNNNACCTGTG | 84 |
| 1 HTS_fwd | GGCATCCTGAGTTGC | |
| BCL11A off-target | TGGAGTTCAGACGTGTGCTCTTCCGATCTTCACGGCCCCACTCC | 85 |
| HTS_rev | TCTCA | |
| BCL11A off-target | ACCTGTGGGCATCCTGAGTTGCTTCTGATGTCCCACCCATCACC | 86 |
| 1 amplicon | TTGACCTGCTCAGAGCAGAGCATTGTTCTGAAATCTGAGGCATT | |
| | GTCCTGCCCACTGGCCTATCACTGGCTCCAGGAAGGGCCTAGT | |
| | GTCTCTGACCAGCTCTAGATCACCTCCTCCTCCTCCTGAGCCCT | |
| | GTACGTTGCCAGGCTGATGAGAGGAGTGGGGCCGTGA | |
| BCL11A off-target | CTTATCATAGGCCCCAGGAA | 87 |
| 2 protospacer | | |
| BCL11A off-target | ACACTCTTTCCCTACACGACGCTCTTCCGATCTNNNNCTTGGCG | 88 |
| 2 HTS_fwd | CAGTTCCTGTGTATG | |
| BCL11A off-target | TGGAGTTCAGACGTGTGCTCTTCCGATCTTACATGCTGTGAGAA | 89 |
| 2 HTS_rev | AATGAAGTGT | |
| BCL11A off-target | CTTGGCGCAGTTCCTGTGTATGGATATTCTTACAGAATCGCTAC | 90 |
| 2 amplicon | TCTCCCTCTCCTTTGAGCTGGCCTAGCTTTGGCTTATCATAGGC | |
| | CCCAGGAAAGGCCAGGGGACTGGGGTACCGGTTAGAGGGATA | |
| | TAAAAGTTCATTCTGCCTTGTACGTATGTTTAATTGATTAGAAC | |
| | ACTTCATTTTCTCACAGCATGTA | |
| HEK2 off-target 1 | GAACACAATGCATAGATTGC | 91 |
| protospacer | | |
| HEK2 off-target 1 | ACACTCTTTCCCTACACGACGCTCTTCCGATCTNNNNGTGTGGA | 92 |
| HTS_fwd | GAGTGAGTAAGCCA | |
| HEK2 off-target 1 | TGGAGTTCAGACGTGTGCTCTTCCGATCTACGGTAGGATGATTT | 93 |
| HTS_rev | CAGGCA | |
| HEK2 off-target 1 | GTGTGGAGAGTGAGTAAGCCAGAACACAATGCATAGATTGCCG | 94 |
| amplicon | GTAAATAGGTTTAGATTCATCCATTTTTAAAAAATGGTGTGGGA | |
| | GCATTAAATATGTATATAGTAGATATGGAAAAATGATTCTCAT | |
| | AATAACTGACATTTCTGTTTCACAAGAAAATTATTTTACATTAT | |
| | ATGTATATTTTACATAAATTATACATAGTCATTTAAAAAGCTCA | |
| | AATAGTGCAAAAACAATATGGAGAATTGCCTGAAATCATCCTA | |
| | CCGT | |
| HEK3 off-target 1 | CACCCAGACTGAGCACGTGC | 95 |
| protospacer | | |
| HEK3 off-target 1 | ACACTCTTTCCCTACACGACGCTCTTCCGATCTNNNNTCCCCTG | 96 |
| HTS_fwd | TTGACCTGGAGAA | |
| HEK3 off-target 1 | TGGAGTTCAGACGTGTGCTCTTCCGATCTCACTGTACTTGCCCT | 97 |
| HTS_rev | GACCA | |
| HEK3 off-target 1 | TCCCCTGTTGACCTGGAGAAGCATGAACCAGTCAAAAAGTTTA | 98 |
| amplicon | AAGACAAGAGCATTAACTGCACCAGTGGGCAGCTCAGCTCAGA | |
| | CACCAGTAGCGTGGGCACCCAGACTGAGCACGTGCTGGAGCCC | |
| | AAGAAATGCAGAGACCTGTGCACCTCTGGTCAGGGCAAGTACA | |
| | GTG | |
| HEK3 off-target 2 | GACACAGACTGGGCACGTGA | 99 |
| protospacer | | |
| HEK3 off-target 2 | ACACTCTTTCCCTACACGACGCTCTTCCGATCTNNNNTTGGTGT | 100 |
| HTS_fwd | TGACAGGGAGCAA | |
| HEK3 off-target 2 | TGGAGTTCAGACGTGTGCTCTTCCGATCTCTGAGATGTGGGCAG | 101 |
| HTS_rev | AAGGG | |
| HEK3 off-target 2 | TTGGTGTTGACAGGGAGCAACTTCACAGTCCCAGGCATCAGGA | 102 |
| amplicon | CACAGACTGGGCACGTGAGGGAAGCCCAAGGGAGAGGACTGG | |
| | TGTAATCGAGGCTGACTCCACTTTTAATGTTTGACTGATGATAG | |
| | GTTTCAAGTCTCACTAAGTCTCCTTCCCCTTCTGCCCACATCTCA | |
| | G | |
| HEK3 off-target 3 | AGCTCAGACTGAGCAAGTGA | 103 |
| protospacer | | |
| HEK3 off-target 3 | ACACTCTTTCCCTACACGACGCTCTTCCGATCTNNNNTGAGAGG | 104 |
| HTS_fwd | GAACAGAAGGGCT | |
| HEK3 off-target 3 | TGGAGTTCAGACGTGTGCTCTTCCGATCTGTCCAAAGGCCCAA | 105 |
| HTS_rev | GAACCT | |
| HEK3 off-target 3 | TGAGAGGGAACAGAAGGGCTAAGACTAAAAGGAACAGAGGAG | 106 |
| amplicon | TTCATAGTGAGCGGTAAAGAGCTCAGACTGAGCAAGTGAGGGG | |
| | CTCAGCCTCCCATGGAGGACAGGGGGCTGGGGCCCCTGGCTGA | |
| | TGTCTGGACTGAAGCCCCCACGCCCAGAGGTTCTTGGGCCTTTG | |
| | GAC | |
| dSaCas9 R-loop 1 | GTGGTAGACAGCATGTGTCCTA | 107 |
| protospacer | | |
| dSaCas9 R-loop 1 | ACACTCTTTCCCTACACGACGCTCTTCCGATCTNNNNTGCAGTC | 108 |
| HTS_fwd | TCCTGCTTCTCTG | |
| dSaCas9 R-loop 1 | TGGAGTTCAGACGTGTGCTCTTCCGATTGGTGGAGTGCTCTGTG | 109 |
| HTS_rev | TTTG | |
| dSaCas9 R-loop 1 | TGGTGGAGTGCTCTGTGTTTGTCTTTATAAACCCAGATGAGAGG | 110 |
| amplicon | ATGAAGGCAACAAGCTTCTGTACCAACATACATGCCCCTTTGC | |
| | CTCAAGTCTGGTTATTTTAGGGGGATGCTAGGTTGCTTTGGGTC | |
| | TACCTTACTGAGAAAATGGCCCCAGGTCATTGTCATGTCCAGTT | |
| | GTGGTAGACAGCATGTGTCCTAAAGGGTATATTCACATGCATG | |
| | TGCAAAAATACAGGGGTCCTTCTAACCCTATCACAGAGAAGCA | |
| | GGAGACTGC | |
| dSaCas9 R-loop 2 | ATTTACAGCCTGGCCTTTGGGG | 111 |
| protospacer | | |
| dSaCas9 R-loop 2 | ACACTCTTTCCCTACACGACGCTCTTCCGATCTNNNNGGACATT | 112 |
| HTS_fwd | TCCACCGCAAAATG | |
| dSaCas9 R-loop 2 | TGGAGTTCAGACGTGTGCTCTTCCGATGCTACAGAAAGGTCAG | 113 |
| HTS_rev | CAGC | |
| dSaCas9 R-loop 2 | GCTACAGAAAGGTCAGCAGCTATATTTAACCTCAGACCAGGGT | 114 |
| amplicon | GCGGTGGGAGATCTGGTTTCCGGAAGACGGAATGGGGAGAAG | |
| | GGCAGGTTCCCCGAGGCGCCCAGACACCCAATCCTCCCGGTGA | |
| | CATTTACAGCCTGGCCTTTGGGGTCGGGTCAACGCTAGGCTGG | |
| | CAGGGGAAGGGCGGGGCCGTGAGGTGAGCCGGCGCTGCAGGA | |
| | AGGGGCCACCACCAGAGGGGCCATTTTGCGGTGGAAATGTCC | |
| dSaCas9 R-loop 3 | GTGTCAGGTAATGTGCTAAACA | 115 |
| protospacer | | |
| dSaCas9 R-loop 3 | ACACTCTTTCCCTACACGACGCTCTTCCGATCTNNNNCTGCACC | 116 |
| HTS_fwd | TAGCCTCCATGTC | |
| dSaCas9 R-loop 3 | TGGAGTTCAGACGTGTGCTCTTCCGATCTGCTGTGGCATCCAGA | 117 |
| HTS_rev | GACAT | |
| dSaCas9 R-loop 3 | AAGTGTTCAGCTGCTTTTCTTTCATTTATTCCACATATAATTACT | 118 |
| amplicon | ATAATTGCTAAACATTTATTTAGTGTCAGGTAATGTGCTAAACA | |
| | GAGAGTTACTGCTCAGACATGTAATAATAATAAATAACACATC | |
| | AAATAACCATACCATTTTAAGCTGTAGTATTATGAAGGGAAAT | |
| | CTGGAGCAAAGAGAATAGACTGTAGGGAAACCAGTTAAGAAA | |
| | TAGGACATGGAGGCTAGGTGCAG | |
| dSaCas9 R-loop 4 | GGTGGAGGAGGGTGCATGGGGT | 119 |
| protospacer | | |
| dSaCas9 R-loop 4 | ACACTCTTTCCCTACACGACGCTCTTCCGATCTNNNNGGAGGTG | 120 |
| HTS_fwd | GAGAGAGGATGT | |
| dSaCas9 R-loop 4 | TGGAGTTCAGACGTGTGCTCTTCCGATCTTCCTGAGGTCTAGGA | 121 |
| HTS_rev | ACCCG | |
| dSaCas9 R-loop 4 | TTTGCTTATCCAGAAAAGGGAGTGATTGCTTCCAGGGGCCTCA | 122 |
| amplicon | GGGGAATAAATCATAGAATCCTGGACAAGGTTTGAAGGACAG | |
| | GTAGGATTTGGGTGGGTGGAGGAGGGTGCATGGGGTCAGAATT | |
| | GTAACCGAAAACTCATTCCAGGTGGATAGAGAAAATTTCTAGT | |
| | GTTGTTGTTTTTAAACTATTTGGGGGACTGGCACAGACCCTTTT | |
| | TGAATACCTGATGGGCTCACATTTCTGTCGAATCCCAG | |
| dSaCas9 R-loop 5 | TCTGCTTCTCCAGCCCTGGC | 123 |
| protospacer | | |
| dSaCas9 R-loop 5 | ACACTCTTTCCCTACACGACGCTCTTCCGATCTNNNNATGTGGG | 72 |
| HTS_fwd | CTGCCTAGAAAGG | |
| dSaCas9 R-loop | TGGAGTTCAGACGTGTGCTCTTCCGATCTCCCAGCCAAACTTGT | 73 |
| HTS_rev | CAACC | |
| dSaCas9 R-loop 5 | ATGTGGGCTGCCTAGAAAGGCATGGATGAGAGAAGCCTGGAG | 74 |
| amplicon | ACAGGGATCCCAGGGAAACGCCCATGCAATTAGTCTATTTCTG | |
| | CTGCAAGTAAGCATGCATTTGTAGGCTTGATGCTTTTTTTCTGC | |
| | TTCTCCAGCCCTGGCCTGGGTCAATCCTTGGGGCCCAGACTGAG | |
| | CACGTGATGGCAGAGGAAAGGAAGCCCTGCTTCCTCCAGAGGG | |
| | CGTCGCAGGACAGCTTTTCCTAGACAGGGGCTAGTATGTGCAG | |
| | CTCCTGCACCGGGATACTGGTTGACAAGTTTGGCTGGG | |
| dSaCas9 R-loop 6 | GATGTTCCAATCAGTACGCA | 124 |
| protospacer | | |
| dSaCas9 R-loop 6 | ACACTCTTTCCCTACACGACGCTCTTCCGATCTNNNNCATTGCA | 125 |
| HTS_fwd | GAGAGGCGTATC | |
| dSaCas9 R-loop 6 | TGGAGTTCAGACGTGTGCTCTTCCGATCTGGGGTCCCAGGTGCT | 126 |
| HTS_rev | GAC | |
| dSaCas9 R-loop 6 | CATTGCAGAGAGGCGTATCATTTCGCGGATGTTCCAATCAGTA | 127 |
| amplicon | CGCAGAGAGTCGCCGTCTCCAAGGTGAAAGCGGAAGTAGGGC | |
| | CTTCGCGCACCTCATGGAATCCCTTCTGCAGCACCTGGATCGCT | |
| | TTTCCGAGCTTCTGGCGGTCTCAAGCACTACCTACGTCAGCACC | |
| | TGGGACCCC | |
| COL7A1(R185X) | CAACTCACTTCAGCTCCTCA | 128 |
| protospacer | | |
| COL7A1(R185X) | ACACTCTTTCCCTACACGACGCTCTTCCGATCTNNNNCTGGTGG | 129 |
| HTS_fwd | ACACAGCTG | |
| COL7A1(R185X) | TGGAGTTCAGACGTGTGCTCTTCCGATCTAGCAGTCGTGCACAC | 130 |
| HTS_rev | | |
| COL7A1(R185X) | CTCTCCGGGAAACGAGGGGCAGTAGTGTCCTCAAGATGCTGAA | 131 |
| amplicon | GTCATTGACGAAGAAGAAGAAGTCACTGGTGGGCTGTGAGGCA | |
| | ACTCACTTCAGCTCCTCAGGGTCAGCATTCTTGATCCCTGAAGT | |
| | GACGACCCATCAGGACTCAGTCACCCACATGCTCTCTGACTGC | |
| | CCCCACCCCCCAGCTGACCTGTCACTCCTGCTCGGTCCTTACCC | |
| | ACAGCAAATAGCTTGACCCCCTGCCCCTTCAGCCTTTGGGCAGC | |
| | TGTGTCCACCAG | |
| Idua(W392X) | ACTCTAGGCAGAGGTCTCAA | 132 |
| protospacer | | |
| Idua(W392X) | ACACTCTTTCCCTACACGACGCTCTTCCGATCTNNNNTTAGGGT | 133 |
| HTS_fwd | AGGAAGCCAGATGCTA | |
| Idua(W392X) | TGGAGTTCAGACGTGTGCTCTTCCGATCTGTGTGCGTGGGTGTC | 134 |
| HTS_rev | ATC | |
| Idua(W392X) | TTAGGGTAGGAAGCCAGATGCTAGGTATGAGAGAGCCAACAG | 135 |
| amplicon | CCTCAGCCCTCTGCTTGGCTTATAGATGGAGAACAACTCTAGGC | |
| | AGAGGTCTCAAAGGCTGGGGCTGTGTTGGACAGCAATCATACA | |
| | GTGGGTGTCCTGGCCAGCACCCATCACCCTGAAGGCTCCGCAG | |
| | CGGCCTGGAGTACCACAGTCCTCATCTACACTAGTGATGACAC | |
| | CCACGCACAC | |
| B2M protospacer | CTTACCCCACTTAACTATCT | 136 |
| B2M HTS_fwd | ACACTCTTTCCCTACACGACGCTCTTCCGATCTNNNNTGTCTTT | 137 |
| | CAGCAAGGACTGGTCT | |
| B2M HTS_rev | TGGAGTTCAGACGTGTGCTCTTCCGATCTGGGACTCATTCAGGG | 138 |
| | TAGTATGGC | |
| B2M amplicon | GGGACTCATTCAGGGTAGTATGGCCATAGACCTTTTTTATATCA | 139 |
| | AAGCAGCTTTATGATATGACTACTCATACACAACTTTCAGCAGC | |
| | TTACAAAAGAATGTAAGACTTACCCCACTTAACTATCTTGGGCT | |
| | GTGACAAAGTCACATGGTTCACACGGCAGGCATACTCATCTTTT | |
| | TCAGTGGGGGTGAATTCAGTGTAGTACAAGAGATAGAAAGACC | |
| | AGTCCTTGCTGAAAGACA | |
| CIITA protospacer | CACTCACCTTAGCCTGAGCA | 140 |
| CIITA HTS_fwd | ACACTCTTTCCCTACACGACGCTCTTCCGATCTNNNNAGGCATG | 141 |
| | CAAGTTTGGTCCTGA | |
| CIITA HTS_rev | TGGAGTTCAGACGTGTGCTCTTCCGATCTCCCTGCAGCCAGCAC | 142 |
| | GATGT | |
| CIITA amplicon | CCCTGCAGCCAGCACGATGTGGGTTCCCTGCGCTCTGCAGCCCC | 143 |
| | CCAGCTCAGCACCTGACCGGTATCCGGGGCCCCACTCACCTTA | |
| | GCCTGAGCAGGGATGCAGCGAGCGAAGGCAGGGCCTCGGCGA | |
| | GTTTGTAGGCACCCAGGTCAGTGATGTTGTTCTGGGACAGACT | |
| | GCGGGGACACAGTGAGGGGGAGGGCTCAGGACCAAACTTGCA | |
| | TGCCT | |
| mPcsk9 protospacer | CCCATACCTTGGAGCAACGG | 144 |
| mPcsk9 HTS_fwd | ACACTCTTTCCCTACACGACGCTCTTCCGATCTNNNNGGCTGCA | 145 |
| | CTTAGAGACCACC | |
| mPcsk9 HTS_rev | TGGAGTTCAGACGTGTGCTCTTCCGATCTATGAAGAGCTGATGC | 146 |
| | TCGCC | |
| mPcsk9 amplicon | GGCTGCACTTAGAGACCACCAGACGGCTAGATGAGCAGAGAA | 147 |
| | GACCCCCGAAGAGCATCACCCCAACCCCAAAGCAACGCCGTTG | |
| | CCTGGCACCCATACCTTGGAGCAACGGCGGAAGGTGGCGGTGG | |
| | CCACATGTGCGGCCTCATCAGCCAGGCCATCCTCCTGGGACGG | |
| | GAGGGCGAGCATCAGCTCTTCAT | |
| mPcsk9 OT1 | CCCCTACCTTGGGGCAACAG | 148 |
| protospacer | | |
| mPcsk9 OT1 | ACACTCTTTCCCTACACGACGCTCTTCCGATCTNNNNAAGTATG | 149 |
| HTS_fwd | TTGGGACCCTTGGCTGG | |
| mPcsk9 OT1 | TGGAGTTCAGACGTGTGCTCTTCCGATCTTGGCCTGTTCTACTG | 150 |
| HTS_rev | ACTATGGGG | |
| mPcsk9 OT1 | AAGTATGTTGGGACCCTTGGCTGGGCTTCTTGCCCTCTCTAGAA | 151 |
| amplicon | CCAAGATGTCACTTCTGCACACCAAGAGCTACCCCTACCTTGG | |
| | GGCAACAGTGGAAGCCATGGCTGGAGAAAGCAAACAATTCCT | |
| | GAAGGTGACAGATTCTCCTGGGAAGGGACTTAGCCCCATAGTC | |
| | AGTAGAACAGGCCA | |
| mPcsk9 OT2 | ACCATACCTAAGAGCAAACT | 152 |
| protospacer | | |
| mPcsk9 OT2 | ACACTCTTTCCCTACACGACGCTCTTCCGATCTNNNNGACAGAC | 153 |
| HTS_fwd | ACAGGGAAGCCTTGGG | |
| mPcsk9 OT2 | TGGAGTTCAGACGTGTGCTCTTCCGATCTAACCTTCCAGGAGAG | 154 |
| HTS_rev | AGAAACCTGT | |
| mPcsk9 OT2 | GACAGACACAGGGAAGCCTTGGGGAGCCGGAGGCTTGGCCAG | 155 |
| amplicon | GAGCTCAGGGGTCCCTGGGCAGATGCTCACACTGGGCAGAAGG | |
| | TCACACCATACCTAAGAGCAAACTGGGGCCCAAACGACTGAGT | |
| | GTTGCTGAGAGCCATCCTTGGCTCATTCTCAAAAAACAGGTTTC | |
| | TCTCTCCTGGAAGGTT | |
| mPcsk9 OT3 | CCCACCCTTTGGAGAACGG | 156 |
| protospacer | | |
| mPcsk9 OT3 | ACACTCTTTCCCTACACGACGCTCTTCCGATCTNNNNTGGCAAG | 157 |
| HTS_fwd | GGACAGGGTCAGC | |
| mPcsk9 OT3 | TGGAGTTCAGACGTGTGCTCTTCCGATCTAGCTGGTGGCAGAG | 158 |
| HTS_rev | GTGTGG | |
| mPcsk9 OT3 | TGGCAAGGGACAGGGTCAGCTCTTCACTCCCATTCCATCTGGG | 159 |
| amplicon | GCAGCTCACCTGCATCCAAGCCAATAGAGACAGCCCTACTGTG | |
| | TTGCTCAGTTGAGGTACGGGGCCCACCCTTTGGAGAACGGTGG | |
| | GGGTGGGAGCTATGCCAACACTTCTGCTCTAACACCCTCACAG | |
| | CTAGCTCACCCACACCTCTGCCACCAGCT | |
| mPcsk9 OT4 | CCCAGCCTTGGGGCAACGG | 160 |
| protospacer | | |
| mPcsk9 OT4 | ACACTCTTTCCCTACACGACGCTCTTCCGATCTNNNNTTCAAGC | 161 |
| HTS_fwd | AATCACGAGACACTCAG | |
| mPcsk9 OT4 | TGGAGTTCAGACGTGTGCTCTTCCGATCTCCCACCACCCAGCAG | 162 |
| HTS_rev | CTTTATTG | |
| mPcsk9 OT4 | TTCAAGCAATCACGAGACACTCAGTTTGGATCCCCAGAGCCCA | 163 |
| amplicon | CATAAAAGATCAGACACAGAGTGCATGCCTGTAACCCCAGCCT | |
| | TGGGGCAACGGAGGCTCTGAAGCTCGTCGGTTAGCCAGCTGAA | |
| | GCATATCCATGAGGTTTAGTGTTGGAGCCTGTCTCAATAAAGCT | |
| | GCTGGGTGGTGGG | |
| mPcsk9 OT5 | CACATATCTAGGAGCAAGG | 164 |
| protospacer | | |
| mPcsk9 OT5 | ACACTCTTTCCCTACACGACGCTCTTCCGATCTNNNNTCTCAGG | 165 |
| HTS_fwd | CGACCTGGTTTCTGC | |
| mPcsk9 OT5 | TGGAGTTCAGACGTGTGCTCTTCCGATCTTCTGCCAGATGCGTC | 166 |
| HTS_rev | CGATCA | |
| mPcsk9 OT5 | TCTCAGGCGACCTGGTTTCTGCAAAGGGCAGGGTTGGCTTTATG | 167 |
| amplicon | CTGAGTCCTACAGATCTTAGACCCCCCCCCCCAAACTTAAACAC | |
| | ATATCTAGGAGCAAGGAGGGGTCATGAAAAGATAGAGCCTGCT | |
| | TTGGCAGACTATAGAACAGAACACTAAGGATTTAACTTACTAG | |
| | TGAAATGATCGGACGCATCTGGCAGA | |
| mPcsk9 OT6 | CCCACACCCGGAGCAACGG | 168 |
| protospacer | | |
| mPcsk9 OT6 | ACACTCTTTCCCTACACGACGCTCTTCCGATCTNNNNGCCAGCC | 169 |
| HTS_fwd | CTGCCTGGAAGTTAG | |
| mPcsk9 OT6 | TGGAGTTCAGACGTGTGCTCTTCCGATCTTGACCTCCGGGATTC | 170 |
| HTS_rev | TCAGCCC | |
| mPcsk9 OT6 | GCCAGCCCTGCCTGGAAGTTAGCCATGGAGGATGGAGCTGAAC | 171 |
| amplicon | TTGACCTTTGCGGTTCACAGCCCACACCCGGAGCAACGGGGAG | |
| | GTCGTCGTGAGCCCAGTCAGTCGTTTGGTTGCAAAGAACTTTTT | |
| | AATAAGGGAAGTTTTCAGTCATGGAATGAGAGGTGAGGTGAAG | |
| | TGGGCTGAGAATCCCGGAGGTCA | |
| mPcsk9 OT7 | TCCATACCCGGAGCAACGA | 172 |
| protospacer | | |
| mPcsk9 OT7 | ACACTCTTTCCCTACACGACGCTCTTCCGATCTNNNNGCTTCCT | 173 |
| HTS_fwd | GTCTGCAATTGGGGTCT | |
| mPcsk9 OT7 | TGGAGTTCAGACGTGTGCTCTTCCGATCTAGTAGGTTGCGGGGC | 174 |
| HTS_rev | TCAGGA | |
| mPcsk9 OT7 | GCTTCCTGTCTGCAATTGGGGTCTTTGTTGTCCTTCTGGCTGTCC | 175 |
| amplicon | TTCTCCTCTTCATCAACAAGAAGCTATGCTCTGAAAACCTAAGA | |
| | GGGCATCCATACCCGGAGCAACGAGGGAAGAGAAAGCACTCG | |
| | AGAGACAAGACTGGAGGCCACACAGGAACTGGTAAGCACCAT | |
| | GCTTTATGTTTTCCTGAGCCCCGCAACCTACT | |
| mPcsk9 OT8 | TTCATCCTTGGAGCAACGG | 176 |
| protospacer | | |
| mPcsk9 OT8 | ACACTCTTTCCCTACACGACGCTCTTCCGATCTNNNNCCAGCAG | 177 |
| HTS_fwd | GTCCCCAGTGACG | |
| mPcsk9 OT8 | TGGAGTTCAGACGTGTGCTCTTCCGATCTTACCCACCTGGGTGT | 178 |
| HTS_rev | GTCCA | |
| mPcsk9 OT8 | CCAGCAGGTCCCCAGTGACGCAAGCCAGCAGGGGGTGGGAAG | 179 |
| amplicon | CTTCAGGAGAAAAGGACATGGAGCAGTAGGGTATGACATTCAA | |
| | AGCCTGACAGCGTCTCTACCAGCCCTTCATCCTTGGAGCAACG | |
| | GTGAGATGAACATTTATGTTCATACTGCAGAGTTGAACAGAAT | |
| | CCAGAACAGCCAGCCTTTTGAGCTACATAACAAAAGTATCATG | |
| | TGCACATGTGGACACACCCAGGTGGGTA | |
| mPcsk9 OT9 | TCTGTACCATGGAGCAAAGG | 180 |
| protospacer | | |
| mPcsk9 OT9 | ACACTCTTTCCCTACACGACGCTCTTCCGATCTNNNNAACCTCC | 181 |
| HTS_fwd | ACGGGGGTATCTGAGG | |
| mPcsk9 OT9 | TGGAGTTCAGACGTGTGCTCTTCCGATCTACCTGGCAAGTGGG | 182 |
| HTS_rev | GTACTGG | |
| mPcsk9 OT9 | AACCTCCACGGGGGTATCTGAGGTCTTCTGCTGTAGTGTGTCCT | 183 |
| amplicon | TTCAGTCATCAATAACATGGGCAGGTACCATCCCCTCCGATGTG | |
| | GGCGAGTACCACAAGTTTGCAAGGTCACAGGGCTGCTCTGTAC | |
| | CATGGAGCAAAGGCGGAAAGGAAACCTTGGGTGTCTGATGCAT | |
| | TGGAACCCAGTACCCCACTTGCCAGGT | |
| mPcsk9 OT10 | ACCATAACCAAGAGCAACAG | 184 |
| protospacer | | |
| mPcsk9 OT10 | ACACTCTTTCCCTACACGACGCTCTTCCGATCTNNNNGTCTAAA | 185 |
| HTS_fwd | TGGGCAAGCAATCCCCT | |
| mPcsk9 OT10 | TGGAGTTCAGACGTGTGCTCTTCCGATCTCCAGGATCCCACAGG | 186 |
| HTS_rev | GTCCTTCT | |
| mPcsk9 OT10 | GTCTAAATGGGCAAGCAATCCCCTGTCCAGGGTCGATTCAGGG | 187 |
| amplicon | CTGTCTGTGAGAAGTCTCGGTGTCTTATGGAGGATTTCTACTGA | |
| | TGAGTAAAACACCATAACCAAGAGCAACAGGGGAGGGAAGGG | |
| | TCTCCTGCAGCTTACATCTGACAGTCATCCAGGGTAGTCAGTGA | |
| | AGGGACTCTCTCAGAAGGACCCTGTGGGATCCTGG | |
| mPcsk9 OT11 | TCCATAACTCAGAGCAACAG | 188 |
| protospacer | | |
| mPcsk9 OT11 | ACACTCTTTCCCTACACGACGCTCTTCCGATCTNNNNTCCCCAG | 189 |
| HTS_fwd | AGCCCAGGGAATATCA | |
| mPcsk9 OT11 | TGGAGTTCAGACGTGTGCTCTTCCGATCTTGTTGCTCCGATGGA | 190 |
| HTS_rev | AGGATGGG | |
| mPcsk9 OT11 | TCCCCAGAGCCCAGGGAATATCATGGGGGAATATAAGAGCTAT | 191 |
| amplicon | AGGATGAGAATTGGTGCTACAGAATGCTGTTTGTGGATAAGAC | |
| | ATGGCTGATGCATCCATAACTCAGAGCAACAGTGGTGACTTGC | |
| | TCAAGACCTTCACAAGACTGAGCTGTCAACCTTCTACCCTGGAT | |
| | GGAAGACGGGATGGTAAGATCCCATCCTTCCATCGGAGCAACA | |
| mPcsk9 OT12 | GCCATACCCTGGGGCAGCAG | 192 |
| protospacer | | |
| mPcsk9 OT12 | ACACTCTTTCCCTACACGACGCTCTTCCGATCTNNNNTGTGGAA | 193 |
| HTS_fwd | CCCACCCCCGATACA | |
| mPcsk9 OT12 | TGGAGTTCAGACGTGTGCTCTTCCGATCTAGTGCTGATGGGCAA | 194 |
| HTS_rev | GGCATTTG | |
| mPcsk9 OT12 | TGTGGAACCCACCCCCGATACACACACACCTTAAGTCGTACCT | 195 |
| amplicon | CTCTCAACATGTCTGCTGAAGCCACCTGCCCCGCGAGAGTAAG | |
| | CAGGCGCCATACCCTGGGGCAGCAGTGGAGGCTATGATTTAGA | |
| | ATAACTGTGGTCCGGTCTCTCTAACATTTGCCGCTGTATTCATT | |
| | CTAAGTTTAATGAGGGACAAATGCCTTGCCCATCAGCACT | |
| mPcsk9 OT13 | GCAACACCTTGGAGCAACTG | 196 |
| protospacer | | |
| mPcsk9 OT13 | ACACTCTTTCCCTACACGACGCTCTTCCGATCTNNNNCCACCAG | 197 |
| HTS_fwd | AAGCGCCCCAGAA | |
| mPcsk9 OT13 | TGGAGTTCAGACGTGTGCTCTTCCGATCTGGGGAATCGCCTCCA | 198 |
| HTS_rev | CTGCC | |
| mPcsk9 OT13 | CCACCAGAAGCGCCCCAGAACTCCTTGCTGGCTAGTTGGCCTCT | 199 |
| amplicon | CATCAGCTCAGCCTGCCCAACTCAGCGTGGGGCTGTAGGTGCA | |
| | ACACCTTGGAGCAACTGAGGTATCAACAGCAGAGATAGAGATG | |
| | GAGGAAGCTGCAGCAACAGAGGCAGTGGAGGCGATTCCCC | |
| mPcsk9 OT14 | GACATCCTTGGAGCAACTG | 200 |
| protospacer | | |
| mPcsk9 OT14 | ACACTCTTTCCCTACACGACGCTCTTCCGATCTNNNNGTTCTTA | 201 |
| HTS_fwd | TTGGCCAGGGAGCCTT | |
| mPcsk9 OT14 | TGGAGTTCAGACGTGTGCTCTTCCGATCTCTCCCCAAGTGACAG | 202 |
| HTS_rev | GAACCACG | |
| mPcsk9 OT14 | GTTCTTATTGGCCAGGGAGCCTTTCTGCAGTTCTTTGTAAATCC | 203 |
| amplicon | AGCTAAAATGCAAACACTGACATCAATCATTTGAAATGAGGTG | |
| | GCTGTCAGGTCCTCAGACATCCTTGGAGCAACTGTGGGTGAGT | |
| | ATTCCTGATGGGAATTTTCTCTCTTCATCCAGGAGTGAGGGCTC | |
| | ACTTGGTGCCCAACCTACAGGCTGGGTGGAGGGCTGGGCACCA | |
| | CGTGGTTCCTGTCACTTGGGGAG | |
| Rpe65(r44X) | ACATCAGAGGAGACTGCCAG | 204 |
| gDNA protospacer | | |
| Rpe65(r44X) | ACACTCTTTCCCTACACGACGCTCTTCCGATCTNNNNAGCTGAC | 205 |
| gDNA HTS_fwd | AAATAACAAATAGGCACA | |
| Rpe65(r44X) | TGGAGTTCAGACGTGTGCTCTTCCGATCTGGCTCTACTCTGGTG | 206 |
| gDNA HTS_rev | AGGTCAG | |
| Rpe65(r44X) | GGCTCTACTCTGGTGAGGTCAGTCATGGACTTACCTTCTGTGGT | 207 |
| gDNA amplicon | ATGTGACATGGCCCTCCTTGAAGTCAAACTTGTGCAAAAGGGC | |
| | TTGTCCATCAAACAGGTGATAGAAAGGCTCAGATCCAACTTCA | |
| | AAGAGCCCTGGCCCACATCAGAGGAGACTGCCAGTGAGCCAG | |
| | AGGGGAATCCTGCCTGCAGCAAAGTGAGATATCAGGTGGTACT | |
| | ACTTACTAGATTTTCTATGTGCCTATTTGTTATTTGTCAGCT | |
| Rpe65(r44X) | ACATCAGAGGAGACTGCCAG | 204 |
| cDNA protospacer | | |
| Rpe65(r44X) | ACACTCTTTCCCTACACGACGCTCTTCCGATCTNNNNTGTCCTC | 208 |
| CDNA HTS_fwd | ACCACTAACAGCT | |
| Rpe65(r44X) | TGGAGTTCAGACGTGTGCTCTTCCGATCTCTTCTCAGTCATTGC | 209 |
| CDNA HTS_rev | TCGAACA | |
| Rpe65(r44X) | CTTCTCAGTCATTGCTCGAACATAAGCATCAGTGCGGATGAATC | 210 |
| cDNA amplicon | TTCTGTGGTATGTGACATGGCCCTCCTTGAAGTCAAACTTGTGC | |
| | AAAAGGGCTTGTCCATCAAACAGGTGATAGAAAGGCTCAGATC | |
| | CAACTTCAAAGAGCCCTGGCCCACATCAGAGGAGACTGCCAGT | |
| | GAGCCAGAGGGGAATCCTGCCTGTGACATGAGCTGTTAGTGGT | |
| | GAGGACA | |
| Mcm3ap cDNA | ACACTCTTTCCCTACACGACGCTCTTCCGATCTNNNNGCTTCCA | 211 |
| HTS_fwd | AAGCCTGCGCCTG | |
| Mcm3ap cDNA | TGGAGTTCAGACGTGTGCTCTTCCGATCTCCATGGAAACTTCCT | 212 |
| HTS_rev | CAGCGGC | |
| Mcm3ap cDNA | GCTTCCAAAGCCTGCGCCTGTGTACTCTGACTCGGACCTGGTAC | 213 |
| amplicon | AGGTGGTGGACGAGCTCATCCAGGAGGCTCTGCAAGTGGACTG | |
| | TGAGGAAGTCAGCTCCGCTGGGGCAGCCTACGTAGCCGCAGCT | |
| | CTGGGCGTTTCCAATGCTGCTGTGGAGGATCTGATTACTGCTGC | |
| | GACCACGGGCATTCTGAGGCACGTTGCCGCTGAGGAAGTTTCC | |
| | ATGG | |
| Perp cDNA | ACACTCTTTCCCTACACGACGCTCTTCCGATCTNNNNGCCATCG | 214 |
| HTS fwd | CCTTCGACATCAT | |
| Perp cDNA | TGGAGTTCAGACGTGTGCTCTTCCGATCTAACAAGCATCTGGG | 215 |
| HTS_rev | GTCCAC | |
| Perp cDNA | GCCATCGCCTTCGACATCATCGCGCTGGCCGGCCGCGGCTGGC | 216 |
| amplicon | TGCAGTCTAGCAACCACATCCAGACATCGTCGCTTTGGTGGAG | |
| | GTGTTTCGACGAGGGCGGCGGCAGCGGCTCCTACGACGATGGC | |
| | TGCCAGAGCCTCATGGAGTACGCATGGGGACGAGCAGCTGCAG | |
| | CCACGCTTTTCTGTGGCTTTATCATCCTGTGCATCTGCTTCATTC | |
| | TCTCGTTCTTCGCCCTGTGTGGACCCCAGATGCTTGTT | |
| qPCR detection of | ACACTCTTTCCCTACACGACGCTCTTCCGATCTNNNNGTTTATC | 217 |
| sgRNA fwd primer | ACAGGCTCCAGGAAG | |
| qPCR detection of | TGGAGTTCAGACGTGTGCTCTTCCGATCTGGTGCCACTTTTTCA | 218 |
| sgDNA rev primer | AGTTGATAAC | |
| qPCR detection of | ACGAGCACATTGCCAATCTG | 219 |
| BE-encoding DNA | | |
| fwd primer | | |
| qPCR detection of | GCCATTTCGATCACGATGTTC | 220 |
| BE-encoding DNA | | |
| rev primer | | |
| On-target (Pcsk9) | CCCATACCTTGGAGCAACGG CGG | 221 |
| spacer | | |
| OT1 (ELP3 intron) | GACATACCTTAAAGCAAAGG AGG | 222 |
| spacer | | |
| OT2 (Intergenic) | CCCCTACCTTGGGGCAACAG TGG | 223 |
| spacer | | |
| OT3 (LINC02006 | CCCACCCTTTGGAGAACGG TGG | 224 |
| IncRNA) spacer | | |
| OT4 (intergenic) | CCCAG-CCTTGGGGCAACGG AGG | 225 |
| spacer | | |
| OT5 (intergenic) | CACATATCTAGGAGCAAGG AGG | 226 |
| spacer | | |
| OT6 (DDX6 intron) | CCCACACCCGGAGCAACGG GGA | 227 |
| spacer | | |
| OT7 (RP11- | TCCATACCC-GGAGCAACGA GGG | 228 |
| 314D7.4 incRNA) | | |
| spacer | | |
| OT8 (FAM66D | TTCATCCTTGGAGCAACGG TGA | 229 |
| IncRNA) spacer | | |
| OT9 (RIKEN | TCTGTACCATGGAGCAAAGG CGG | 230 |
| cDNA 4933424G05 | | |
| gene IncRNA) | | |
| spacer | | |
| OT10 (Klh13 intron) | ACCATAACCAAGAGCAACAG GGG | 231 |
| spacer | | |
| OT11 (intergenic) | TCCATAACTCAGAGCAACAG TGG | 232 |
| spacer | | |
| OT12 (NCAM1 | GCCATACCCTGGGGCAGCAG TGG | 233 |
| intron) spacer | | |
| OT13 (SNRNP40 | GCAACACCTTGGAGCAACTG AGG | 234 |
| intron) spacer | | |
| OT14 (Fry intron) | GACATCCTTGGAGCAACTG TGG | 235 |
| spacer | | |

### REFERENCES

Abifadel, M., Varret, M., Rabes, J. P., Allard, D., Ouguerram, K., Devillers, M., Cruaud, C., Benjannet, S., Wickham, L., Erlich, D., et al. (2003). Mutations in PCSK9 cause autosomal dominant hypercholesterolemia. Nat Genet 34, 154-156.
Akcakaya, P., Bobbin, M. L., Guo, J. A., Malagon-Lopez, J., Clement, K., Garcia, S. P., Fellows, M. D., Porritt, M. J., Firth, M. A., Carreras, A., et al. (2018). In vivo CRISPR editing with no detectable genome-wide off-target mutations. Nature 561, 416-419.
Alanis-Lobato, G., Zohren, J., McCarthy, A., Fogarty, N. M. E., Kubikova, N., Hardman, E., Greco, M., Wells, D., Turner, J. M. A., and Niakan, K. K. (2021). Frequent loss of heterozygosity in CRISPR-Cas9-edited early human embryos. Proc Natl Acad Sci USA 118.
Anzalone, A. V., Koblan, L. W., and Liu, D. R. (2020). Genome editing with CRISPR-Cas nucleases, base editors, transposases and prime editors. Nat Biotechnol 38, 824-844.
Campbell, L. A., Coke, L. M., Richie, C. T., Fortuno, L. V., Park, A. Y., and Harvey, B. K. (2019). Gesicle-Mediated Delivery of CRISPR/Cas9 Ribonucleoprotein Complex for Inactivating the HIV Provirus. Mol Ther 27, 151-163.
Chandler, R. J., Sands, M. S., and Venditti, C. P. (2017). Recombinant Adeno-Associated Viral Integration and Genotoxicity: Insights from Animal Models. Hum Gene Ther 28, 314-322.
Chen, P. J., Hussmann, J. A., Yan, J., Knipping, F., Ravisankar, P., Chen, P. F., Chen, C., Nelson, J. W., Newby, G. A., Sahin, M., et al. (2021). Enhanced prime editing systems by manipulating cellular determinants of editing outcomes. Cell 184, 5635-5652 e5629.
Choi, J. G., Dang, Y., Abraham, S., Ma, H., Zhang, J., Guo, H., Cai, Y., Mikkelsen, J. G., Wu, H., Shankar, P., et al. (2016). Lentivirus pre-packed with Cas9 protein for safer gene editing. Gene Ther 23, 627-633.
Cideciyan, A. V. (2010). Leber congenital amaurosis due to RPE65 mutations and its treatment with gene therapy. Prog Retin Eye Res 29, 398-427.
Clement, K., Rees, H., Canver, M. C., Gehrke, J. M., Farouni, R., Hsu, J. Y., Cole, M. A., Liu, D. R., Joung, J. K., Bauer, D. E., et al. (2019). CRISPResso2 provides accurate and rapid genome editing sequence analysis. Nat Biotechnol 37, 224-226.
Cohen, J., Pertsemlidis, A., Kotowski, I. K., Graham, R., Garcia, C. K., and Hobbs, H. H. (2005). Low LDL cholesterol in individuals of African descent resulting from frequent nonsense mutations in PCSK9. Nat Genet 37, 161-165.
Cohen, J. C., Boerwinkle, E., Mosley, T. H., Jr., and Hobbs, H. H. (2006). Sequence variations in PCSK9, low LDL, and protection against coronary heart disease. N Engl J Med 354, 1264-1272.
Cronin, J., Zhang, X. Y., and Reiser, J. (2005). Altering the tropism of lentiviral vectors through pseudotyping. Curr Gene Ther 5, 387-398.
David, R. M., and Doherty, A. T. (2017). Viral Vectors: The Road to Reducing Genotoxicity. Toxicol Sci 155, 315-325.
Davis, K. M., Pattanayak, V., Thompson, D. B., Zuris, J. A., and Liu, D. R. (2015). Small molecule-triggered Cas9 protein with improved genome-editing specificity. Nat Chem Biol 11, 316-318.
den Hollander, A. I., Roepman, R., Koenekoop, R. K., and Cremers, F. P. (2008). Leber congenital amaurosis: genes, proteins and disease mechanisms. Prog Retin Eye Res 27, 391-419.
Deverman, B. E., Pravdo, P. L., Simpson, B. P., Kumar, S. R., Chan, K. Y., Banerjee, A., Wu, W. L., Yang, B., Huber, N., Pasca, S. P., et al. (2016). Cre-dependent selection yields AAV variants for widespread gene transfer to the adult brain. Nat Biotechnol 34, 204-209.
Doman, J. L., Raguram, A., Newby, G. A., and Liu, D. R. (2020). Evaluation and minimization of Cas9-independent off-target DNA editing by cytosine base editors. Nat Biotechnol 38, 620-628.
Doudna, J. A. (2020). The promise and challenge of therapeutic genome editing. Nature 578, 229-236.
Feher, A., Boross, P., Sperka, T., Miklossy, G., Kadas, J., Bagossi, P., Oroszlan, S., Weber, I. T., and Tozser, J. (2006). Characterization of the murine leukemia virus protease and its comparison with the human immunodeficiency virus type 1 protease. J Gen Virol 87, 1321-1330.
Fitzgerald, K., Frank-Kamenetsky, M., Shulga-Morskaya, S., Liebow, A., Bettencourt, B. R., Sutherland, J. E., Hutabarat, R. M., Clausen, V. A., Karsten, V., Cehelsky, J., et al. (2014). Effect of an RNA interference drug on the synthesis of proprotein convertase subtilisin/kexin type 9 (PCSK9) and the concentration of serum LDL cholesterol in healthy volunteers: a randomised, single-blind, placebo-controlled, phase 1 trial. Lancet 383, 60-68.
Gaudelli, N. M., Komor, A. C., Rees, H. A., Packer, M. S., Badran, A. H., Bryson, D. I., and Liu, D. R. (2017). Programmable base editing of A*T to G*C in genomic DNA without DNA cleavage. Nature 551, 464-471.
Gaudelli, N. M., Lam, D. K., Rees, H. A., Sola-Esteves, N. M., Barrera, L. A., Born, D. A., Edwards, A., Gehrke, J. M., Lee, S., Liquori, A. J., et al. (2020). Directed evolution of adenine base editors with increased activity and therapeutic application. Nat Biotechnol 38, 892-900.
Gee, P., Lung, M. S. Y., Okuzaki, Y., Sasakawa, N., Iguchi, T., Makita, Y., Hozumi, H., Miura, Y., Yang, L. F., Iwasaki, M., et al. (2020). Extracellular nanovesicles for packaging of CRISPR-Cas9 protein and sgRNA to induce therapeutic exon skipping. Nat Commun 11, 1334.
Giannoukos, G., Ciulla, D. M., Marco, E., Abdulkerim, H. S., Barrera, L. A., Bothmer, A., Dhanapal, V., Gloskowski, S. W., Jayaram, H., Maeder, M. L., et al. (2018). UDiTaS, a genome editing detection method for indels and genome rearrangements. BMC Genomics 19, 212.
Golczak, M., Kiser, P. D., Lodowski, D. T., Maeda, A., and Palczewski, K. (2010). Importance of Membrane Structural Integrity for RPE65 Retinoid Isomerization Activity. Journal of Biological Chemistry 285, 9667-9682.
Hamilton, J. R., Tsuchida, C. A., Nguyen, D. N., Shy, B. R., McGarrigle, E. R., Sandoval Espinoza, C. R., Carr, D., Blaeschke, F., Marson, A., and Doudna, J. A. (2021). Targeted delivery of CRISPR-Cas9 and transgenes enables complex immune cell engineering. Cell Rep 35, 109207.
Hooper, A. J., Marais, A. D., Tanyanyiwa, D. M., and Burnett, J. R. (2007). The C679X mutation in PCSK9 is present and lowers blood cholesterol in a Southern African population. Atherosclerosis 193, 445-448.
Huang, T. P., Zhao, K. T., Miller, S. M., Gaudelli, N. M., Oakes, B. L., Fellmann, C., Savage, D. F., and Liu, D. R. (2019). Circularly permuted and PAM-modified Cas9 variants broaden the targeting scope of base editors. Nat Biotechnol 37, 626-631.
Humbel, M., Ramosaj, M., Zimmer, V., Regio, S., Aeby, L., Moser, S., Boizot, A., Sipion, M., Rey, M., and Deglon, N. (2021). Maximizing lentiviral vector gene transfer in the CNS. Gene Ther 28, 75-88.
Indikova, I., and Indik, S. (2020). Highly efficient ‘hit-and-run’ genome editing with unconcentrated lentivectors carrying Vpr.Prot.Cas9 protein produced from RRE-containing transcripts. Nucleic Acids Res 48, 8178-8187.
Jang, H. K., Jo, D. H., Lee, S. N., Cho, C. S., Jeong, Y. K., Jung, Y., Yu, J., Kim, J. H., Woo, J. S., and Bae, S. (2021). High-purity production and precise editing of DNA base editing ribonucleoproteins. Sci Adv 7.
Jo, D. H., Jang, H.-K., Cho, C. S., Han, J. H., Ryu, G., Jung, Y., Bae, S., and Kim, J. H. (2021). Therapeutic adenine base editing corrects nonsense mutation and improves visual function in a mouse model of Leber congenital amaurosis. bioRxiv.
Johnson, S., Wheeler, J. X., Thorpe, R., Collins, M., Takeuchi, Y., and Zhao, Y. (2018). Mass spectrometry analysis reveals differences in the host cell protein species found in pseudotyped lentiviral vectors. Biologicals 52, 59-66.
Kaczmarczyk, S., Sitaraman, K., Young, H. A., Hughes, S. H., and Chatterjee, D. K. (2011). Protein delivery using engineered virus-like particles. Proc Natl Acad Sci USA 108, 16998-17003.
Kato, S., Kuramochi, M., Kobayashi, K., Fukabori, R., Okada, K., Uchigashima, M., Watanabe, M., Tsutsui, Y., and Kobayashi, K. (2011). Selective neural pathway targeting reveals key roles of thalamostriatal projection in the control of visual discrimination. J Neurosci 31, 17169-17179.
Koblan, L. W., Doman, J. L., Wilson, C., Levy, J. M., Tay, T., Newby, G. A., Maianti, J. P., Raguram, A., and Liu, D. R. (2018). Improving cytidine and adenine base editors by expression optimization and ancestral reconstruction. Nat Biotechnol 36, 843-846.
Koblan, L. W., Erdos, M. R., Wilson, C., Cabral, W. A., Levy, J. M., Xiong, Z. M., Tavarez, U. L., Davison, L. M., Gete, Y. G., Mao, X., et al. (2021). In vivo base editing rescues Hutchinson-Gilford progeria syndrome in mice. Nature 589, 608-614.
Komor, A. C., Kim, Y. B., Packer, M. S., Zuris, J. A., and Liu, D. R. (2016). Programmable editing of a target base in genomic DNA without double-stranded DNA cleavage. Nature 533, 420-424.
Kosicki, M., Tomberg, K., and Bradley, A. (2018). Repair of double-strand breaks induced by CRISPR-Cas9 leads to large deletions and complex rearrangements. Nat Biotechnol 36, 765-771.
Lazzarotto, C. R., Malinin, N. L., Li, Y., Zhang, R., Yang, Y., Lee, G., Cowley, E., He, Y., Lan, X., Jividen, K., et al. (2020). CHANGE-seq reveals genetic and epigenetic effects on CRISPR-Cas9 genome-wide activity. Nature Biotechnology 38, 1317-1327.
Leibowitz, M. L., Papathanasiou, S., Doerfler, P. A., Blaine, L. J., Sun, L., Yao, Y., Zhang, C. Z., Weiss, M. J., and Pellman, D. (2021). Chromothripsis as an on-target consequence of CRISPR-Cas9 genome editing. Nat Genet 53, 895-905.
LeibundGut-Landmann, S., Waldburger, J. M., Krawczyk, M., Otten, L. A., Suter, T., Fontana, A., Acha-Orbea, H., and Reith, W. (2004). Mini-review: Specificity and expression of CIITA, the master regulator of MHC class II genes. Eur J Immunol 34, 1513-1525.
Levy, J. M., Yeh, W. H., Pendse, N., Davis, J. R., Hennessey, E., Butcher, R., Koblan, L. W., Comander, J., Liu, Q., and Liu, D. R. (2020). Cytosine and adenine base editing of the brain, liver, retina, heart and skeletal muscle of mice via adeno-associated viruses. Nat Biomed Eng 4, 97-110.
Lyu, P., Javidi-Parsijani, P., Atala, A., and Lu, B. (2019). Delivering Cas9/sgRNA ribonucleoprotein (RNP) by lentiviral capsid-based bionanoparticles for efficient ‘hit-and-run’ genome editing. Nucleic Acids Res 47, e99.
Lyu, P., Lu, Z., Cho, S. I., Yadav, M., Yoo, K. W., Atala, A., Kim, J. S., and Lu, B. (2021).
Adenine Base Editor Ribonucleoproteins Delivered by Lentivirus-Like Particles Show High On-Target Base Editing and Undetectable RNA Off-Target Activities. CRISPR J 4, 69-81.
Mangeot, P. E., Dollet, S., Girard, M., Ciancia, C., Joly, S., Peschanski, M., and Lotteau, V. (2011). Protein transfer into human cells by VSV-G-induced nanovesicles. Mol Ther 19, 1656-1666.
Mangeot, P. E., Risson, V., Fusil, F., Marnef, A., Laurent, E., Blin, J., Mournetas, V., Massourides, E., Sohier, T. J. M., Corbin, A., et al. (2019). Genome editing in primary cells and in vivo using viral-derived Nanoblades loaded with Cas9-sgRNA ribonucleoproteins. Nat Commun 10, 45.
Mercuri, E., Darras, B. T., Chiriboga, C. A., Day, J. W., Campbell, C., Connolly, A. M., Iannaccone, S. T., Kirschner, J., Kuntz, N. L., Saito, K., et al. (2018). Nusinersen versus Sham Control in Later-Onset Spinal Muscular Atrophy. N Engl J Med 378, 625-635.
Meunier, L., and Larrey, D. (2019). Drug-Induced Liver Injury: Biomarkers, Requirements, Candidates, and Validation. Front Pharmacol 10, 1482.
Milone, M. C., and O'Doherty, U. (2018). Clinical use of lentiviral vectors. Leukemia 32, 1529-1541.
Musunuru, K., Chadwick, A. C., Mizoguchi, T., Garcia, S. P., DeNizio, J. E., Reiss, C. W., Wang, K., Iyer, S., Dutta, C., Clendaniel, V., et al. (2021). In vivo CRISPR base editing of PCSK9 durably lowers cholesterol in primates. Nature 593, 429-434.
Newby, G. A., and Liu, D. R. (2021). In vivo somatic cell base editing and prime editing. Mol Ther. 29 (11), 3107-3124.
Newby, G. A., Yen, J. S., Woodard, K. J., Mayuranathan, T., Lazzarotto, C. R., Li, Y., Sheppard-Tillman, H., Porter, S. N., Yao, Y., Mayberry, K., et al. (2021). Base editing of haematopoietic stem cells rescues sickle cell disease in mice. Nature 595, 295-302.
Nishida, K., Arazoe, T., Yachie, N., Banno, S., Kakimoto, M., Tabata, M., Mochizuki, M., Miyabe, A., Araki, M., Hara, K. Y., et al. (2016). Targeted nucleotide editing using hybrid prokaryotic and vertebrate adaptive immune systems. Science 353.
Osborn, M. J., Newby, G. A., McElroy, A. N., Knipping, F., Nielsen, S. C., Riddle, M. J., Xia, L., Chen, W., Eide, C. R., Webber, B. R., et al. (2020). Base Editor Correction of COL7A1 in Recessive Dystrophic Epidermolysis Bullosa Patient-Derived Fibroblasts and iPSCs. J Invest Dermatol 140, 338-347 e335.
Pan, D., Gunther, R., Duan, W., Wendell, S., Kaemmerer, W., Kafri, T., Verma, I. M., and Whitley, C. B. (2002). Biodistribution and toxicity studies of VSVG-pseudotyped lentiviral vector after intravenous administration in mice with the observation of in vivo transduction of bone marrow. Mol Ther 6, 19-29.
Pang, J. J., Chang, B., Hawes, N. L., Hurd, R. E., Davisson, M. T., Li, J., Noorwez, S. M., Malhotra, R., McDowell, J. H., Kaushal, S., et al. (2005). Retinal degeneration 12 (rd12): a new, spontaneously arising mouse model for human Leber congenital amaurosis (LCA). Mol Vis 11, 152-162.
Parr-Brownlie, L. C., Bosch-Bouju, C., Schoderboeck, L., Sizemore, R. J., Abraham, W. C., and Hughes, S. M. (2015). Lentiviral vectors as tools to understand central nervous system biology in mammalian model organisms. Front Mol Neurosci 8, 14.
Puppo, A., Cesi, G., Marrocco, E., Piccolo, P., Jacca, S., Shayakhmetov, D. M., Parks, R. J., Davidson, B. L., Colloca, S., Brunetti-Pierri, N., et al. (2014). Retinal transduction profiles by high-capacity viral vectors. Gene Ther 21, 855-865.
Rao, A. S., Lindholm, D., Rivas, M. A., Knowles, J. W., Montgomery, S. B., and Ingelsson, E. (2018). Large-Scale Phenome-Wide Association Study of PCSK9 Variants Demonstrates Protection Against Ischemic Stroke. Circ Genom Precis Med 11, e002162.
Rees, H. A., Komor, A. C., Yeh, W. H., Caetano-Lopes, J., Warman, M., Edge, A. S. B., and Liu, D. R. (2017). Improving the DNA specificity and applicability of base editing through protein engineering and protein delivery. Nat Commun 8, 15790.
Rees, H. A., and Liu, D. R. (2018). Base editing: precision chemistry on the genome and transcriptome of living cells. Nat Rev Genet 19, 770-788.
Renner, T. M., Tang, V. A., Burger, D., and Langlois, M. A. (2020). Intact Viral Particle Counts Measured by Flow Virometry Provide Insight into the Infectivity and Genome Packaging Efficiency of Moloney Murine Leukemia Virus. J Virol 94.
Richter, M. F., Zhao, K. T., Eton, E., Lapinaite, A., Newby, G. A., Thuronyi, B. W., Wilson, C., Koblan, L. W., Zeng, J., Bauer, D. E., et al. (2020). Phage-assisted evolution of an adenine base editor with improved Cas domain compatibility and activity. Nat Biotechnol. 38, 883-891.
Rothgangl, T., Dennis, M. K., Lin, P. J. C., Oka, R., Witzigmann, D., Villiger, L., Qi, W., Hruzova, M., Kissling, L., Lenggenhager, D., et al. (2021). In vivo adenine base editing of PCSK9 in macaques reduces LDL cholesterol levels. Nat Biotechnol 39, 949-957.
Serreze, D. V., Leiter, E. H., Christianson, G. J., Greiner, D., and Roopenian, D. C. (1994). Major histocompatibility complex class I-deficient NOD-B2mnull mice are diabetes and insulitis resistant. Diabetes 43, 505-509.
Sodi, A., Banfi, S., Testa, F., Della Corte, M., Passerini, I., Pelo, E., Rossi, S., Simonelli, F., and Italian, I.R.D.W.G. (2021). RPE65-associated inherited retinal diseases: consensus recommendations for eligibility to gene therapy. Orphanet J Rare Dis 16, 257.
Song, Y., Liu, Z., Zhang, Y., Chen, M., Sui, T., Lai, L., and Li, Z. (2020). Large-Fragment Deletions Induced by Cas9 Cleavage while Not in the BEs System. Mol Ther Nucleic Acids 21, 523-526.
Stadtmauer, E. A., Fraietta, J. A., Davis, M. M., Cohen, A. D., Weber, K. L., Lancaster, E., Mangan, P. A., Kulikovskaya, I., Gupta, M., Chen, F., et al. (2020). CRISPR-engineered T cells in patients with refractory cancer. Science 367.
Suh, S., Choi, E. H., Leinonen, H., Foik, A. T., Newby, G. A., Yeh, W. H., Dong, Z., Kiser, P. D., Lyon, D. C., Liu, D. R., et al. (2021). Restoration of visual function in adult mice with an inherited retinal disease via adenine base editing. Nat Biomed Eng 5, 169-178.
Swiech, L., Heidenreich, M., Banerjee, A., Habib, N., Li, Y., Trombetta, J., Sur, M., and Zhang, F. (2015). In vivo interrogation of gene function in the mammalian brain using CRISPR-Cas9. Nat Biotechnol 33, 102-106.
Taylor, A. W. (2009). Ocular immune privilege. Eye (Lond) 23, 1885-1889. Thorne, R. G., and Nicholson, C. (2006). In vivo diffusion analysis with quantum dots and dextrans predicts the width of brain extracellular space. Proc Natl Acad Sci USA 103, 5567-5572.
Tsai, S. Q., Nguyen, N. T., Malagon-Lopez, J., Topkar, V. V., Aryee, M. J., and Joung, J. K. (2017). CIRCLE-seq: a highly sensitive in vitro screen for genome-wide CRISPR-Cas9 nuclease off-targets. Nat Methods 14, 607-614.
Turchiano, G., Andrieux, G., Klermund, J., Blattner, G., Pennucci, V., El Gaz, M., Monaco, G., Poddar, S., Mussolino, C., Cornu, T. I., et al. (2021). Quantitative evaluation of chromosomal rearrangements in gene-edited human stem cells by CAST-Seq. Cell Stem Cell 28, 1136-1147 e1135.
Voelkel, C., Galla, M., Maetzig, T., Warlich, E., Kuehle, J., Zychlinski, D., Bode, J., Cantz, T., Schambach, A., and Baum, C. (2010). Protein transduction from retroviral Gag precursors. Proc Natl Acad Sci USA 107, 7805-7810.
Wang, D., Shukla, C., Liu, X., Schoeb, T. R., Clarke, L. A., Bedwell, D. M., and Keeling, K. M. (2010). Characterization of an MPS I-H knock-in mouse that carries a nonsense mutation analogous to the human IDUA-W402X mutation. Mol Genet Metab 99, 62-71.
Wang, D., Zhang, F., and Gao, G. (2020). CRISPR-Based Therapeutic Genome Editing: Strategies and In Vivo Delivery by AAV Vectors. Cell 181, 136-150.
Webber, B. R., Lonetree, C. L., Kluesner, M. G., Johnson, M. J., Pomeroy, E. J., Diers, M. D., Lahr, W. S., Draper, G. M., Slipek, N. J., Smeester, B. A., et al. (2019). Highly efficient multiplex human T cell engineering without double-strand breaks using Cas9 base editors. Nat Commun 10, 5222.
Wei, T., Cheng, Q., Min, Y. L., Olson, E. N., and Siegwart, D. J. (2020). Systemic nanoparticle delivery of CRISPR-Cas9 ribonucleoproteins for effective tissue specific genome editing. Nat Commun 11, 3232.
Wheeler, J. X., Jones, C., Thorpe, R., and Zhao, Y. (2007). Proteomics analysis of cellular components in lentiviral vector production using Gel-LC-MS/MS. Proteomics Clin Appl 1, 224-230.
Wu, D. T., and Roth, M. J. (2014). MLV based viral-like-particles for delivery of toxic proteins and nuclear transcription factors. Biomaterials 35, 8416-8426.
Yao, X., Lyu, P., Yoo, K., Yadav, M. K., Singh, R., Atala, A., and Lu, B. (2021). Engineered extracellular vesicles as versatile ribonucleoprotein delivery vehicles for efficient and safe CRISPR genome editing. J Extracell Vesicles 10, e12076.
Yeh, W. H., Chiang, H., Rees, H. A., Edge, A. S. B., and Liu, D. R. (2018). In vivo base editing of post-mitotic sensory cells. Nat Commun 9, 2184.
Yeh, W. H., Shubina-Oleinik, O., Levy, J. M., Pan, B., Newby, G. A., Wornow, M., Burt, R., Chen, J. C., Holt, J. R., and Liu, D. R. (2020). In vivo base editing restores sensory transduction and transiently improves auditory function in a mouse model of recessive deafness. Sci Transl Med 12.
Yu, Y., Leete, T. C., Born, D. A., Young, L., Barrera, L. A., Lee, S., Rees, H. A., Ciaramella, G., and Gaudelli, N. M. (2020). Cytosine base editors with minimized unguided DNA and RNA off-target events and high on-target activity. Nat Commun 11, 2052.
Zeng, J., Wu, Y., Ren, C., Bonanno, J., Shen, A. H., Shea, D., Gehrke, J. M., Clement, K., Luk, K., Yao, Q., et al. (2020). Therapeutic base editing of human hematopoietic stem cells. Nat Med 26, 535-541.
Zhang, W., Cao, S., Martin, J. L., Mueller, J. D., and Mansky, L. M. (2015). Morphology and ultrastructure of retrovirus particles. AIMS Biophys 2, 343-369.
Zhong, Z., Rong, F., Dai, Y., Yibulayin, A., Zeng, L., Liao, J., Wang, L., Huang, Z., Zhou, Z., and Chen, J. (2019). Seven novel variants expand the spectrum of RPE65-related Leber congenital amaurosis in the Chinese population. Mol Vis 25, 204-214.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1-179. (canceled)

180. A polynucleotide encoding a fusion protein, wherein the fusion protein comprises:
(i) a plasma membrane localization protein,
(ii) a therapeutic cargo,
(iii) a nuclear export sequence (NES); wherein
a) the NES is located between the plasma membrane localization protein and the therapeutic cargo, or
b) the NES is N-terminal to the therapeutic cargo; and
(iv) a cleavable linker located between the therapeutic cargo and the NES.

181. The polynucleotide of claim 180, wherein the NES is located between the plasma membrane localization protein and the therapeutic cargo.

182. The polynucleotide of claim 180, wherein the NES is N-terminal to the therapeutic cargo.

183. The polynucleotide of claim 180, wherein the NES is located between the plasma membrane localization protein and the therapeutic cargo, and the NES is N-terminal to the therapeutic cargo.

184. The polynucleotide of claim 180, wherein the plasma membrane localization protein comprises a retroviral gag protein.

185. The polynucleotide of claim 184, wherein the retroviral gag protein comprises a gag nucleocapsid protein.

186. The polynucleotide of claim 180, wherein the therapeutic cargo comprises a nucleic acid molecule, a protein, a small molecule compound, or any combination thereof.

187. The polynucleotide of claim 180, wherein the therapeutic cargo comprises a nuclease, a base editor, a prime editor, an epigenetic editor, a restriction endonuclease, a recombinase, a transcription factor, an antibody, a chimeric antigen receptor, a T cell receptor, an organelle, a DNA, an RNA, a RNP, a retrotransposon, a reverse transcriptase, an oligonucleotide, an aptazyme, an aptamer, a ribozyme, or any combination thereof.

188. The polynucleotide of claim 180, wherein the therapeutic cargo comprises an epigenetic editor.

189. The polynucleotide of claim 180, wherein the therapeutic cargo comprises a Cas protein.

190. The polynucleotide of claim 189, wherein the Cas protein is a dead Cas protein.

191. The polynucleotide of claim 189, wherein the Cas protein is a Cas nickase.

192. The polynucleotide of claim 180, wherein the cleavable linker comprises a protease cleavage site.

193. The polynucleotide of claim 192, wherein the protease cleavage site is a Moloney murine leukemia virus (MMLV) protease cleavage site or a Friend murine leukemia virus (FMLV) protease cleavage site.

194. The polynucleotide of claim 180, wherein the fusion protein comprises at least three NESs.

195. The polynucleotide of claim 180, wherein the fusion protein further comprises at least one nuclear localization sequence (NLS).

196. The polynucleotide of claim 195, wherein the NLS is positioned at or near the N-terminus or the C-terminus of the therapeutic cargo.

197. The polynucleotide of claim 180, wherein the fusion protein comprises from N-terminus to C-terminus: [plasma membrane localization protein]-[n*NES]-[cleavable linker]-[m₁*NLS]-[therapeutic cargo]-[m₂*NLS], wherein n is an integer in the range of 1 to 10, wherein m₁ and m₂ are integers in the range of from 0 to 10, and wherein n, m₁, and m₂ denote the number of repeats of the respective sequences to which they refer.

198. The polynucleotide of claim 195, wherein the fusion protein comprises from N-terminus to C-terminus: [plasma membrane localization protein]-[3×NES]-[cleavable linker]-[NLS]-[therapeutic cargo]-[NLS].

199. The polynucleotide of claim 180, wherein the fusion protein comprises from N-terminus to C-terminus: the plasma membrane localization protein, the NES, and the therapeutic cargo.

200. The polynucleotide of claim 180, wherein the plasma membrane localization protein comprises an MMLV gag nucleocapsid protein or an FMLV gag nucleocapsid protein.

201. The polynucleotide of claim 180, wherein the plasma membrane localization protein comprises a human endogenous retroviral (HERV) structural protein, a humanized viral structural protein, a pleckstrin homology (PH) domain, or a non-immunogenic plasma membrane recruitment protein.

202. A fusion protein encoded by the polynucleotide of claim 180.

203. A vector comprising the polynucleotide of claim 180.
